(19) United States
(12) Patent Application Publication
(10) Pub. No.: US 2025/0129150 A1
(43) Pub. Date: Apr. 24, 2025

(54) ANTIBODIES THAT BIND INTERLEUKIN 13 AND METHODS OF USE

(71) Applicant: Apogee Biologics, Inc., Waltham, MA (US)

(72) Inventors: Hussam H. SHAHEEN, Waltham, MA (US); Kenneth Evan THOMPSON, Waltham, MA (US); Peter HARWIN, Waltham, MA (US); Tomas KISELAK, Waltham, MA (US)

(21) Appl. No.: 18/979,795

(22) Filed: Dec. 13, 2024

Related U.S. Application Data
(63) Continuation of application No. PCT/US2023/068621, filed on Jun. 16, 2023.
(60) Provisional application No. 63/469,167, filed on May 26, 2023.
(60) Provisional application No. 63/462,822, filed on Apr. 28, 2023.
(60) Provisional application No. 63/353,367, filed on Jun. 17, 2022.

(51) Int. Cl. C07K 16/24; A61K 39/00

(52) U.S. Cl. CPC C07K 16/244; A61K 2039/505; C07K 2317/24; C07K 2317/76; C07K 2317/92; C07K 2317/94

(57) ABSTRACT

Described herein are novel and improved antibodies that bind Interleukin 13 (IL-13) and methods of use thereof. In certain aspects, described herein are methods of inhibiting IL-13 biological activity. In certain aspects, described herein are pharmaceutical compositions comprising the anti-IL-13 antibodies. In certain aspects, the antibodies and methods described herein are used for treatment of an inflammatory disease or disorder associated with elevated levels of IL-13 and/or IgE.

### CROSS REFERENCE TO RELATED APPLICATIONS

This application is a Continuation of International Application No. PCT/US2023/068621, filed on Jun. 16, 2023, which claims priority to, and the benefit to U.S. Provisional Application No. 63/353,367, filed Jun. 17, 2022, U.S. Provisional Application No. 63/462,822, filed Apr. 28, 2023, and U.S. Provisional Application No. 63/469,167, filed May 26, 2023, the entire disclosures of which are hereby incorporated by reference in their entirety for all purposes.

### SEQUENCE LISTING

The instant application contains a Sequence Listing which has been submitted electronically in XML file format and is hereby incorporated by reference in its entirety. Said XML copy, created on Jun. 15, 2023, is named AOE-001WO_SL.xml and is 417 KB in size.

### BACKGROUND

Interleukin (IL)-13 is a T helper cell subclass 2 (Th2) cytokine and belongs to a family of type I cytokines, exhibiting pleiotropic effects across multiple cellular pathways. IL-13 is involved in the differentiation of naïve T cells into Th2 cells. IL-13 promotes B-cell proliferation and induces immunoglobulin isotype class switching to IgG4 and IgE when co-stimulated with CD40/CD40L. It also up-regulates FcεRI, and thus, helps in IgE priming of mast cells. In monocytes/macrophages, IL-13 up-regulates expression of CD23 and MHC class I and class II antigens, down-regulates the expression of CD14, inhibits antibody-dependent cytotoxicity, and promotes eosinophil survival, activation, and recruitment. IL-13 also manifests important functions on nonhematopoietic cells, such as smooth muscle cells, epithelial cells, endothelial cells, and fibroblast cells. IL-13 enhances proliferation and cholinergic-induced contractions of smooth muscles. In epithelial cells, IL-13 is a potent inducer of chemokine production, alters mucociliary differentiation, decreases ciliary beat frequency of ciliated epithelial cells, and results in goblet cell metaplasia. In endothelial cells, IL-13 is a potent inducer of vascular cell adhesion molecule 1 (VCAM-1), which is important for recruitment of eosinophils. In epithelial keratinocytes, IL-13 reduces the expression of barrier integrity molecules, such as filaggrin and loricrin, while stimulating CCL26 and CCL2 secretion responsible for the recruitment of several inflammatory cells of myeloid lineages. In human dermal fibroblasts, IL-13 induces type 1 collagen synthesis in human dermal fibroblasts.

The inhibition of IL-13 may be used to treat or prevent inflammatory diseases and conditions, such as those related to elevated levels of IgE, including but not limited to asthma, allergic rhinitis, urticaria, and allergic or atopic dermatitis. Thus, the development of potent and specific inhibitors of IL-13, for example, inhibitors that remain active for longer terms when administered to subjects, are needed for the prevention and/or treatment IL-13- and IgE-mediated diseases or conditions.

### SUMMARY

In certain aspects, described herein is an isolated antibody that binds IL-13, i) comprising a variable heavy (VH) chain sequence comprising three heavy chain CDR sequences, CDR-H1, CDR-H2, and CDR-H3; and ii) a variable light (VL) chain sequence comprising three light chain CDR sequences, CDR-L1, CDR-L2, and CDR-L3; wherein: a) CDR-H1 comprises a sequence selected from the sequences set forth in SEQ ID NOs: 58-99 and 121; b) CDR-H2 comprises a sequence selected from the sequences set forth in SEQ ID NOs: 100-111; c) CDR-H3 comprises a sequence selected from the sequences set forth in SEQ ID NOs: 112-120 and 130-140; d) CDR-L1 comprises a sequence selected from the sequences set forth in SEQ ID NOs: 141-144 and 149-152; e) CDR-L2 comprises a sequence selected from the sequences set forth in SEQ ID NOs: 153-158 and the amino acid sequence LAS; and f) CDR-L3 comprises a sequence selected from the sequences set forth in SEQ ID NOs: 165-172.

In certain embodiments, the isolated antibody comprises: a) CDR-H1 comprising a sequence selected from the sequences set forth in SEQ ID NOs: 58-66; b) CDR-H2 comprises a sequence selected from the sequences set forth in SEQ ID NOs: 100-103; c) CDR-H3 comprises a sequence selected from the sequences set forth in SEQ ID NOs: 112-120; d) CDR-L1 comprises a sequence selected from the sequences set forth in SEQ ID NOs: 141-144; e) CDR-L2 comprises a sequence selected from the sequences set forth in SEQ ID NOs: 153-158; and f) CDR-L3 comprises a sequence selected from the sequences set forth in SEQ ID NOs: 165-172.

In certain embodiments, the isolated antibody comprises: a) CDR-H1 comprising a sequence selected from the sequences set forth in SEQ ID NOs: 67-83; b) CDR-H2 comprises a sequence selected from the sequences set forth in SEQ ID NOs: 104-107; c) CDR-H3 comprises a sequence selected from the sequences set forth in SEQ ID NOs: 112-120; d) CDR-L1 comprises a sequence selected from the sequences set forth in SEQ ID NOs: 141-144; e) CDR-L2 comprises a sequence selected from the sequences set forth in SEQ ID NOs: 153-158; and f) CDR-L3 comprises a sequence selected from the sequences set forth in SEQ ID NOs: 165-172.

In certain embodiments, the isolated antibody comprises: a) CDR-H1 comprising a sequence selected from the sequences set forth in SEQ ID NOs: 84-99 and 121; b) CDR-H2 comprises a sequence selected from the sequences set forth in SEQ ID NOs: 108-111; c) CDR-H3 comprises a sequence selected from the sequences set forth in SEQ ID NOs: 130-140; d) CDR-L1 comprises a sequence selected from the sequences set forth in SEQ ID NOs: 149-152; e) CDR-L2 comprises the amino acid sequence LAS; and f) CDR-L3 comprises a sequence selected from the sequences set forth in SEQ ID NOs: 165-172.

In certain embodiments, the isolated antibody does not comprise: a) CDR-H1 set forth in SEQ ID NO: 58; CDR-H2 set forth in SEQ ID NO: 100; CDR-H3 set forth in SEQ ID NO: 112; CDR-L1 set forth in SEQ ID NO: 141; CDR-L2 set forth in SEQ ID NO: 153; and CDR-L3 set forth in SEQ ID NO: 165; or b) CDR-H1 set forth in SEQ ID NO: 67; CDR-H2 set forth in SEQ ID NO: 104; CDR-H3 set forth in SEQ ID NO: 112; CDR-L1 set forth in SEQ ID NO: 141; CDR-L2 set forth in SEQ ID NO: 153; and CDR-L3 set forth in SEQ ID NO: 165; or c) CDR-H1 set forth in SEQ ID NO: 84; CDR-H2 set forth in SEQ ID NO: 108; CDR-H3 set forth in SEQ ID NO: 130; CDR-L1 set forth in SEQ ID NO: 149; CDR-L2 set forth by amino acid sequence LAS; and CDR-L3 set forth in SEQ ID NO: 165.

In certain embodiments, the antibody does not comprise any combination of: a) CDR-H1 set forth in any of SEQ ID NOs: 58, 67, or 84; b) a CDR-H2 set forth in any of SEQ ID NOs: 100, 104, or 108; c) a CDR-H3 set forth in any of SEQ ID NOs: 112 or 130; d) a CDR-L1 set forth in any of SEQ ID NOs: 141 or 149; e) a CDR-L2 set forth in any of SEQ ID NOs: 153 or 154; and f) a CDR-L3 set forth in SEQ ID NO: 165.

In certain embodiments, the isolated antibody comprises a CDR-H1 comprising the sequence set forth in any of SEQ ID NOs: 58, 67, or 68; a CDR-H2 comprising the sequence set forth in any of SEQ ID NOs: 100 or 104; a CDR-H3 comprising the sequence set forth in SEQ ID NO: 112; a CDR-L1 comprising the sequence set forth in any of SEQ ID NOs: 141 or 149; a CDR-L2 comprising the sequence set forth in any of SEQ ID NO: 153 or the amino acid sequence of LAS; and a CDR-L3 comprising the sequence set forth in SEQ ID NO: 165.

In certain embodiments, the isolated antibody comprises a CDR-H1 comprising the sequence set forth in SEQ ID NO: 58; a CDR-H2 comprising the sequence set forth in SEQ ID NO: 100; a CDR-H3 comprising the sequence set forth in SEQ ID NO: 112; a CDR-L1 comprising the sequence set forth in SEQ ID NO: 141; a CDR-L2 comprising the sequence set forth in SEQ ID NO: 153; and a CDR-L3 comprising the sequence set forth in SEQ ID NO: 165.

In certain embodiments, the isolated antibody comprises a CDR-H1 comprising the sequence set forth in SEQ ID NO: 67; a CDR-H2 comprising the sequence set forth in SEQ ID NO: 104; a CDR-H3 comprising the sequence set forth in SEQ ID NO: 112; a CDR-L1 comprising the sequence set forth in SEQ ID NO: 141; a CDR-L2 comprising the sequence set forth in SEQ ID NO: 153; and a CDR-L3 comprising the sequence set forth in SEQ ID NO: 165.

In certain embodiments, the isolated antibody comprises a CDR-H1 comprising the sequence set forth in SEQ ID NO: 68; a CDR-H2 comprising the sequence set forth in SEQ ID NO: 104; a CDR-H3 comprising the sequence set forth in SEQ ID NO: 112; a CDR-L1 comprising the sequence set forth in SEQ ID NO: 141; a CDR-L2 comprising the sequence set forth in SEQ ID NO: 153; and a CDR-L3 comprising the sequence set forth in SEQ ID NO: 165.

In certain embodiments, the isolated antibody comprises a CDR-H1 comprising the sequence set forth in SEQ ID NO: 67; a CDR-H2 comprising the sequence set forth in SEQ ID NO: 104; a CDR-H3 comprising the sequence set forth in SEQ ID NO: 112; a CDR-L1 comprising the sequence set forth in SEQ ID NO: 149; a CDR-L2 comprising the amino acid sequence LAS; and a CDR-L3 comprising the sequence set forth in SEQ ID NO: 165.

In certain embodiments, the isolated antibody comprises a CDR-H1 comprising the sequence set forth in SEQ ID NO: 68; a CDR-H2 comprising the sequence set forth in SEQ ID NO: 104; a CDR-H3 comprising the sequence set forth in SEQ ID NO: 112; a CDR-L1 comprising the sequence set forth in SEQ ID NO: 149; a CDR-L2 comprising the amino acid sequence LAS; and a CDR-L3 comprising the sequence set forth in SEQ ID NO: 165.

In certain embodiments, the isolated antibody comprises a CDR-H1 comprising the sequence set forth in any of SEQ ID NOs: 58, 67, 68, 84, or 85; a CDR-H2 comprising the sequence set forth in any of SEQ ID NOs: 100, 104, or 108; a CDR-H3 comprising the sequence set forth in any of SEQ ID NOs: 112 or 130; a CDR-L1 comprising the sequence set forth in any of SEQ ID NOs: 141 or 149; a CDR-L2 comprising the sequence set forth in any of SEQ ID NO: 153 or the amino acid sequence LAS; and a CDR-L3 comprising the sequence set forth in SEQ ID NO: 165.

In certain embodiments, the isolated antibody comprises a CDR-H1 comprising the sequence set forth in SEQ ID NO: 68; a CDR-H2 comprising the sequence set forth in SEQ ID NO: 104; a CDR-H3 comprising the sequence set forth in SEQ ID NO: 112; a CDR-L1 comprising the sequence set forth in SEQ ID NO: 141; a CDR-L2 comprising the sequence set forth in SEQ ID NO: 153; and a CDR-L3 comprising the sequence set forth in SEQ ID NO: 165.

In certain embodiments, the isolated antibody comprises a CDR-H1 comprising the sequence set forth in SEQ ID NO: 84; a CDR-H2 comprising the sequence set forth in SEQ ID NO: 108; a CDR-H3 comprising the sequence set forth in SEQ ID NO: 130; a CDR-L1 comprising the sequence set forth in SEQ ID NO: 149; a CDR-L2 comprising the amino acid sequence LAS; and a CDR-L3 comprising the sequence set forth in SEQ ID NO: 165.

In certain embodiments, the isolated antibody comprises a CDR-H1 comprising the sequence set forth in SEQ ID NO: 85; a CDR-H2 comprising the sequence set forth in SEQ ID NO: 108; a CDR-H3 comprising the sequence set forth in SEQ ID NO: 130; a CDR-L1 comprising the sequence set forth in SEQ ID NO: 149; a CDR-L2 comprising the amino acid sequence LAS; and a CDR-L3 comprising the sequence set forth in SEQ ID NO: 165.

In certain embodiments, the isolated antibody comprises a CDR-H1 comprising the sequence set forth in any of SEQ ID NOs: 58, 67, 68, 84, or 85; a CDR-H2 comprising the sequence set forth in any of SEQ ID NOs: 100, 104, or 108; a CDR-H3 comprising the sequence set forth in any of SEQ ID NOs: 112 or 130; a CDR-L1 comprising the sequence set forth in any of SEQ ID NOs: 141 or 149; a CDR-L2 comprising the sequence set forth in any of SEQ ID NO: 157 or the amino acid sequence LAS; and a CDR-L3 comprising the sequence set forth in SEQ ID NO: 165.

In certain embodiments, the isolated antibody comprises a CDR-H1 comprising the sequence set forth in SEQ ID NO: 58; a CDR-H2 comprising the sequence set forth in SEQ ID NO: 100; a CDR-H3 comprising the sequence set forth in SEQ ID NO: 112; a CDR-L1 comprising the sequence set forth in SEQ ID NO: 141; a CDR-L2 comprising the sequence set forth in SEQ ID NO: 157; and a CDR-L3 comprising the sequence set forth in SEQ ID NO: 165.

In certain embodiments, the isolated antibody comprises a CDR-H1 comprising the sequence set forth in SEQ ID NO: 68; a CDR-H2 comprising the sequence set forth in SEQ ID NO: 104; a CDR-H3 comprising the sequence set forth in SEQ ID NO: 112; a CDR-L1 comprising the sequence set forth in SEQ ID NO: 141; a CDR-L2 comprising the sequence set forth in SEQ ID NO: 157; and a CDR-L3 comprising the sequence set forth in SEQ ID NO: 165.

In certain embodiments, the isolated antibody comprises a CDR-H1 comprising the sequence set forth in any of SEQ ID NOs: 58, 67, 68, 84, or 85; a CDR-H2 comprising the sequence set forth in any of SEQ ID NOs: 100, 104, or 108; a CDR-H3 comprising the sequence set forth in any of SEQ ID NOs: 112 or 130; a CDR-L1 comprising the sequence set forth in any of SEQ ID NOs: 141 or 149; a CDR-L2 comprising the sequence set forth in any of SEQ ID NO: 157 or the amino acid sequence LAS; and a CDR-L3 comprising the sequence set forth in SEQ ID NO: 165.

In certain embodiments, the isolated antibody comprises a CDR-H1 comprising the sequence set forth in SEQ ID NO: 68; a CDR-H2 comprising the sequence set forth in SEQ ID NO: 104; a CDR-H3 comprising the sequence set forth in SEQ ID NO: 112; a CDR-L1 comprising the sequence set forth in SEQ ID NO: 141; a CDR-L2 comprising the sequence set forth in SEQ ID NO: 157; and a CDR-L3 comprising the sequence set forth in SEQ ID NO: 165.

In certain embodiments, the isolated antibody comprises a CDR-H1 comprising the sequence set forth in SEQ ID NO: 84; a CDR-H2 comprising the sequence set forth in SEQ ID NO: 108; a CDR-H3 comprising the sequence set forth in SEQ ID NO: 130; a CDR-L1 comprising the sequence set forth in SEQ ID NO: 149; a CDR-L2 comprising the amino acid sequence LAS; and a CDR-L3 comprising the sequence set forth in SEQ ID NO: 165.

In certain embodiments, the isolated antibody comprises a CDR-H1 comprising the sequence set forth in SEQ ID NO: 85; a CDR-H2 comprising the sequence set forth in SEQ ID NO: 108; a CDR-H3 comprising the sequence set forth in SEQ ID NO: 130; a CDR-L1 comprising the sequence set forth in SEQ ID NO: 149; a CDR-L2 comprising the amino acid sequence LAS; and a CDR-L3 comprising the sequence set forth in SEQ ID NO: 165.

In certain embodiments, the isolated antibody comprises a VH sequence selected from the sequences set forth in SEQ ID NOs: 1-32 and 470.

In certain embodiments, the isolated antibody comprises a VL sequence selected from the sequences set forth in SEQ ID NOs: 33-57 and 471.

In certain embodiments, the isolated antibody comprises a VH sequence selected from the sequences set forth in SEQ ID NOs: 1-32 and 470 and a VL sequence selected from the sequences set forth in SEQ ID NOs: 33-57 and 471.

In certain embodiments, the isolated antibody comprises a VH sequence selected from the sequences set forth in SEQ ID NOs: 1-32 and 470 and a VL sequence set forth in SEQ ID NO: 49.

In certain embodiments, the isolated antibody comprises a VH sequence selected from the sequences set forth in SEQ ID NOs: 1-32 and 470 and a VL sequence set forth in SEQ ID NO: 51.

In certain embodiments, the isolated antibody comprises a VH sequence set forth in SEQ ID NO: 1 and a VL sequence set forth in SEQ ID NO: 33.

In certain embodiments, the isolated antibody comprises a VH sequence set forth in SEQ ID NO: 2 and a VL sequence set forth in SEQ ID NO: 33.

In certain embodiments, the isolated antibody comprises a VH sequence set forth in SEQ ID NO: 3 and a VL sequence set forth in SEQ ID NO: 35.

In certain embodiments, the isolated antibody comprises a VH sequence set forth in SEQ ID NO: 4 and a VL sequence set forth in SEQ ID NO: 35.

In certain embodiments, the isolated antibody comprises a VH sequence set forth in SEQ ID NO: 5 and a VL sequence set forth in SEQ ID NO: 35.

In certain embodiments, the isolated antibody comprises a VH sequence set forth in SEQ ID NO: 6 and a VL sequence set forth in SEQ ID NO: 35.

In certain embodiments, the isolated antibody comprises a VH sequence set forth in SEQ ID NO: 7 and a VL sequence set forth in SEQ ID NO: 35.

In certain embodiments, the isolated antibody comprises a VH sequence set forth in SEQ ID NO: 3 and a VL sequence set forth in SEQ ID NO: 36.

In certain embodiments, the isolated antibody comprises a VH sequence set forth in SEQ ID NO: 4 and a VL sequence set forth in SEQ ID NO: 36.

In certain embodiments, the isolated antibody comprises a VH sequence set forth in SEQ ID NO: 5 and a VL sequence set forth in SEQ ID NO: 36.

In certain embodiments, the isolated antibody comprises a VH sequence set forth in SEQ ID NO: 6 and a VL sequence set forth in SEQ ID NO: 36.

In certain embodiments, the isolated antibody comprises a VH sequence set forth in SEQ ID NO: 7 and a VL sequence set forth in SEQ ID NO: 36.

In certain embodiments, the isolated antibody comprises a VH sequence set forth in SEQ ID NO: 3 and a VL sequence set forth in SEQ ID NO: 39.

In certain embodiments, the isolated antibody comprises a VH sequence set forth in SEQ ID NO: 4 and a VL sequence set forth in SEQ ID NO: 39.

In certain embodiments, the isolated antibody comprises a VH sequence set forth in SEQ ID NO: 5 and a VL sequence set forth in SEQ ID NO: 39.

In certain embodiments, the isolated antibody comprises a VH sequence set forth in SEQ ID NO: 6 and a VL sequence set forth in SEQ ID NO: 39.

In certain embodiments, the isolated antibody comprises a VH sequence set forth in SEQ ID NO: 7 and a VL sequence set forth in SEQ ID NO: 39.

In certain embodiments, the isolated antibody comprises a VH sequence set forth in SEQ ID NO: 3 and a VL sequence set forth in SEQ ID NO: 40.

In certain embodiments, the isolated antibody comprises a VH sequence set forth in SEQ ID NO: 4 and a VL sequence set forth in SEQ ID NO: 40.

In certain embodiments, the isolated antibody comprises a VH sequence set forth in SEQ ID NO: 5 and a VL sequence set forth in SEQ ID NO: 40.

In certain embodiments, the isolated antibody comprises a VH sequence set forth in SEQ ID NO: 6 and a VL sequence set forth in SEQ ID NO: 40.

In certain embodiments, the isolated antibody comprises a VH sequence set forth in SEQ ID NO: 7 and a VL sequence set forth in SEQ ID NO: 40.

In certain embodiments, the isolated antibody comprises a VH sequence set forth in SEQ ID NO: 8 and a VL sequence set forth in SEQ ID NO: 42.

In certain embodiments, the isolated antibody comprises a VH sequence set forth in SEQ ID NO: 9 and a VL sequence set forth in SEQ ID NO: 43.

In certain embodiments, the isolated antibody comprises a VH sequence set forth in SEQ ID NO: 7 and a VL sequence set forth in SEQ ID NO: 39.

In certain embodiments, the isolated antibody comprises a VH sequence set forth in SEQ ID NO: 7 and a VL sequence set forth in SEQ ID NO: 44.

In certain embodiments, the isolated antibody comprises a VH sequence set forth in SEQ ID NO: 7 and a VL sequence set forth in SEQ ID NO: 45.

In certain embodiments, the isolated antibody comprises a VH sequence set forth in SEQ ID NO: 7 and a VL sequence set forth in SEQ ID NO: 46.

In certain embodiments, the isolated antibody comprises a VH sequence set forth in SEQ ID NO: 7 and a VL sequence set forth in SEQ ID NO: 47.

In certain embodiments, the isolated antibody comprises a VH sequence set forth in SEQ ID NO: 7 and a VL sequence set forth in SEQ ID NO: 48.

In certain embodiments, the isolated antibody comprises a VH sequence set forth in SEQ ID NO: 7 and a VL sequence set forth in SEQ ID NO: 49.

In certain embodiments, the isolated antibody comprises a VH sequence set forth in SEQ ID NO: 7 and a VL sequence set forth in SEQ ID NO: 50.

In certain embodiments, the isolated antibody comprises a VH sequence set forth in SEQ ID NO: 3 and a VL sequence set forth in SEQ ID NO: 51.

In certain embodiments, the isolated antibody comprises a VH sequence set forth in SEQ ID NO: 7 and a VL sequence set forth in SEQ ID NO: 51.

In certain embodiments, the isolated antibody comprises a VH sequence set forth in SEQ ID NO: 7 and a VL sequence set forth in SEQ ID NO: 52.

In certain embodiments, the isolated antibody comprises a VH sequence set forth in SEQ ID NO: 7 and a VL sequence set forth in SEQ ID NO: 53.

In certain embodiments, the isolated antibody comprises a VH sequence set forth in SEQ ID NO: 7 and a VL sequence set forth in SEQ ID NO: 54.

In certain embodiments, the isolated antibody comprises a VH sequence set forth in SEQ ID NO: 7 and a VL sequence set forth in SEQ ID NO: 55.

In certain embodiments, the isolated antibody comprises a VH sequence set forth in SEQ ID NO: 7 and a VL sequence set forth in SEQ ID NO: 56.

In certain embodiments, the isolated antibody comprises a VH sequence set forth in SEQ ID NO: 7 and a VL sequence set forth in SEQ ID NO: 57.

In certain embodiments, the isolated antibody comprises a VH sequence set forth in SEQ ID NO: 10 and a VL sequence set forth in SEQ ID NO: 39.

In certain embodiments, the isolated antibody comprises a VH sequence set forth in SEQ ID NO: 11 and a VL sequence set forth in SEQ ID NO: 39.

In certain embodiments, the isolated antibody comprises a VH sequence set forth in SEQ ID NO: 12 and a VL sequence set forth in SEQ ID NO: 39.

In certain embodiments, the isolated antibody comprises a VH sequence set forth in SEQ ID NO: 13 and a VL sequence set forth in SEQ ID NO: 39.

In certain embodiments, the isolated antibody comprises a VH sequence set forth in SEQ ID NO: 14 and a VL sequence set forth in SEQ ID NO: 39.

In certain embodiments, the isolated antibody comprises a VH sequence set forth in SEQ ID NO: 15 and a VL sequence set forth in SEQ ID NO: 39.

In certain embodiments, the isolated antibody comprises a VH sequence set forth in SEQ ID NO: 16 and a VL sequence set forth in SEQ ID NO: 39.

In certain embodiments, the isolated antibody comprises a VH sequence set forth in SEQ ID NO: 17 and a VL sequence set forth in SEQ ID NO: 39.

In certain embodiments, the isolated antibody comprises a VH sequence set forth in SEQ ID NO: 18 and a VL sequence set forth in SEQ ID NO: 39.

In certain embodiments, the isolated antibody comprises a VH sequence set forth in SEQ ID NO: 19 and a VL sequence set forth in SEQ ID NO: 39.

In certain embodiments, the isolated antibody comprises a VH sequence set forth in SEQ ID NO: 20 and a VL sequence set forth in SEQ ID NO: 39.

In certain embodiments, the isolated antibody comprises a VH sequence set forth in SEQ ID NO: 21 and a VL sequence set forth in SEQ ID NO: 39.

In certain embodiments, the isolated antibody comprises a VH sequence set forth in SEQ ID NO: 22 and a VL sequence set forth in SEQ ID NO: 39.

In certain embodiments, the isolated antibody comprises a VH sequence set forth in SEQ ID NO: 23 and a VL sequence set forth in SEQ ID NO: 39.

In certain embodiments, the isolated antibody comprises a VH sequence set forth in SEQ ID NO: 24 and a VL sequence set forth in SEQ ID NO: 39.

The isolated antibody of claim 21, wherein the antibody comprises a VH sequence set forth in SEQ ID NO: 25 and a VL sequence set forth in SEQ ID NO: 39.

In certain embodiments, the isolated antibody comprises a VH sequence set forth in SEQ ID NO: 26 and a VL sequence set forth in SEQ ID NO: 39.

In certain embodiments, the isolated antibody comprises a VH sequence set forth in SEQ ID NO: 27 and a VL sequence set forth in SEQ ID NO: 39.

In certain embodiments, the isolated antibody comprises a VH sequence set forth in SEQ ID NO: 28 and a VL sequence set forth in SEQ ID NO: 39.

In certain embodiments, the isolated antibody comprises a VH sequence set forth in SEQ ID NO: 28 and a VL sequence set forth in SEQ ID NO: 39.

In certain embodiments, the isolated antibody comprises a VH sequence set forth in SEQ ID NO: 29 and a VL sequence set forth in SEQ ID NO: 39.

In certain embodiments, the isolated antibody comprises a VH sequence set forth in SEQ ID NO: 30 and a VL sequence set forth in SEQ ID NO: 39.

In certain embodiments, the isolated antibody comprises a VH sequence set forth in SEQ ID NO: 31 and a VL sequence set forth in SEQ ID NO: 39.

In certain embodiments, the isolated antibody comprises a VH sequence set forth in SEQ ID NO: 32 and a VL sequence set forth in SEQ ID NO: 39.

In certain embodiments, the isolated antibody comprises a VH sequence set forth in SEQ ID NO: 8 and a VL sequence set forth in SEQ ID NO: 39.

In certain embodiments, the isolated antibody comprises a VH sequence set forth in SEQ ID NO: 8 and a VL sequence set forth in SEQ ID NO: 51.

In certain embodiments, the isolated antibody comprises a VH sequence set forth in SEQ ID NO: 470 and a VL sequence set forth in SEQ ID NO: 471.

In certain embodiments, the isolated antibody is a humanized, human, or chimeric antibody. In certain embodiments, the isolated is a humanized antibody. In certain embodiments, the isolated antibody comprises a heavy chain human constant region of a class selected from IgG, IgA, IgD, IgE, and IgM. In certain embodiments, the human Fc region comprises a human heavy chain constant region of the class IgG and a subclass selected from IgG1, IgG2, IgG3, and IgG4. In certain embodiments, the human Fc region comprises a human IgG1 Fc. In certain embodiments, the human Fc region comprises a human IgG4 Fc. In certain embodiments, the human Fc region comprises a human IgG2 Fc.

In certain embodiments of the antibodies described herein, the heavy chain comprises a constant heavy chain sequence selected from the sequences set forth in SEQ ID NOs: 425-468 and 484-539.

In certain embodiments, the isolated antibody comprises a VH sequence set forth in SEQ ID NO: 1 and a VL sequence set forth in SEQ ID NO: 33; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 2 and a VL sequence set forth in SEQ ID NO: 33; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 3 and a VL sequence set forth in SEQ ID NO: 35; and wherein the human Fc region comprises a human IgG sequence selected from a sequence set forth in SEQ ID NOS: 436-468 and 484-539.

In certain embodiments, the isolated antibody comprises a VH sequence set forth in SEQ ID NO: 4 and a VL sequence set forth in SEQ ID NO: 35; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 5 and a VL sequence set forth in SEQ ID NO: 35; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 6 and a VL sequence set forth in SEQ ID NO: 35; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 35; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 3 and a VL sequence set forth in SEQ ID NO: 36; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 4 and a VL sequence set forth in SEQ ID NO: 36; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 5 and a VL sequence set forth in SEQ ID NO: 36; and wherein the human Fc region comprises a human IgG sequence selected from a sequence set forth in SEQ ID NOS: 436-468 and 484-539.

In certain embodiments, the isolated antibody comprises a VH sequence set forth in SEQ ID NO: 6 and a VL sequence set forth in SEQ ID NO: 36; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 36; and wherein the human Fc region comprises a human IgG sequence selected from a sequence set forth in SEQ ID NOs: 439, 440, 446, 457, and 460.

In certain embodiments, the isolated antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 4 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 5 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 6 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 3 and a VL sequence set forth in SEQ ID NO: 40; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 4 and a VL sequence set forth in SEQ ID NO: 40; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 5 and a VL sequence set forth in SEQ ID NO: 40; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 6 and a VL sequence set forth in SEQ ID NO: 40; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 40; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 8 and a VL sequence set forth in SEQ ID NO: 42; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 9 and a VL sequence set forth in SEQ ID NO: 43; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 44; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 45; and wherein the human Fc region comprises a human IgG sequence selected from a sequence set forth in SEQ ID NOS: 436-468 and 484-539.

In certain embodiments, the isolated antibody comprises a VH sequence set forth in SEQ ID NO: 7 and a VL sequence set forth in SEQ ID NO: 46; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 47; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 48; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 49; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 50; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 52; and wherein the human Fc region comprises a human IgG sequence selected from a sequence set forth in SEQ ID NOS: 436-468 and 484-539.

In certain embodiments, the isolated antibody comprises a VH sequence set forth in SEQ ID NO: 7 and a VL sequence set forth in SEQ ID NO: 53; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 54; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 55; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 56; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 7 and a VL sequence set forth in SEQ ID NO: 57; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 10 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 11 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 12 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 13 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated comprises a VH sequence set forth in SEQ ID NO: 14 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 15 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 16 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 17 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 18 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 19 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 20 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 21 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 22 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 23 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 24 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 25 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 26 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 27 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 28 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 28 and a VL sequence set forth in SEQ ID NO: 39; and wherein the human Fc region comprises a human IgG sequence set forth in SEQ ID NOs: 436-468 and 484-539.

In certain embodiments, the isolated antibody comprises a VH sequence set forth in SEQ ID NO: 29 and a VL sequence selected from a sequence set forth in SEQ ID NO: 39; and wherein the human Fc region comprises a human IgG sequence set forth in SEQ ID NOs: 436-468 and 484-539.

In certain embodiments, the isolated antibody comprises a VH sequence set forth in SEQ ID NO: 30 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 31 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 32 and a VL sequence set forth in SEQ ID NO: 39; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 8 and a VL sequence set forth in SEQ ID NO: 39; and wherein the human Fc region comprises a human IgG sequence selected from a sequence set forth in SEQ ID NOS: 436-468 and 484-539.

In certain embodiments, the isolated antibody comprises a VH sequence set forth in SEQ ID NO: 8 and a VL sequence set forth in SEQ ID NO: 51; and wherein the human Fc region comprises a human IgG sequence selected from a sequence set forth in SEQ ID NOs: 436-468 and 484-539. In certain embodiments, the isolated antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NOs: 436-468 and 484-539.

In certain embodiments, the isolated antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NO: 439.

In certain embodiments, the isolated antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NO: 446.

In certain embodiments, the isolated antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NO: 457.

In certain embodiments, the isolated antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NO: 460.

In certain embodiments, the isolated antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NO: 439.

In certain embodiments, the isolated antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NO: 446.

In certain embodiments, the isolated antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NO: 457.

In certain embodiments, the isolated antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NO: 460.

In certain embodiments, the isolated antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NO: 439.

In certain embodiments, the isolated antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NO: 446.

In certain embodiments, the isolated antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NO: 457.

In certain embodiments, the isolated antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NO: 460.

In certain embodiments, the isolated antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NO: 439.

In certain embodiments, the isolated antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NO: 446.

In certain embodiments, the isolated antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NO: 457.

In certain embodiments, the isolated antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NO: 460.

In certain embodiments, the isolated antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NO: 439.

In certain embodiments, the isolated antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NO: 446.

In certain embodiments, the isolated antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NO: 457.

In certain embodiments, the isolated antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NO: 460.

In certain embodiments, the isolated antibody comprises a VH sequence set forth in SEQ ID NO: 15 and a VL sequence set forth in SEQ ID NO: 39; and wherein the human Fc region comprises a human IgG sequence set forth in SEQ ID NO: 439.

In certain embodiments, the isolated antibody comprises a VH sequence set forth in SEQ ID NO: 15 and a VL sequence set forth in SEQ ID NO: 39; and wherein the human Fc region comprises a human IgG sequence set forth in SEQ ID NO: 457.

In certain embodiments, the isolated antibody comprises a VH sequence set forth in SEQ ID NO: 15 and a VL sequence set forth in SEQ ID NO: 39; and wherein the human Fc region comprises a human IgG sequence set forth in SEQ ID NO: 460.

In certain embodiments, the isolated antibody comprises a VH sequence set forth in SEQ ID NO: 15 and a VL sequence set forth in SEQ ID NO: 39; and wherein the human Fc region comprises a human IgG sequence set forth in SEQ ID NO: 446.

In certain embodiments, the isolated antibody comprises a VH sequence set forth in SEQ ID NO: 3 and a VL sequence set forth in SEQ ID NO: 39; and wherein the human Fc region comprises a human IgG sequence set forth in SEQ ID NO: 439.

In certain embodiments, the isolated antibody comprises a VH sequence set forth in SEQ ID NO: 3 and a VL sequence set forth in SEQ ID NO: 39; and wherein the human Fc region comprises a human IgG sequence set forth in SEQ ID NO: 457.

In certain embodiments, the isolated antibody comprises a VH sequence set forth in SEQ ID NO: 3 and a VL sequence set forth in SEQ ID NO: 39; and wherein the human Fc region comprises a human IgG sequence set forth in SEQ ID NO: 460.

In certain embodiments, the isolated antibody comprises a VH sequence set forth in SEQ ID NO: 3 and a VL sequence set forth in SEQ ID NO: 39; and wherein the human Fc region comprises a human IgG sequence set forth in SEQ ID NO: 446.

In certain embodiments, the isolated antibody comprises a VH sequence set forth in SEQ ID NO: 7 and a VL sequence set forth in SEQ ID NO: 51; and wherein the human Fc region comprises a human IgG sequence set forth in SEQ ID NO: 439.

In certain embodiments, the isolated antibody comprises a VH sequence set forth in SEQ ID NO: 7 and a VL sequence set forth in SEQ ID NO: 51; and wherein the human Fc region comprises a human IgG sequence set forth in SEQ ID NO: 457.

In certain embodiments, the isolated antibody comprises a VH sequence set forth in SEQ ID NO: 7 and a VL sequence set forth in SEQ ID NO: 51; and wherein the human Fc region comprises a human IgG sequence set forth in SEQ ID NO: 460.

In certain embodiments, the isolated antibody comprises a VH sequence set forth in SEQ ID NO: 7 and a VL sequence set forth in SEQ ID NO: 51; and wherein the human Fc region comprises a human IgG sequence set forth in SEQ ID NO: 446.

In certain embodiments, the isolated antibody comprises a VH sequence set forth in SEQ ID NO: 8 and a VL sequence set forth in SEQ ID NO: 39; and wherein the human Fc region comprises a human IgG sequence set forth in SEQ ID NO: 439.

In certain embodiments, the isolated antibody comprises a VH sequence set forth in SEQ ID NO: 8 and a VL sequence set forth in SEQ ID NO: 39; and wherein the human Fc region comprises a human IgG sequence set forth in SEQ ID NO: 457.

In certain embodiments, the isolated antibody comprises a VH sequence set forth in SEQ ID NO: 8 and a VL sequence set forth in SEQ ID NO: 51; and wherein the human Fc region comprises a human IgG sequence set forth in SEQ ID NO: 460.

In certain embodiments, the isolated antibody comprises a VH sequence set forth in SEQ ID NO: 8 and a VL sequence set forth in SEQ ID NO: 51; and wherein the human Fc region comprises a human IgG sequence set forth in SEQ ID NO: 446.

In certain embodiments, the isolated antibody comprises a VH sequence set forth in SEQ ID NO: 470 and a VL sequence set forth in SEQ ID NO: 471; and wherein the human Fc region comprises a human IgG sequence set forth in SEQ ID NO: 439.

In certain embodiments, the isolated antibody comprises a VH sequence set forth in SEQ ID NO: 470 and a VL sequence set forth in SEQ ID NO: 471; and wherein the human Fc region comprises a human IgG sequence set forth in SEQ ID NO: 457.

In certain embodiments, the isolated antibody comprises a VH sequence set forth in SEQ ID NO: 470 and a VL sequence set forth in SEQ ID NO: 471; and wherein the human Fc region comprises a human IgG sequence set forth in SEQ ID NO: 460.

In certain embodiments, the isolated antibody comprises a VH sequence set forth in SEQ ID NO: 470 and a VL sequence set forth in SEQ ID NO: 471; and wherein the human Fc region comprises a human IgG sequence set forth in SEQ ID NO: 446. In certain embodiments, the isolated antibody light chain comprises a constant light chain sequence set forth by SEQ ID NO: 469.

In certain embodiments, the isolated antibody Fc region comprises one or more amino acid substitutions, wherein the one or more substitutions result in a change (e.g., an increase or a decrease) in antibody half-life, ADCC activity, ADCP activity, or CDC activity as compared to an otherwise equivalent antibody comprising an Fc without the one or more substitutions. In certain embodiments, the change is an increase in antibody half-life, an increase or a decrease in ADCC activity, an increase in ADCP activity or an increase in CDC activity as compared to an otherwise equivalent antibody comprising an Fc without the one or more substitutions. In certain embodiments, the one or more amino acid substitutions results in increased antibody half-life compared to an antibody comprising a wild-type Fc region. In certain embodiments, the isolated antibody comprising an Fc region with one or more amino acid substitutions has a half-life of about 80 to 110 days in a human.

In certain embodiments, the change is an increase or a decrease in antibody half-life, an increase or a decrease in ADCC activity, an increase or a decrease in ADCP activity, or an increase or a decrease in CDC activity as compared to an otherwise equivalent antibody comprising an Fc without the one or more substitutions. In certain embodiments, the change is an increase in antibody half-life as compared to an otherwise equivalent antibody comprising an Fc without the one or more substitutions. In certain embodiments, the change is a decrease in antibody half-life as compared to an otherwise equivalent antibody comprising an Fc without the one or more substitutions. In certain embodiments, the change is an increase in ADCC activity as compared to an otherwise equivalent antibody comprising an Fc without the one or more substitutions. In certain embodiments, the change is a decrease in ADCC activity as compared to an otherwise equivalent antibody comprising an Fc without the one or more substitutions. In certain embodiments, the change is an increase in ADCP activity as compared to an otherwise equivalent antibody comprising an Fc without the one or more substitutions. In certain embodiments, the change is a decrease in ADCP activity as compared to an otherwise equivalent antibody comprising an Fc without the one or more substitutions. In certain embodiments, the change is an increase in CDC activity as compared to an otherwise equivalent antibody comprising an Fc without the one or more substitutions. In certain embodiments, the change is a decrease in CDC activity as compared to an otherwise equivalent antibody comprising an Fc without the one or more substitutions. In certain embodiments, the change is an increase in antibody half-life, an increase in ADCC activity, an increase in ADCP activity and an increase in CDC activity as compared to an otherwise equivalent antibody comprising an Fc without the one or more substitutions. In certain embodiments, the change is an increase in antibody half-life, a decrease in ADCC activity, an increase in ADCP activity and an increase in CDC activity as compared to an otherwise equivalent antibody comprising an Fc without the one or more substitutions.

In certain embodiments, the change is an increase in antibody half-life as compared to an otherwise equivalent antibody comprising an Fc without the one or more substitutions.

In certain embodiments, the change is an increase in ADCC activity as compared to an otherwise equivalent antibody comprising an Fc without the one or more substitutions.

In certain embodiments, the change is a decrease in ADCC activity as compared to an otherwise equivalent antibody comprising an Fc without the one or more substitutions.

In certain embodiments, the Fc region binds to Neonatal Fc receptor (FcRn). In certain embodiments, the Fc region binds an FcRn with higher affinity at pH 6.0 compared to an antibody comprising a wild-type Fc region. In certain embodiments, the Fc region binds to FcRn with a K_{D} of <1×10⁻⁷ M at pH 6.0.

In certain embodiments, the isolated antibody is a monoclonal antibody.

In certain embodiments, the antibody binds an IL-13 sequence set forth in SEQ ID NOs: 472-475.

In certain embodiments, the isolated antibody binds to an IL-13 sequence set forth in SEQ ID NOs: 472-475 with a K_{D} of less than or equal to about 1, 2, 3, 4, 5, 6, 7, 8, 9×10⁻⁹ M, as measured by surface plasmon resonance (SPR). In certain embodiments, the isolated antibody binds to an IL-13 sequence set forth in SEQ ID NOs: 472-475 with a K_{D} of less than or equal to about 1×10⁻¹⁰ M, as measured by SPR. In certain embodiments, the antibody binds to human IL-13 with a K_{D} of less than or equal to about 1×10⁻⁹M, as measured by SPR.

In certain embodiments, the isolated antibody exhibits a melting temperature greater than 68° C. as measured by Differential Scanning Fluorometry (DSF). In certain embodiments, the antibody exhibits a melting temperature greater than 75° C. as measured by DSF. In certain embodiments, the antibody exhibits a aggregation temperature equal to or greater than 71.2° C. as measured by DSF.

In certain embodiments, the isolated antibody has a retention time of 15.2 minutes or less as measured by hydrophobic interaction chromatography.

In certain embodiments, the isolated antibody does not have a heavy chain variable region sequence set forth in SEQ ID NO: 470.

In certain embodiments, the isolated antibody is used in the treatment of an inflammatory disorder or disease. In certain embodiments, the isolated antibody is used in the treatment of atopic dermatitis. In certain embodiments, the treatment reduces disease severity in a subject and wherein disease severity is assessed by an Atopic Dermatitis Disease Severity Outcome Measure. In certain embodiments, the isolated antibody is used in the treatment of asthma. In certain embodiments, the isolated antibody is used in the treatment of idiopathic pulmonary fibrosis. In certain embodiments, the isolated antibody is used in the treatment of alopecia areata. In certain embodiments, the isolated antibody is used in the treatment of chronic sinusitis with nasal polyps. In certain embodiments, the isolated antibody is used in the treatment of Chronic Rhinosinusitis without Nasal Polyps (CRSsNP). In certain embodiments, the isolated antibody is used in the treatment of eosinophilic esophagitis (EoE). In certain embodiments, the isolated antibody is used in the treatment of an Eosinophilic gastrointestinal disorder or disease (ENID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the isolated antibody is used in the treatment of Churg-Strauss syndrome/Eosinophilic granulomatosis with polyangiitis (EGPA). In certain embodiments, the isolated antibody is used in the treatment of Prurigo Nodularis (PN). In certain embodiments, the isolated antibody is used in the treatment of Chronic Spontaneous Urticaria (CSU). In certain embodiments, the isolated antibody is used in the treatment of Chronic Pruritis of Unknown Origin (CPUO). In certain embodiments, the isolated antibody is used in the treatment of Bullous Pemphigoid (BP). In certain embodiments, the isolated antibody is used in the treatment of Cold Inducible Urticaria (ColdU). In certain embodiments, the isolated antibody is used in the treatment of Allergic Fungal Rhinosinusitis (AFRS). In certain embodiments, the isolated antibody is used in the treatment of Allergic Bronchopulmonary Aspergillosis (ABPA). In certain embodiments, the isolated antibody is used in the treatment of Chronic Obstructive Pulmonary Disease (COPD). In certain embodiments, the isolated antibody is used in the treatment of inflammatory bowel disease, such as Crohn disease or ulcerative colitis. In certain embodiments, the isolated antibody is used in the treatment of psoriasis. In certain embodiments, the isolated antibody is used in the treatment of lupus. In certain embodiments, the isolated antibody is used in the treatment of rheumatoid arthritis.

In certain aspects, described herein is an isolated polynucleotide or set of polynucleotides encoding an antibody described herein, a VH thereof, a VL thereof, a light chain thereof, a heavy chain thereof, or an antigen-binding portion thereof, and optionally, wherein the polynucleotide or set of polynucleotides comprises cDNA. In certain aspects, described herein is a vector or set of vectors comprising the polynucleotide or set of polynucleotides. In certain aspects, described herein is a host cell comprising the polynucleotide or set of polynucleotides or the vector or set of vectors.

In certain aspects, described herein is a method of producing an antibody, the method comprising expressing the antibody with the host cell described herein and isolating the expressed antibody.

In certain aspects, described herein is a pharmaceutical composition comprising an antibody described herein and a pharmaceutically acceptable excipient.

In certain aspects, described herein is a kit comprising an antibody described herein or a pharmaceutical composition described herein and instructions for use.

In certain aspects, described herein is a method for treating an inflammatory disorder or disease in a mammalian subject in need thereof, the method comprising administering to the mammalian subject a therapeutically effective amount an antibody described herein or a pharmaceutical composition described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (ENID) selected from the group consisting of Eosinophilic Gastritis (EoG), Eosinophilic enteritis (EoN), Eosinophilic colitis (EoC), and Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn disease or ulcerative colitis. In certain embodiments, the inflammatory disorder or disease is psoriasis. In certain embodiments, the inflammatory disorder or disease is lupus. In certain embodiments, the inflammatory disorder or disease is rheumatoid arthritis.

In certain aspects, described herein is a method for treating a pathology associated with elevated levels of IL-13 in a mammalian subject in need thereof, the method comprising administering to the mammalian subject a therapeutically effective amount an antibody described herein or a pharmaceutical composition described herein.

In certain aspects, described herein is a method of reducing biological activity of IL-13 in a mammalian subject in need thereof, the method comprising administering to the mammalian subject a therapeutically effective amount an antibody described herein or a pharmaceutical composition described herein.

In certain aspects, described herein is a method of inhibiting the TH2 type allergic response in a mammalian subject in need thereof, the method comprising administering to the mammalian subject a therapeutically effective amount an antibody described herein or a pharmaceutical composition described herein.

In certain aspects, described herein is a method of reducing levels of Thymus and Activation Regulated Chemokine (TARC)/CCL17 in a mammalian subject in need thereof, the method comprising administering to the mammalian subject a therapeutically effective amount an antibody described herein or a pharmaceutical composition described herein.

In certain aspects, described herein is a method of preventing an inflammatory disorder or disease in a mammalian subject in need thereof, the method comprising administering to the mammalian subject a therapeutically effective amount an antibody of described herein or a pharmaceutical composition described herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a three-dimensional rendering of human IL-13. The “1” gray highlights the epitope of lebrikizumab, which overlaps with the epitope of Construct 133 disclosed herein (see e.g., Tables 2-8). These epitopes also overlap with the IL-4Ra epitope on IL-13, shown in “2” gray. The epitope of tralokunumab-ldrm (Adbry™) is shown in “3” gray. The IL-13Rα1/IL-13Rα2 overlapping epitope is shown in “4” gray, and the IL-13Rα2 (non-overlapping) epitope is shown in “5” gray.

FIG. 2 is a graph depicting the percentage of inhibition of IL-13 binding to IL13Rα1/IL-4Ra overexpressing HEK293 cells that have been incubated with the indicated engineered anti-IL-13 antibodies, as determined by FACs.

FIG. 3 is a graph depicting the percentage of inhibition of IL-13-induced phosphorylation of STAT6 in HT-29 cells that have been incubated with the indicated engineered anti-IL-13 antibodies, as determined by FACs.

FIG. 4 is a graph depicting the percentage of inhibition of IL-13-induced release of thymus- and activation-regulated chemokine (TARC/CCL17) in the supernatant of A549 cell cultures that have been incubated with the indicated engineered anti-IL-13 antibodies, as determined by enzyme-linked immunoassay (ELISA).

FIG. 5 is a graph depicting the percentage of inhibition of IL-13-induced release of TARC in the supernatant of A549 cell cultures that have been incubated with the indicated engineered anti-IL-13 antibodies, as determined by ELISA.

FIG. 6 is a graph depicting the percentage of inhibition of IL-13-induced proliferation of TF-1 cells that have been incubated with the indicated engineered anti-IL-13 antibodies, as quantified by a CellTiter-Glo assay.

FIG. 7 is a graph depicting the percentage of inhibition of IL-13-induced phosphorylation of STAT6 in human peripheral blood mononuclear cells (PBMCs) cells that have been incubated with the indicated engineered anti-IL-13 antibodies, as determined by FACs.

FIG. 8 is a graph depicting the percentage of inhibition of IL-13-induced CD23 expression in human peripheral blood mononuclear cells (PBMCs) cells that have been incubated with the indicated engineered anti-IL-13 antibodies, as determined by FACs.

FIG. 9 is a graph depicting the serum concentration (ng/ml) of Construct 133 and lebrikizumab over time (days post injection) in non-human primates (NHPs). The half-life of Construct 15 was 27.6 days, as compared to 17 to 18 days for lebrikizumab.

FIG. 10 is a graph depicting normalized AUC_{0-∞} (C_{norm*day}), or area under the curve (AUC) from dosing to infinity, among antibodies with the YTE substitution.

FIG. 11 is a graph depicting the serum concentration (ng/ml) of the indicated engineered anti-IL-13 antibodies administered intravenously (IV) in NHPs.

FIG. 12 is a graph depicting the serum concentration (ng/ml) of the indicated engineered anti-IL-13 antibodies administered subcutaneously (SQ) in NHPs.

FIG. 13 is a graph depicting the percentage of inhibition of IL-13-induced CCL2 secretion in HaCaT cells that have been incubated with the indicated engineered anti-IL-13 antibodies, as determined by Luminex.

FIG. 14 is a graph depicting the percentage of inhibition of CCL26 secretion in HaCaT cells that have been incubated with the indicated engineered anti-IL-13 antibodies, as determined by Luminex.

FIG. 15 is a graph depicting the percentage of inhibition of NTRK1 gene expression in HaCaT cells that have been incubated with the indicated engineered anti-IL-13 antibodies, as determined by QuantiGene assay.

### DETAILED DESCRIPTION

IL-13 signaling begins with the binding of IL-13 to IL-13Rα1, forming an inactive complex that then binds to IL-4Ra to form the complete, active receptor heterodimer. This active receptor heterodimer contributes to the pathogenesis of atopic dermatitis. The instant disclosure relates, in part, to anti-IL-13 antibodies that prevent the formation of this heterodimer.

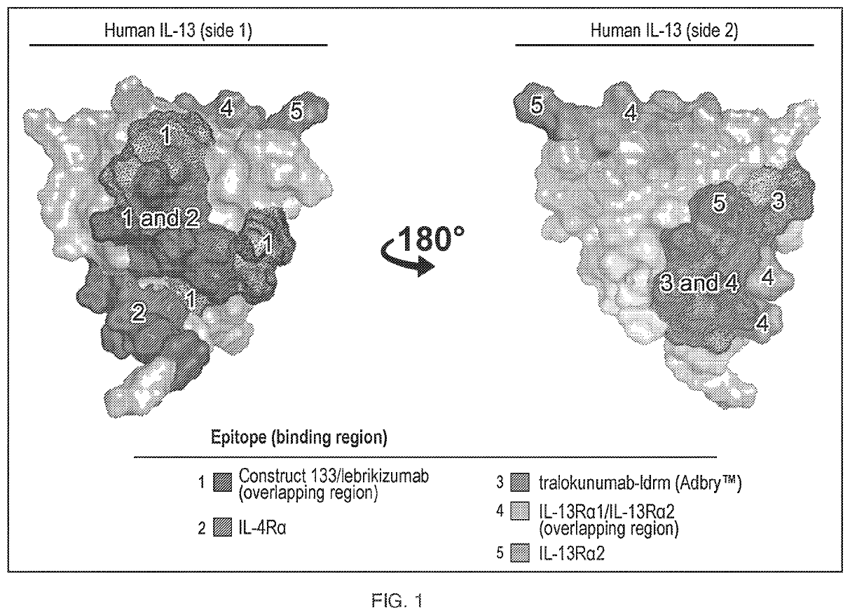

As shown in FIG. 1, a three-dimensional rendering of human IL-13, “1” gray highlights the epitope of lebrikizumab, which overlaps with the epitope of certain antibodies disclosed herein. Importantly, these epitopes also overlap with the IL-4Rα epitope on IL-13. Without wishing to be bound by theory, it is believed that antibodies that bind to this region are likely to prevent the formation of the IL-13Rα1-IL-4Rα heterodimer, limiting the inflammatory signaling that leads to atopic dermatitis. In contrast, the epitope of tralokunumab-ldrm (Adbry™), highlighted in “3” gray, does not overlap with the IL-4Rα epitope on IL-13 and therefore may have a more limited ability to prevent heterodimerization.

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular form “a,” “an,” and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

An “effective amount” or “therapeutically effective amount” as used herein refers to an amount of therapeutic compound, such as an anti-IL-13 antibody, administered to an individual, either as a single dose or as part of a series of doses, which is effective to produce or contribute to a desired therapeutic effect, either alone or in combination with another therapeutic modality. Examples of a desired therapeutic effect is enhancing an immune response, slowing or delaying tumor development; stabilization of disease; amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the term “subject” or “individual” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody provided herein. In some embodiments, the disease or condition is a cancer. In some embodiments, the disease or condition is a viral infection.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “co-administration,” “co-administer,” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “kd” (sec-1), as used herein, refers to the dissociation rate constant of a particular antibody-antigen interaction. This value is also referred to as the koff value.

The term “ka” (M-1×sec-1), as used herein, refers to the association rate constant of a particular antibody-antigen interaction. This value is also referred to as the kon value.

The term “KD” (M), as used herein, refers to the dissociation equilibrium constant of a particular antibody-antigen interaction. KD=kd/ka. In some embodiments, the affinity of an antibody is described in terms of the KD for an interaction between such antibody and its antigen. For clarity, as known in the art, a smaller KD value indicates a higher affinity interaction, while a larger KD value indicates a lower affinity interaction.

The term “KA” (M-1), as used herein, refers to the association equilibrium constant of a particular antibody-antigen interaction. KA=ka/kd.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

A “anti-IL-13 antibody,” “IL-13 antibody,” or “IL-13 specific antibody” is an antibody, as provided herein, which specifically binds to the antigen IL-13.

The term “epitope” means a portion of an antigen that specifically binds to an antibody.

The term “hypervariable region” or “HVR,” as used herein, refers to each of the regions of an antibody variable domain which are hypervariable in sequence and/or form structurally defined loops (“hypervariable loops”).

The term “antigen-binding domain” means the portion of an antibody that is capable of specifically binding to an antigen or epitope.

The term “chimeric antibody” refers to an antibody in which a portion of the heavy and/or light chain is derived from a particular source or species, while the remainder of the heavy and/or light chain is derived from a different source or species.

The term “human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” refers to an antibody that comprises two or more different antigen-binding domains that collectively specifically bind two or more different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77 (1): 13-22).

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

The term “Fc domain” or “Fc region” herein is used to define a C-terminal region of an immunoglobulin heavy chain that contains at least a portion of the constant region. The term includes native sequence Fc regions and variant Fc regions.

The term “substantially purified” refers to a construct described herein, or variant thereof that may be substantially or essentially free of components that normally accompany or interact with the protein as found in its naturally occurring environment, i.e. a native cell, or host cell in the case of recombinantly produced antibody that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

### Anti-IL-13 Antibodies

### Antibody Structure

The present application provides antibodies and compositions comprising an antibody which binds IL-13.

The recognized immunoglobulin genes include the kappa, lambda, alpha, gamma, delta, epsilon, and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises of the VH, CH1, CH2, and CH3 domains respectively from the N- to C-terminus. The light chain comprises of the VL and CL domains from N- to C-terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or derived from an immunoglobulin based construct such as a diabody or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody, or any chimeric antibody.

In some embodiments, the antibodies provided herein comprise a heavy chain. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody.

Generally, native four-chain antibodies comprise six hypervariable regions (HVRs); three in the VH (H1, H2, and H3), and three in the VL (L1, L2, and L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. HVRs are also referred to as CDRs, and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 1
Table 1. Residues in CDRs according to
Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding and other amino acid residues, which are not directly involved in the binding. The epitope to which an antibody binds can be determined using known techniques for epitope determination such as, for example, testing for antibody binding to IL-13 variants with different point-mutations or to chimeric IL-13 variants.

To screen for antibodies which bind to an epitope on a target antigen bound by an antibody of interest (e.g., IL-13), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Chimeric antibodies are antibodies in which a portion of the heavy and/or light chain is derived from a particular source or species, while the remainder of the heavy and/or light chain is derived from a different source or species.

Human antibodies are antibodies which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152 and 5,877,293. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

The two or more different epitopes may be epitopes on the same antigen (e.g., a single IL-13) or on different antigens (e.g., different IL-13 molecules, or a IL-13 molecule and a non-IL-13 molecule). In some embodiments, a multi-specific antibody binds two different epitopes (i.e., a “bispecific antibody”). In some embodiments, a multi-specific antibody binds three different epitopes (i.e., a “trispecific antibody”).

Anti-IL-13 antibodies can include those described herein such as the clones set forth in the drawings and/or tables. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment.

In some embodiments the antibodies are monoclonal antibodies.

In some embodiments the antibodies are polyclonal antibodies.

In some embodiments the antibodies are produced by hybridomas. In other embodiments, the antibodies are produced by recombinant cells engineered to express the desired variable and constant domains.

In some embodiments the antibodies may be single chain antibodies or other antibody derivatives retaining the antigen specificity and the lower hinge region or a variant thereof.

In some embodiments the antibodies may be polyfunctional antibodies, recombinant antibodies, human antibodies, humanized antibodies, fragments or variants thereof. In particular embodiments, the antibody fragment or a derivative thereof is selected from a Fab fragment, a Fab′2 fragment, a CDR, and ScFv.

In some embodiments, the antibodies are capable of forming an immune complex. For example, an immune complex can be a tumor cell covered by antibodies.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### Sequences of IL-13 Antibodies

### VH Domains

In some embodiments, an antibody provided herein comprises a VH sequence selected from SEQ ID NOs: 1-32 and 470.

In some embodiments, an antibody provided herein comprises a VH sequence having at least about 80%, 90%, 91%, 92%, 93%, 94%, 95%, 96%, 97%, 98% or 99% identity to an illustrative VH sequence provided in SEQ ID NOs: 1-32 and 470. In some embodiments, an antibody provided herein comprises a VH sequence provided in SEQ ID NOs: 1-32 and 470, with up to 1, 2, 3, 4, 5, 6, 7, 8, 9, 10, 11, 12, 13, 14, 15, 16, 17, 18, 19, or 20 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VL Domains

In some embodiments, an antibody provided herein comprises a VL sequence selected from SEQ ID NOs: 33-57 and 471.

In some embodiments, an antibody provided herein comprises a VL sequence having at least about 80%, 90%, 91%, 92%, 93%, 94%, 95%, 96%, 97%, 98% or 99% identity to an illustrative VL sequence provided in SEQ ID NOs: 33-57 and 471. In some embodiments, an antibody provided herein comprises a VL sequence provided in SEQ ID NOs: 33-57 and 471 with up to 1, 2, 3, 4, 5, 6, 7, 8, 9, 10, 11, 12, 13, 14, 15, 16, 17, 18, 19, or 20 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VH-VL Combinations

In some embodiments, an antibody provided herein comprises a VH sequence selected from SEQ ID NOs: 1-32 and 470; and a VL sequence selected from SEQ ID NOs: 33-57 and 471, such as the VH-VL combination set forth in Table 2, below.

In certain aspects, any of SEQ ID NOs: 1-32 and 470 can be combined with any of SEQ ID NOs: 33-57 and 471.

In certain embodiments, the antibody comprises a VH sequence selected from the sequences set forth in SEQ ID NOs: 1-32 and 470 and a VL sequence set forth in SEQ ID NO: 49.

In certain embodiments, the antibody comprises a VH sequence selected from the sequences set forth in SEQ ID NOs: 1-32 and 470 and a VL sequence set forth in SEQ ID NO: 51.

In some embodiments, an antibody provided herein comprises a VH sequence having at least about 80%, 90%, 91%, 92%, 93%, 94%, 95%, 96%, 97%, 98% or 99% identity to an illustrative VH sequence provided in SEQ ID NOs: 1-32 and 470; and a VL sequence having at least about 80%, 90%, 91%, 92%, 93%, 94%, 95%, 96%, 97%, 98% or 99% identity to an illustrative VL sequence provided in SEQ ID NOs: 33-57 and 471. In some embodiments, an antibody provided herein comprises a VH sequence provided in SEQ ID NOs: 1-32 and 470, with up to 1, 2, 3, 4, 5, 6, 7, 8, 9, 10, 11, 12, 13, 14, 15, 16, 17, 18, 19, or 20 amino acid substitutions; and a VL sequence provided in SEQ ID NOs: 33-57 and 471, with up to 1, 2, 3, 4, 5, 6, 7, 8, 9, 10, 11, 12, 13, 14, 15, 16, 17, 18, 19, or 20 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a VH sequence and a VL sequence selected from combinations set forth in Table 2, below. In certain embodiments, the antibody comprises a VH sequence set forth in SEQ ID NO: 1 and a VL sequence set forth in SEQ ID NO: 33. In certain embodiments, the antibody comprises a VH sequence set forth in SEQ ID NO: 2 and a VL sequence set forth in SEQ ID NO: 33. In certain embodiments, the antibody comprises a VH sequence set forth in SEQ ID NO: 3 and a VL sequence set forth in SEQ ID NO: 35. In certain embodiments, the antibody comprises a VH sequence set forth in SEQ ID NO: 4 and a VL sequence set forth in SEQ ID NO: 35. In certain embodiments, the antibody comprises a VH sequence set forth in SEQ ID NO: 5 and a VL sequence set forth in SEQ ID NO: 35. In certain embodiments, the antibody comprises a VH sequence set forth in SEQ ID NO: 6 and a VL sequence set forth in SEQ ID NO: 35. In certain embodiments, the antibody comprises a VH sequence set forth in SEQ ID NO: 7 and a VL sequence set forth in SEQ ID NO: 35. In certain embodiments, the antibody comprises a VH sequence set forth in SEQ ID NO: 3 and a VL sequence set forth in SEQ ID NO: 36. In certain embodiments, the antibody comprises a VH sequence set forth in SEQ ID NO: 4 and a VL sequence set forth in SEQ ID NO: 36. In certain embodiments, the antibody comprises a VH sequence set forth in SEQ ID NO: 5 and a VL sequence set forth in SEQ ID NO: 36. In certain embodiments, the antibody comprises a VH sequence set forth in SEQ ID NO: 6 and a VL sequence set forth in SEQ ID NO: 36. In certain embodiments, the antibody comprises a VH sequence set forth in SEQ ID NO: 7 and a VL sequence set forth in SEQ ID NO: 36. In certain embodiments, the antibody comprises a VH sequence set forth in SEQ ID NO: 3 and a VL sequence set forth in SEQ ID NO: 39. In certain embodiments, the antibody comprises a VH sequence set forth in SEQ ID NO: 4 and a VL sequence set forth in SEQ ID NO: 39. In certain embodiments, the antibody comprises a VH sequence set forth in SEQ ID NO: 5 and a VL sequence set forth in SEQ ID NO: 39. In certain embodiments, the antibody comprises a VH sequence set forth in SEQ ID NO: 6 and a VL sequence set forth in SEQ ID NO: 39. In certain embodiments, the antibody comprises a VH sequence set forth in SEQ ID NO: 7 and a VL sequence set forth in SEQ ID NO: 39. In certain embodiments, the antibody comprises a VH sequence set forth in SEQ ID NO: 3 and a VL sequence set forth in SEQ ID NO: 40. In certain embodiments, the antibody comprises a VH sequence set forth in SEQ ID NO: 4 and a VL sequence set forth in SEQ ID NO: 40. In certain embodiments, the antibody comprises a VH sequence set forth in SEQ ID NO: 5 and a VL sequence set forth in SEQ ID NO: 40. In certain embodiments, the antibody comprises a VH sequence set forth in SEQ ID NO: 6 and a VL sequence set forth in SEQ ID NO: 40. In certain embodiments, the antibody comprises a VH sequence set forth in SEQ ID NO: 7 and a VL sequence set forth in SEQ ID NO: 40. In certain embodiments, the antibody comprises a VH sequence set forth in SEQ ID NO: 8 and a VL sequence set forth in SEQ ID NO: 42. In certain embodiments, the antibody comprises a VH sequence set forth in SEQ ID NO: 9 and a VL sequence set forth in SEQ ID NO: 43. In certain embodiments, the antibody comprises a VH sequence set forth in SEQ ID NO: 7 and a VL sequence set forth in SEQ ID NO: 39. In certain embodiments, the antibody comprises a VH sequence set forth in SEQ ID NO: 7 and a VL sequence set forth in SEQ ID NO: 44. In certain embodiments, the antibody comprises a VH sequence set forth in SEQ ID NO: 7 and a VL sequence set forth in SEQ ID NO: 45. In certain embodiments, the antibody comprises a VH sequence set forth in SEQ ID NO: 7 and a VL sequence set forth in SEQ ID NO: 46. In certain embodiments, the antibody comprises a VH sequence set forth in SEQ ID NO: 7 and a VL sequence set forth in SEQ ID NO: 47. In certain embodiments, the antibody comprises a VH sequence set forth in SEQ ID NO: 7 and a VL sequence set forth in SEQ ID NO: 48. In certain embodiments, the antibody comprises a VH sequence set forth in SEQ ID NO: 7 and a VL sequence set forth in SEQ ID NO: 49. In certain embodiments, the antibody comprises a VH sequence set forth in SEQ ID NO: 7 and a VL sequence set forth in SEQ ID NO: 50. In certain embodiments, the antibody comprises a VH sequence set forth in SEQ ID NO: 7 and a VL sequence set forth in SEQ ID NO: 51. In certain embodiments, the antibody comprises a VH sequence set forth in SEQ ID NO: 7 and a VL sequence set forth in SEQ ID NO: 52. In certain embodiments, the antibody comprises a VH sequence set forth in SEQ ID NO: 7 and a VL sequence set forth in SEQ ID NO: 53. In certain embodiments, the antibody comprises a VH sequence set forth in SEQ ID NO: 7 and a VL sequence set forth in SEQ ID NO: 54. In certain embodiments, the antibody comprises a VH sequence set forth in SEQ ID NO: 7 and a VL sequence set forth in SEQ ID NO: 55. In certain embodiments, the antibody comprises a VH sequence set forth in SEQ ID NO: 7 and a VL sequence set forth in SEQ ID NO: 56. In certain embodiments, the antibody comprises a VH sequence set forth in SEQ ID NO: 7 and a VL sequence set forth in SEQ ID NO: 57. In certain embodiments, the antibody comprises a VH sequence set forth in SEQ ID NO: 10 and a VL sequence set forth in SEQ ID NO: 39. In certain embodiments, the antibody comprises a VH sequence set forth in SEQ ID NO: 11 and a VL sequence set forth in SEQ ID NO: 39. In certain embodiments, the antibody comprises a VH sequence set forth in SEQ ID NO: 12 and a VL sequence set forth in SEQ ID NO: 39. In certain embodiments, the antibody comprises a VH sequence set forth in SEQ ID NO: 13 and a VL sequence set forth in SEQ ID NO: 39; and wherein the human Fc region comprises a human IgG sequence selected from a sequence set forth in SEQ ID NO: 436-468 and 484-539. In certain embodiments, the antibody comprises a VH sequence set forth in SEQ ID NO: 14 and a VL sequence set forth in SEQ ID NO: 39. In certain embodiments, the antibody comprises a VH sequence set forth in SEQ ID NO: 15 and a VL sequence set forth in SEQ ID NO: 39. In certain embodiments, the antibody comprises a VH sequence set forth in SEQ ID NO: 16 and a VL sequence set forth in SEQ ID NO: 39. In certain embodiments, the antibody comprises a VH sequence set forth in SEQ ID NO: 17 and a VL sequence set forth in SEQ ID NO: 39. In certain embodiments, the antibody comprises a VH sequence set forth in SEQ ID NO: 18 and a VL sequence set forth in SEQ ID NO: 39. In certain embodiments, the antibody comprises a VH sequence set forth in SEQ ID NO: 19 and a VL sequence set forth in SEQ ID NO: 39. In certain embodiments, the antibody comprises a VH sequence set forth in SEQ ID NO: 20 and a VL sequence set forth in SEQ ID NO: 39. In certain embodiments, the antibody comprises a VH sequence set forth in SEQ ID NO: 21 and a VL sequence set forth in SEQ ID NO: 39. In certain embodiments, the antibody comprises a VH sequence set forth in SEQ ID NO: 22 and a VL sequence set forth in SEQ ID NO: 39. In certain embodiments, the antibody comprises a VH sequence set forth in SEQ ID NO: 23 and a VL sequence set forth in SEQ ID NO: 39. In certain embodiments, the antibody comprises a VH sequence set forth in SEQ ID NO: 24 and a VL sequence set forth in SEQ ID NO: 39. In certain embodiments, the antibody comprises a VH sequence set forth in SEQ ID NO: 25 and a VL sequence set forth in SEQ ID NO: 39. In certain embodiments, the antibody comprises a VH sequence set forth in SEQ ID NO: 26 and a VL sequence set forth in SEQ ID NO: 39. In certain embodiments, the antibody comprises a VH sequence set forth in SEQ ID NO: 27 and a VL sequence set forth in SEQ ID NO: 39. In certain embodiments, the antibody comprises a VH sequence set forth in SEQ ID NO: 28 and a VL sequence set forth in SEQ ID NO: 39. In certain embodiments, the antibody comprises a VH sequence set forth in SEQ ID NO: 28 and a VL sequence set forth in SEQ ID NO: 39. In certain embodiments, the antibody comprises a VH sequence set forth in SEQ ID NO: 29 and a VL sequence set forth in SEQ ID NO: 39. In certain embodiments, the antibody comprises a VH sequence set forth in SEQ ID NO: 30 and a VL sequence set forth in SEQ ID NO: 39. In certain embodiments, the antibody comprises a VH sequence set forth in SEQ ID NO: 31 and a VL sequence set forth in SEQ ID NO: 39. In certain embodiments, the antibody comprises a VH sequence set forth in SEQ ID NO: 32 and a VL sequence set forth in SEQ ID NO: 39. In certain embodiments, the antibody comprises a VH sequence set forth in SEQ ID NO: 8 and a VL sequence set forth in SEQ ID NO: 39. In certain embodiments, the antibody comprises a VH sequence set forth in SEQ ID NO: 8 and a VL sequence set forth in SEQ ID NO: 51. In certain embodiments, the antibody comprises a VH sequence set forth in SEQ ID NO: 3 and a VL sequence set forth in SEQ ID NO: 51. In certain embodiments, the antibody comprises a VH sequence set forth in SEQ ID NO: 470 and a VL sequence set forth in SEQ ID NO: 471.

In certain embodiments, the isolated antibody comprises a heavy chain variable domain comprising a framework region sequence selected from a sequence set forth in SEQ ID NOs: 198-229, 255-256, 258-259, 261-285, 311-315, 317-342, 368-369, 371-399, and 540-580. In certain embodiments, the isolated antibody comprises a heavy chain variable domain comprising 1, 2, 3, or 4 framework region sequences selected from a sequence set forth in SEQ ID NOs: 198-229, 255-256, 258-259, 261-285, 311-315, 317-342, 368-369, 371-399, and 540-580.

In certain embodiments, the isolated antibody comprises a light chain variable domain comprising a framework region sequence selected from a sequence set forth in SEQ ID NOs: 230-231, 233-235, 239, 241-254, 286, 288, 290-291, 293, 296-310, 343-345, 347, 400-424, and 581-609. In certain embodiments, the isolated antibody comprises a light chain variable domain comprising 1, 2, 3, or 4 framework region sequences selected from a sequence set forth in SEQ ID NOs: 230-231, 233-235, 239, 241-254, 286, 288, 290-291, 293, 296-310, 343-345, 347, 400-424, and 581-609.

In certain embodiments, the isolated antibody comprises a heavy chain variable domain comprising 1, 2, 3, or 4 framework region sequences selected from a sequence set forth in SEQ ID NOs: 198-229, 255-256, 258-259, 261-285, 311-315, 317-342, 368-369, 371-399, and 540-580, and comprises a light chain variable domain comprising 1, 2, 3, or 4 framework region sequences selected from a sequence set forth in SEQ ID NOs: 230-231, 233-235, 239, 241-254, 286, 288, 290-291, 293, 296-310, 343-345, 347, 400-424, and 581-609.

TABLE 2
Anti-interleukin (IL)-13 antibody VH-VL sequences

| | VH; VL; HC | | |
|---|---|---|---|
| | constant; | | |
| | and LC | | |
| | constant | | |
| Construct | names, | | |
| ID | respectively* | VH sequence | VL sequence |
| 1 | Lebrikizumab- | QVTLRESGPALVKPTQ | DIVMTQSPDSLSVSLGER |
| (Lebrikizumab) | HC; | TLTLTCTVSGFSLSAYS | ATINCRASKSVDSYGNSF |
| | Lebrikizumab- | VNWIRQPPGKALEWLA | MHWYQQKPGQPPKLLIY |
| | LC; | MIWGDGKIVYNSALKS | LASNLESGVPDRFSGSGS |
| | IgG4-SP; | RLTISKDTSKNQVVLT | GTDFTLTISSLQAEDVAV |
| | Human | MTNMDPVDTATYYCA | YYCQQNNEDPRTFGGGT |
| | kappa LC | GDGYYPYAMDNWGQ | KVEIK (SEQ ID NO: 471) |
| | | GSLVTVSS (SEQ ID NO: | |
| | | 470) | |
| 2 | Lebrikizumab- | QVTLRESGPALVKPTQ | DIVMTQSPDSLSVSLGER |
| | HC; | TLTLTCTVSGFSLSAYS | ATINCRASKSVDSYGNSF |
| | Lebrikizumab- | VNWIRQPPGKALEWLA | MHWYQQKPGQPPKLLIY |
| | LC; | MIWGDGKIVYNSALKS | LASNLESGVPDRFSGSGS |
| | hIgG1- | RLTISKDTSKNQVVLT | GTDFTLTISSLQAEDVAV |
| | LAGA YTE; | MTNMDPVDTATYYCA | YYCQQNNEDPRTFGGGT |
| | Human | GDGYYPYAMDNWGQ | KVEIK (SEQ ID NO: 471) |
| | kappa LC | GSLVTVSS (SEQ ID NO: | |
| | | 470) | |
| 3 | HC0; LC0; | QVQLQESGPGLVAPSQ | NIVLTQSPASLAVSLGQR |
| | hIgG1- | SLSITCTVSGFSLNAYS | ATISCRASKSVDSYGNSF |
| | LAGA YTE; | VNWVRQPPGKGLEWL | MHWYQQKPGQPPKLLIY |
| | Human | GMIWGDGKIVYNSALK | LASNLESGVPARFSGSGS |
| | kappa LC | SRLNISKDSSKSQVFLK | RTDFTLTIDPVEADDAAS |
| | | MSSLQSDDTARYYCAG | YYCQQNNEDPRTFGGGT |
| | | DGYYPYAMDNWGHGT | KLEIK (SEQ ID NO: 33) |
| | | SVTVSS (SEQ ID NO: 1) | |
| 4 | HC0_M; | QVQLQESGPGLVAPSQ | NIVLTQSPASLAVSLGQR |
| | LC0; hIgG1- | SLSITCTVSGFSLNAYS | ATISCRASKSVDSYGNSF |
| | LAGA YTE; | VNWVRQPPGKGLEWL | MHWYQQKPGQPPKLLIY |
| | Human | GMIWGDGKIVYNSALK | LASNLESGVPARFSGSGS |
| | kappa LC | SRLTISKDSSKSQVFLK | RTDFTLTIDPVEADDAAS |
| | | MSSLQSDDTARYYCAG | YYCQQNNEDPRTFGGGT |
| | | DGYYPYAMDNWGHGT | KLEIK (SEQ ID NO: 33) |
| | | SVTVSS (SEQ ID NO: 2) | |
| 5 | HC1; LC2; | EVQLQESGPGLVKPSET | DIQLTQSPSSLSASVGDRV |
| | hIgG1- | LSLTCTVSGFSLNAYSV | TITCRASKSVDSYGNSFM |
| | LAGA YTE; | NWIRQPPGKGLEWLG | HWYQQKPGKAPKLLIYL |
| | Human | MIWGDGKIVYNSALKS | ASNLESGVPSRFSGSGSRT |
| | kappa LC | RLTISKDSSKNQVSLKL | DFTLTISSLQPEDFATYYC |
| | | SSVTAADTAVYYCAGD | QQNNEDPRTFGGGTKVEI |
| | | GYYPYAMDNWGQGTT | K (SEQ ID NO: 35) |
| | | VTVSS (SEQ ID NO: 3) | |
| 6 | HC2; LC2; | EVQLVQSGAEVKKPGA | DIQLTQSPSSLSASVGDRV |
| | hIgG1- | SVKVSCKASGFSLNAY | TITCRASKSVDSYGNSFM |
| | LAGA YTE; | SVNWVRQAPGQGLEW | HWYQQKPGKAPKLLIYL |
| | Human | LGMIWGDGKIVYNSAL | ASNLESGVPSRFSGSGSRT |
| | kappa LC | KSRLTITKDSSTSTVYM | DFTLTISSLQPEDFATYYC |
| | | ELSSLRSEDTAVYYCA | QQNNEDPRTFGGGTKVEI |
| | | GDGYYPYAMDNWGQ | K (SEQ ID NO: 35) |
| | | GTTVTVSS (SEQ ID NO: | |
| | | 4) | |
| 7 | HC3; LC2; | EVQLVQSGAEVKKPGS | DIQLTQSPSSLSASVGDRV |
| | hIgG1- | SVKVSCKASGFSLNAY | TITCRASKSVDSYGNSFM |
| | LAGA YTE; | SVNWVRQAPGQGLEW | HWYQQKPGKAPKLLIYL |
| | Human | LGMIWGDGKIVYNSAL | ASNLESGVPSRFSGSGSRT |
| | kappa LC | KSRLTITKDSSTSTVYM | DFTLTISSLQPEDFATYYC |
| | | ELSSLRSEDTAVYYCA | QQNNEDPRTFGGGTKVEI |
| | | GDGYYPYAMDNWGQ | K (SEQ ID NO: 35) |
| | | GTTVTVSS (SEQ ID NO: | |
| | | 5) | |
| 8 | HC4; LC2; | EVQLVESGGGLVKPGG | DIQLTQSPSSLSASVGDRV |
| | hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSRT |
| | kappa LC | SRLTISKDSSKNTVYLQ | DFTLTISSLQPEDFATYYC |
| | | MNSLKTEDTAVYYCA | QQNNEDPRTFGGGTKVEI |
| | | GDGYYPYAMDNWGQ | K (SEQ ID NO: 35) |
| | | GTTVTVSS (SEQ ID NO: | |
| | | 6) | |
| 9 | HC5; LC2; | EVQLLESGGGLVQPGG | DIQLTQSPSSLSASVGDRV |
| | hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSRT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 35) |
| | | GTTVTVSS (SEQ ID NO: | |
| | | 7) | |
| 10 | HC1; LC3; | EVQLQESGPGLVKPSET | EIVLTQSPATLSVSPGERA |
| | hIgG1- | LSLTCTVSGFSLNAYSV | TLSCRASKSVDSYGNSFM |
| | LAGA YTE; | NWIRQPPGKGLEWLG | HWYQQKPGQAPRLLIYL |
| | Human | MIWGDGKIVYNSALKS | ASNLESGIPARFSGSGSRT |
| | kappa LC | RLTISKDSSKNQVSLKL | EFTLTISSLQSEDFAVYYC |
| | | SSVTAADTAVYYCAGD | QQNNEDPRTFGGGTKVEI |
| | | GYYPYAMDNWGQGTT | K (SEQ ID NO: 36) |
| | | VTVSS (SEQ ID NO: 3) | |
| 11 | HC2; LC3; | EVQLVQSGAEVKKPGA | EIVLTQSPATLSVSPGERA |
| | hIgG1- | SVKVSCKASGFSLNAY | TLSCRASKSVDSYGNSFM |
| | LAGA YTE; | SVNWVRQAPGQGLEW | HWYQQKPGQAPRLLIYL |
| | Human | LGMIWGDGKIVYNSAL | ASNLESGIPARFSGSGSRT |
| | kappa LC | KSRLTITKDSSTSTVYM | EFTLTISSLQSEDFAVYYC |
| | | ELSSLRSEDTAVYYCA | QQNNEDPRTFGGGTKVEI |
| | | GDGYYPYAMDNWGQ | K (SEQ ID NO: 36) |
| | | GTTVTVSS (SEQ ID NO: | |
| | | 4) | |
| 12 | HC3; LC3; | EVQLVQSGAEVKKPGS | EIVLTQSPATLSVSPGERA |
| | hIgG1- | SVKVSCKASGFSLNAY | TLSCRASKSVDSYGNSFM |
| | LAGA YTE; | SVNWVRQAPGQGLEW | HWYQQKPGQAPRLLIYL |
| | Human | LGMIWGDGKIVYNSAL | ASNLESGIPARFSGSGSRT |
| | kappa LC | KSRLTITKDSSTSTVYM | EFTLTISSLQSEDFAVYYC |
| | | ELSSLRSEDTAVYYCA | QQNNEDPRTFGGGTKVEI |
| | | GDGYYPYAMDNWGQ | K (SEQ ID NO: 36) |
| | | GTTVTVSS (SEQ ID NO: | |
| | | 5) | |
| 13 | HC4; LC3; | EVQLVESGGGLVKPGG | EIVLTQSPATLSVSPGERA |
| | hIgG1- | SLRLSCAASGFSLNAYS | TLSCRASKSVDSYGNSFM |
| | LAGA YTE; | VNWVRQAPGKGLEWL | HWYQQKPGQAPRLLIYL |
| | Human | GMIWGDGKIVYNSALK | ASNLESGIPARFSGSGSRT |
| | kappa LC | SRLTISKDSSKNTVYLQ | EFTLTISSLQSEDFAVYYC |
| | | MNSLKTEDTAVYYCA | QQNNEDPRTFGGGTKVEI |
| | | GDGYYPYAMDNWGQ | K (SEQ ID NO: 36) |
| | | GTTVTVSS (SEQ ID NO: | |
| | | 6) | |
| 14 | HC5; LC3; | EVQLLESGGGLVQPGG | EIVLTQSPATLSVSPGERA |
| | hIgG1- | SLRLSCAASGFSLNAYS | TLSCRASKSVDSYGNSFM |
| | LAGA YTE; | VNWVRQAPGKGLEWL | HWYQQKPGQAPRLLIYL |
| | Human | GMIWGDGKIVYNSALK | ASNLESGIPARFSGSGSRT |
| | kappa LC | SRLTISKDSSKNTVYLQ | EFTLTISSLQSEDFAVYYC |
| | | MNSLRAEDTAVYYCA | QQNNEDPRTFGGGTKVEI |
| | | GDGYYPYAMDNWGQ | K (SEQ ID NO: 36) |
| | | GTTVTVSS (SEQ ID NO: | |
| | | 7) | |
| 15 | HC1; LC6; | EVQLQESGPGLVKPSET | DIQLTQSPSSLSASVGDRV |
| | hIgG1- | LSLTCTVSGFSLNAYSV | TITCRASKSVDSYGNSFM |
| | LAGA YTE; | NWIRQPPGKGLEWLG | HWYQQKPGKAPKLLIYL |
| | Human | MIWGDGKIVYNSALKS | ASNLESGVPSRFSGSGSGT |
| | kappa LC | RLTISKDSSKNQVSLKL | DFTLTISSLQPEDFATYYC |
| | | SSVTAADTAVYYCAGD | QQNNEDPRTFGGGTKVEI |
| | | GYYPYAMDNWGQGTT | K (SEQ ID NO: 39) |
| | | VTVSS (SEQ ID NO: 3) | |
| 16 | HC2; LC6; | EVQLVQSGAEVKKPGS | DIQLTQSPSSLSASVGDRV |
| | hIgG1- | SVKVSCKASGFSLNAY | TITCRASKSVDSYGNSFM |
| | LAGA YTE; | SVNWVRQAPGQGLEW | HWYQQKPGKAPKLLIYL |
| | Human | LGMIWGDGKIVYNSAL | ASNLESGVPSRFSGSGSGT |
| | kappa LC | KSRLTITKDSSTSTVYM | DFTLTISSLQPEDFATYYC |
| | | ELSSLRSEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 4) | |
| 17 | HC3; LC6; | EVQLVQSGAEVKKPGS | DIQLTQSPSSLSASVGDRV |
| | hIgG1- | SVKVSCKASGFSLNAY | TITCRASKSVDSYGNSFM |
| | LAGA YTE; | SVNWVRQAPGQGLEW | HWYQQKPGKAPKLLIYL |
| | Human | LGMIWGDGKIVYNSAL | ASNLESGVPSRFSGSGSGT |
| | kappa LC | KSRLTITKDSSTSTVYM | DFTLTISSLQPEDFATYYC |
| | | ELSSLRSEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 5) | |
| 18 | HC4; LC6; | EVQLVESGGGLVKPGG | DIQLTQSPSSLSASVGDRV |
| | hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLKTEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 6) | |
| 19 | HC5; LC6; | EVQLLESGGGLVQPGG | DIQLTQSPSSLSASVGDRV |
| | hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 7) | |
| 22 | HC1; LC7; | EVQLQESGPGLVKPSET | EIVLTQSPATLSVSPGERA |
| | hIgG1- | LSLTCTVSGFSLNAYSV | TLSCRASKSVDSYGNSFM |
| | LAGA YTE; | NWIRQPPGKGLEWLG | HWYQQKPGQAPRLLIYL |
| | Human | MIWGDGKIVYNSALKS | ASNLESGIPARFSGSGSGT |
| | kappa LC | RLTISKDSSKNQVSLKL | EFTLTISSLQSEDFAVYYC |
| | | SSVTAADTAVYYCAGD | QQNNEDPRTFGGGTKVEI |
| | | GYYPYAMDNWGQGTT | K (SEQ ID NO: 40) |
| | | VTVSS (SEQ ID NO: 3) | |
| 21 | HC2; LC7; | EVQLVQSGAEVKKPGS | EIVLTQSPATLSVSPGERA |
| | hIgG1- | SVKVSCKASGFSLNAY | TLSCRASKSVDSYGNSFM |
| | LAGA YTE; | SVNWVRQAPGQGLEW | HWYQQKPGQAPRLLIYL |
| | Human | LGMIWGDGKIVYNSAL | ASNLESGIPARFSGSGSGT |
| | kappa LC | KSRLTITKDSSTSTVYM | EFTLTISSLQSEDFAVYYC |
| | | ELSSLRSEDTAVYYCA | QQNNEDPRTFGGGTKVEI |
| | | GDGYYPYAMDNWGQ | K (SEQ ID NO: 40) |
| | | GTTVTVSS (SEQ ID NO: | |
| | | 4) | |
| 22 | HC3; LC7; | EVQLVQSGAEVKKPGS | EIVLTQSPATLSVSPGERA |
| | hIgG1- | SVKVSCKASGFSLNAY | TLSCRASKSVDSYGNSFM |
| | LAGA YTE; | SVNWVRQAPGQGLEW | HWYQQKPGQAPRLLIYL |
| | Human | LGMIWGDGKIVYNSAL | ASNLESGIPARFSGSGSGT |
| | kappa LC | KSRLTITKDSSTSTVYM | EFTLTISSLQSEDFAVYYC |
| | | ELSSLRSEDTAVYYCA | QQNNEDPRTFGGGTKVEI |
| | | GDGYYPYAMDNWGQ | K (SEQ ID NO: 40) |
| | | GTTVTVSS (SEQ ID NO: | |
| | | 5) | |
| 23 | HC4; LC7; | EVQLVESGGGLVKPGG | EIVLTQSPATLSVSPGERA |
| | hIgG1- | SLRLSCAASGFSLNAYS | TLSCRASKSVDSYGNSFM |
| | LAGA YTE; | VNWVRQAPGKGLEWL | HWYQQKPGQAPRLLIYL |
| | Human | GMIWGDGKIVYNSALK | ASNLESGIPARFSGSGSGT |
| | kappa LC | SRLTISKDSSKNTVYLQ | EFTLTISSLQSEDFAVYYC |
| | | MNSLKTEDTAVYYCA | QQNNEDPRTFGGGTKVEI |
| | | GDGYYPYAMDNWGQ | K (SEQ ID NO: 40) |
| | | GTTVTVSS (SEQ ID NO: | |
| | | 6) | |
| 24 | HC5; LC7; | EVQLLESGGGLVQPGG | EIVLTQSPATLSVSPGERA |
| | hIgG1- | SLRLSCAASGFSLNAYS | TLSCRASKSVDSYGNSFM |
| | LAGA YTE; | VNWVRQAPGKGLEWL | HWYQQKPGQAPRLLIYL |
| | Human | GMIWGDGKIVYNSALK | ASNLESGIPARFSGSGSGT |
| | kappa LC | SRLTISKDSSKNTVYLQ | EFTLTISSLQSEDFAVYYC |
| | | MNSLRAEDTAVYYCA | QQNNEDPRTFGGGTKVEI |
| | | GDGYYPYAMDNWGQ | K (SEQ ID NO: 40) |
| | | GTTVTVSS (SEQ ID NO: | |
| | | 7) | |
| 25 | HC6; LC9; | EVQLQESGPGLVKPSET | DIVLTQSPASLAVSPGERA |
| | hIgG1- | LSLTCTVSGGSLNAYSV | TISCRASKSVDSYGNSFM |
| | LAGA YTE; | NWVRQPPGKGLEWLG | HWYQQKPGQPPKLLIYLA |
| | Human | MIWGDGKIVYNSALKS | SNLESGVPDRFSGSGSGT |
| | kappa LC | RLTISLDTSKSQVFLKM | DFTLTISRVEADDVAVYY |
| | | SSLTAADTAVYYCARD | CQQNNEDPRTFGGGTKLE |
| | | GYYPYAMDNWGQGTT | IK (SEQ ID NO: 42) |
| | | VTVSS (SEQ ID NO: 8) | |
| 26 | HC7; LC10; | QVQLQESGPGLVKPSE | DIVLTQSPASLAVSPGERA |
| | hIgG1- | TLSLTCTVSGGSLNAYS | TISCRASQSVDSNGNNFL |
| | LAGA YTE; | WNWVRQPPGKGLEWL | HWYQQKPGQPPKLLIYLA |
| | Human | GYIYGDGKTNYNPALK | SNRESGVPDRFSGSGSGT |
| | kappa LC | SRLTISLDTSKSQVFLK | DFTLTISRVEADDVAVYY |
| | | MSSLTAADTAVYYCAR | CQQNNHTPRTFGGGTKLE |
| | | DGYYYYAMDVWGQG | IK (SEQ ID NO: 43) |
| | | TTVTVSS (SEQ ID NO: | |
| | | 9) | |
| 90 | HC5; LC6; | EVQLLESGGGLVQPGG | DIQLTQSPSSLSASVGDRV |
| | hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 7) | |
| 91 | HC5; | EVQLLESGGGLVQPGG | DIQLTQSPSSLSASVGDRV |
| | LC6_m1; | SLRLSCAASGFSLNAYS | TITCRASKSVDSYGNSRM |
| | hIgG1- | VNWVRQAPGKGLEWL | HWYQQKPGKAPKLLIYL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNNEDPRTFGGGTKVEI |
| | | GDGYYPYAMDNWGQ | K (SEQ ID NO: 44) |
| | | GTTVTVSS (SEQ ID NO: | |
| | | 7) | |
| 92 | HC5; | EVQLLESGGGLVQPGG | DIQLTQSPSSLSASVGDRV |
| | LC6_m2; | SLRLSCAASGFSLNAYS | TITCRASKSVDSYGNSSM |
| | hIgG1- | VNWVRQAPGKGLEWL | HWYQQKPGKAPKLLIYL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNNEDPRTFGGGTKVEI |
| | | GDGYYPYAMDNWGQ | K (SEQ ID NO: 45) |
| | | GTTVTVSS (SEQ ID NO: | |
| | | 7) | |
| 93 | HC5; | EVQLLESGGGLVQPGG | DIQLTQSPSSLSASVGDRV |
| | LC6_m3; | SLRLSCAASGFSLNAYS | TITCRASKSVDSYGNSFM |
| | hIgG1- | VNWVRQAPGKGLEWL | HWYQQKPGKAPKLLIRL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNNEDPRTFGGGTKVEI |
| | | GDGYYPYAMDNWGQ | K (SEQ ID NO: 46) |
| | | GTTVTVSS (SEQ ID NO: | |
| | | 7) | |
| 94 | HC5; | EVQLLESGGGLVQPGG | DIQLTQSPSSLSASVGDRV |
| | LC6_m4; | SLRLSCAASGFSLNAYS | TITCRASKSVDSYGNSFM |
| | hIgG1- | VNWVRQAPGKGLEWL | HWYQQKPGKAPKLLIFLA |
| | LAGA YTE; | GMIWGDGKIVYNSALK | SNLESGVPSRFSGSGSGTD |
| | Human | SRLTISKDSSKNTVYLQ | FTLTISSLQPEDFATYYCQ |
| | kappa LC | MNSLRAEDTAVYYCA | QNNEDPRTFGGGTKVEIK |
| | | GDGYYPYAMDNWGQ | (SEQ ID NO: 47) |
| | | GTTVTVSS (SEQ ID NO: | |
| | | 7) | |
| 95 | HC5; | EVQLLESGGGLVQPGG | DIQLTQSPSSLSASVGDRV |
| | LC6_m5; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASHLESGVPSRFSGSGSGT |
| | Human | SRLTISKDSSKNTVYLQ | DFTLTISSLQPEDFATYYC |
| | kappa LC | MNSLRAEDTAVYYCA | QQNNEDPRTFGGGTKVEI |
| | | GDGYYPYAMDNWGQ | K (SEQ ID NO: 48) |
| | | GTTVTVSS (SEQ ID NO: | |
| | | 7) | |
| 96 | HC5; | EVQLLESGGGLVQPGG | DIQLTQSPSSLSASVGDRV |
| | LC6_m6; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASDLESGVPSRFSGSGSGT |
| | Human | SRLTISKDSSKNTVYLQ | DFTLTISSLQPEDFATYYC |
| | kappa LC | MNSLRAEDTAVYYCA | QQNNEDPRTFGGGTKVEI |
| | | GDGYYPYAMDNWGQ | K (SEQ ID NO: 49) |
| | | GTTVTVSS (SEQ ID NO: | |
| | | 7) | |
| 97 | HC5; | EVQLLESGGGLVQPGG | DIQLTQSPSSLSASVGDRV |
| | LC6_m7; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASQLESGVPSRFSGSGSGT |
| | Human | SRLTISKDSSKNTVYLQ | DFTLTISSLQPEDFATYYC |
| | kappa LC | MNSLRAEDTAVYYCA | QQNNEDPRTFGGGTKVEI |
| | | GDGYYPYAMDNWGQ | K (SEQ ID NO: 50) |
| | | GTTVTVSS (SEQ ID NO: | |
| | | 7) | |
| 98 | HC5; | EVQLLESGGGLVQPGG | DIQLTQSPSSLSASVGDRV |
| | LC6_m8; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASELESGVPSRFSGSGSGT |
| | Human | SRLTISKDSSKNTVYLQ | DFTLTISSLQPEDFATYYC |
| | kappa LC | MNSLRAEDTAVYYCA | QQNNEDPRTFGGGTKVEI |
| | | GDGYYPYAMDNWGQ | K (SEQ ID NO: 51) |
| | | GTTVTVSS (SEQ ID NO: | |
| | | 7) | |
| 99 | HC5; | EVQLLESGGGLVQPGG | DIQLTQSPSSLSASVGDRV |
| | LC6_m9; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNHEDPRTFGGGTKVEI |
| | | GDGYYPYAMDNWGQ | K (SEQ ID NO: 52) |
| | | GTTVTVSS (SEQ ID NO: | |
| | | 7) | |
| 100 | HC5; | EVQLLESGGGLVQPGG | DIQLTQSPSSLSASVGDRV |
| | LC6_m10; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNYEDPRTFGGGTKVEI |
| | | GDGYYPYAMDNWGQ | K (SEQ ID NO: 53) |
| | | GTTVTVSS (SEQ ID NO: | |
| | | 7) | |
| 101 | HC5; | EVQLLESGGGLVQPGG | DIQLTQSPSSLSASVGDRV |
| | LC6_m11; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNSEDPRTFGGGTKVEI |
| | | GDGYYPYAMDNWGQ | K (SEQ ID NO: 54) |
| | | GTTVTVSS (SEQ ID NO: | |
| | | 7) | |
| 102 | HC5; | EVQLLESGGGLVQPGG | DIQLTQSPSSLSASVGDRV |
| | LC6_m12; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNNRDPRTFGGGTKVEI |
| | | GDGYYPYAMDNWGQ | K (SEQ ID NO: 55) |
| | | GTTVTVSS (SEQ ID NO: | |
| | | 7) | |
| 103 | HC5; | EVQLLESGGGLVQPGG | DIQLTQSPSSLSASVGDRV |
| | LC6_m13; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNNDDPRTFGGGTKVEI |
| | | GDGYYPYAMDNWGQ | K (SEQ ID NO: 56) |
| | | GTTVTVSS (SEQ ID NO: | |
| | | 7) | |
| 104 | HC5; | EVQLLESGGGLVQPGG | DIQLTQSPSSLSASVGDRV |
| | LC6_m14; | SLRLSCAASGFSLNAYS | TITCRASKSVDSYGNSFM |
| | hIgG1- | VNWVRQAPGKGLEWL | HWYQQKPGKAPKLLIYL |
| | LAGA YTE; | GMIWGDGKIVYNSALK | ASNLESGVPSRFSGSGSGT |
| | Human | SRLTISKDSSKNTVYLQ | DFTLTISSLQPEDFATYYC |
| | kappa LC | MNSLRAEDTAVYYCA | QQNNQDPRTFGGGTKVEI |
| | | GDGYYPYAMDNWGQ | K (SEQ ID NO: 57) |
| | | GTTVTVSS (SEQ ID NO: | |
| | | 7) | |
| 105 | HC5_m1; | EVQLLESGGGLVQPGG | DIQLTQSPSSLSASVGDRV |
| | LC6; hIgG1- | SLRLSCAASGYSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 10) | |
| 106 | HC5_m2; | EVQLLESGGGLVQPGG | DIQLTQSPSSLSASVGDRV |
| | LC6; hIgG1- | SLRLSCAASGFSLRAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 11) | |
| 107 | HC5_m3; | EVQLLESGGGLVQPGG | DIQLTQSPSSLSASVGDRV |
| | LC6; hIgG1- | SLRLSCAASGFSLHAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 12) | |
| 108 | HC5_m4; | EVQLLESGGGLVQPGG | DIQLTQSPSSLSASVGDRV |
| | LC6; hIgG1- | SLRLSCAASGFSLDAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 13) | |
| 109 | HC5_m5; | EVQLLESGGGLVQPGG | DIQLTQSPSSLSASVGDRV |
| | LC6; hIgG1- | SLRLSCAASGFSLYAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 14) | |
| 110 | HC5_m6; | EVQLLESGGGLVQPGG | DIQLTQSPSSLSASVGDRV |
| | LC6; hIgG1- | SLRLSCAASGFSLSAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 15) | |
| 111 | HC5_m7; | EVQLLESGGGLVQPGG | DIQLTQSPSSLSASVGDRV |
| | LC6; hIgG1- | SLRLSCAASGFSLNRYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 16) | |
| 112 | HC5_m8; | EVQLLESGGGLVQPGG | DIQLTQSPSSLSASVGDRV |
| | LC6; hIgG1- | SLRLSCAASGFSLNKYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 17) | |
| 113 | HC5_m9; | EVQLLESGGGLVQPGG | DIQLTQSPSSLSASVGDRV |
| | LC6; hIgG1- | SLRLSCAASGFSLNHYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 18) | |
| 114 | HC5_m10; | EVQLLESGGGLVQPGG | DIQLTQSPSSLSASVGDRV |
| | LC6; hIgG1- | SLRLSCAASGFSLNQYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 19) | |
| 115 | HC5_m11; | EVQLLESGGGLVQPGG | DIQLTQSPSSLSASVGDRV |
| | LC6; hIgG1- | SLRLSCAASGFSLNEYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 20) | |
| 116 | HC5_m12; | EVQLLESGGGLVQPGG | DIQLTQSPSSLSASVGDRV |
| | LC6; hIgG1- | SLRLSCAASGFSLNSYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 21) | |
| 117 | HC5_m13; | EVQLLESGGGLVQPGG | DIQLTQSPSSLSASVGDRV |
| | LC6; hIgG1- | SLRLSCAASGFSLNYYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 22) | |
| 118 | HC5_m14; | EVQLLESGGGLVQPGG | DIQLTQSPSSLSASVGDRV |
| | LC6; hIgG1- | SLRLSCAASGFSLNAES | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 23) | |
| 119 | HC5_m15; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWSDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 24) | |
| 120 | HC5_m16; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWADGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYPYAMDNWGQ | K (SEQ ID NO: 39) |
| | | GTTVTVSS (SEQ ID NO: | |
| | | 25) | |
| 121 | HC5_m17; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GHGYYPYAMDNWGQ | K (SEQ ID NO: 39) |
| | | GTTVTVSS (SEQ ID NO: | |
| | | 26) | |
| 122 | HC5_m18; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDLYYPYAMDNWGQG | K (SEQ ID NO: 39) |
| | | TTVTVSS (SEQ ID NO: | |
| | | 27) | |
| 123 | HC5_m19; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDKYYPYAMDNWGQ | K (SEQ ID NO: 39) |
| | | GTTVTVSS (SEQ ID NO: | |
| | | 28) | |
| 124 | HC5_m20; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYGYAMDNWGQ | K (SEQ ID NO: 39) |
| | | GTTVTVSS (SEQ ID NO: | |
| | | 29) | |
| 125 | HC5_m21; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYAYAMDNWGQ | K (SEQ ID NO: 39) |
| | | GTTVTVSS (SEQ ID NO: | |
| | | 30) | |
| 126 | HC5_m22; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYSYAMDNWGQ | K (SEQ ID NO: 39) |
| | | GTTVTVSS (SEQ ID NO: | |
| | | 31) | |
| 127 | HC5_m23; | EVQLLESGGGLVQPGG | DIQLTQSPSSLSASVGDRV |
| | LC6; hIgG1- | SLRLSCAASGFSLNAYS | TITCRASKSVDSYGNSFM |
| | LAGA YTE; | VNWVRQAPGKGLEWL | HWYQQKPGKAPKLLIYL |
| | Human | GMIWGDGKIVYNSALK | ASNLESGVPSRFSGSGSGT |
| | kappa LC | SRLTISKDSSKNTVYLQ | DFTLTISSLQPEDFATYYC |
| | | MNSLRAEDTAVYYCA | QQNNEDPRTFGGGTKVEI |
| | | GDGYYTYAMDNWGQ | K (SEQ ID NO: 39) |
| | | GTTVTVSS (SEQ ID NO: | |
| | | 32) | |
| 128 | Lebrikizumab- | QVTLRESGPALVKPTQ | DIVMTQSPDSL |
| | HC; | TLTLTCTVSGFSLSAYS | SVSLGERATIN |
| | Lebrikizumab- | VNWIRQPPGKALEWL | CRASKSVDSY |
| | LC; | AMIWGDGKIVYNSAL | GNSFMHWYQ |
| | hIgG4-YTE; | KSRLTISKDTSKNQVV | QKPGQPPKLLI |
| | Human | LTMTNMDPVDTATYY | YLASNLESGVP |
| | kappa LC | CAGDGYYPYAMDNW | DRFSGSGSGTD |
| | | GQGSLVTVSS (SEQ ID | FTLTISSLQAED |
| | | NO: 470) | VAVYYCQQNN |
| | | | EDPRTFGGGTK |
| | | | VEIK (SEQ ID |
| | | | NO: 471) |
| 129 | Lebrikizumab- | QVTLRESGPALVKPTQ | DIVMTQSPDSL |
| | HC; | TLTLTCTVSGFSLSAYS | SVSLGERATIN |
| | Lebrikizumab- | VNWIRQPPGKALEWL | CRASKSVDSY |
| | LC; | AMIWGDGKIVYNSAL | GNSFMHWYQ |
| | hIgG4-LS; | KSRLTISKDTSKNQVV | QKPGQPPKLLI |
| | Human | LTMTNMDPVDTATYY | YLASNLESGVP |
| | kappa LC | CAGDGYYPYAMDNW | DRFSGSGSGTD |
| | | GQGSLVTVSS (SEQ ID | FTLTISSLQAED |
| | | NO: 470) | VAVYYCQQNN |
| | | | EDPRTFGGGTK |
| | | | VEIK (SEQ ID |
| | | | NO: 471) |
| 130 | Lebrikizumab- | QVTLRESGPALVKPTQ | DIVMTQSPDSL |
| | HC; | TLTLTCTVSGFSLSAYS | SVSLGERATIN |
| | Lebrikizumab- | VNWIRQPPGKALEWL | CRASKSVDSY |
| | LC; | AMIWGDGKIVYNSAL | GNSFMHWYQ |
| | hIgG1- | KSRLTISKDTSKNQVV | QKPGQPPKLLI |
| | LALA-YTE; | LTMTNMDPVDTATYY | YLASNLESGVP |
| | Human | CAGDGYYPYAMDNW | DRFSGSGSGTD |
| | kappa LC | GQGSLVTVSS (SEQ ID | FTLTISSLQAED |
| | | NO: 470) | VAVYYCQQNN |
| | | | EDPRTFGGGTK |
| | | | VEIK (SEQ ID |
| | | | NO: 471) |
| 131 | Lebrikizumab- | QVTLRESGPALVKPTQ | DIVMTQSPDSL |
| | HC; | TLTLTCTVSGFSLSAYS | SVSLGERATIN |
| | Lebrikizumab- | VNWIRQPPGKALEWL | CRASKSVDSY |
| | LC; | AMIWGDGKIVYNSAL | GNSFMHWYQ |
| | hIgG1- | KSRLTISKDTSKNQVV | QKPGQPPKLLI |
| | LALA-LS; | LTMTNMDPVDTATYY | YLASNLESGVP |
| | Human | CAGDGYYPYAMDNW | DRFSGSGSGTD |
| | kappa LC | GQGSLVTVSS (SEQ ID | FTLTISSLQAED |
| | | NO: 470) | VAVYYCQQNN |
| | | | EDPRTFGGGTK |
| | | | VEIK (SEQ ID |
| | | | NO: 471) |
| 132 | HC5; LC6; | EVQLLESGGGLVQPGG | DIQLTQSPSSLS |
| | hIgG1- | SLRLSCAASGFSLNAY | ASVGDRVTITC |
| | LALA YTE; | SVNWVRQAPGKGLEW | RASKSVDSYG |
| | Human | LGMIWGDGKIVYNSA | NSFMHWYQQ |
| | kappa LC | LKSRLTISKDSSKNTVY | KPGKAPKLLIY |
| | | LQMNSLRAEDTAVYY | LASNLESGVPS |
| | | CAGDGYYPYAMDNW | RFSGSGSGTDF |
| | | GQGTTVTVSS (SEQ ID | TLTISSLQPEDF |
| | | NO: 7) | ATYYCQQNNE |
| | | | DPRTFGGGTK |
| | | | VEIK (SEQ ID |
| | | | NO: 39) |
| 133 | HC1; LC6; | EVQLQESGPGLVKPSE | DIQLTQSPSSLS |
| | hIgG1- | TLSLTCTVSGFSLNAYS | ASVGDRVTITC |
| | LALA YTE; | VNWIRQPPGKGLEWL | RASKSVDSYG |
| | Human | GMIWGDGKIVYNSAL | NSFMHWYQQ |
| | kappa LC | KSRLTISKDSSKNQVSL | KPGKAPKLLIY |
| | | KLSSVTAADTAVYYC | LASNLESGVPS |
| | | AGDGYYPYAMDNWG | RFSGSGSGTDF |
| | | QGTTVTVSS (SEQ ID | TLTISSLQPEDF |
| | | NO: 3) | ATYYCQQNNE |
| | | | DPRTFGGGTK |
| | | | VEIK (SEQ ID |
| | | | NO: 39) |
| 134 | HC1; LC6; | EVQLQESGPGLVKPSE | DIQLTQSPSSLS |
| | hIgG4-YTE; | TLSLTCTVSGFSLNAYS | ASVGDRVTITC |
| | Human | VNWIRQPPGKGLEWL | RASKSVDSYG |
| | kappa LC | GMIWGDGKIVYNSAL | NSFMHWYQQ |
| | | KSRLTISKDSSKNQVSL | KPGKAPKLLIY |
| | | KLSSVTAADTAVYYC | LASNLESGVPS |
| | | AGDGYYPYAMDNWG | RFSGSGSGTDF |
| | | QGTTVTVSS (SEQ ID | TLTISSLQPEDF |
| | | NO: 3) | ATYYCQQNNE |
| | | | DPRTFGGGTK |
| | | | VEIK (SEQ ID |
| | | | NO: 39) |
| 135 | HC1; LC6; | EVQLQESGPGLVKPSE | DIQLTQSPSSLS |
| | hIgG4-LS; | TLSLTCTVSGFSLNAYS | ASVGDRVTITC |
| | Human | VNWIRQPPGKGLEWL | RASKSVDSYG |
| | kappa LC | GMIWGDGKIVYNSAL | NSFMHWYQQ |
| | | KSRLTISKDSSKNQVSL | KPGKAPKLLIY |
| | | KLSSVTAADTAVYYC | LASNLESGVPS |
| | | AGDGYYPYAMDNWG | RFSGSGSGTDF |
| | | QGTTVTVSS (SEQ ID | TLTISSLQPEDF |
| | | NO: 3) | ATYYCQQNNE |
| | | | DPRTFGGGTK |
| | | | VEIK (SEQ ID |
| | | | NO: 39) |
| 136 | HC1; LC6; | EVQLQESGPGLVKPSE | DIQLTQSPSSLS |
| | hIgG1- | TLSLTCTVSGFSLNAYS | ASVGDRVTITC |
| | LALA LS; | VNWIRQPPGKGLEWL | RASKSVDSYG |
| | Human | GMIWGDGKIVYNSAL | NSFMHWYQQ |
| | kappa LC | KSRLTISKDSSKNQVSL | KPGKAPKLLIY |
| | | KLSSVTAADTAVYYC | LASNLESGVPS |
| | | AGDGYYPYAMDNWG | RFSGSGSGTDF |
| | | QGTTVTVSS (SEQ ID | TLTISSLQPEDF |
| | | NO: 3) | ATYYCQQNNE |
| | | | DPRTFGGGTK |
| | | | VEIK (SEQ ID |
| | | | NO: 39) |
| 137 | HC5; | EVQLLESGGGLVQPGG | DIQLTQSPSSLS |
| | LC6_m8; | SLRLSCAASGFSLNAY | ASVGDRVTITC |
| | hIgG1- | SVNWVRQAPGKGLEW | RASKSVDSYG |
| | LALA YTE; | LGMIWGDGKIVYNSA | NSFMHWYQQ |
| | Human | LKSRLTISKDSSKNTVY | KPGKAPKLLIY |
| | kappa LC | LQMNSLRAEDTAVYY | LASELESGVPS |
| | | CAGDGYYPYAMDNW | RFSGSGSGTDF |
| | | GQGTTVTVSS (SEQ ID | TLTISSLQPEDF |
| | | NO: 7) | ATYYCQQNNE |
| | | | DPRTFGGGTK |
| | | | VEIK (SEQ ID |
| | | | NO: 51) |
| 138 | HC5; | EVQLLESGGGLVQPGG | DIQLTQSPSSLS |
| | LC6_m8; | SLRLSCAASGFSLNAY | ASVGDRVTITC |
| | hIgG4-YTE; | SVNWVRQAPGKGLEW | RASKSVDSYG |
| | Human | LGMIWGDGKIVYNSA | NSFMHWYQQ |
| | kappa LC | LKSRLTISKDSSKNTVY | KPGKAPKLLIY |
| | | LQMNSLRAEDTAVYY | LASELESGVPS |
| | | CAGDGYYPYAMDNW | RFSGSGSGTDF |
| | | GQGTTVTVSS (SEQ ID | TLTISSLQPEDF |
| | | NO: 7) | ATYYCQQNNE |
| | | | DPRTFGGGTK |
| | | | VEIK (SEQ ID |
| | | | NO: 51) |
| 139 | HC5; | EVQLLESGGGLVQPGG | DIQLTQSPSSLS |
| | LC6_m8; | SLRLSCAASGFSLNAY | ASVGDRVTITC |
| | hIgG4-LS; | SVNWVRQAPGKGLEW | RASKSVDSYG |
| | Human | LGMIWGDGKIVYNSA | NSFMHWYQQ |
| | kappa LC | LKSRLTISKDSSKNTVY | KPGKAPKLLIY |
| | | LQMNSLRAEDTAVYY | LASELESGVPS |
| | | CAGDGYYPYAMDNW | RFSGSGSGTDF |
| | | GQGTTVTVSS (SEQ ID | TLTISSLQPEDF |
| | | NO: 7) | ATYYCQQNNE |
| | | | DPRTFGGGTK |
| | | | VEIK (SEQ ID |
| | | | NO: 51) |
| 140 | HC5; | EVQLLESGGGLVQPGG | DIQLTQSPSSLS |
| | LC6_m8; | SLRLSCAASGFSLNAY | ASVGDRVTITC |
| | hIgG1- | SVNWVRQAPGKGLEW | RASKSVDSYG |
| | LALA LS; | LGMIWGDGKIVYNSA | NSFMHWYQQ |
| | Human | LKSRLTISKDSSKNTVY | KPGKAPKLLIY |
| | kappa LC | LQMNSLRAEDTAVYY | LASELESGVPS |
| | | CAGDGYYPYAMDNW | RFSGSGSGTDF |
| | | GQGTTVTVSS (SEQ ID | TLTISSLQPEDF |
| | | NO: 7) | ATYYCQQNNE |
| | | | DPRTFGGGTK |
| | | | VEIK (SEQ ID |
| | | | NO: 51) |
| 141 | HC1; | EVQLQESGPGLVKPSE | DIQLTQSPSSLS |
| | LC6_m8; | TLSLTCTVSGFSLNAYS | ASVGDRVTITC |
| | hIgG1- | VNWIRQPPGKGLEWL | RASKSVDSYG |
| | LALA YTE; | GMIWGDGKIVYNSAL | NSFMHWYQQ |
| | Human | KSRLTISKDSSKNQVSL | KPGKAPKLLIY |
| | kappa LC | KLSSVTAADTAVYYC | LASELESGVPS |
| | | AGDGYYPYAMDNWG | RFSGSGSGTDF |
| | | QGTTVTVSS (SEQ ID | TLTISSLQPEDF |
| | | NO: 3) | ATYYCQQNNE |
| | | | DPRTFGGGTK |
| | | | VEIK (SEQ ID |
| | | | NO: 51) |
| 142 | HC1; | EVQLQESGPGLVKPSE | DIQLTQSPSSLS |
| | LC6_m8; | TLSLTCTVSGFSLNAYS | ASVGDRVTITC |
| | hIgG4 YTE; | VNWIRQPPGKGLEWL | RASKSVDSYG |
| | Human | GMIWGDGKIVYNSAL | NSFMHWYQQ |
| | kappa LC | KSRLTISKDSSKNQVSL | KPGKAPKLLIY |
| | | KLSSVTAADTAVYYC | LASELESGVPS |
| | | AGDGYYPYAMDNWG | RFSGSGSGTDF |
| | | QGTTVTVSS (SEQ ID | TLTISSLQPEDF |
| | | NO: 3) | ATYYCQQNNE |
| | | | DPRTFGGGTK |
| | | | VEIK (SEQ ID |
| | | | NO: 51) |
| 143 | HC1; | EVQLQESGPGLVKPSE | DIQLTQSPSSLS |
| | LC6_m8; | TLSLTCTVSGFSLNAYS | ASVGDRVTITC |
| | hIgG4 LS; | VNWIRQPPGKGLEWL | RASKSVDSYG |
| | Human | GMIWGDGKIVYNSAL | NSFMHWYQQ |
| | kappa LC | KSRLTISKDSSKNQVSL | KPGKAPKLLIY |
| | | KLSSVTAADTAVYYC | LASELESGVPS |
| | | AGDGYYPYAMDNWG | RFSGSGSGTDF |
| | | QGTTVTVSS (SEQ ID | TLTISSLQPEDF |
| | | NO: 3) | ATYYCQQNNE |
| | | | DPRTFGGGTK |
| | | | VEIK (SEQ ID |
| | | | NO: 51) |
| 144 | HC1; | EVQLQESGPGLVKPSE | DIQLTQSPSSLS |
| | LC6_m8; | TLSLTCTVSGFSLNAYS | ASVGDRVTITC |
| | hIgG1- | VNWIRQPPGKGLEWL | RASKSVDSYG |
| | LALA LS; | GMIWGDGKIVYNSAL | NSFMHWYQQ |
| | Human | KSRLTISKDSSKNQVSL | KPGKAPKLLIY |
| | kappa LC | KLSSVTAADTAVYYC | LASELESGVPS |
| | | AGDGYYPYAMDNWG | RFSGSGSGTDF |
| | | QGTTVTVSS (SEQ ID | TLTISSLQPEDF |
| | | NO: 3) | ATYYCQQNNE |
| | | | DPRTFGGGTK |
| | | | VEIK (SEQ ID |
| | | | NO: 51) |

*Names correspond with name in informal sequence listing

In some embodiments, such a IgG4-SP HC constant domain has the sequence:

(SEQ ID NO: 427)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFLGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK.

In some embodiments, such a hIgG1-LALA-YTE HC constant domain has the sequence:

(SEQ ID NO: 439)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLY
ITREPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG.

In some embodiments, such a hIgG1-LAGA YTE HC constant domain has the sequence:

(SEQ ID NO: 440)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELAGAPSVFLFPPKPKDTLY
ITREPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG.

In some embodiments, such a hIgG1-LALA-LS HC constant domain has the sequence:

(SEQ ID NO: 446)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVLHEAL
HSHYTQKSLSLSPG.

In some embodiments, such a IgG4-YTE HC constant domain has the sequence:

(SEQ ID NO: 457)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPSCPAPEFLGGPSVFLFPPKPKDTLYITR
EPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK.

In some embodiments, such a IgG4-LS HC constant domain has the sequence:

(SEQ ID NO: 460)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPSCPAPEFLGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVLHEALHSY
TQKSLSLSLGK.

In some embodiments, such a human kappa LC constant domain has the sequence:

(SEQ ID NO: 469)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC.

### CDRs

In some embodiments, an antibody provided herein comprises one to three CDRs of a VH domain selected from SEQ ID NOs: 1-32 and 470, such as any of the CDRs listed in Table 3, Table 4, or Table 5, below. In some embodiments, an antibody provided herein comprises two to three CDRs of a VH domain selected from SEQ ID NOs: 1-32 and 470. In some embodiments, an antibody provided herein comprises three CDRs of a VH domain selected from SEQ ID NOs: 1-32 and 470. In some embodiments, the CDRs are Exemplary CDRs. In some embodiments, the CDRs are Kabat CDRs. In some embodiments, the CDRs are Chothia CDRs. In some embodiments, the CDRs are IMGT CDRs. In some embodiments, the CDRs are AbM CDRs. In some embodiments, the CDRs are Contact CDRs.

In some embodiments, the CDRs are CDRs having at least about 80%, 90%, 91%, 92%, 93%, 94%, 95%, 96%, 97%, 98%, 99% or 100% identity with a CDR-H1, CDR-H2, or CDR-H3 of SEQ ID NOs: 58-140. In some embodiments, the CDR-H1 is a CDR-H1 of a VH domain selected from SEQ ID NOs: 1-32 and 470, with up to 1, 2, 3, 4, or 5 amino acid substitutions. In some embodiments, the CDR-H2 is a CDR-H2 of a VH domain of SEQ ID NO: 1-32 and 470, with up to 1, 2, 3, 4, 5, 6, 7, or 8 amino acid substitutions. In some embodiments, the CDR-H3 is a CDR-H3 of a VH domain selected from SEQ ID NOs: 1-32 and 470, with up to 1, 2, 3, 4, 5, 6, 7, or 8 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a VL domain of SEQ ID NOs: 33-57 and 471, such as any of the CDRs listed in Table 6, Table 7, or Table 8, below. In some embodiments, an antibody provided herein comprises two to three CDRs of a VL domain of SEQ ID NOs: 33-57 and 471. In some embodiments, an antibody provided herein comprises three CDRs of a VL domain of SEQ ID NOs: 33-57 and 471. In some embodiments, the CDRs are Exemplary CDRs. In some embodiments, the CDRs are Kabat CDRs. In some embodiments, the CDRs are Chothia CDRs. In some embodiments, the CDRs are IMGT CDRs. In some embodiments, the CDRs are AbM CDRs. In some embodiments, the CDRs are Contact CDRs.

In some embodiments, the CDRs are CDRs having at least about 80%, 90%, 91%, 92%, 93%, 94%, 95%, 96%, 97%, 98%, 99% or 100% identity with a CDR-L1, CDR-L2, or CDR-L3 of SEQ ID NOs: 141-188. In some embodiments, the CDR-L1 is a CDR-L1 of a VL domain of SEQ ID NOs: 33-57 and 471, with up to 1, 2, 3, 4, or 5 amino acid substitutions. In some embodiments, the CDR-L2 is a CDR-L2 of a VL domain of SEQ ID NOs: 33-57 and 471, with up to 1, 2, 3, 4, 5, 6, 7, or 8 amino acid substitutions. In some embodiments, the CDR-L3 is a CDR-L3 of a VL domain of SEQ ID NOs: 33-57 and 471, with up to 1, 2, 3, 4, 5, 6, 7, or 8 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a VH domain selected from SEQ ID NOs: 1-32 and 470 and one to three CDRs of a VL domain of SEQ ID NOs: 33-57 and 471. In some embodiments, an antibody provided herein comprises two to three CDRs of a VH domain selected from SEQ ID NOs: 1-32 and 470 and two to three CDRs of a VL domain of SEQ ID NOs: 33-57 and 471. In some embodiments, an antibody provided herein comprises three CDRs of a VH domain selected from SEQ ID NOs: 1-32 and 470 and three CDRs of a VL domain of SEQ ID NOs: 33-57 and 471. In some embodiments, the CDRs are Exemplary CDRs. In some embodiments, the CDRs are Kabat CDRs. In some embodiments, the CDRs are Chothia CDRs. In some embodiments, the CDRs are IMGT CDRs. In some embodiments, the CDRs are AbM CDRs. In some embodiments, the CDRs are Contact CDRs.

In some embodiments, an antibody provided herein comprises a CDR-H3 selected of SEQ ID NOs: 112-120 and 130-40. In some embodiments, the CDR-H3 has at least about 80%, 90%, 91%, 92%, 93%, 94%, 95%, 96%, 97%, 98% or 99% identity with a CDR-H3 of SEQ ID NOs: 112-120 or 130-40. In some embodiments, the CDR-H3 is a CDR-H3 selected of SEQ ID NOs: 112-120 and 130-40, with up to 1, 2, 3, 4, 5, 6, 7, or 8 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 of SEQ ID NOs: 58-99 and 121. In some embodiments, the CDR-H1 has at least about 80%, 90%, 91%, 92%, 93%, 94%, 95%, 96%, 97%, 98% or 99% identity with a CDR-H1 of SEQ ID NOs: 58-99 or 121. In some embodiments, the CDR-H1 is a CDR-H1 of SEQ ID NOs: 58-99 or 121, with up to 1, 2, 3, 4, 5, 6, 7, or 8 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H2 of any one of SEQ ID NOs: 100-111. In some embodiments, the CDR-H2 has at least about 80%, 90%, 91%, 92%, 93%, 94%, 95%, 96%, 97%, 98% or 99% identity with a CDR-H2 of any one of SEQ ID NOs: 100-111. In some embodiments, the CDR-H2 is a CDR-H2 of any one of SEQ ID NOs: 100-111, with up to 1, 2, 3, 4, 5, 6, 7, or 8 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 selected from SEQ ID NOs: 165-172. In some embodiments, the CDR-L3 has at least about 80%, 90%, 91%, 92%, 93%, 94%, 95%, 96%, 97%, 98% or 99% identity with a CDR-L3 of SEQ ID NOs: 165-172. In some embodiments, the CDR-L3 is a CDR-L3 of SEQ ID NOs: 165-172, with up to 1, 2, 3, 4, 5, 6, 7, or 8 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L2 selected from SEQ ID NOs: 153-158 and the amino acid sequence LAS. In some embodiments, the CDR-L2 has at least about 80%, 90%, 91%, 92%, 93%, 94%, 95%, 96%, 97%, 98% or 99% identity with a CDR-L2 selected from SEQ ID NOs: 153-158 and the amino acid sequence LAS. In some embodiments, the CDR-L2 is a CDR-L2 selected from SEQ ID NOs: 153-158 and the amino acid sequence LAS, with up to 1, 2, 3, 4, 5, 6, 7, or 8 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L1 selected from SEQ ID NOs: 141-144 and 149-152. In some embodiments, the CDR-L1 has at least about 80%, 90%, 91%, 92%, 93%, 94%, 95%, 96%, 97%, 98% or 99% identity with a CDR-L1 selected from SEQ ID NOs: 141-144 and 149-152. In some embodiments, the CDR-L1 is a CDR-L1 selected from SEQ ID NOs: 141-144 and 149-152, with up to 1, 2, 3, 4, 5, 6, 7, or 8 amino acid substitutions. In some embodiments, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 112-120 and 130-140, a CDR-H2 of SEQ ID NOs: 100-111, a CDR-H1 selected from SEQ ID NOs: 58-99 and 121, a CDR-L3 selected from SEQ ID NOs: 165-172, a CDR-L2 selected from SEQ ID NOs: 153-158 and the amino acid sequence LAS, and a CDR-L1 selected from SEQ ID NOs: 141-144 and 149-152. In some embodiments, the CDR-H3 has at least about 80%, 90%, 91%, 92%, 93%, 94%, 95%, 96%, 97%, 98% or 99% identity with a CDR-H3 selected from SEQ ID NOs: 112-120 and 130-140, the CDR-H2 has at least about 80%, 90%, 91%, 92%, 93%, 94%, 95%, 96%, 97%, 98% or 99% identity with a CDR-H2 of SEQ ID NOs: 100-111, the CDR-H1 has at least about 80%, 90%, 91%, 92%, 93%, 94%, 95%, 96%, 97%, 98% or 99% identity with a CDR-H1 selected from SEQ ID NOs: 58-99 and 121, the CDR-L3 has at least about 80%, 90%, 91%, 92%, 93%, 94%, 95%, 96%, 97%, 98% or 99% identity with a CDR-L3 selected from SEQ ID NOs: 165-172, the CDR-L2 has at least about 80%, 90%, 91%, 92%, 93%, 94%, 95%, 96%, 97%, 98% or 99% identity with a CDR-L2 selected from SEQ ID NOs: 153-158 and the amino acid sequence LAS, and the CDR-L1 has at least about 80%, 90%, 91%, 92%, 93%, 94%, 95%, 96%, 97%, 98% or 99% identity with a CDR-L1 selected from SEQ ID NOs: 141-144 and 149-152. In some embodiments, the CDR-H3 is a CDR-H3 selected from SEQ ID NOs: 112-120 and 130-140, with up to 1, 2, 3, 4, 5, 6, 7, or 8 amino acid substitutions; the CDR-H2 is a CDR-H2 of SEQ ID NOs: 100-111, with up to 1, 2, 3, 4, 5, 6, 7, or 8 amino acid substitutions; the CDR-H1 is a CDR-H1 selected from SEQ ID NOs: 58-99 and 121, with up to 1, 2, 3, 4, or 5 amino acid substitutions; the CDR-L3 is a CDR-L3 selected from SEQ ID NOs: 165-172, with up to 1, 2, 3, 4, or 5 amino acid substitutions; the CDR-L2 is a CDR-L2 selected from SEQ ID NOs: 153-158 and the amino acid sequence LAS, with up to 1, 2, 3, or 4 amino acid substitutions; and the CDR-L1 is a CDR-L1 selected from SEQ ID NOs: 141-144 and 149-152, with up to 1, 2, 3, 4, 5, or 6 amino acid substitutions.

In some embodiments, an antibody provided herein comprises a CDR-H3 of SEQ ID NOs: 112, 121, and 130, a CDR-H2 of SEQ ID NOs: 100, 104, and 108, a CDR-H1 of SEQ ID NOs: 58, 68, and 85, a CDR-L3 of SEQ ID NOs: 168, 173, and 181, a CDR-L2 of SEQ ID NOs: 153 and the amino acid sequence LAS, and a CDR-L1 of SEQ ID NOs: 141 and 149. In some embodiments, the CDR-H3 has at least about 80%, 90%, 91%, 92%, 93%, 94%, 95%, 96%, 97%, 98% or 99% identity with a CDR-H3 of SEQ ID NOs: 112 or 130, the CDR-H2 has at least about 80%, 90%, 91%, 92%, 93%, 94%, 95%, 96%, 97%, 98% or 99% identity with a CDR-H2 of SEQ ID NOs: 100, 104 or 108, the CDR-H1 has at least about 80%, 90%, 91%, 92%, 93%, 94%, 95%, 96%, 97%, 98% or 99% identity with a CDR-H1 of SEQ ID NOs: 58, 68 or 85, the CDR-L3 has at least about 80%, 90%, 91%, 92%, 93%, 94%, 95%, 96%, 97%, 98% or 99% identity with a CDR-L3 of SEQ ID NO: 168, the CDR-L2 has at least about 80%, 90%, 91%, 92%, 93%, 94%, 95%, 96%, 97%, 98% or 99% identity with a CDR-L2 of SEQ ID NOs: 153 or the amino acid sequence LAS, and the CDR-L1 has at least about 80%, 90%, 91%, 92%, 93%, 94%, 95%, 96%, 97%, 98% or 99% identity with a CDR-L1 of SEQ ID NOs: 141 or 149. In some embodiments, the CDR-H3 is a CDR-H3 of SEQ ID NOs: 112 or 130, with up to 1, 2, 3, 4, 5, 6, 7, or 8 amino acid substitutions; the CDR-H2 is a CDR-H2 of SEQ ID NOs: 100, 104 or 108, with up to 1, 2, 3, 4, 5, 6, 7, or 8 amino acid substitutions; the CDR-H1 is a CDR-H1 of SEQ ID NOs: 58, 68 or 85, with up to 1, 2, 3, 4, or 5 amino acid substitutions; the CDR-L3 is a CDR-L3 of SEQ ID NO: 168 with up to 1, 2, 3, 4, or 5 amino acid substitutions; the CDR-L2 is a CDR-L2 of SEQ ID NOs: 153 or the amino acid sequence LAS, with up to 1, 2, 3, or 4 amino acid substitutions; and the CDR-L1 is a CDR-L1 of SEQ ID NOs: 141 or 149, with up to 1, 2, 3, 4, 5, or 6 amino acid substitutions.

In some embodiments, an antibody provided herein comprises a CDR-H3 of SEQ ID NOs: 112, 121 or 130, a CDR-H2 of SEQ ID NOs: 100, 104 or 108, a CDR-H1 of SEQ ID NOs: 58, 68, or 85, a CDR-L3 of SEQ ID NO: 165, a CDR-L2 of SEQ ID NOs: 153 or the amino acid sequence LAS, and a CDR-L1 of SEQ ID NOs: 141 or 149. In some embodiments, the CDR-H3 has at least about 80%, 90%, 91%, 92%, 93%, 94%, 95%, 96%, 97%, 98% or 99% identity with a CDR-H3 of SEQ ID NOs: 112 or 130, the CDR-H2 has at least about 80%, 90%, 91%, 92%, 93%, 94%, 95%, 96%, 97%, 98% or 99% identity with a CDR-H2 of SEQ ID NOs: 100, 104 or 108, the CDR-H1 has at least about 80%, 90%, 91%, 92%, 93%, 94%, 95%, 96%, 97%, 98% or 99% identity with a CDR-H1 of SEQ ID NOs: 58, 68 or 85, the CDR-L3 has at least about 80%, 90%, 91%, 92%, 93%, 94%, 95%, 96%, 97%, 98% or 99% identity with a CDR-L3 of SEQ ID NO: 165, the CDR-L2 has at least about 80%, 90%, 91%, 92%, 93%, 94%, 95%, 96%, 97%, 98% or 99% identity with a CDR-L2 of SEQ ID NOs: 153 or the amino acid sequence LAS, and the CDR-L1 has at least about 80%, 90%, 91%, 92%, 93%, 94%, 95%, 96%, 97%, 98% or 99% identity with a CDR-L1 of SEQ ID NOs: 141 or 149. In some embodiments, the CDR-H3 is a CDR-H3 of SEQ ID NOs: 112 or 130, with up to 1, 2, 3, 4, 5, 6, 7, or 8 amino acid substitutions; the CDR-H2 is a CDR-H2 of SEQ ID NOs: 100, 104 or 108, with up to 1, 2, 3, 4, 5, 6, 7, or 8 amino acid substitutions; the CDR-H1 is a CDR-H1 of SEQ ID NOs: 58, 68 or 85, with up to 1, 2, 3, 4, or 5 amino acid substitutions; the CDR-L3 is a CDR-L3 of SEQ ID NO: 165, with up to 1, 2, 3, 4, or 5 amino acid substitutions; the CDR-L2 is a CDR-L2 of SEQ ID NO: 153 or the amino acid sequence LAS, with up to 1, 2, 3, or 4 amino acid substitutions; and the CDR-L1 is a CDR-L1 of SEQ ID NOs: 141 or 149, with up to 1, 2, 3, 4, 5, or 6 amino acid substitutions.

In some embodiments, an antibody provided herein comprises a CDR-H3 of SEQ ID NOs: 112 or 130, a CDR-H2 of SEQ ID NOs: 100, 104, or 108, a CDR-H1 of SEQ ID NOs: 58, 68, or 85, a CDR-L3 of SEQ ID NO: 165, a CDR-L2 of SEQ ID NO: 158 or the amino acid sequence LAS, and a CDR-L1 of SEQ ID NOs: 141 or 149. In some embodiments, the CDR-H3 has at least about 80%, 90%, 91%, 92%, 93%, 94%, 95%, 96%, 97%, 98% or 99% identity with a CDR-H3 of SEQ ID NOs: 112 or 130, the CDR-H2 has at least about 80%, 90%, 91%, 92%, 93%, 94%, 95%, 96%, 97%, 98% or 99% identity with a CDR-H2 of SEQ ID NOs: 100, 104, or 108, the CDR-H1 has at least about 80%, 90%, 91%, 92%, 93%, 94%, 95%, 96%, 97%, 98% or 99% identity with a CDR-H1 of SEQ ID NOs: 58, 68 or 85, the CDR-L3 has at least about 80%, 90%, 91%, 92%, 93%, 94%, 95%, 96%, 97%, 98% or 99% identity with a CDR-L3 of SEQ ID NO: 165, the CDR-L2 has at least about 80%, 90%, 91%, 92%, 93%, 94%, 95%, 96%, 97%, 98% or 99% identity with a CDR-L2 of SEQ ID NO: 158 or the amino acid sequence LAS, and the CDR-L1 has at least about 80%, 90%, 91%, 92%, 93%, 94%, 95%, 96%, 97%, 98% or 99% identity with a CDR-L1 of SEQ ID NOs: 141 or 149. In some embodiments, the CDR-H3 is a CDR-H3 of SEQ ID NOs: 112 or 130, with up to 1, 2, 3, 4, 5, 6, 7, or 8 amino acid substitutions; the CDR-H2 is a CDR-H2 of SEQ ID NOs: 100, 104, or 108, with up to 1, 2, 3, 4, 5, 6, 7, or 8 amino acid substitutions; the CDR-H1 is a CDR-H1 of SEQ ID NOs: 58, 68, or 85, with up to 1, 2, 3, 4, or 5 amino acid substitutions; the CDR-L3 is a CDR-L3 of SEQ ID NO: 165, with up to 1, 2, 3, 4, or 5 amino acid substitutions; the CDR-L2 is a CDR-L2 of SEQ ID NO: 158 or the amino acid sequence LAS, with up to 1, 2, 3, or 4 amino acid substitutions; and the CDR-L1 is a CDR-L1 of SEQ ID NOs: 141 or 149, with up to 1, 2, 3, 4, 5, or 6 amino acid substitutions.

In some embodiments, an antibody provided herein comprises a CDR-H3 of SEQ ID NOs: 112, 121 or 130, a CDR-H2 of SEQ ID NOs: 100, 104 or 108, a CDR-H1 of SEQ ID NOs: 58, 67, or 84, a CDR-L3 of SEQ ID NO: 165, a CDR-L2 of SEQ ID NOs: 153 or the amino acid sequence LAS, and a CDR-L1 of SEQ ID NOs: 141 or 149. In some embodiments, the CDR-H3 has at least about 80%, 90%, 91%, 92%, 93%, 94%, 95%, 96%, 97%, 98% or 99% identity with a CDR-H3 of SEQ ID NOs: 112 or 130, the CDR-H2 has at least about 80%, 90%, 91%, 92%, 93%, 94%, 95%, 96%, 97%, 98% or 99% identity with a CDR-H2 of SEQ ID NOs: 100, 104 or 108, the CDR-H1 has at least about 80%, 90%, 91%, 92%, 93%, 94%, 95%, 96%, 97%, 98% or 99% identity with a CDR-H1 of SEQ ID NOs: 58, 67 or 84, the CDR-L3 has at least about 80%, 90%, 91%, 92%, 93%, 94%, 95%, 96%, 97%, 98% or 99% identity with a CDR-L3 of SEQ ID NO: 165, the CDR-L2 has at least about 80%, 90%, 91%, 92%, 93%, 94%, 95%, 96%, 97%, 98% or 99% identity with a CDR-L2 of SEQ ID NOs: 153 or the amino acid sequence LAS, and the CDR-L1 has at least about 80%, 90%, 91%, 92%, 93%, 94%, 95%, 96%, 97%, 98% or 99% identity with a CDR-L1 of SEQ ID NOs: 141 or 149. In some embodiments, the CDR-H3 is a CDR-H3 of SEQ ID NOs: 112 or 130, with up to 1, 2, 3, 4, 5, 6, 7, or 8 amino acid substitutions; the CDR-H2 is a CDR-H2 of SEQ ID NOs: 100, 104 or 108, with up to 1, 2, 3, 4, 5, 6, 7, or 8 amino acid substitutions; the CDR-H1 is a CDR-H1 of SEQ ID NOs: 58, 67 or 84, with up to 1, 2, 3, 4, or 5 amino acid substitutions; the CDR-L3 is a CDR-L3 of SEQ ID NO: 165, with up to 1, 2, 3, 4, or 5 amino acid substitutions; the CDR-L2 is a CDR-L2 of SEQ ID NO: 153 or the amino acid sequence LAS, with up to 1, 2, 3, or 4 amino acid substitutions; and the CDR-L1 is a CDR-L1 of SEQ ID NOs: 141 or 149, with up to 1, 2, 3, 4, 5, or 6 amino acid substitutions.

In some embodiments, an antibody provided herein comprises a CDR-H3 of SEQ ID NOs: 112 or 130, a CDR-H2 of SEQ ID NOs: 100, 104, or 108, a CDR-H1 of SEQ ID NOs: 58, 67, or 84, a CDR-L3 of SEQ ID NO: 165, a CDR-L2 of SEQ ID NO: 158 or the amino acid sequence LAS, and a CDR-L1 of SEQ ID NOs: 141 or 149. In some embodiments, the CDR-H3 has at least about 80%, 90%, 91%, 92%, 93%, 94%, 95%, 96%, 97%, 98% or 99% identity with a CDR-H3 of SEQ ID NOs: 112 or 130, the CDR-H2 has at least about 80%, 90%, 91%, 92%, 93%, 94%, 95%, 96%, 97%, 98% or 99% identity with a CDR-H2 of SEQ ID NOs: 100, 104, or 108, the CDR-H1 has at least about 80%, 90%, 91%, 92%, 93%, 94%, 95%, 96%, 97%, 98% or 99% identity with a CDR-H1 of SEQ ID NOs: 58, 67 or 84, the CDR-L3 has at least about 80%, 90%, 91%, 92%, 93%, 94%, 95%, 96%, 97%, 98% or 99% identity with a CDR-L3 of SEQ ID NO: 165, the CDR-L2 has at least about 80%, 90%, 91%, 92%, 93%, 94%, 95%, 96%, 97%, 98% or 99% identity with a CDR-L2 of SEQ ID NO: 158 or the amino acid sequence LAS, and the CDR-L1 has at least about 80%, 90%, 91%, 92%, 93%, 94%, 95%, 96%, 97%, 98% or 99% identity with a CDR-L1 of SEQ ID NOs: 141 or 149. In some embodiments, the CDR-H3 is a CDR-H3 of SEQ ID NOs: 112 or 130, with up to 1, 2, 3, 4, 5, 6, 7, or 8 amino acid substitutions; the CDR-H2 is a CDR-H2 of SEQ ID NOs: 100, 104, or 108, with up to 1, 2, 3, 4, 5, 6, 7, or 8 amino acid substitutions; the CDR-H1 is a CDR-H1 of SEQ ID NOs: 58, 67, or 84, with up to 1, 2, 3, 4, or 5 amino acid substitutions; the CDR-L3 is a CDR-L3 of SEQ ID NO: 165, with up to 1, 2, 3, 4, or 5 amino acid substitutions; the CDR-L2 is a CDR-L2 of SEQ ID NO: 158 or the amino acid sequence LAS, with up to 1, 2, 3, or 4 amino acid substitutions; and the CDR-L1 is a CDR-L1 of SEQ ID NOs: 141 or 149, with up to 1, 2, 3, 4, 5, or 6 amino acid substitutions.

In some embodiments, the amino acid substitutions are conservative amino acid substitutions. In some embodiments, the antibodies described in this disclosure are referred to herein as “variants” or “clones”. In some embodiments, such variants or clones are derived from a sequence provided herein, for example, by affinity maturation, site directed mutagenesis, random mutagenesis, or any other method known in the art or described herein. In some embodiments, such variants or cones are not derived from a sequence provided herein and may, for example, be isolated de novo according to the methods provided herein for obtaining antibodies.

In certain aspects, the antibodies disclosed herein do not include antibodies disclosed in U.S. Pat. No. 9,067,994.

TABLE 3
Anti-interleukin (IL)-13 antibody Heavy Chain Kabat CDRs

| | | | SEQ | | SEQ | | SEQ | | SEQ | | SEQ | | SEQ | | SEQ |
|---|---|---|---|---|---|---|---|---|---|---|---|---|---|---|---|
| Construct | | | ID | | ID | | ID | | ID | | ID | | ID | | ID |
| ID | VH Name* | FR1 | NO: | CDR1 | NO: | FR2 | NO: | CDR2 | NO: | FR3 | NO: | CDR3 | NO: | FR4 | NO: |
| 1 | Lebrikizumab | QVTLR | 540 | AYSVN | 58 | WIRQP | 255 | MIWGD | 100 | RLTIS | 311 | DGYYP | 112 | WGQGS | 368 |
| (lebrikizumab), | -HC | ESGPA | | | | PGKAL | | GKIVY | | KDTSK | | YAMDN | | LVTVS | |
| 2, and 128- | | LVKPT | | | | EWLA | | NSALK | | NQVVL | | | | S | |
| 131 | | QTLTL | | | | | | S | | TMTNM | | | | | |
| | | TCTVS | | | | | | | | DPVDT | | | | | |
| | | GFSLS | | | | | | | | ATYYC | | | | | |
| | | | | | | | | | | AG | | | | | |
| 3 | HC0 | QVQLQ | 198 | AYSVN | 58 | WVRQP | 256 | MIWGD | 100 | RLNIS | 312 | DGYYP | 112 | WGHGT | 369 |
| | | ESGPG | | | | PGKGL | | GKIVY | | KDSSK | | YAMDN | | SVTVS | |
| | | LVAPS | | | | EWLG | | NSALK | | SQVFL | | | | S | |
| | | QSLSI | | | | | | S | | KMSSL | | | | | |
| | | TCTVS | | | | | | | | QSDDT | | | | | |
| | | GFSLN | | | | | | | | ARYYC | | | | | |
| | | | | | | | | | | AG | | | | | |
| 4 | HC0_M | QVQLQ | 198 | AYSVN | 58 | WVRQP | 256 | MIWGD | 100 | RLTIS | 313 | DGYYP | 112 | WGHGT | 369 |
| | | ESGPG | | | | PGKGL | | GKIVY | | KDSSK | | YAMDN | | SVTVS | |
| | | LVAPS | | | | EWLG | | NSALK | | SQVFL | | | | S | |
| | | QSLSI | | | | | | S | | KMSSL | | | | | |
| | | TCTVS | | | | | | | | QSDDT | | | | | |
| | | GFSLN | | | | | | | | ARYYC | | | | | |
| | | | | | | | | | | AG | | | | | |
| 5, 10, 15, | HC1 | EVQLQ | 200 | AYSVN | 58 | WIRQP | 258 | MIWGD | 100 | RLTIS | 314 | DGYYP | 112 | WGQGT | 371 |
| 20, 133- | | ESGPG | | | | PGKGL | | GKIVY | | KDSSK | | YAMDN | | TVTVS | |
| 136, and | | LVKPS | | | | EWLG | | NSALK | | NQVSL | | | | S | |
| 141-144 | | ETLSL | | | | | | S | | KLSSV | | | | | |
| | | TCTVS | | | | | | | | TAADT | | | | | |
| | | GFSLN | | | | | | | | AVYYC | | | | | |
| | | | | | | | | | | AG | | | | | |
| 6, 11, 16, | HC2 | EVQLV | 201 | AYSVN | 58 | WVRQA | 259 | MIWGD | 100 | RLTIT | 315 | DGYYP | 112 | WGQGT | 371 |
| and 21 | | QSGAE | | | | PGQGL | | GKIVY | | KDSST | | YAMDN | | TVTVS | |
| | | VKKPG | | | | EWLG | | NSALK | | STVYM | | | | S | |
| | | ASVKV | | | | | | S | | ELSSL | | | | | |
| | | SCKAS | | | | | | | | RSEDT | | | | | |
| | | GFSLN | | | | | | | | AVYYC | | | | | |
| | | | | | | | | | | AG | | | | | |
| 7, 12, 17, | HC3 | EVQLV | 202 | AYSVN | 58 | WVRQA | 259 | MIWGD | 100 | RLTIT | 315 | DGYYP | 112 | WGQG | 371 |
| and 22 | | QSGAE | | | | PGQGL | | GKIVY | | KDSST | | | | TTVTV | |
| | | VKKPG | | | | EWLG | | NSALK | | STVYM | | YAMD | | | |
| | | SSVKV | | | | | | S | | ELSSL | | N | | SS | |
| | | SCKAS | | | | | | | | RSEDT | | | | | |
| | | GFSLN | | | | | | | | AVYYC | | | | | |
| | | | | | | | | | | AG | | | | | |
| 8, 13, 18, | HC4 | EVQLV | 203 | AYSVN | 58 | WVRQA | 262 | MIWGD | 100 | RLTIS | 317 | DGYYP | 112 | WGQGT | 371 |
| and 23 | | ESGGG | | | | PGKGL | | GKIVY | | KDSSK | | YAMDN | | TVTVS | |
| | | LVKPG | | | | EWLG | | NSALK | | NTVYL | | | | S | |
| | | GSLRL | | | | | | S | | QMNSL | | | | | |
| | | SCAAS | | | | | | | | KTEDT | | | | | |
| | | GFSLN | | | | | | | | AVYYC | | | | | |
| | | | | | | | | | | AG | | | | | |
| 9, 14, 19, | HC5 | EVQLL | 204 | AYSVN | 58 | WVRQA | 262 | MIWGD | 100 | RLTIS | 318 | DGYYP | 112 | WGQGT | 371 |
| 24, 90- | | ESGGG | | | | PGKGL | | GKIVY | | KDSSK | | YAMDN | | TVTVS | |
| 104, 132, | | LVQPG | | | | EWLG | | NSALK | | NTVYL | | | | S | |
| and 137- | | GSLRL | | | | | | S | | QMNSL | | | | | |
| 140 | | SCAAS | | | | | | | | RAEDT | | | | | |
| | | GFSLN | | | | | | | | AVYYC | | | | | |
| | | | | | | | | | | AG | | | | | |
| 25 | HC6 | EVQLQ | 205 | AYSVN | 58 | WVRQP | 256 | MIWGD | 100 | RLTIS | 319 | DGYYP | 112 | WGQGT | 371 |
| | | ESGPG | | | | PGKGL | | GKIVY | | LDTSK | | YAMDN | | TVTVS | |
| | | LVKPS | | | | EWLG | | NSALK | | SQVFL | | | | S | |
| | | ETLSL | | | | | | S | | KMSSL | | | | | |
| | | TCTVS | | | | | | | | TAADT | | | | | |
| | | GGSLN | | | | | | | | AVYYC | | | | | |
| | | | | | | | | | | AR | | | | | |
| 26 | HC7 | QVQLQ | 206 | AYSWN | 541 | WVRQP | 256 | YIYGD | 101 | RLTIS | 319 | DGYYY | 113 | WGQGT | 371 |
| | | ESGPG | | | | PGKGL | | GKTNY | | LDTSK | | YAMDV | | TVTVS | |
| | | LVKPS | | | | EWLG | | NPALK | | SQVFL | | | | S | |
| | | ETLSL | | | | | | S | | KMSSL | | | | | |
| | | TCTVS | | | | | | | | TAADT | | | | | |
| | | GGSLN | | | | | | | | AVYYC | | | | | |
| | | | | | | | | | | AR | | | | | |
| 105 | HC5_m1 | EVQLL | 207 | AYSVN | 58 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GYSLN | | | | | | | | AVYYC | | | | | |
| | | | | | | | | | | AG | | | | | |
| 106 | HC5_m2 | EVQLL | 208 | AYSVN | 58 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLR | | | | | | | | AVYYC | | | | | |
| | | | | | | | | | | AG | | | | | |
| 107 | HC5_m3 | EVQLL | 209 | AYSVN | 58 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLH | | | | | | | | AVYYC | | | | | |
| | | | | | | | | | | AG | | | | | |
| 108 | HC5_m4 | EVQLL | 210 | AYSVN | 58 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLD | | | | | | | | AVYYC | | | | | |
| | | | | | | | | | | AG | | | | | |
| 109 | HC5_m5 | EVQLL | 211 | AYSVN | 58 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLY | | | | | | | | AVYYC | | | | | |
| | | | | | | | | | | AG | | | | | |
| 110 | HC5_m6 | EVQLL | 212 | AYSVN | 58 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLS | | | | | | | | AVYYC | | | | | |
| | | | | | | | | | | AG | | | | | |
| 111 | HC5_m7 | EVQLL | 204 | RYSVN | 59 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 112 | HC5_m8 | EVQLL | 204 | KYSVN | 60 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 113 | HC5_m9 | EVQLL | 204 | HYSVN | 61 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 114 | HC5_m10 | EVQLL | 204 | QYSVN | 62 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 115 | HC5_ml1 | EVQLL | 204 | EYSVN | 63 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 116 | HC5_m12 | EVQLL | 204 | SYSVN | 64 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 117 | HC5_m13 | EVQLL | 204 | YYSVN | 65 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 118 | HC5_m14 | EVQLL | 204 | AESVN | 66 | WVRQA | 262 | MIWGD | 100 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 119 | HC5_m15 | EVQLL | 204 | AYSVN | 58 | WVRQA | 262 | MIWSD | 102 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 120 | HC5_m16 | EVQLL | 204 | AYSVN | 58 | WVRQA | 262 | MIWAD | 103 | RLTIS | 318 | DGYYP | 112 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 121 | HC5_m17 | EVQLL | 204 | AYSVN | 58 | WVRQA | 262 | MIWGD | 100 | RLTIS | 318 | HGYYP | 114 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 122 | HC5_m18 | EVQLL | 204 | AYSVN | 58 | WVRQA | 262 | MIWGD | 100 | RLTIS | 318 | DLYYP | 115 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 123 | HC5_m19 | EVQLL | 204 | AYSVN | 58 | WVRQA | 262 | MIWGD | 100 | RLTIS | 318 | DKYYP | 116 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 124 | HC5_m20 | EVQLL | 204 | AYSVN | 58 | WVRQA | 262 | MIWGD | 100 | RLTIS | 318 | DGYYG | 117 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 125 | HC5_m21 | EVQLL | 204 | AYSVN | 58 | WVRQA | 262 | MIWGD | 100 | RLTIS | 318 | DGYYA | 118 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 126 | HC5_m22 | EVQLL | 204 | AYSVN | 58 | WVRQA | 262 | MIWGD | 100 | RLTIS | 318 | DGYYS | 119 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |
| 127 | HC5_m24 | EVQLL | 204 | AYSVN | 58 | WVRQA | 262 | MIWGD | 100 | RLTIS | 318 | DGYYT | 120 | WGQGT | 371 |
| | | ESGGG | | | | PGKGL | | GKIVY | | KDSSK | | YAMDN | | TVTVS | |
| | | LVQPG | | | | EWLG | | NSALK | | NTVYL | | | | S | |
| | | GSLRL | | | | | | S | | QMNSL | | | | | |
| | | SCAAS | | | | | | | | RAEDT | | | | | |
| | | GFSLN | | | | | | | | AVYYC | | | | | |
| | | | | | | | | | | AG | | | | | |

*Names correspond with name in informal sequence listing.

TABLE 4
Anti-interleukin (IL)-13 antibody Heavy Chain Chothia CDRs

| | | | SEQ | | SEQ | | SEQ | | SEQ | | SEQ | | SEQ | | SEQ |
|---|---|---|---|---|---|---|---|---|---|---|---|---|---|---|---|
| Construct | | | ID | | ID | | ID | | ID | | ID | | ID | | ID |
| ID | VH Name* | FR1 | NO: | CDR1 | NO: | FR2 | NO: | CDR2 | NO: | FR3 | NO: | CDR3 | NO: | FR4 | NO: |
| 1 | Lebrikizumab | QVTLR | 542 | GFSLS | 67 | SVNWI | 550 | WGDG | 104 | IVYNS | 556 | DGYYP | 112 | WGQG | 368 |
| (lebrikizumab), | -HC | ESGPA | | AY | | RQPPG | | K | | ALKSR | | YAMD | | SLVT | |
| 2, and | | LVKPT | | | | KALE | | | | LTISKD | | N | | VSS | |
| 128-131 | | QTLTL | | | | WLAMI | | | | TSKNQ | | | | | |
| | | TCTVS | | | | | | | | VVLTM | | | | | |
| | | | | | | | | | | TNMDP | | | | | |
| | | | | | | | | | | VDTAT | | | | | |
| | | | | | | | | | | YYCAG | | | | | |
| 3 | HC0 | QVQLQ | 543 | GFSLN | 68 | SVNW | 551 | WGDG | 104 | IVYNS | 557 | DGYYP | 112 | WGHG | 369 |
| | | ESGPG | | AY | | VRQPP | | K | | ALKSR | | YAMD | | TSVT | |
| | | LVAPS | | | | GKGLE | | | | LNISK | | N | | VSS | |
| | | QSLSIT | | | | WLGMI | | | | DSSKS | | | | | |
| | | CTVS | | | | | | | | QVFLK | | | | | |
| | | | | | | | | | | MSSLQ | | | | | |
| | | | | | | | | | | SDDTA | | | | | |
| | | | | | | | | | | RYYCA | | | | | |
| | | | | | | | | | | G | | | | | |
| 4 | HC0_M | QVQLQ | 543 | GFSLN | 68 | SVNW | 551 | WGDG | 104 | IVYNS | 558 | DGYYP | 112 | WGHG | 369 |
| | | ESGPG | | AY | | VRQPP | | K | | ALKSR | | YAMD | | TSVTV | |
| | | LVAPS | | | | GKGLE | | | | LTISKD | | N | | SS | |
| | | QSLSIT | | | | WLGMI | | | | SSKSQ | | | | | |
| | | CTVS | | | | | | | | VFLKM | | | | | |
| | | | | | | | | | | SSLQS | | | | | |
| | | | | | | | | | | DDTAR | | | | | |
| | | | | | | | | | | YYCAG | | | | | |
| 5, 10, 15, | HC1 | EVQLQ | 544 | GFSLN | 68 | SVNWI | 552 | WGDG | 104 | IVYNS | 559 | DGYYP | 112 | WGQG | 371 |
| 20, 133- | | ESGPG | | AY | | RQPPG | | K | | ALKSR | | YAMD | | TTVT | |
| 136, and | | LVKPS | | | | KGLE | | | | LTISKD | | N | | VSS | |
| 141-144 | | ETLSL | | | | WLGMI | | | | SSKNQ | | | | | |
| | | TCTVS | | | | | | | | VSLKL | | | | | |
| | | | | | | | | | | SSVTA | | | | | |
| | | | | | | | | | | ADTAV | | | | | |
| | | | | | | | | | | YYCAG | | | | | |
| 6, 11, 16, | HC2 | EVQLV | 545 | GFSLN | 68 | SVNW | 553 | WGDG | 104 | IVYNS | 560 | DGYYP | 112 | WGQG | 371 |
| and 21 | | QSGAE | | AY | | VRQAP | | K | | ALKSR | | YAMD | | TTVT | |
| | | VKKPG | | | | GQGLE | | | | LTITK | | N | | VSS | |
| | | ASVKV | | | | WLGMI | | | | DSSTS | | | | | |
| | | SCKAS | | | | | | | | TVYME | | | | | |
| | | | | | | | | | | LSSLRS | | | | | |
| | | | | | | | | | | EDTAV | | | | | |
| | | | | | | | | | | YYCAG | | | | | |
| 7, 12, 17, | HC3 | EVQLV | 546 | GFSLN | 68 | SVNW | 553 | WGDG | 104 | IVYNS | 560 | DGYYP | 112 | WGQG | 371 |
| and 22 | | QSGAE | | AY | | VRQAP | | K | | ALKSR | | YAMD | | TTVT | |
| | | VKKPG | | | | GQGLE | | | | LTITK | | N | | VSS | |
| | | SSVKV | | | | WLGMI | | | | DSSTS | | | | | |
| | | SCKAS | | | | | | | | TVYME | | | | | |
| | | | | | | | | | | LSSLRS | | | | | |
| | | | | | | | | | | EDTAV | | | | | |
| | | | | | | | | | | YYCAG | | | | | |
| 8, 13, 18, | HC4 | EVQLV | 547 | GFSLN | 68 | SVNW | 554 | WGDG | 104 | IVYNS | 561 | DGYYP | 112 | WGQG | 371 |
| and 23 | | ESGGG | | AY | | VRQAP | | K | | ALKSR | | YAMD | | TTVT | |
| | | LVKPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLK | | | | | |
| | | | | | | | | | | TEDTA | | | | | |
| | | | | | | | | | | VYYCA | | | | | |
| | | | | | | | | | | G | | | | | |
| 9, 14, 19, | HC5 | EVQLL | 548 | GFSLN | 68 | SVNW | 554 | WGDG | 104 | IVYNS | 562 | DGYYP | 112 | WGQG | 371 |
| 24, 90-104, | | ESGGG | | AY | | VRQAP | | K | | ALKSR | | YAMD | | TTVT | |
| 132, and | | LVQPG | | | | GKGLE | | | | LTISKD | | N | | VSS | |
| 137-140 | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 25 | HC6 | EVQLQ | 544 | GGSLN | 69 | SVNW | 551 | WGDG | 104 | IVYNS | 563 | DGYYP | 112 | WGQG | 371 |
| | | ESGPG | | AY | | VRQPP | | K | | ALKSR | | YAMD | | TTVT | |
| | | LVKPS | | | | GKGLE | | | | LTISLD | | N | | VSS | |
| | | ETLSL | | | | WLGMI | | | | TSKSQ | | | | | |
| | | TCTVS | | | | | | | | VFLKM | | | | | |
| | | | | | | | | | | SSLTA | | | | | |
| | | | | | | | | | | ADTAV | | | | | |
| | | | | | | | | | | YYCAR | | | | | |
| 26 | HC7 | QVQLQ | 549 | GGSLN | 69 | SWNW | 555 | YGDG | 105 | TNYNP | 564 | DGYY | 113 | WGQG | 371 |
| | | ESGPG | | AY | | VRQPP | | K | | ALKSR | | YYAM | | TTVT | |
| | | LVKPS | | | | GKGLE | | | | LTISLD | | DV | | VSS | |
| | | ETLSL | | | | WLGYI | | | | TSKSQ | | | | | |
| | | TCTVS | | | | | | | | VFLKM | | | | | |
| | | | | | | | | | | SSLTA | | | | | |
| | | | | | | | | | | ADTAV | | | | | |
| | | | | | | | | | | YYCAR | | | | | |
| 105 | HC5_m1 | EVQLL | 548 | GYSLN | 71 | SVNW | 554 | WGDG | 104 | IVYNS | 562 | DGYYP | 112 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 106 | HC5_m2 | EVQLL | 548 | GFSLR | 72 | SVNW | 554 | WGDG | 104 | IVYNS | 562 | DGYYP | 112 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 107 | HC5_m3 | EVQLL | 548 | GFSLH | 73 | SVNW | 554 | WGDG | 104 | IVYNS | 562 | DGYYP | 112 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 108 | HC5_m4 | EVQLL | 548 | GFSLD | 74 | SVNW | 554 | WGDG | 104 | IVYNS | 562 | DGYYP | 112 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 109 | HC5_m5 | EVQLL | 548 | GFSLY | 75 | SVNW | 554 | WGDG | 104 | IVYNS | 562 | DGYYP | 112 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 110 | HC5_m6 | EVQLL | 548 | GFSLS | 67 | SVNW | 554 | WGDG | 104 | IVYNS | 562 | DGYYP | 112 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 111 | HC5_m7 | EVQLL | 548 | GFSLN | 76 | SVNW | 554 | WGDG | 104 | IVYNS | 562 | DGYYP | 112 | WGQG | 371 |
| | | ESGGG | | RY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 112 | HC5_m8 | EVQLL | 548 | GFSLN | 77 | SVNW | 554 | WGDG | 104 | IVYNS | 562 | DGYYP | 112 | WGQG | 371 |
| | | ESGGG | | KY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 113 | HC5_m9 | EVQLL | 548 | GFSLN | 78 | SVNW | 554 | WGDG | 104 | IVYNS | 562 | DGYYP | 112 | WGQG | 371 |
| | | ESGGG | | HY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 114 | HC5_m10 | EVQLL | 548 | GFSLN | 79 | SVNW | 554 | WGDG | 104 | IVYNS | 562 | DGYYP | 112 | WGQG | 371 |
| | | ESGGG | | QY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 115 | HC5_m11 | EVQLL | 548 | GFSLN | 80 | SVNW | 554 | WGDG | 104 | IVYNS | 562 | DGYYP | 112 | WGQG | 371 |
| | | ESGGG | | EY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 116 | HC5_m12 | EVQLL | 548 | GFSLN | 81 | SVNW | 554 | WGDG | 104 | IVYNS | 562 | DGYYP | 112 | WGQG | 371 |
| | | ESGGG | | SY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 117 | HC5_m13 | EVQLL | 548 | GFSLN | 82 | SVNW | 554 | WGDG | 104 | IVYNS | 562 | DGYYP | 112 | WGQG | 371 |
| | | ESGGG | | YY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 118 | HC5_m14 | EVQLL | 548 | GFSLN | 83 | SVNW | 554 | WGDG | 104 | IVYNS | 562 | DGYYP | 112 | WGQG | 371 |
| | | ESGGG | | AE | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 119 | HC5_m15 | EVQLL | 548 | GFSLN | 68 | SVNW | 554 | WSDG | 106 | IVYNS | 562 | DGYYP | 112 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 120 | HC5_m16 | EVQLL | 548 | GFSLN | 68 | SVNW | 554 | WADG | 107 | IVYNS | 562 | DGYYP | 112 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 121 | HC5_m17 | EVQLL | 548 | GFSLN | 68 | SVNW | 554 | WGDG | 104 | IVYNS | 562 | HGYYP | 114 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 122 | HC5_m18 | EVQLL | 548 | GFSLN | 68 | SVNW | 554 | WGDG | 104 | IVYNS | 562 | DLYYP | 115 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 123 | HC5_m19 | EVQLL | 548 | GFSLN | 68 | SVNW | 554 | WGDG | 104 | IVYNS | 562 | DKYYP | 116 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 124 | HC5_m20 | EVQLL | 548 | GFSLN | 68 | SVNW | 554 | WGDG | 104 | IVYNS | 562 | DGYY | 117 | WGQG | 371 |
| | | ESGGG | | AY | | VRQAP | | K | | ALKSR | | GYAM | | TTVT | |
| | | LVQPG | | | | GKGLE | | | | LTISKD | | DN | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 125 | HC5_m21 | EVQLL | 548 | GFSLN | 68 | SVNW | 554 | WGDG | 104 | IVYNS | 562 | DGYY | 118 | WGQG | 371 |
| | | ESGGG | | AY | | VRQAP | | K | | ALKSR | | AYAM | | TTVT | |
| | | LVQPG | | | | GKGLE | | | | LTISKD | | DN | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 126 | HC5_m22 | EVQLL | 548 | GFSLN | 68 | SVNW | 554 | WGDG | 104 | IVYNS | 562 | DGYYS | 119 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |
| 127 | HC5_m24 | EVQLL | 548 | GFSLN | 68 | SVNW | 554 | WGDG | 104 | IVYNS | 562 | DGYYT | 120 | WGQG | 371 |
| | | ESGGG | | AY | | VRQAP | | K | | ALKSR | | YAMD | | TTVT | |
| | | LVQPG | | | | GKGLE | | | | LTISKD | | N | | VSS | |
| | | GSLRL | | | | WLGMI | | | | SSKNT | | | | | |
| | | SCAAS | | | | | | | | VYLQ | | | | | |
| | | | | | | | | | | MNSLR | | | | | |
| | | | | | | | | | | AEDTA | | | | | |
| | | | | | | | | | | VYYCA | | | | | |
| | | | | | | | | | | G | | | | | |

*Names correspond with name in informal sequence listing.

TABLE 5
Anti-interleukin (IL)-13 antibody Heavy Chain IMGT CDRs

| | | | SEQ | | SEQ | | SEQ | | SEQ | | SEQ | | SEQ | | SEQ |
|---|---|---|---|---|---|---|---|---|---|---|---|---|---|---|---|
| Construct | | | ID | | ID | | ID | | ID | | ID | | ID | | ID |
| ID | VH Name* | FR1 | NO: | CDR1 | NO: | FR2 | NO: | CDR2 | NO: | FR3 | NO: | CDR3 | NO: | FR4 | NO: |
| 1 | Lebrikiz- | QVTLR | 542 | GFSLS | 84 | VNWI | 566 | IWGDG | 108 | VYNSA | 572 | AGDG | 130 | WGQG | 368 |
| (lebrikiz- | umab - HC | ESGPA | | AYS | | RQPP | | KI | | LKSRL | | YYPY | | SLVT | |
| umab), 2, | | LVKPT | | | | GKAL | | | | TISKD | | AMDN | | VSS | |
| and 128- | | QTLTL | | | | EWLA | | | | TSKNQ | | | | | |
| 131 | | TCTVS | | | | M | | | | VVLTM | | | | | |
| | | | | | | | | | | TNMDP | | | | | |
| | | | | | | | | | | VDTAT | | | | | |
| | | | | | | | | | | YYC | | | | | |
| 3 | HC0 | QVQLQ | 543 | GFSLN | 85 | VNWV | 567 | IWGDG | 108 | VYNSA | 573 | AGDG | 130 | WGHG | 369 |
| | | ESGPG | | AYS | | RQPP | | KI | | LKSRL | | YYPY | | TSVT | |
| | | LVAPS | | | | GKGL | | | | NISKD | | AMDN | | VSS | |
| | | QSLSI | | | | EWLG | | | | SSKSQ | | | | | |
| | | TCTVS | | | | M | | | | VFLKM | | | | | |
| | | | | | | | | | | SSLQS | | | | | |
| | | | | | | | | | | DDTAR | | | | | |
| | | | | | | | | | | YYC | | | | | |
| 4 | HC0_M | QVQLQ | 543 | GFSLN | 85 | VNWV | 567 | IWGDG | 108 | VYNSA | 574 | AGDG | 130 | WGHG | 369 |
| | | ESGPG | | AYS | | RQPP | | KI | | LKSRL | | YYPY | | TSVT | |
| | | LVAPS | | | | GKGL | | | | TISKD | | AMDN | | VSS | |
| | | QSLSI | | | | EWLG | | | | SSKSQ | | | | | |
| | | TCTVS | | | | M | | | | VFLKM | | | | | |
| | | | | | | | | | | SSLQS | | | | | |
| | | | | | | | | | | DDTAR | | | | | |
| | | | | | | | | | | YYC | | | | | |
| 5, 10, 15, | HC1 | EVQLQ | 544 | GFSLN | 85 | VNWI | 568 | IWGDG | 108 | VYNSA | 575 | AGDG | 130 | WGQG | 371 |
| 20, and | | ESGPG | | AYS | | RQPP | | KI | | LKSRL | | YYPY | | TTVT | |
| 133-136 | | LVKPS | | | | GKGL | | | | TISKD | | AMDN | | VSS | |
| | | ETLSL | | | | EWLG | | | | SSKNQ | | | | | |
| | | TCTVS | | | | M | | | | VSLKL | | | | | |
| | | | | | | | | | | SSVTA | | | | | |
| | | | | | | | | | | ADTAV | | | | | |
| | | | | | | | | | | YYC | | | | | |
| 6, 11, 16, | HC2 | EVQLV | 545 | GFSLN | 85 | VNWV | 569 | IWGDG | 108 | VYNSA | 576 | AGDG | 130 | WGQG | 371 |
| and 21 | | QSGAE | | AYS | | RQAP | | KI | | LKSRL | | YYPY | | TTVT | |
| | | VKKPG | | | | GQGL | | | | TITKD | | AMDN | | VSS | |
| | | ASVKV | | | | EWLG | | | | SSTST | | | | | |
| | | SCKAS | | | | M | | | | VYMEL | | | | | |
| | | | | | | | | | | SSLRS | | | | | |
| | | | | | | | | | | EDTAV | | | | | |
| | | | | | | | | | | YYC | | | | | |
| 7, 12, 17, | HC3 | EVQLV | 546 | GFSLN | 85 | VNWV | 569 | IWGDG | 108 | VYNSA | 576 | AGDG | 130 | WGQG | 371 |
| and 22 | | QSGAE | | AYS | | RQAP | | KI | | LKSRL | | YYPY | | TTVT | |
| | | VKKPG | | | | GQGL | | | | TITKD | | AMDN | | VSS | |
| | | SSVKV | | | | EWLG | | | | SSTST | | | | | |
| | | SCKAS | | | | M | | | | VYMEL | | | | | |
| | | | | | | | | | | SSLRS | | | | | |
| | | | | | | | | | | EDTAV | | | | | |
| | | | | | | | | | | YYC | | | | | |
| 8, 13, 18, | HC4 | EVQLV | 547 | GFSLN | 85 | VNWV | 570 | IWGDG | 108 | VYNSA | 577 | AGDG | 130 | WGQG | 371 |
| and 23 | | ESGGG | | AYS | | RQAP | | KI | | LKSRL | | YYPY | | TTVT | |
| | | LVKPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLKT | | | | | |
| | | | | | | | | | | EDTAV | | | | | |
| | | | | | | | | | | YYC | | | | | |
| 9, 14, | HC5 | EVQLL | 548 | GFSLN | 85 | VNWV | 570 | IWGDG | 108 | VYNSA | 578 | AGDG | 130 | WGQG | 371 |
| 19, 24, | | ESGGG | | AYS | | RQAP | | KI | | LKSRL | | YYPY | | TTVT | |
| 90-104, | | LVQPG | | | | GKGL | | | | TISKD | | AMDN | | VSS | |
| 132, and | | GSLRL | | | | EWLG | | | | SSKNT | | | | | |
| 137-140 | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 25 | HC6 | EVQLQ | 544 | GGSLN | 86 | VNWV | 567 | IWGDG | 108 | VYNSA | 579 | ARDG | 131 | WGQG | 371 |
| | | ESGPG | | AYS | | RQPP | | KI | | LKSRL | | YYPY | | TTVT | |
| | | LVKPS | | | | GKGL | | | | TISLD | | AMDN | | VSS | |
| | | ETLSL | | | | EWLG | | | | TSKSQ | | | | | |
| | | TCTVS | | | | M | | | | VFLKM | | | | | |
| | | | | | | | | | | SSLTA | | | | | |
| | | | | | | | | | | ADTAV | | | | | |
| | | | | | | | | | | YYC | | | | | |
| 26 | HC7 | QVQLQ | 549 | GGSLN | 86 | WNWV | 571 | IYGDG | 109 | NYNPA | 580 | ARDG | 132 | WGQG | 371 |
| | | ESGPG | | AYS | | RQPP | | KT | | LKSRL | | YYYY | | TTVT | |
| | | LVKPS | | | | GKGL | | | | TISLD | | AMDV | | VSS | |
| | | ETLSL | | | | EWLG | | | | TSKSQ | | | | | |
| | | TCTVS | | | | Y | | | | VFLKM | | | | | |
| | | | | | | | | | | SSLTA | | | | | |
| | | | | | | | | | | ADTAV | | | | | |
| | | | | | | | | | | YYC | | | | | |
| 105 | HC5_m1 | EVQLL | 548 | GYSLN | 87 | VNWV | 570 | IWGDG | 108 | VYNSA | 578 | AGDG | 130 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 106 | HC5_m2 | EVQLL | 548 | GFSLR | 88 | VNWV | 570 | IWGDG | 108 | VYNSA | 578 | AGDG | 130 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 107 | HC5_m3 | EVQLL | 548 | GFSLH | 89 | VNWV | 570 | IWGDG | 108 | VYNSA | 578 | AGDG | 130 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 108 | HC5_m4 | EVQLL | 548 | GFSLD | 90 | VNWV | 570 | IWGDG | 108 | VYNSA | 578 | AGDG | 130 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 109 | HC5_m5 | EVQLL | 548 | GFSLY | 91 | VNWV | 570 | IWGDG | 108 | VYNSA | 578 | AGDG | 130 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 110 | HC5_m6 | EVQLL | 548 | GFSLS | 84 | VNWV | 570 | IWGDG | 108 | VYNSA | 578 | AGDG | 130 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 111 | HC5_m7 | EVQLL | 548 | GFSLN | 93 | VNWV | 570 | IWGDG | 108 | VYNSA | 578 | AGDG | 130 | WGQG | 371 |
| | | ESGGG | | RYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 112 | HC5_m8 | EVQLL | 548 | GFSLN | 94 | VNWV | 570 | IWGDG | 108 | VYNSA | 578 | AGDG | 130 | WGQG | 371 |
| | | ESGGG | | KYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 113 | HC5_m9 | EVQLL | 548 | GFSLN | 95 | VNWV | 570 | IWGDG | 108 | VYNSA | 578 | AGDG | 130 | WGQG | 371 |
| | | ESGGG | | HYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 114 | HC5_m10 | EVQLL | 548 | GFSLN | 96 | VNWV | 570 | IWGDG | 108 | VYNSA | 578 | AGDG | 130 | WGQG | 371 |
| | | ESGGG | | QYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 115 | HC5_m11 | EVQLL | 548 | GFSLN | 97 | VNWV | 570 | IWGDG | 108 | VYNSA | 578 | AGDG | 130 | WGQG | 371 |
| | | ESGGG | | EYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 116 | HC5_m12 | EVQLL | 548 | GFSLN | 565 | VNWV | 570 | IWGDG | 108 | VYNSA | 578 | AGDG | 130 | WGQG | 371 |
| | | ESGGG | | SYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 117 | HC5_m13 | EVQLL | 548 | GFSLN | 98 | VNWV | 570 | IWGDG | 108 | VYNSA | 578 | AGDG | 130 | WGQG | 371 |
| | | ESGGG | | YYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 118 | HC5_m14 | EVQLL | 548 | GFSLN | 99 | VNWV | 570 | IWGDG | 108 | VYNSA | 578 | AGDG | 130 | WGQG | 371 |
| | | ESGGG | | AES | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 119 | HC5_m15 | EVQLL | 548 | GFSLN | 85 | VNWV | 570 | IWSDG | 110 | VYNSA | 578 | AGDG | 130 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 120 | HC5_m16 | EVQLL | 548 | GFSLN | 85 | VNWV | 570 | IWADG | 111 | VYNSA | 578 | AGDG | 130 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 121 | HC5_m17 | EVQLL | 548 | GFSLN | 85 | VNWV | 570 | IWGDG | 108 | VYNSA | 578 | AGHG | 133 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 122 | HC5_m18 | EVQLL | 548 | GFSLN | 85 | VNWV | 570 | IWGDG | 108 | VYNSA | 578 | AGDL | 134 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 123 | HC5_m19 | EVQLL | 548 | GFSLN | 85 | VNWV | 570 | IWGDG | 108 | VYNSA | 578 | AGDK | 135 | WGQG | 371 |
| | | ESGGG | | AYS | | RQAP | | KI | | LKSRL | | YYPY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 124 | HC5_m20 | EVQLL | 548 | GFSLN | 85 | VNWV | 570 | IWGDG | 108 | VYNSA | 578 | AGDG | 136 | WGQG | 371 |
| | | ESGGG | | AYS | | RQAP | | KI | | LKSRL | | YYGY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 125 | HC5_m21 | EVQLL | 548 | GFSLN | 85 | VNWV | 570 | IWGDG | 108 | VYNSA | 578 | AGDG | 137 | WGQG | 371 |
| | | ESGGG | | AYS | | RQAP | | KI | | LKSRL | | YYAY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 126 | HC5_m22 | EVQLL | 548 | GFSLN | 85 | VNWV | 570 | IWGDG | 108 | VYNSA | 578 | AGDG | 138 | WGQG | 371 |
| | | ESGGG | | AYS | | RQAP | | KI | | LKSRL | | YYSY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |
| 127 | HC5_m24 | EVQLL | 548 | GFSLN | 85 | VNWV | 570 | IWGDG | 108 | VYNSA | 578 | AGDG | 139 | WGQG | 371 |
| | | ESGGG | | AYS | | RQAP | | KI | | LKSRL | | YYTY | | TTVT | |
| | | LVQPG | | | | GKGL | | | | TISKD | | AMDN | | VSS | |
| | | GSLRL | | | | EWLG | | | | SSKNT | | | | | |
| | | SCAAS | | | | M | | | | VYLQM | | | | | |
| | | | | | | | | | | NSLRA | | | | | |
| | | | | | | | | | | EDTAV | | | | | |
| | | | | | | | | | | YYC | | | | | |

*Names correspond with name in informal sequence listing.

TABLE 6
Anti-interleukin (IL)-13 antibody Light Chain Kabat CDRs

| | | | SEQ | | SEQ | | SEQ | | SEQ | | SEQ | | SEQ | | SEQ |
|---|---|---|---|---|---|---|---|---|---|---|---|---|---|---|---|
| Construct | | | ID | | ID | | ID | | ID | | ID | | ID | | ID |
| ID | VL Name* | FR1 | NO: | CDR1 | NO: | FR2 | NO: | CDR2 | NO: | FR3 | NO: | CDR3 | NO: | FR4 | NO: |
| 1 | Lebrikizumab- | DIVMT | 581 | RASKS | 141 | WYQQ | 286 | LASNL | 153 | GVPDR | 343 | QQNNE | 165 | FGGGT | 400 |
| (lebrikizumab), | LC | QSPDS | | VDSYG | | KPGQP | | ES | | FSGSG | | DPRT | | KVEIK | |
| 2, | | LSVSL | | NSFMH | | PKLLIY | | | | SGTDF | | | | | |
| and 128- | | GERAT | | | | | | | | TLTISS | | | | | |
| 131 | | INC | | | | | | | | LQAED | | | | | |
| | | | | | | | | | | VAVY | | | | | |
| | | | | | | | | | | YC | | | | | |
| 3 and 4 | LC0 | NIVLT | 230 | RASKS | 141 | WYQQ | 286 | LASNL | 153 | GVPAR | 344 | QQNNE | 165 | FGGGT | 401 |
| | | QSPAS | | VDSYG | | KPGQP | | ES | | FSGSG | | DPRT | | KLEIK | |
| | | LAVSL | | NSFMH | | PKLLIY | | | | SRTDF | | | | | |
| | | GQRAT | | | | | | | | TLTIDP | | | | | |
| | | ISC | | | | | | | | VEADD | | | | | |
| | | | | | | | | | | AASYY | | | | | |
| | | | | | | | | | | C | | | | | |
| — | LC1 | DIQLT | 231 | RASKS | 141 | WYQQ | 288 | LASNL | 153 | GVPSR | 345 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SRTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 5-9 | LC2 | DIQLT | 231 | RASKS | 141 | WYQQ | 288 | LASNL | 153 | GVPSR | 345 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SRTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 10-14 | LC3 | EIVLT | 233 | RASKS | 141 | WYQQ | 290 | LASNL | 153 | GIPARF | 347 | QQNNE | 165 | FGGGT | 400 |
| | | QSPAT | | VDSYG | | KPGQA | | ES | | SGSGS | | DPRT | | KVEIK | |
| | | LSVSP | | NSFMH | | PRLLIY | | | | RTEFT | | | | | |
| | | GERAT | | | | | | | | LTISSL | | | | | |
| | | LSC | | | | | | | | QSEDF | | | | | |
| | | | | | | | | | | AVYYC | | | | | |
| — | LC4 | DIVLT | 234 | RASKS | 141 | WYLQ | 291 | LASNL | 153 | GVPDR | 348 | QQNNE | 165 | FGGGT | 400 |
| | | QSPLS | | VDSYG | | KPGQS | | ES | | FSGSG | | DPRT | | KVEIK | |
| | | LPVTP | | NSFMH | | PQLLIY | | | | SRTDF | | | | | |
| | | GEPASI | | | | | | | | TLKISR | | | | | |
| | | SC | | | | | | | | VEAED | | | | | |
| | | | | | | | | | | VGVY | | | | | |
| | | | | | | | | | | YC | | | | | |
| — | LC5 | DIVLT | 235 | RASKS | 141 | WYQQ | 286 | LASNL | 153 | GVPDR | 343 | QQNNE | 165 | FGGGT | 400 |
| | | QSPDS | | VDSYG | | KPGQP | | ES | | FSGSG | | DPRT | | KVEIK | |
| | | LAVSL | | NSFMH | | PKLLIY | | | | SGTDF | | | | | |
| | | GERAT | | | | | | | | TLTISS | | | | | |
| | | INC | | | | | | | | LQAED | | | | | |
| | | | | | | | | | | VAVY | | | | | |
| | | | | | | | | | | YC | | | | | |
| 15-19, 90, | LC6 | DIQLT | 231 | RASKS | 141 | WYQQ | 288 | LASNL | 153 | GVPSR | 349 | QQNNE | 165 | FGGGT | 400 |
| 105-127, | | QSPSSL | | VDSYG | | KPGKA | | ES | | FSGSG | | DPRT | | KVEIK | |
| and 132- | | SASVG | | NSFMH | | PKLLIY | | | | SGTDF | | | | | |
| 136 | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 20-24 | LC7 | EIVLT | 233 | RASKS | 141 | WYQQ | 290 | LASNL | 153 | GIPARF | 351 | QQNNE | 165 | FGGGT | 400 |
| | | QSPAT | | VDSYG | | KPGQA | | ES | | SGSGS | | DPRT | | KVEIK | |
| | | LSVSP | | NSFMH | | PRLLIY | | | | GTEFT | | | | | |
| | | GERAT | | | | | | | | LTISSL | | | | | |
| | | LSC | | | | | | | | QSEDF | | | | | |
| | | | | | | | | | | AVYYC | | | | | |
| — | LC8 | DIVLT | 234 | RASKS | 141 | WYLQ | 291 | LASNL | 153 | GVPDR | 352 | QQNNE | 165 | FGGGT | 400 |
| | | QSPLS | | VDSYG | | KPGQS | | ES | | FSGSG | | DPRT | | KVEIK | |
| | | LPVTP | | NSFMH | | PQLLIY | | | | SGTDF | | | | | |
| | | GEPASI | | | | | | | | TLKISR | | | | | |
| | | SC | | | | | | | | VEAED | | | | | |
| | | | | | | | | | | VGVY | | | | | |
| | | | | | | | | | | YC | | | | | |
| 25 | LC9 | DIVLT | 239 | RASKS | 141 | WYQQ | 286 | LASNL | 153 | GVPDR | 353 | QQNNE | 165 | FGGGT | 401 |
| | | QSPAS | | VDSYG | | KPGQP | | ES | | FSGSG | | DPRT | | KLEIK | |
| | | LAVSP | | NSFMH | | PKLLIY | | | | SGTDF | | | | | |
| | | GERAT | | | | | | | | TLTISR | | | | | |
| | | ISC | | | | | | | | VEADD | | | | | |
| | | | | | | | | | | VAVY | | | | | |
| | | | | | | | | | | YC | | | | | |
| 26 | LC10 | DIVLT | 239 | RASQS | 142 | WYQQ | 286 | LASNR | 158 | GVPDR | 353 | QQNN | 166 | FGGGT | 401 |
| | | QSPAS | | VDSNG | | KPGQP | | ES | | FSGSG | | HTPRT | | KLEIK | |
| | | LAVSP | | NNFLH | | PKLLIY | | | | SGTDF | | | | | |
| | | GERAT | | | | | | | | TLTISR | | | | | |
| | | ISC | | | | | | | | VEADD | | | | | |
| | | | | | | | | | | VAVY | | | | | |
| | | | | | | | | | | YC | | | | | |
| 91 | LC6_m1 | DIQLT | 231 | RASKS | 143 | WYQQ | 288 | LASNL | 153 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSRMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 92 | LC6_m2 | DIQLT | 231 | RASKS | 144 | WYQQ | 288 | LASNL | 153 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSSMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 93 | LC6_m3 | DIQLT | 231 | RASKS | 141 | WYQQ | 300 | LASNL | 153 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIR | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 94 | LC6_m4 | DIQLT | 231 | RASKS | 141 | WYQQ | 301 | LASNL | 153 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIF | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 95 | LC6_m5 | DIQLT | 231 | RASKS | 141 | WYQQ | 288 | LASHL | 154 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 96 | LC6_m6 | DIQLT | 231 | RASKS | 141 | WYQQ | 288 | LASDL | 155 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 97 | LC6_m7 | DIQLT | 231 | RASKS | 141 | WYQQ | 288 | LASQL | 156 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 98 and | LC6_m8 | DIQLT | 231 | RASKS | 141 | WYQQ | 288 | LASEL | 157 | GVPSR | 349 | QQNNE | 165 | FGGGT | 400 |
| 137-140 | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 99 | LC6_m9 | DIQLT | 231 | RASKS | 141 | WYQQ | 288 | LASNL | 153 | GVPSR | 349 | QQNHE | 167 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 100 | LC6_m10 | DIQLT | 231 | RASKS | 141 | WYQQ | 288 | LASNL | 153 | GVPSR | 349 | QQNYE | 168 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 101 | LC6_m11 | DIQLT | 231 | RASKS | 141 | WYQQ | 288 | LASNL | 153 | GVPSR | 349 | QQNSE | 169 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 102 | LC6_m12 | DIQLT | 231 | RASKS | 141 | WYQQ | 288 | LASNL | 153 | GVPSR | 349 | QQNNR | 170 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 103 | LC6_m13 | DIQLT | 231 | RASKS | 141 | WYQQ | 288 | LASNL | 153 | GVPSR | 349 | QQNN | 171 | FGGGT | 400 |
| | | QSPSSL | | VDSYG | | KPGKA | | ES | | FSGSG | | DDPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 104 | LC6_m14 | DIQLT | 231 | RASKS | 141 | WYQQ | 288 | LASNL | 153 | GVPSR | 349 | QQNN | 172 | FGGGT | 400 |
| | | QSPSSL | | VDSYG | | KPGKA | | ES | | FSGSG | | QDPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |

*Names correspond with name in informal sequence listing.

TABLE 7
Anti-interleukin (IL)-13 antibody Light Chain Chothia CDRs

| | | | SEQ | | SEQ | | SEQ | | SEQ | | SEQ | | SEQ | | SEQ |
|---|---|---|---|---|---|---|---|---|---|---|---|---|---|---|---|
| | | | ID | | ID | | ID | | ID | | ID | | ID | | ID |
| IDConstruct | VL Name* | FR1 | NO: | CDR1 | NO: | FR2 | NO: | CDR2 | NO: | FR3 | NO: | CDR3 | NO: | FR4 | NO: |
| 1 | Lebrikizumab- | DIVMT | 581 | RASKS | 141 | WYQQ | 286 | LASNL | 153 | GVPDR | 343 | QQNNE | 165 | FGGGT | 400 |
| (lebrikizumab), | LC | QSPDS | | VDSYG | | KPGQP | | ES | | FSGSG | | DPRT | | KVEIK | |
| 2, | | LSVSL | | NSFMH | | PKLLIY | | | | SGTDF | | | | | |
| and 128- | | GERAT | | | | | | | | TLTISS | | | | | |
| 131 | | INC | | | | | | | | LQAED | | | | | |
| | | | | | | | | | | VAVY | | | | | |
| | | | | | | | | | | YC | | | | | |
| 3 and 4 | LC0 | NIVLT | 230 | RASKS | 141 | WYQQ | 286 | LASNL | 153 | GVPAR | 344 | QQNNE | 165 | FGGGT | 401 |
| | | QSPAS | | VDSYG | | KPGQP | | ES | | FSGSG | | DPRT | | KLEIK | |
| | | LAVSL | | NSFMH | | PKLLIY | | | | SRTDF | | | | | |
| | | GQRAT | | | | | | | | TLTIDP | | | | | |
| | | ISC | | | | | | | | VEADD | | | | | |
| | | | | | | | | | | AASYY | | | | | |
| | | | | | | | | | | C | | | | | |
| — | LC1 | DIQLT | 231 | RASKS | 141 | WYQQ | 288 | LASNL | 153 | GVPSR | 345 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SRTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 5-9 | LC2 | DIQLT | 231 | RASKS | 141 | WYQQ | 288 | LASNL | 153 | GVPSR | 345 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SRTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 10-14 | LC3 | EIVLT | 233 | RASKS | 141 | WYQQ | 290 | LASNL | 153 | GIPARF | 347 | QQNNE | 165 | FGGGT | 400 |
| | | QSPAT | | VDSYG | | KPGQA | | ES | | SGSGS | | DPRT | | KVEIK | |
| | | LSVSP | | NSFMH | | PRLLIY | | | | RTEFT | | | | | |
| | | GERAT | | | | | | | | LTISSL | | | | | |
| | | LSC | | | | | | | | QSEDF | | | | | |
| | | | | | | | | | | AVYYC | | | | | |
| — | LC4 | DIVLT | 234 | RASKS | 141 | WYLQ | 291 | LASNL | 153 | GVPDR | 348 | QQNNE | 165 | FGGGT | 400 |
| | | QSPLS | | VDSYG | | KPGQS | | ES | | FSGSG | | DPRT | | KVEIK | |
| | | LPVTP | | NSFMH | | PQLLIY | | | | SRTDF | | | | | |
| | | GEPASI | | | | | | | | TLKISR | | | | | |
| | | SC | | | | | | | | VEAED | | | | | |
| | | | | | | | | | | VGVY | | | | | |
| | | | | | | | | | | YC | | | | | |
| — | LC5 | DIVLT | 235 | RASKS | 141 | WYQQ | 286 | LASNL | 153 | GVPDR | 343 | QQNNE | 165 | FGGGT | 400 |
| | | QSPDS | | VDSYG | | KPGQP | | ES | | FSGSG | | DPRT | | KVEIK | |
| | | LAVSL | | NSFMH | | PKLLIY | | | | SGTDF | | | | | |
| | | GERAT | | | | | | | | TLTISS | | | | | |
| | | INC | | | | | | | | LQAED | | | | | |
| | | | | | | | | | | VAVY | | | | | |
| | | | | | | | | | | YC | | | | | |
| 15-19, 90, | LC6 | DIQLT | 231 | RASKS | 141 | WYQQ | 288 | LASNL | 153 | GVPSR | 349 | QQNNE | 165 | FGGGT | 400 |
| 105-127, | | QSPSSL | | VDSYG | | KPGKA | | ES | | FSGSG | | DPRT | | KVEIK | |
| and 132- | | SASVG | | NSFMH | | PKLLIY | | | | SGTDF | | | | | |
| 136 | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 20-24 | LC7 | EIVLT | 233 | RASKS | 141 | WYQQ | 290 | LASNL | 153 | GIP ARF | 351 | QQNNE | 165 | FGGGT | 400 |
| | | QSPAT | | VDSYG | | KPGQA | | ES | | SGSGS | | DPRT | | KVEIK | |
| | | LSVSP | | NSFMH | | PRLLIY | | | | GTEFT | | | | | |
| | | GERAT | | | | | | | | LTISSL | | | | | |
| | | LSC | | | | | | | | QSEDF | | | | | |
| | | | | | | | | | | AVYYC | | | | | |
| — | LC8 | DIVLT | 234 | RASKS | 141 | WYLQ | 291 | LASNL | 153 | GVPDR | 352 | QQNNE | 165 | FGGGT | 400 |
| | | QSPLS | | VDSYG | | KPGQS | | ES | | FSGSG | | DPRT | | KVEIK | |
| | | LPVTP | | NSFMH | | PQLLIY | | | | SGTDF | | | | | |
| | | GEPASI | | | | | | | | TLKISR | | | | | |
| | | SC | | | | | | | | VEAED | | | | | |
| | | | | | | | | | | VGVY | | | | | |
| | | | | | | | | | | YC | | | | | |
| 25 | LC9 | DIVLT | 239 | RASKS | 141 | WYQQ | 286 | LASNL | 153 | GVPDR | 353 | QQNNE | 165 | FGGGT | 401 |
| | | QSPAS | | VDSYG | | KPGQP | | ES | | FSGSG | | DPRT | | KLEIK | |
| | | LAVSP | | NSFMH | | PKLLIY | | | | SGTDF | | | | | |
| | | GERAT | | | | | | | | TLTISR | | | | | |
| | | ISC | | | | | | | | VEADD | | | | | |
| | | | | | | | | | | VAVY | | | | | |
| | | | | | | | | | | YC | | | | | |
| 26 | LC10 | DIVLT | 239 | RASQS | 142 | WYQQ | 286 | LASNR | 158 | GVPDR | 353 | QQNN | 166 | FGGGT | 401 |
| | | QSPAS | | VDSNG | | KPGQP | | ES | | FSGSG | | HTPRT | | KLEIK | |
| | | LAVSP | | NNFLH | | PKLLIY | | | | SGTDF | | | | | |
| | | GERAT | | | | | | | | TLTISR | | | | | |
| | | ISC | | | | | | | | VEADD | | | | | |
| | | | | | | | | | | VAVY | | | | | |
| | | | | | | | | | | YC | | | | | |
| 91 | LC6_m1 | DIQLT | 231 | RASKS | 143 | WYQQ | 288 | LASNL | 153 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSRMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 92 | LC6_m2 | DIQLT | 231 | RASKS | 144 | WYQQ | 288 | LASNL | 153 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSSMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 93 | LC6_m3 | DIQLT | 231 | RASKS | 141 | WYQQ | 300 | LASNL | 153 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIR | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 94 | LC6_m4 | DIQLT | 231 | RASKS | 141 | WYQQ | 301 | LASNL | 153 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIF | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 95 | LC6_m5 | DIQLT | 231 | RASKS | 141 | WYQQ | 288 | LASHL | 154 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 96 | LC6_m6 | DIQLT | 231 | RASKS | 141 | WYQQ | 288 | LASDL | 155 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 97 | LC6_m7 | DIQLT | 231 | RASKS | 141 | WYQQ | 288 | LASQL | 156 | GVPSR | 349 | QQNNE | 165 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 98 and | LC6_m8 | DIQLT | 231 | RASKS | 141 | WYQQ | 288 | LASEL | 157 | GVPSR | 349 | QQNNE | 165 | FGGGT | 400 |
| 137-144 | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 99 | LC6_m9 | DIQLT | 231 | RASKS | 141 | WYQQ | 288 | LASNL | 153 | GVPSR | 349 | QQNHE | 167 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 100 | LC6_m10 | DIQLT | 231 | RASKS | 141 | WYQQ | 288 | LASNL | 153 | GVPSR | 349 | QQNYE | 168 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 101 | LC6_m11 | DIQLT | 231 | RASKS | 141 | WYQQ | 288 | LASNL | 153 | GVPSR | 349 | QQNSE | 169 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 102 | LC6_m12 | DIQLT | 231 | RASKS | 141 | WYQQ | 288 | LASNL | 153 | GVPSR | 349 | QQNNR | 170 | FGGGT | 400 |
| | | QSPSSL | | VDSYG | | KPGKA | | ES | | FSGSG | | DPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 103 | LC6_m13 | DIQLT | 231 | RASKS | 141 | WYQQ | 288 | LASNL | 153 | GVPSR | 349 | QQNN | 171 | FGGGT | 400 |
| | | QSPSSL | | VDSYG | | KPGKA | | ES | | FSGSG | | DDPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |
| 104 | LC6_m14 | DIQLT | 231 | RASKS | 141 | WYQQ | 288 | LASNL | 153 | GVPSR | 349 | QQNN | 172 | FGGGT | 400 |
| | | QSPSSL | | VDSYG | | KPGKA | | ES | | FSGSG | | QDPRT | | KVEIK | |
| | | SASVG | | NSFMH | | PKLLIY | | | | SGTDF | | | | | |
| | | DRVTI | | | | | | | | TLTISS | | | | | |
| | | TC | | | | | | | | LQPED | | | | | |
| | | | | | | | | | | FATYY | | | | | |
| | | | | | | | | | | C | | | | | |

*Names correspond with name in informal sequence listing.

TABLE 8
Anti-interleukin (IL)-13 antibody Light Chain IMGT CDRs

| | | | SEQ | | SEQ | | SEQ | | | SEQ | | SEQ | | SEQ |
|---|---|---|---|---|---|---|---|---|---|---|---|---|---|---|
| Construct | | | ID | | ID | | ID | | | ID | | ID | | ID |
| ID | VL Name* | FR1 | NO: | CDR1 | NO: | FR2 | NO: | CDR2 | FR3 | NO: | CDR3 | NO: | FR4 | NO: |
| 1 | Lebrikizumab- | DIVMT | 582 | KSVDS | 149 | MHWY | 589 | LAS | NLESG | 596 | QQNNE | 165 | FGGGT | 400 |
| (lebrikizumab), | LC | QSPDS | | YGNSF | | QQKPG | | | VPDRF | | DPRT | | KVEIK | |
| 2, | | LSVSL | | | | QPPKL | | | SGSGS | | | | | |
| and 128- | | GERAT | | | | LIY | | | GTDFT | | | | | |
| 131 | | INCRA | | | | | | | LTISSL | | | | | |
| | | S | | | | | | | QAEDV | | | | | |
| | | | | | | | | | AVYYC | | | | | |
| 3 and 4 | LC0 | NIVLT | 583 | KSVDS | 149 | MHWY | 589 | LAS | NLESG | 597 | QQNNE | 165 | FGGGT | 401 |
| | | QSPAS | | YGNSF | | QQKPG | | | VPARF | | DPRT | | KLEIK | |
| | | LAVSL | | | | QPPKL | | | SGSGS | | | | | |
| | | GQRAT | | | | LIY | | | RTDFT | | | | | |
| | | ISCRAS | | | | | | | LTIDPV | | | | | |
| | | | | | | | | | EADDA | | | | | |
| | | | | | | | | | ASYYC | | | | | |
| — | LC1 | DIQLT | 584 | KSVDS | 149 | MHWY | 590 | LAS | NLESG | 598 | QQNNE | 165 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | RTDFT | | | | | |
| | | TCRAS | | | | | | | LTISSL | | | | | |
| | | | | | | | | | QPEDF | | | | | |
| | | | | | | | | | ATYYC | | | | | |
| 5-9 | LC2 | DIQLT | 584 | KSVDS | 149 | MHWY | 590 | LAS | NLESG | 598 | QQNNE | 165 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | RTDFT | | | | | |
| | | TCRAS | | | | | | | LTISSL | | | | | |
| | | | | | | | | | QPEDF | | | | | |
| | | | | | | | | | ATYYC | | | | | |
| 10-14 | LC3 | EIVLT | 585 | KSVDS | 149 | MHWY | 591 | LAS | NLESGI | 599 | QQNNE | 165 | FGGGT | 400 |
| | | QSPAT | | YGNSF | | QQKPG | | | PARFS | | DPRT | | KVEIK | |
| | | LSVSP | | | | QAPRL | | | GSGSR | | | | | |
| | | GERAT | | | | LIY | | | TEFTL | | | | | |
| | | LSCRA | | | | | | | TISSLQ | | | | | |
| | | S | | | | | | | SEDFA | | | | | |
| | | | | | | | | | VYYC | | | | | |
| — | LC4 | DIVLT | 586 | KSVDS | 149 | MHWY | 592 | LAS | NLESG | 600 | QQNNE | 165 | FGGGT | 400 |
| | | QSPLS | | YGNSF | | LQKPG | | | VPDRF | | DPRT | | KVEIK | |
| | | LPVTP | | | | QSPQL | | | SGSGS | | | | | |
| | | GEPASI | | | | LIY | | | RTDFT | | | | | |
| | | SCRAS | | | | | | | LKISR | | | | | |
| | | | | | | | | | VEAED | | | | | |
| | | | | | | | | | VGVY | | | | | |
| | | | | | | | | | YC | | | | | |
| — | LC5 | DIVLT | 587 | KSVDS | 149 | MHWY | 589 | LAS | NLESG | 596 | QQNNE | 165 | FGGGT | 400 |
| | | QSPDS | | YGNSF | | QQKPG | | | VPDRF | | DPRT | | KVEIK | |
| | | LAVSL | | | | QPPKL | | | SGSGS | | | | | |
| | | GERAT | | | | LIY | | | GTDFT | | | | | |
| | | INCRA | | | | | | | LTISSL | | | | | |
| | | S | | | | | | | QAEDV | | | | | |
| | | | | | | | | | AVYYC | | | | | |
| 15-19, 90, | LC6 | DIQLT | 584 | KSVDS | 149 | MHWY | 590 | LAS | NLESG | 601 | QQNNE | 165 | FGGGT | 400 |
| 105-127, | | QSPSSL | | YGNSF | | QQKPG | | | VPSRF | | DPRT | | KVEIK | |
| and 132- | | SASVG | | | | KAPKL | | | SGSGS | | | | | |
| 136 | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 20-24 | LC7 | EIVLT | 585 | KSVDS | 149 | MHWY | 591 | LAS | NLESGI | 602 | QQNNE | 165 | FGGGT | 400 |
| | | QSPAT | | YGNSF | | QQKPG | | | PARFS | | DPRT | | KVEIK | |
| | | LSVSP | | | | QAPRL | | | GSGSG | | | | | |
| | | GERAT | | | | LIY | | | TEFTL | | | | | |
| | | LSCRA | | | | | | | TISSLQ | | | | | |
| | | S | | | | | | | SEDFA | | | | | |
| | | | | | | | | | VYYC | | | | | |
| — | LC8 | DIVLT | 586 | KSVDS | 149 | MHWY | 592 | LAS | NLESG | 603 | QQNNE | 165 | FGGGT | 400 |
| | | QSPLS | | YGNSF | | LQKPG | | | VPDRF | | DPRT | | KVEIK | |
| | | LPVTP | | | | QSPQL | | | SGSGS | | | | | |
| | | GEPASI | | | | LIY | | | GTDFT | | | | | |
| | | SCRAS | | | | | | | LKISR | | | | | |
| | | | | | | | | | VEAED | | | | | |
| | | | | | | | | | VGVY | | | | | |
| | | | | | | | | | YC | | | | | |
| 25 | LC9 | DIVLT | 588 | KSVDS | 149 | MHWY | 589 | LAS | NLESG | 604 | QQNNE | 165 | FGGGT | 401 |
| | | QSPAS | | YGNSF | | QQKPG | | | VPDRF | | DPRT | | KLEIK | |
| | | LAVSP | | | | QPPKL | | | SGSGS | | | | | |
| | | GERAT | | | | LIY | | | GTDFT | | | | | |
| | | ISCRAS | | | | | | | LTISRV | | | | | |
| | | | | | | | | | EADDV | | | | | |
| | | | | | | | | | AVYYC | | | | | |
| 26 | LC10 | DIVLT | 588 | QSVDS | 150 | LHWY | 593 | LAS | NRESG | 605 | QQNN | 166 | FGGGT | 401 |
| | | QSPAS | | NGNNF | | QQKPG | | | VPDRF | | HTPRT | | KLEIK | |
| | | LAVSP | | | | QPPKL | | | SGSGS | | | | | |
| | | GERAT | | | | LIY | | | GTDFT | | | | | |
| | | ISCRAS | | | | | | | LTISRV | | | | | |
| | | | | | | | | | EADDV | | | | | |
| | | | | | | | | | AVYYC | | | | | |
| 91 | LC6_m1 | DIQLT | 584 | KSVDS | 151 | MHWY | 590 | LAS | NLESG | 601 | QQNNE | 165 | FGGGT | 400 |
| | | QSPSSL | | YGNSR | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 92 | LC6_m2 | DIQLT | 584 | KSVDS | 152 | MHWY | 590 | LAS | NLESG | 601 | QQNNE | 165 | FGGGT | 400 |
| | | QSPSSL | | YGNSS | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 93 | LC6_m3 | DIQLT | 584 | KSVDS | 149 | MHWY | 594 | LAS | NLESG | 601 | QQNNE | 165 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIR | | | GTDFT | | | | | |
| | | TCRAS | | | | | | | LTISSL | | | | | |
| | | | | | | | | | QPEDF | | | | | |
| | | | | | | | | | ATYYC | | | | | |
| 94 | LC6_m4 | DIQLT | 584 | KSVDS | 149 | MHWY | 595 | LAS | NLESG | 601 | QQNNE | 165 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIF | | | GTDFT | | | | | |
| | | TCRAS | | | | | | | LTISSL | | | | | |
| | | | | | | | | | QPEDF | | | | | |
| | | | | | | | | | ATYYC | | | | | |
| 95 | LC6_m5 | DIQLT | 584 | KSVDS | 149 | MHWY | 590 | LAS | HLESG | 606 | QQNNE | 165 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 96 | LC6_m6 | DIQLT | 584 | KSVDS | 149 | MHWY | 590 | LAS | DLESG | 607 | QQNNE | 165 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 97 | LC6_m7 | DIQLT | 584 | KSVDS | 149 | MHWY | 590 | LAS | QLESG | 608 | QQNNE | 165 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 98 and | LC6_m8 | DIQLT | 584 | KSVDS | 149 | MHWY | 590 | LAS | ELESG | 609 | QQNNE | 165 | FGGGT | 400 |
| 137-144 | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 99 | LC6_m9 | DIQLT | 584 | KSVDS | 149 | MHWY | 590 | LAS | NLESG | 601 | QQNHE | 167 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 100 | LC6_m10 | DIQLT | 584 | KSVDS | 149 | MHWY | 590 | LAS | NLESG | 601 | QQNYE | 168 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 101 | LC6_m11 | DIQLT | 584 | KSVDS | 149 | MHWY | 590 | LAS | NLESG | 601 | QQNSE | 169 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 102 | LC6_m12 | DIQLT | 584 | KSVDS | 149 | MHWY | 590 | LAS | NLESG | 601 | QQNNR | 170 | FGGGT | 400 |
| | | QSPSSL | | YGNSF | | QQKPG | | | VPSRF | | DPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 103 | LC6_m13 | DIQLT | 584 | KSVDS | 149 | MHWY | 590 | LAS | NLESG | 601 | QQNN | 171 | FGGGT | 400 |
| | | QSPSSL | | YGNSF | | QQKPG | | | VPSRF | | DDPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |
| 104 | LC6_m14 | DIQLT | 584 | KSVDS | 149 | MHWY | 590 | LAS | NLESG | 601 | QQNN | 172 | FGGGT | 400 |
| | | QSPSSL | | YGNSF | | QQKPG | | | VPSRF | | QDPRT | | KVEIK | |
| | | SASVG | | | | KAPKL | | | SGSGS | | | | | |
| | | DRVTI | | | | LIY | | | GTDFT | | | | | |
| | | TCRAS | | | | | | | LTISSL | | | | | |
| | | | | | | | | | QPEDF | | | | | |
| | | | | | | | | | ATYYC | | | | | |

*Names correspond with name in informal sequence listing.

### Fc Region

The structures of the Fc regions of various immunoglobulins, and the glycosylation sites contained therein, are known in the art. See Schroeder and Cavacini, J. Allergy Clin. Immunol., 2010, 125: S41-52, incorporated by reference in its entirety. The Fc region may be a naturally occurring Fc region or an Fc region modified as described in the art or elsewhere in this disclosure.

Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e., a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG1, IgG2, IgG3, IgG4, IgA1, and IgA2.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known in the art for selectively altering the affinity of the Fc for different Fcgamma receptors. In some embodiments, the Fc comprises one or more modifications to promote selective binding of Fc-gamma receptors.

Exemplary mutations that alter the binding of FcRs to the Fc are listed below:
S298A/E333A/K334A, S298A/E333A/K334A/K326A (Lu Y, Vernes J M, Chiang N, et al. J Immunol Methods. 2011 Feb. 28; 365 (1-2): 132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67 (18): 8882-90; Nordstrom J L, Gorlatov S, Zhang W, et al. Breast Cancer Res. 2011 Nov. 30; 13 (6): R123);
F243L (Stewart R, Thom G, Levens M, et al. Protein Eng Des Sel. 2011 September; 24 (9): 671-8.), S298A/E333A/K334A (Shields R L, Namenuk A K, Hong K, et al. J Biol Chem. 2001 Mar. 2; 276 (9): 6591-604);
S239D/1332E/A330L, S239D/1332E (Lazar G A, Dang W, Karki S, et al. Proc Natl Acad Sci USA. 2006 Mar. 14; 103 (11): 4005-10);
S239D/S267E, S267E/L328F (Chu S Y, Vostiar I, Karki S, et al. Mol Immunol. 2008 September; 45 (15): 3926-33); and
S239D/D265S/S298A/1332E, S239E/S298A/K326A/A327H, G237F/S298A/A330L/1332E, S239D/1332E/S298A, S239D/K326E/A330L/1332E/S298A, G236A/S239D/D270L/1332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments an antibody described herein includes modifications to improve its ability to mediate effector function. Such modifications are known in the art and include afucosylation, or engineering of the affinity of the Fc towards an activating receptor, mainly FCGR3a for ADCC, and towards C1q for CDC. The following Table 9 summarizes various designs reported in the literature for effector function engineering.

Methods of producing antibodies with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells (von Horsten et al. (2010) Glycobiology. 2010 December; 20 (12): 1607-18). Another approach to obtaining antibodies with lowered levels of fucosylation can be found in U.S. Pat. No. 8,409,572, which teaches selecting cell lines for antibody production for their ability to yield lower levels of fucosylation on antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15%, or less than 5% of the amount of fucose normally detected for a similar antibody produced by a mammalian expression system.

Thus, in one embodiment, an antibody described herein can include a dimeric Fc that comprises one or more amino acid modifications as noted in Table 9 that confer improved effector function. In another embodiment, the antibody can be afucosylated to improve effector function.

TABLE 9
CH2 domains and effector function engineering

| Reference | Mutations | Effect |
|---|---|---|
| Lu, 2011, Ferrara | Afucosylated | Increased ADCC |
| 2011, Mizushima | | |
| 2011 | | |
| Lu, 2011 | S298A/E333A/K334A | Increased ADCC |
| Lu, 2011 | S298A/E333A/K334A/K326A | Increased ADCC |
| Stavenhagen, | F243L/R292P/Y300L/V305I/ | Increased ADCC |
| 2007 | P396L | |
| Nordstrom, 2011 | F243L/R292P/Y300L/L235V/ | Increased ADCC |
| | P396L | |
| Stewart, 2011 | F243L | Increased ADCC |
| Shields, 2001 | S298A/E333A/K334A | Increased ADCC |
| Lazar, 2006 | S239D/1332E/A330L | Increased ADCC |
| Lazar, 2006 | S239D/1332E | Increased ADCC |
| Bowles, 2006 | AME-D, not specified mutations | Increased ADCC |
| Heider, 2011 | 37.1, mutations not disclosed | Increased ADCC |
| Moore, 2010 | S267E/H268F/S324T | Increased CDC |

Fc modifications reducing FcgR and/or complement binding and/or effector function are known in the art. Recent publications describe strategies that have been used to engineer antibodies with reduced or silenced effector activity (see Strohl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, U.S. Patent Publication No. 2011/0212087 (Strohl), International Patent Publication No. WO 2006/105338 (Xencor), U.S. Patent Publication No. 2012/0225058 (Xencor), U.S. Patent Publication No. 2012/0251531 (Genentech), and Strop et al. ((2012) J. Mol. Biol. 420:204-219) describe specific modifications to reduce FcgR or complement binding to the Fc.

Specific, non-limiting examples of known amino acid modifications to reduce FcgR or complement binding to the Fc include those identified in the following Table 10:

TABLE 10
Modifications to reduce FcgR or complement binding to the Fc

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IGG4combo |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glyco |
| Medimune | L234F/L235E/P331S |
| Trubion | Hinge mutant, possibly C226S/P230S |

Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87:614-622; Kanda et al., Biotechnol. Bioeng., 2006, 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies.

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15%, or less than 5% of the amount of fucose normally detected for a similar antibody produced by a mammalian expression system.

In some embodiments, an antibody provided herein comprises an IgG1 domain with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some embodiments, such antibodies do not comprise any fucose at position Asn 297. The amount of fucose may be determined using any suitable method, for example as described in WO 2008/077546, incorporated by reference in its entirety.

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al., Biotech. Bioeng., 2004, 87:614-622; each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which his incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

In certain embodiments, an antibody provided herein comprises a heavy chain comprising a constant heavy chain sequence selected from the sequences set forth in SEQ ID NOs: 425-468 and 484-539.

In certain embodiments, the antibody comprises a VH sequence set forth in SEQ ID NO: 1 and a VL sequence set forth in SEQ ID NO: 33; and wherein the human Fc region comprises a human IgG sequence selected from a sequence set forth in SEQ ID NOs: 436-468 and 484-539. In certain embodiments, the antibody comprises a VH sequence set forth in SEQ ID NO: 2 and a VL sequence set forth in SEQ ID NO: 33; and wherein the human Fc region comprises a human IgG sequence selected from a sequence set forth in SEQ ID NOs: 436-468 and 484-539. In certain embodiments, the antibody comprises a VH sequence set forth in SEQ ID NO: 3 and a VL sequence set forth in SEQ ID NO: 35; and wherein the human Fc region comprises a human IgG sequence selected from a sequence set forth in SEQ ID NOs: 436-468 and 484-539. In certain embodiments, the antibody comprises a VH sequence set forth in SEQ ID NO: 4 and a VL sequence set forth in SEQ ID NO: 35; and wherein the human Fc region comprises a human IgG sequence selected from a sequence set forth in SEQ ID NOs: 436-468 and 484-539. In certain embodiments, the antibody comprises a VH sequence set forth in SEQ ID NO: 5 and a VL sequence set forth in SEQ ID NO: 35; and wherein the human Fc region comprises a human IgG sequence selected from a sequence set forth in SEQ ID NOs: 436-468 and 484-539. In certain embodiments, the antibody comprises a VH sequence set forth in SEQ ID NO: 6 and a VL sequence set forth in SEQ ID NO: 35; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 35; and wherein the human Fc region comprises a human IgG sequence selected from a sequence set forth in SEQ ID NOs: 436-468 and 484-539. In certain embodiments, the antibody comprises a VH sequence set forth in SEQ ID NO: 3 and a VL sequence set forth in SEQ ID NO: 36; and wherein the human Fc region comprises a human IgG sequence selected from a sequence set forth in SEQ ID NOs: 436-468 and 484-539. In certain embodiments, the antibody comprises a VH sequence set forth in SEQ ID NO: 4 and a VL sequence set forth in SEQ ID NO: 36; and wherein the human Fc region comprises a human IgG sequence selected from a sequence set forth in SEQ ID NOs: 436-468 and 484-539. In certain embodiments, the antibody comprises a VH sequence set forth in SEQ ID NO: 5 and a VL sequence set forth in SEQ ID NO: 36; and wherein the human Fc region comprises a human IgG sequence selected from a sequence set forth in SEQ ID NOs: 436-468 and 484-539. In certain embodiments, the antibody comprises a VH sequence set forth in SEQ ID NO: 6 and a VL sequence set forth in SEQ ID NO: 36; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 36; and wherein the human Fc region comprises a human IgG sequence selected from a sequence set forth in SEQ ID NOs: 439, 440, 446, 457 and 460. In certain embodiments, the antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 4 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 5 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 6 and a VL sequence set forth in SEQ ID NO: 39; and wherein the human Fc region comprises a human IgG sequence selected from a sequence set forth in SEQ ID NOs: 436-468. In certain embodiments, the antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 3 and a VL sequence set forth in SEQ ID NO: 40; and wherein the human Fc region comprises a human IgG sequence selected from a sequence set forth in SEQ ID NOs: 436-468 and 484-539. In certain embodiments, the antibody comprises a VH sequence set forth in SEQ ID NO: 4 and a VL sequence set forth in SEQ ID NO: 40; and wherein the human Fc region comprises a human IgG sequence selected from a sequence set forth in SEQ ID NOs: 436-468 and 484-539. In certain embodiments, the antibody comprises a VH sequence set forth in SEQ ID NO: 5, and a VL sequence set forth in SEQ ID NO: 40; and wherein the human Fc region comprises a human IgG sequence selected from a sequence set forth in SEQ ID NOs: 436-468 and 484-539. In certain embodiments, the antibody comprises a VH sequence set forth in SEQ ID NO: 6 and a VL sequence set forth in SEQ ID NO: 40; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 40; and wherein the human Fc region comprises a human IgG sequence selected from a sequence set forth in SEQ ID NOs: 436-468 and 484-539. In certain embodiments, the antibody comprises a VH sequence set forth in SEQ ID NO: 8 and a VL sequence set forth in SEQ ID NO: 42; and wherein the human Fc region comprises a human IgG sequence selected from a sequence set forth in SEQ ID NOs: 436-468 and 484-539. In certain embodiments, the antibody comprises a VH sequence set forth in SEQ ID NO: 9 and a VL sequence set forth in SEQ ID NO: 43; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7, and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 44; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 45; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 46; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 47; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 48; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 49; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 50; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 52; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 53; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 54; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 55; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 56; and wherein the human Fc region comprises a human IgG sequence selected from a sequence set forth in SEQ ID NOs: 436-468 and 484-539. In certain embodiments, the antibody comprises a VH sequence set forth in SEQ ID NO: 7 and a VL sequence set forth in SEQ ID NO: 57; and wherein the human Fc region comprises a human IgG sequence selected from a sequence set forth in SEQ ID NOs: 436-468 and 484-539. In certain embodiments, the antibody comprises a VH sequence set forth in SEQ ID NO: 10 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 11 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 12 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 13 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 14 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 15 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 16 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 17 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 18 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 19 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 20 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 21 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 22 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 23 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 24 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 25 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 26 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 27 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 28 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 28 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 29 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 30 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 31 and a VL sequence set forth in SEQ ID NO: 39; and wherein the human Fc region comprises a human IgG sequence selected from a sequence set forth in SEQ ID NOS: 436-468 and 484-539. In certain embodiments, the antibody comprises a VH sequence set forth in SEQ ID NO: 32 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 8 and a VL sequence set forth in SEQ ID NO: 39; and wherein the human Fc region comprises a human IgG sequence selected from a sequence set forth in SEQ ID NOs: 436-468 and 484-539. In certain embodiments, the antibody comprises a VH sequence set forth in SEQ ID NO: 8 and a VL sequence set forth in SEQ ID NO: 51; and wherein the human Fc region comprises a human IgG sequence selected from a sequence set forth in SEQ ID NOs: 436-468 and 484-539. In certain embodiments, the antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NOs: 436-468 and 484-539. In certain embodiments, the antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NOs: 436-468 and 484-539.

In certain embodiments, the antibody comprises a VH sequence set forth in SEQ ID NO: 15 and a VL sequence set forth in SEQ ID NO: 39; and wherein the human Fc region comprises a human IgG sequence set forth in SEQ ID NO: 439. In certain embodiments, the antibody comprises a VH sequence set forth in SEQ ID NO: 15 and a VL sequence set forth in SEQ ID NO: 39; and wherein the human Fc region comprises a human IgG sequence set forth in SEQ ID NO: 457. In certain embodiments, the antibody comprises a VH sequence set forth in SEQ ID NO: 15 and a VL sequence set forth in SEQ ID NO: 39; and wherein the human Fc region comprises a human IgG sequence set forth in SEQ ID NO: 460. In certain embodiments, the antibody comprises a VH sequence set forth in SEQ ID NO: 15 and a VL sequence set forth in SEQ ID NO: 39; and wherein the human Fc region comprises a human IgG sequence set forth in SEQ ID NO: 446. In certain embodiments, the antibody comprises a VH sequence set forth in SEQ ID NO: 3 and a VL sequence set forth in SEQ ID NO: 39; and wherein the human Fc region comprises a human IgG sequence set forth in SEQ ID NO: 439. In certain embodiments, the antibody comprises a VH sequence set forth in SEQ ID NO: 3 and a VL sequence set forth in SEQ ID NO: 39; and wherein the human Fc region comprises a human IgG sequence set forth in SEQ ID NO: 457. In certain embodiments, the antibody comprises a VH sequence set forth in SEQ ID NO: 3 and a VL sequence set forth in SEQ ID NO: 39; and wherein the human Fc region comprises a human IgG sequence set forth in SEQ ID NO: 460. In certain embodiments, the antibody comprises a VH sequence set forth in SEQ ID NO: 3 and a VL sequence set forth in SEQ ID NO: 39; and wherein the human Fc region comprises a human IgG sequence set forth in SEQ ID NO: 446. In certain embodiments, the antibody comprises a VH sequence set forth in SEQ ID NO: 7 and a VL sequence set forth in SEQ ID NO: 51; and wherein the human Fc region comprises a human IgG sequence set forth in SEQ ID NO: 439. In certain embodiments, the antibody comprises a VH sequence set forth in SEQ ID NO: 7 and a VL sequence set forth in SEQ ID NO: 51; and wherein the human Fc region comprises a human IgG sequence set forth in SEQ ID NO: 457. In certain embodiments, the antibody comprises a VH sequence set forth in SEQ ID NO: 7 and a VL sequence set forth in SEQ ID NO: 51; and wherein the human Fc region comprises a human IgG sequence set forth in SEQ ID NO: 460. In certain embodiments, the antibody comprises a VH sequence set forth in SEQ ID NO: 7 and a VL sequence set forth in SEQ ID NO: 51; and wherein the human Fc region comprises a human IgG sequence set forth in SEQ ID NO: 446. In certain embodiments, the antibody comprises a VH sequence set forth in SEQ ID NO: 8 and a VL sequence set forth in SEQ ID NO: 39; and wherein the human Fc region comprises a human IgG sequence set forth in SEQ ID NO: 439. In certain embodiments, the antibody comprises a VH sequence set forth in SEQ ID NO: 8 and a VL sequence set forth in SEQ ID NO: 39; and wherein the human Fc region comprises a human IgG sequence set forth in SEQ ID NO: 457. In certain embodiments, the antibody comprises a VH sequence set forth in SEQ ID NO: 8 and a VL sequence set forth in SEQ ID NO: 51; and wherein the human Fc region comprises a human IgG sequence set forth in SEQ ID NO: 460. In certain embodiments, the antibody comprises a VH sequence set forth in SEQ ID NO: 8 and a VL sequence set forth in SEQ ID NO: 51; and wherein the human Fc region comprises a human IgG sequence set forth in SEQ ID NO: 446. In certain embodiments, the antibody comprises a VH sequence set forth in SEQ ID NO: 470 and a VL sequence set forth in SEQ ID NO: 471; and wherein the human Fc region comprises a human IgG sequence set forth in SEQ ID NO: 439. In certain embodiments, the antibody comprises a VH sequence set forth in SEQ ID NO: 470 and a VL sequence set forth in SEQ ID NO: 471; and wherein the human Fc region comprises a human IgG sequence set forth in SEQ ID NO: 457. In certain embodiments, the antibody comprises a VH sequence set forth in SEQ ID NO: 470 and a VL sequence set forth in SEQ ID NO: 471; and wherein the human Fc region comprises a human IgG sequence set forth in SEQ ID NO: 460. In certain embodiments, the antibody comprises a VH sequence set forth in SEQ ID NO: 470 and a VL sequence set forth in SEQ ID NO: 471; and wherein the human Fc region comprises a human IgG sequence set forth in SEQ ID NO: 446. In certain embodiments, the antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NO: 439. In certain embodiments, the antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NO: 446. In certain embodiments, the antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NO: 457. In certain embodiments, the antibody comprises a VH sequence set forth in SEQ ID NO: 3 and a VL sequence set forth in SEQ ID NO: 39; and wherein the human Fc region comprises a human IgG sequence selected from a sequence set forth in SEQ ID NO: 460. In certain embodiments, the antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NO: 439. In certain embodiments, the antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NO: 446. In certain embodiments, the antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NO: 457. In certain embodiments, the antibody comprises a VH sequence set forth in SEQ ID NO: 7 and a VL sequence set forth in SEQ ID NO: 39; and wherein the human Fc region comprises a human IgG sequence selected from a sequence set forth in SEQ ID NO: 460. In certain embodiments, the antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NO: 439. In certain embodiments, the antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NO: 446. In certain embodiments, the antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NO: 457. In certain embodiments, the antibody comprises a VH sequence set forth in SEQ ID NO: 3 and a VL sequence set forth in SEQ ID NO: 51; and wherein the human Fc region comprises a human IgG sequence selected from a sequence set forth in SEQ ID NO: 460. In certain embodiments, the antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NO: 439. In certain embodiments, the antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NO: 446. In certain embodiments, the antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NO: 457. In certain embodiments, the antibody comprises a VH sequence set forth in SEQ ID NO: 7 and a VL sequence set forth in SEQ ID NO: 51; and wherein the human Fc region comprises a human IgG sequence selected from a sequence set forth in SEQ ID NO: 460. In certain embodiments, the antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NO: 439. In certain embodiments, the antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NO: 446. In certain embodiments, the antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NO: 457. In certain embodiments, the antibody comprises a VH sequence set forth in SEQ ID NO: 470 and a VL sequence set forth in SEQ ID NO: 471; and wherein the human Fc region comprises a human IgG sequence selected from a sequence set forth in SEQ ID NO: 460.

In certain embodiments, the isolated antibody described herein comprises a constant light chain sequence set forth by SEQ ID NO: 469.

In certain embodiments, the Fc region comprises one or more amino acid substitutions, wherein the one or more substitutions result in increased antibody half-life, increased ADCC activity, increased ADCP activity, or increased CDC activity compared with the Fc without the one or more substitutions. In certain embodiments, the one or more amino acid substitutions results in increased antibody half-life at pH 6.0 compared to an antibody comprising a wild-type Fc region. In certain embodiments, the isolated antibody comprising an Fc region with one or more amino acid substitutions has a half-life of about 80 to 110 days in a human.

In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to lebrikizumab.

In certain embodiments, the Fc region comprises one or more amino acid substitutions, wherein the one or more substitutions result in increased antibody half-life, a decrease in one or more of ADCC activity, ADCP activity, or CDC activity compared with the Fc without the one or more substitutions. In certain embodiments, the one or more amino acid substitutions results in increased antibody half-life at pH 6.0 compared to an antibody comprising a wild-type Fc region. In certain embodiments, the isolated antibody comprising an Fc region with one or more amino acid substitutions has a half-life of about 80 to 110 days in a human.

In certain embodiments, the one or more amino acid substitutions is selected from the group consisting of S228P (SP); M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W. In certain embodiments, the one or more amino acid substitutions comprises a plurality of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LA), M428L/N434A L234A/G237A (LALA), L234A/L235A/G237A, L234A/L235A/P329G, N297A, D265A/YTE, LALA/YTE, LAGA/YTE, LALAGA/YTE, LALAPG/YTE, N297A/LS; D265A/LS; LALA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS, SP/DHS; SPLE/DHS; N297A/LA; D265A/LA, LALA/LA, LAGA/LA, LALAGA/LA, LALAPG/LA, N297A/N434A; D265A/N434A; LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

In certain embodiments, the one or more amino acid substitutions is selected from the group consisting of LALA/YTE, LAGA/YTE, LALA/LS, YTE, and LS.

In certain embodiments, the one or more amino acid substitutions comprises or consists of LALA/YTE. In certain embodiments, the one or more amino acid substitutions comprises or consists of LAGA/YTE. In certain embodiments, the one or more amino acid substitutions comprises or consists of LALA/LS. In certain embodiments, the one or more amino acid substitutions comprises or consists of YTE. In certain embodiments, the one or more amino acid substitutions comprises or consists of LS.

In certain embodiments, the Fc region binds an Fcγ Receptor selected from the group consisting of: FcγRI, FcγRIIa, FcγRIIb, FcγRIIc, FcγRIIIa, and FcγRIIIb. In certain embodiments, the Fc region binds an Fcγ Receptor with higher affinity at pH 6.0 compared to an antibody comprising a wild-type Fc region.

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule (i.e., IL-13) and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of an anti-IL-13 antibody for a non-target molecule is less than about 50% of the affinity for IL-13. In some embodiments, the affinity of an anti-IL-13 antibody for a non-target molecule is less than about 40% of the affinity for IL-13. In some embodiments, the affinity of an anti-IL-13 antibody for a non-target molecule is less than about 30% of the affinity for IL-13. In some embodiments, the affinity of an anti-IL-13 antibody for a non-target molecule is less than about 20% of the affinity for IL-13. In some embodiments, the affinity of an anti-IL-13 antibody for a non-target molecule is less than about 10% of the affinity for IL-13. In some embodiments, the affinity of an anti-IL-13 antibody for a non-target molecule is less than about 1% of the affinity for IL-13. In some embodiments, the affinity of an anti-IL-13 antibody for a non-target molecule is less than about 0.1% of the affinity for IL-13.

When used herein in the context of two or more antibodies, the term “competes with” or “cross-competes with” indicates that the two or more antibodies compete for binding to an antigen (e.g., IL-13). In one exemplary assay, IL-13 is coated on a surface and contacted with a first anti-IL-13 antibody, after which a second anti-IL-13 antibody is added. In another exemplary assay, a first anti-IL-13 antibody is coated on a surface and contacted with IL-13, and then a second anti-IL-13 antibody is added. If the presence of the first anti-IL-13 antibody reduces binding of the second anti-IL-13 antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, at least 95%, or at least 99% as measured in a competitive binding assay. A skilled artisan can select the concentrations of the antibodies used in the competition assays based on the affinities of the antibodies for IL-13 and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Antibodies identified by competition assay (competing antibody) include antibodies binding to the same epitope as the reference antibody and antibodies binding to an adjacent epitope sufficiently proximal to the epitope bound by the reference antibody for steric hindrance to occur. For example, a second, competing antibody can be identified that competes for binding to IL-13 with a first antibody described herein. In certain instances, the second antibody can block or inhibit binding of the first antibody by, e.g., at least 50%, 60%, 70%, 75%, 80%, 85%, 90%, 95%, or 99% as measured in a competitive binding assay. In certain instances, the second antibody can displace the first antibody by greater than 50%, 60%, 70%, 75%, 80%, 85%, 90%, 95%, or 99%.

In certain embodiments, the antibody binds a human IL-13.

In certain embodiments, the antibody binds an IL-13 sequence set forth in SEQ ID NOs: 472-475.

In certain embodiments, the antibody is cross-reactive to cynomolgus monkey IL-13.

In certain embodiments, the antibody binds to an IL-13 sequence set forth in SEQ ID NOs: 472-475 with a K_{D} of less than or equal to about 1, 2, 3, 4, 5, 6, 7, 8, 9×10⁻⁹ M, as measured by SPR. In certain embodiments, the antibody binds to an IL-13 sequence set forth in SEQ ID NOs: 472-475 with a K_{D} of less than or equal to about 1×10⁻¹⁰ M, as measured by SPR. In certain embodiments, the antibody binds to human IL-13 with a K_{D} of less than or equal to about 1×10⁻⁹ M, as measured by SPR.

In some embodiments, an antibody provided herein binds IL-13 with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁸ M, as measured by ELISA or any other suitable method known in the art. In some embodiments, an antibody provided herein binds IL-13 with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁹ M, as measured by ELISA or any other suitable method known in the art.

In some embodiments, the K_{D} of the antibody provided herein for the binding of IL-13 is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁸ M, as measured by ELISA or any other suitable method known in the art. In some embodiments, an antibody provided herein binds IL-13 with a K_{D} of less than or equal to about 1×10⁻⁸ M, or less than or equal to above 1×10⁻⁹ M as measured by ELISA or any other suitable method known in the art.

In some embodiments, the antibody provided herein binds IL-13 with a K_{D} of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁸ M, or less, as measured by ELISA or any other suitable method known in the art. In some embodiments, the antibody provided herein binds IL-13 with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

### Function

“Effector functions” refer to those biological activities mediated by the Fc region of an antibody, which activities may vary depending on the antibody isotype. Examples of antibody effector functions include receptor ligand blocking, agonism, or antagonism, C1q binding to activate complement dependent cytotoxicity (CDC), Fc receptor binding to activate antibody-dependent cellular cytotoxicity (ADCC), and antibody dependent cellular phagocytosis (ADCP). In some embodiments, the effector function of the anti-IL-13 antibody described herein is antagonism and blocks the IL-13 receptor binding to IL-13.

### Pharmaceutical Compositions

The present application provides compositions comprising the antibodies including pharmaceutical compositions comprising any one or more of the antibodies described herein with one or more pharmaceutically acceptable excipients. In some embodiments the composition is sterile. The pharmaceutical compositions generally comprise an effective amount of an antibody.

These compositions can comprise, in addition to one or more of the antibodies disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

The anti-IL-13 antibody that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16^{th} edition, Osol, A. (ed), 1980.

A composition can be administered alone or in combination with other treatments, either simultaneously or sequentially dependent upon the condition to be treated.

### Methods

### Methods of Preparation

Antibodies described herein can be produced using recombinant methods and compositions, e.g., as described in U.S. Pat. No. 4,816,567. In one embodiment, an isolated nucleic acid encoding an antibody described herein is provided. Such a nucleic acid may encode an amino acid sequence comprising the VL and/or an amino acid sequence comprising the VH of the antibody (e.g., the light and/or heavy chains of the antibody) or an amino acid sequence comprising the VHH of a single domain antibody. In a further embodiment, one or more vectors (e.g., expression vectors) comprising such nucleic acid are provided. In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g., a Chinese Hamster Ovary (CHO) cell, or human embryonic kidney (HEK) cell, or lymphoid cell (e.g., Y0, NS0, Sp20 cell). In one embodiment, a method of making an antibody is provided, wherein the method comprises culturing a host cell comprising nucleic acid encoding the antibody, as provided above, under conditions suitable for expression of the antibody, and optionally recovering the antibody from the host cell (or host cell culture medium).

For recombinant production of the antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

When an antibody or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the antibody or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” antibody produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp2O, CV-1, VERO-76, HeLa, HepG2, Per.C6, or BHK.

For example, antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the antibodies described herein are produced in stable mammalian cells, by a method comprising: transfecting at least one stable mammalian cell with: nucleic acid encoding the antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antibody in the expressed product.

In some embodiments, is the method of producing an antibody in stable mammalian cells as described herein wherein the expression product of the at least one stable mammalian cell comprises a larger percentage of the desired glycosylated antibody as compared to the monomeric heavy or light chain polypeptides, or other antibodies.

In some embodiments, is the method of producing a glycosylated antibody in stable mammalian cells described herein, said method comprising identifying and purifying the desired glycosylated antibody. In some embodiments, the said identification is by one or both of liquid chromatography and mass spectrometry.

If required, the antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed or immobilized anti-flag antibody if a flag-tag is used. For general guidance in suitable purification techniques, see, e.g., incorporated entirely by reference Protein Purification: Principles and Practice, 3^{rd} Ed., Scopes, Springer-Verlag, NY, 1994, incorporated entirely by reference. The degree of purification necessary will vary depending on the use of the antibodies. In some instances, no purification is necessary.

In certain embodiments, the antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antibodies described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

In certain embodiments, an antibody described herein has an aggregation temperature greater than about 69° C., greater than about 70° C., greater than about 71° C., greater than about 72° C., greater than about 73° C., greater than about 74° C., greater than about 75° C., or greater than about 76° C., for example, between about 69° C. and about 77° C., between about 70° C. and about 76° C., between about 71° C. and about 75° C. In certain embodiments, aggregation temperature is measured using DSF.

In certain embodiments, an antibody described herein has reduced hydrophobicity as compared to lebrikizumab as measured by hydrophobic interaction chromatography (HIC). In certain embodiments, the antibody exhibits an HIC retention time that is less than about 15.2 min. In certain embodiments, the antibody exhibits an HIC retention time that is between about 13 min and about 15 min.

### Methods of Use

In an aspect, the present application provides methods of contacting IL-13 with an anti-IL-13 antibody, such as a human or humanized antibody, which results in inhibition of IL-13 binding to an IL-13 receptor expressed on a cell.

In an aspect, the present application provides methods of using the isolated anti-IL-13 antibodies described herein for treatment of a disorder or disease in a subject. In certain aspects, described herein is a method for treating a subject in need thereof with an anti-IL-13 antibody, the method comprising administering to a mammalian subject a therapeutically effective amount of an anti-IL-13 antibody or pharmaceutical composition comprising an anti-IL-13 antibody described herein. In certain embodiments, the present application provides methods of treating a disorder or disease associated with elevated levels of IL-13 and/or IgE in a subject.

In certain aspects, described herein are methods for treating a pathology associated with IL-13 activity, the method comprising administering to a mammalian subject a therapeutically effective amount an isolated anti-IL-13 antibody or a pharmaceutical composition comprising an isolated anti-IL-13 antibody described herein.

In certain aspects, described herein is a method for treating an inflammatory disorder or disease in a mammalian subject in need thereof, the method comprising administering to the mammalian subject a therapeutically effective amount an antibody described herein or a pharmaceutical composition described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments of the methods described herein, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (ENID) selected from the group consisting of Eosinophilic Gastritis (EoG), Eosinophilic enteritis (EoN), Eosinophilic colitis (EoC), and Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn disease or ulcerative colitis. In certain embodiments, the inflammatory disorder or disease is psoriasis. In certain embodiments, the inflammatory disorder or disease is lupus. In certain embodiments, the inflammatory disorder or disease is rheumatoid arthritis.

In certain aspects, described herein are methods for treating a pathology associated with elevated levels of IL-13 in a mammalian subject in need thereof, the method comprising administering to the mammalian subject a therapeutically effective amount an antibody or a pharmaceutical composition described herein.

In certain aspects, described herein are methods of reducing biological activity of IL-13 in a mammalian subject in need thereof, the method comprising administering to the mammalian subject a therapeutically effective amount an antibody or a pharmaceutical composition described herein.

In certain aspects, described herein are methods for inhibiting the TH2 type allergic response in a mammalian subject in need thereof, the method comprising administering to the mammalian subject a therapeutically effective amount an antibody or a pharmaceutical composition described herein.

In certain aspects, described herein are methods for inhibiting IL-13-induced phosphorylation of STAT6 in a cell, the method comprising contacting the cell with an antibody described herein.

In certain aspects, described herein are methods for inhibiting IL-13-induced CD23 expression in a cell, the method comprising contacting the cell with an antibody described herein.

In certain aspects, described herein are methods for inhibiting IL-13-induced secretion of CCL2 and CCL26 from a cell, the method comprising contacting the cell with an antibody described herein.

In certain aspects, described herein are methods for inhibiting IL-13-induced NTRK1 expression in a cell, the method comprising contacting the cell with an antibody described herein.

In certain aspects, described herein are methods for reducing levels of Thymus and Activation Regulated Chemokine (TARC)/CCL17 in a mammalian subject in need thereof, the method comprising administering to the mammalian subject a therapeutically effective amount an antibody or a pharmaceutical composition described herein.

In certain aspects, described herein are methods of preventing an inflammatory disorder or disease in a mammalian subject in need thereof, the method comprising administering to the mammalian subject a therapeutically effective amount an antibody or a pharmaceutical composition described herein.

### Methods of Administration

In some embodiments, the methods provided herein are useful for the treatment of a disease or disorder in an individual. In an embodiment, the individual is a human and the antibody is an anti-IL-13 antibody described herein.

In some embodiments, an antibody is administered intravenously, intramuscularly, subcutaneously, topically, orally, transdermally, intraperitoneally, intraorbitally, by implantation, by inhalation, intrathecally, intraventricularly, or intranasally. An effective amount of an anti-IL-13 antibody may be administered for the treatment of a disease or disorder. The appropriate dosage of the anti-IL-13 antibody may be determined based on the type of disease or disorder to be treated, the type of the anti-IL-13 antibody, the severity and course of the disease or disorder, the clinical condition of the individual, the individual's clinical history and response to the treatment, and the discretion of the attending physician.

In some embodiments, an antibody provided herein is administered with at least one additional therapeutic agent. Any suitable additional therapeutic or immunotherapeutic agent may be administered with an antibody provided herein. Additional therapeutic agents include agents that are used to treat or prevent a disease or disorder such as, but not limited to, an inflammatory disease or disorder associated with elevated levels of IL-13 and/or IgE.

The additional therapeutic agent can be administered by any suitable means. In some embodiments, an antibody provided herein and the additional therapeutic agent are included in the same pharmaceutical composition. In some embodiments, an antibody provided herein and the additional therapeutic agent are included in different pharmaceutical compositions.

In embodiments where an antibody provided herein and the additional therapeutic agent are included in different pharmaceutical compositions, administration of the antibody can occur prior to, simultaneously, and/or following, administration of the additional therapeutic agent. In some embodiments, administration of an antibody provided herein and the additional therapeutic agent occur within about one month of each other. In some embodiments, administration of an antibody provided herein and the additional therapeutic agent occur within about one week of each other. In some embodiments, administration of an antibody provided herein and the additional therapeutic agent occur within about one day of each other. In some embodiments, administration of an antibody provided herein and the additional therapeutic agent occur within about twelve hours of each other. In some embodiments, administration of an antibody provided herein and the additional therapeutic agent occur within about one hour of each other.

### Kits and Articles of Manufacture

The present application provides kits comprising any one or more of the antibody compositions described herein and instructions for use. In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Methods

### Humanization of Mouse Hybridoma Sequence of Anti-IL-13 Antibody 228B/C-1

Complementarity-determining region (CDR) grafting technology was used to humanize the parental mouse anti-human IL-13 228B/C-1, the parental monoclonal antibody of Lebrikizumab. The parental mouse heavy and light sequences were modeled onto a human antibody framework as described below. A set of human heavy and light chains were selected for humanization. The goal was to design pairs of these heavy and light chains that resulted in improved biophysical properties of the parental antibody while retaining binding. These humanized molecules were designed for improved developability profile during scale up in bioprocess.

### Humanization of Light Chains

The parental mAb light chain sequence of mouse hybridoma sequence of anti-IL13 antibody (Lebrikizumab) was compared to a group of human variable region light chain (VK) germlines amino acid sequences (Lefranc, M.-P. IMGT, the international ImMunoGeneTics database Nucleic Acids Res., 29, D207-209 (2001). DOI: 10.1093/nar/29.1.207. PMID: 11125093.). A total of 4 human VK germlines were selected. Of these, one belonged to Vk4 family (IGKV4-1), two belonged to VK1 family (IGKV1-39 and IGKV3-15) and one belonged to VK2 family (IGKV2-28). One substitution on light chain framework 3, R to G was also designed. This back mutation from human to mouse can alter binding. Human germline KJ4 was selected for the J region based on sequence similarity with the mouse sequence. The humanized VL domains were cloned into a vector encoding for a kappa light chain constant domain.

The following nomenclature was used for the light chains: “LC0” corresponds to the mouse hybridoma sequence. “LC1” corresponds to IGKV4-1_KJ4. “LC2” corresponds to IGKV1-39_KJ4. “LC3” corresponds to IGKV3-15_KJ4. “LC4” corresponds to IGKV2-28_KJ4. “LC5” corresponds to IGKV4-1_R to G_KJ. “LC6” corresponds to IGK V1-39_R to G_KJ4. “LC7” corresponds to IGKV3-15_R to G_KJ4. “LC8” corresponds to IGKV2-28_R to G_KJ4.

### Humanization of Heavy Chain

The parental mAb heavy chain sequence of mouse hybridoma sequence of anti-IL13 antibody (Lebrikizumab) was compared to a group of human variable region heavy chain (VH) germline amino acid sequences. A total of 5 human VH germlines were selected. Of these, one belonged to VH4 family (IGHV4-59), two belonged to VH1 family (IGHV1-46, IGHV1-69) and two belonged to VH3 family (IGHV3-15, IGHV3-23). The N-terminal Q in heavy chain was substituted with E to prevent potential pyroglutamate conversion. Human germline HJ6 was selected for the J region based on sequence similarity with the mouse sequence. The humanized VH domains were cloned into a vector encoding for human IgG1 HC constant domain.

The following nomenclature was used for the heavy chains: “HC0”-corresponds to the mouse hybridoma heavy chain. “HC0_M” corresponds to HC0_NIS to TIS in FR3 (to prevent potential glycosylation). “HC1” corresponds to humanized sequence IGHV4-59_HJ6. “HC2” corresponds to humanized sequence IGHV1-46_HJ6. “HC3” corresponds to humanized sequence IGHV1-69_HJ6. “HC4” corresponds to humanized sequence IGHV3-15_HJ6. “HC5” corresponds to humanized sequence IGHV3-23_HJ6.

### Gene Synthesis and Plasmid Construction

The coding sequences for HC and LC of the antibody were generated by DNA synthesis and PCR, subsequently subcloned into pTT5-based plasmid for protein expression in mammalian cell system. The gene sequences in the expression vectors were confirmed by DNA sequencing.

### Expression of Antibody Constructs

Transient expression of antibodies was performed by co-transfection of paired HC and LC constructs into CHO cells using PEI method. Briefly, CHO cells at approximately 5.5×106/mL in a shake flask was used as the host. Transfection was initiated by adding a mixture of 1 mg/L DNA and 7 mg/L PEI in OptiMEM™ medium (Invitrogen) to the cells followed by gentle mixing. Cells were then cultured in an incubator shaker at 120 rpm, 37° C., and 8% CO₂, for 9 days. Feeding with peptone and glucose was carried out 24 h later and every 2-3 days thereafter depending on the cell density and viability. The cell culture was terminated on day 9 when cell viability reduced to <80%. The conditioned medium was harvested for protein purification.

### Purification of Antibody Construct

Protein purification by affinity chromatography, and ion exchange chromatography was performed using an AKTA pure instrument (GE Lifesciences). Conditional medium expressing target antibody was harvested by centrifugation at 4000 rpm, 50 min, and filtered with a 0.22 μm filter. The harvested supernatants were loaded to a column of Mabselect™ SuRe™ (GE Healthcare). After washing column with Buffer A (PBS, PH 7.4), the protein was eluted with Buffer B (1 M Glycine, pH 2.7), and immediately neutralized with 1/10 volume of Buffer D (1 M sodium citrate, pH 6.0). The affinity purified antibody was then buffer exchanged into 20 mM sodium acetate pH 5.5.

TABLE 11
Size exclusion chromatograph of anti-IL-13 constructs

| Construct ID* | Percent Monomer |
|---|---|
| lebrikizumab (Construct 1) | 99% |
| Construct 2 | 96% |
| Construct 3 | 95% |
| Construct 4 | 93% |
| Construct 5 | 95% |
| Construct 6 | 97% |
| Construct 7 | 97% |
| Construct 8 | 98% |
| Construct 9 | 96% |
| Construct 10 | 99% |
| Construct 11 | 98% |
| Construct 12 | 98% |
| Construct 13 | 98% |
| Construct 14 | 98% |
| Construct 15 | 98% |
| Construct 16 | 98% |
| Construct 17 | 99% |
| Construct 18 | 98% |
| Construct 19 | 98% |
| Construct 20 | 99% |
| Construct 21 | 98% |
| Construct 22 | 98% |
| Construct 23 | 99% |
| Construct 24 | 100% |
| Construct 25 | 99% |
| Construct 26 | 97% |
| Construct 90 | 98% |
| Construct 91 | 98% |
| Construct 92 | 98% |
| Construct 93 | 98% |
| Construct 94 | 99% |
| Construct 95 | 98% |
| Construct 96 | 99% |
| Construct 97 | 97% |
| Construct 98 | 98% |
| Construct 99 | 98% |
| Construct 100 | 97% |
| Construct 101 | 99% |
| Construct 102 | 98% |
| Construct 103 | 100% |
| Construct 104 | 100% |
| Construct 105 | 100% |
| Construct 106 | 99% |
| Construct 107 | 100% |
| Construct 108 | 99% |
| Construct 109 | 99% |
| Construct 110 | 99% |
| Construct 111 | 100% |
| Construct 112 | 98% |
| Construct 113 | 98% |
| Construct 114 | 98% |
| Construct 115 | 97% |
| Construct 116 | 98% |
| Construct 117 | 98% |
| Construct 118 | 97% |
| Construct 119 | 98% |
| Construct 120 | 97% |
| Construct 121 | 96% |
| Construct 122 | 98% |
| Construct 123 | 95% |
| Construct 124 | 98% |
| Construct 125 | 98% |
| Construct 126 | 97% |
| Construct 127 | 98% |

*See construct sequences in Tables 2-8.

### SEC-HPLC Analysis of Antibody Construct

Analytical SEC-HPLC was performed using Shimadzu LC-10 HPLC instrument (Shimadzu Corp.). 20 μl sample on 1 mg/mL was loaded to a Superdex® 200 Increase 5/150GL column (GE Lifesciences). The mobile phase was 2*PBS with a flow rate of 0.3 mL/min, 15 min.

### Measuring Antibody-IL13 Binding Kinetics Using Surface Plasmon Resonance

A Biacore 8K SPR system (GE HealthCare) equipped with Series S Sensor Chip Protein G (Cytiva, Cat. 29179315) was used to determine the binding kinetic rate and affinity constants at 25° C. and in a running buffer of HBS-EP+ (10 mM HEPES pH 7.4, 150 mM NaCl, 3 mM EDTA, 0.05% Surfactant P20). Following a stabilization period in running buffer, the anti-IL13 mAb constructs (diluted to 1 μg/mL were captured onto flow cell 2 (active) for 60 sec at a flow rate of 10 μL/min. Recombinant Human IL-13 Protein, His Tag (Acro Cat. IL3-H52H4) was prepared at concentrations of 0, 0.39, 0.78, 1.56, 3.13, 6.25, 12.5 and 0 nM and injected over flow cell 1 (reference) and flow cell 2 (active) for 180 sec at a flow rate of 30 μL/min. Recombinant Cynomolgus IL-13 Protein, His Tag (SINO BIOLOGICAL, Cat. 11057-C07H) was prepared at concentrations of 0, 0.39, 0.78, 1.56, 3.13, 6.25, 12.5, 25 and 0 nM and injected over flow cell 1 (reference) and flow cell 2 (active) for 180 sec at a flow rate of 30 μL/min. Samples were injected in a multi-cycle manner over freshly captured mAb, by regenerating the capture surfaces with injection of glycine pH 1.5 for 30 sec at a flow rate of 30 μL/min. The data was processed and analyzed with Biacore Insight Evaluation Software Version 2.0.15.12933 (GE Healthcare) as follows. Responses from flow cell 1 (reference) were subtracted from the responses from flow cell 2 (active). The responses from the two buffer blank injections were then subtracted from the reference subtracted data (2-1) to yield double-referenced data, which were fit to an 1:1 binding model to determine the apparent association (ka) and dissociation rate constants (kd). Their ratio provided the apparent equilibrium dissociation constant or affinity constant (KD=kd/ka).

### Determination of Antibody Affinity to Fc Receptors and C1q

Binding affinity (K_{D}) of antibodies to Fc receptors and C1q were determined through surface plasmon resonance (SPR) using a Biacore 8K. Briefly, an SPR chip functionalized with an anti-kappa light-chain antibody was used to capture purified antibodies normalized to 5 mg/mL, at a flow rate of 10 uL/min for 90 seconds or 120 seconds. A paired channel with only buffer was used as reference. Subsequently, varying concentrations of recombinant human CD32a (167H), CD32a (167R), CD32b, CD16a (176V), CD16a (176F), FcRn, CD64, and C1q were injected over the surface with captured purified antibody as well as the reference channel. Regeneration of the chip between different concentrations of different antigens were performed with 10 mM Glycine HCl, pH 1.5 and antibody was again captured. Association and dissociation rate constants were subsequently determined through fitting to a 1:1 Langmuir binding model or steady-state analysis model, whichever applicable, using the Biacore Insight Evaluation Software from which a K_{D} value was derived.

### Assessing Blockade by Cell-Line-Based Assays

Multiple assays were used to assess blockade of the full signalling complex of IL-13/IL13Rα1/IL-4Rα and prevention of downstream signalling. Briefly, HEK293 previously transduced to stably express both hIL-13Rα and hIL-4Rα were cultured and harvested. Cells were seeded at 200,000 cells in 100 uL per well. Cells were washed and the supernatant was discarded. A 100 uL mixture of biotinylated hIL-13 and purified antibody (1:1 by volume) that had been previously made and incubated for 1 hour was added to resuspend the cells, resulting in a final concentration of 0.05 ug/mL of hIL-13 and 0-100 nM of purified antibody. The cells were stained in this mixture at 4° C. for 1 hour. Cells were then washed and stained with 100 μL of Alexa Fluor 488-conjugated streptavidin at a 1:1000 dilution to detect binding of biotinylated hIL-13 on the cell surface. Cells were incubated at 4° C. for 1 hour, protected from light. Cells were then washed and the median fluorescence intensity (MFI) of cells in each well were recorded by FACS using a BD FACSCanto II. Subsequent data were analyzed using GraphPad Prism. IC₅₀ values were determined as the concentration of antibody required to inhibit 50% of the maximum MFI of biotinylated hIL-13 surface detected with incubation of 0.05 ug/mL of hIL-13 alone.

Cell-line-based assays included: inhibition of phosphorylation of STAT6 in HT-29 cells, inhibition of release of TARC in A549 cells, and inhibition of proliferation of TF-1 cells. Primary human lymphocyte-based assays included: inhibition of phosphorylation of STAT6 and inhibition of CD23 expression.

### Inhibition of Phosphorylation of STAT6 in HT-29 Cells

Inhibition of STAT6 phosphorylation in HT-29 cells was used to evaluate the functional activity of antibodies to block IL-13-induced biological activity. Briefly, HT-29 cells were starved in RMPI 1640+0.1% FBS overnight. Cells were collected and seeded at 50,000 cells per well in 100 μL. Concurrently, a 100 μL mixture of hIL-13 and purified antibody (1:1 by volume) was added to the same well, resulting in a final concentration of 10 ng/ml of hIL-13 and 0-50 nM of purified antibody. Cells were incubated at 37° C. for 1 hour and subsequently fixed, permeabilized, and stained with a PE-conjugated anti-pSTAT6 antibody. The MFI of cells in each well were recorded by FACS using a BD FACSCanto II and subsequent data were analyzed using GraphPad Prism. IC50 values were determined as the concentration of antibody required to inhibit 50% of the maximum MFI of pSTAT6 detected with incubation of 10 ng/ml of hIL-13 alone. Results are summarized below.

### Inhibition of Release of TARC in A549 Cells

Inhibition of TARC secretion by A549 cells was used to evaluate the functional activity of antibodies to block IL-13-induced biological activity. Briefly, A549 cells were seeded at 20,000 cells in 100 μL of DMEM+10% FBS and cultured overnight at 37° C. The next day, the cell culture media was discarded and cells were gently washed with fresh media. A 150 μL mixture of hIL-13, purified antibody, and hTNFα (1:1:1 by volume) were added to the wells, resulting in a final concentration of 20 ng/ml hIL-13, 0-100 nM purified antibody, and 200 ng/mL. Cells were incubated in this mixture at 37° C. for 20-24 hour. Following incubation, culture supernatant was collected and the amount of TARC present was analyzed using a commercial TARC ELISA kit (R&D Systems), analyzed according to manufacturer's instructions. The determined concentrations of TARC in each well were analyzed using GraphPad Prism. IC50 values were determined as the concentration of antibody required to inhibit 50% of the maximum TARC concentration detected with incubation of only 20 ng/ml of hIL-13 and 200 ng/ml hTNFα. Results are summarized below.

### Inhibition of Proliferation of TF-1 Cells

The proliferation or inhibition thereof of TF-1 cells was used to evaluate the functional activity of antibodies to block IL-13-induced biological activity. Briefly, TF-1 cells were harvested and starved in RPMI1640+10% FBS without additional cytokine for 4 hours. During this time, a mixture of hIL-13 and purified antibody (1:1 by volume) was prepared 50 μL was added per well. Following starvation, TF-1 cells were again harvested and seeded at 15,000 cells in 50 μL per well, resulting in a final concentration of 4 ng/ml of hIL-13 and 0-5 nM purified antibody. Cells were subsequently incubated at 37° C. for 72 hours and proliferation of cells was quantified using CellTiter-Glo (Promega) according to manufacturer's instructions. Luminescence was recorded by SpectraMax M5 Multimode Plate Reader and data was analyzed using GraphPad Prism. IC50 values were determined as the concentration of antibody required to result in 50% of the maximum luminescence detected when TF-1 cells are incubated and cultured with 4 ng/mL of hIL-13 alone. Results are summarized below.

### Inhibition of STAT6 Phosphorylation and CD23 Expression in Primary Human Lymphocytes

To confirm the antagonistic activity of antibodies herein in primary cells, human peripheral blood mononuclear cells (PBMCs) obtained from healthy donors were used to evaluate the ability of antibodies herein to inhibit IL-13-induced phosphorylation of STAT6 and upregulation of CD23 expression.

Frozen human PBMCs from a previously-identified IL-13 responsive donors were thawed and revived. To determine the in vitro potency of antibodies herein in inhibiting STAT6 phosphorylation, total PBMCs were stimulated with 10 ng/mL of IL-13 and purified antibody (1:1 by volume). These cells were allowed to incubate for 15 minutes at 37° C. Phosphorylation of STAT6 was subsequently analysed by flow cytometry using a commercial anti-phosphoSTAT6 antibody, staining assessed within the specific immune population gated by lineage-specific markers CD14 and CD19. In a separate set of PBMCs, cells were allowed to incubate for 24 hours at 37° C. CD23 expression was subsequently analyzed by flow cytometry using an anti-CD23 antibody and its intensity of staining assessed within the specific immune population gated by lineage-specific markers CD14 and CD19.

Median fluorescence intensity (MFI) data from either pSTAT6 or CD23 staining was analyzed using GraphPad Prism. IC50 values were determined as the concentration of antibody required to result in 50% of the maximum MFI detected for each marker when primary human PBMCs are incubated and cultured with 10 ng/ml of hIL-13 alone.

### Protein Thermal Stability Test by Differential Scanning Fluorimetry (DSF)

SYPRO® Orange (Thermo Fisher #S6651) was supplied at 5000× concentration in 100% DMSO and diluted to 40× in the appropriate formulation buffer. The antibodies were mixed with the dye, and nine microliters of this mixture was loaded into a UNi (Unchained Labs, Cat No. 201-1010) and run with the “Tₘ using SYPRO” application on UNCLE (Unchained Labs). Samples were subjected to a thermal ramp from 25-95° C., with a ramp rate of 0.5° C./minute and excitation at 473 nm. Full spectra were collected from 250-720 nm and UNCLE software was used to measure the area under the curve between 510-680 nm to calculate the inflection points (Tₘ) of the transition curves.

### HIC-HPLC Analysis of Antibody Construct

Analytical HIC-HPLC was performed using Thermo UltiMate™ 3000 instrument. A 20 μl sample at 1 mg/mL was loaded to a Thermo Scientific™ MAbPac™ HIC-Butyl HPLC column (5 μm, 4.6 mm×100 mm; Cat No. 088558). The mobile phase A was 1.5 M Ammonium sulfate+50 mM PB buffer+5% (v/v) isopropyl alcohol, pH 6.95 and the mobile phase B was 50 mM PB buffer+20% (v/v) isopropyl alcohol, pH 6.95. The gradient was 0% to 100% mobile phase B over 20 min, and flow rate was 0.5 mL/min.

### Inhibition of CCL26 and CCL2 Secretion and NTRK1 Expression

Inhibition of CCL26 and CCL2 secretion and NTRK1 expression by HaCaT cells was used to evaluate the functional activity of antibodies to block IL-13-induced biological activity. HaCaT cells were seeded at 20,000 cells in 100 uL of DMEM+10% FBS and cultured overnight at 37° C. The next day, a 150 uL mixture of hIL-13 and purified antibody were added to the wells, resulting in a final concentration of 50 ng/mL of IL-13 with 0-206.5 nM purified antibody. Cells were then further incubated at 37° C. for 48 hours. Following incubation, culture supernatant was collected and levels of secreted CCL26 and CCL2 were measured using a commercial Luminex-based immunoassay kit (R&D Systems) and analyzed according to manufacturer's instructions. Determined concentrations of CCL26 and CCL2 in each well were analyzed using GraphPad Prism. IC50 values were determined as the concentration of antibody required to inhibit 50% of the maximum concentration detected with incubation of 50 ng/ml of IL-13 alone.

Cells remaining in the assay plates were lysed and mRNA extracted for analysis of NTRK1 gene expression using a commercial Quantigene kit (ThermoFisher). Levels of NTRK1 mRNA were determined according to the manufacturer's protocol and analyzed in GraphPad Prism. NTRK1 gene expression was quantified as a ratio of NTRK1 mRNA levels relative to the housekeeping gene, PPIB, and IC50 values calculated as the concentration of antibody required to inhibit 50% of the maximum gene expression detected using 50 ng/ml of hIL-13 alone.

### Example 1: Engineered Anti-IL-13 Antibodies Exhibit Improved Affinity and Potency of Blockade of IL-13

### Results

### Determination of Antibody Affinity to IL-13

Using the methods described above, the affinity of Construct 133 (see construct sequence in Tables 2-8) to IL-13 and the binding kinetics thereof were assessed using surface plasmon resonance (SPR) as compared to dupilumab, lebrikizumab, and a variant of lebrikizumab with one or more amino acid substitutions in the heavy chain constant region (Construct 2 (Lebrikizumab-HC; Lebrikizumab-LC; hIgG1-LAGA YTE; Human kappa LC); see construct sequence in Tables 2-8), and tralokinumab.

As measured by SPR, Construct 133 had an affinity of 77 pM compared to 131 pM and 116 pM for lebrikizumab and tralokinumab, respectively.

The affinity of variants of lebrikizumab with one or more amino acid substitutions in the heavy chain constant region (Constructs 128-131), variants of Constructs 15 or 98 with one or more amino acid substitutions in the heavy chain constant region (Constructs 133-136 or 137-140, respectively), and variants with one or more amino acid substitutions in the heavy chain constant region (Constructs 132 and 141-144) to human and cynomolgus monkey IL-13 and the binding kinetics thereof were also assessed using SPR.

It was observed that all antibodies bound to human IL-13 with low picomolar affinity comparable to the variants of lebrikizumab. Additionally, all antibodies tested were cross-reactive to cynomolgus monkey IL-13 with sub-nanomolar affinities (Table 12).

TABLE 12
Antibody Affinity to IL-13

| | SPR Hu | SPR Cyno | ELISA Hu |
|---|---|---|---|
| | IL-13 K_{D} | IL-13 K_{D} | IL-13 K_{D} |
| Construct ID* | (pM) | (pM) | (pM) |
| Lebrikizumab | 131 | 309 | N.T. |
| Tralokinumab | 116 | 1480 | N.T. |
| Construct 128 | 91.5 | 298 | 67 |
| Construct 129 | 239 | 882 | 36 |
| Construct 130 | 112 | 227 | 27 |
| Construct 131 | 75.6 | 123 | 19 |
| Construct 132 | 135 | 331 | N.T. |
| Construct 133 | 77.7 | 208 | 19 |
| Construct 134 | 105 | 330 | 50 |
| Construct 135 | 130 | 588 | N.T. |
| Construct 136 | 69.8 | 161 | 18 |
| Construct 137 | 42.1 | 246 | 26 |
| Construct 138 | 108 | 348 | N.T. |
| Construct 139 | 114 | 705 | N.T. |
| Construct 140 | 47.5 | 252 | 20 |
| Construct 141 | 107 | 329 | 25 |
| Construct 142 | 108 | 326 | 54 |
| Construct 143 | 119 | 685 | N.T. |
| Construct 144 | 26.3 | 228 | 14 |

*See construct sequences in Tables 2-8.
N.T.—Not Tested

### Antibody Affinity to Fc Receptors and C1q

All antibody hIgG1-LALA YTE variants compared to lebrikizumab showed near or complete ablation of binding to all Fc gamma receptors, significantly decreased binding to C1q, and significantly increased binding to FcRn at pH 5.8 (Table 13).

TABLE 13
Antibody Affinity to Fc Receptors and C1q

| | | | | | | | | cRn, | cRn, |
|---|---|---|---|---|---|---|---|---|---|
| | D32a | D32a | | D16a | D16a | | | pH | pH |
| | (167H) | (167R) | D32b | (176V) | (176F) | D64 | C1q (Signal | 7.4 | 5.8 |
| Construct | K_{D} | K_{D} | K_{D} | K_{D} | K_{D} | K_{D} | Relative to | K_{D} | K_{D} |
| ID* | (mM) | (mM) | (mM) | (mM) | (mM) | (nM) | Rituximab) | (mM) | (mM) |
| Lebrikizumab | 5.27 | 2.38 | 2.93 | n.d. | n.d. | 1.12 | 39.6% | n.d. | 1.06 |
| Construct | n.d. | n.d. | n.d. | n.d. | n.d. | n.d. | 15.9% | n.d. | 0.15 |
| 133 | | | | | | | | | |
| Construct | n.d. | 8.14 | 8.62 | n.d. | n.d. | 2.80 | 25.3% | n.d. | 0.16 |
| 134 | | | | | | | | | |
| Construct | 4.54 | 2.40 | 2.17 | n.d. | n.d. | 0.86 | 30.8% | n.d. | 0.10 |
| 135 | | | | | | | | | |
| Construct | n.d. | n.d. | n.d. | 4.67 | n.d. | n.d. | 25.4% | n.d. | 0.09 |
| 136 | | | | | | | | | |

*See construct sequences in Tables 2-8.
n.d.—not detectable

### Blockade

A. Inhibition of IL-13 Binding to hIL-13Rα/hIL-4Rα Overexpressing Cells

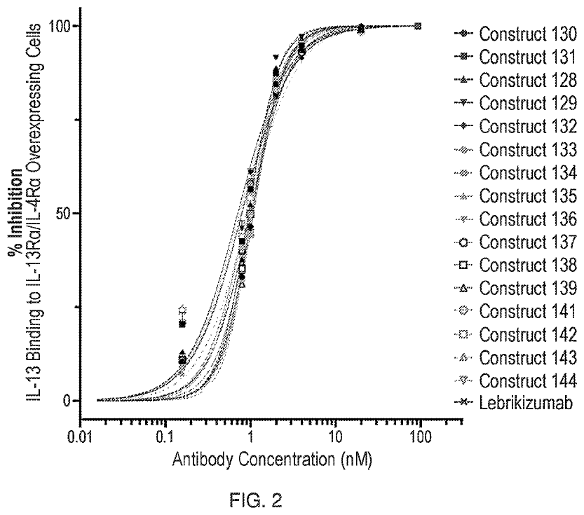

IL-13 binding to cells overexpressing hIL-13Rα/hIL-4Rα was used to evaluate the functional blockade of antibodies against this binding interaction. The results of the functional blockade of antibodies described herein in blocking IL-13 binding to cells overexpressing hIL-13Rα/hIL-4Rα are provided in Table 14 and FIG. 2. In the cell-line-based assays, Construct 133 exhibited an IC₅₀ of 0.89 nM and inhibited IL-13 binding on an IL-13Rα1/IL-4Rα overexpression cell line, as compared to an IC₅₀ 1.11 nM for lebrikizumab.

TABLE 14
Blockade of IL-13 Binding to hIL-13Rα/hIL-
4Rα Overexpressing Cells

| | Blockade of IL-13 |
|---|---|
| Construct ID* | Binding IC₅₀ (nM) |
| Lebrikizumab | 1.11 |
| Construct 128 | 0.95 |
| Construct 129 | 0.71 |
| Construct 130 | 1.05 |
| Construct 131 | 0.79 |
| Construct 132 | 0.99 |
| Construct 133 | 0.89 |
| Construct 134 | 1.08 |
| Construct 135 | 1.03 |
| Construct 136 | 1.02 |
| Construct 137 | 0.89 |
| Construct 138 | 1.01 |
| Construct 139 | 1.09 |
| Construct 141 | 0.81 |
| Construct 142 | 0.77 |
| Construct 143 | 0.74 |
| Construct 144 | 0.94 |

*See construct sequences in Tables 2-8.

### B. Inhibition of IL-13-Induced Phosphorylation of STAT6 in HT-29 Cells

Inhibition of STAT6 phosphorylation in HT-29 cells was used to evaluate the functional activity of antibodies to block IL-13-induced biological activity. An IC₅₀ of 0.28 nM of Construct 133 was observed for inhibiting phosphorylation of STAT6 in HT-29 cells, as compared to 0.16 nM for dupilumab, 0.23 nM for lebrikizumab, and 0.41 nM for tralokinumab, respectively.

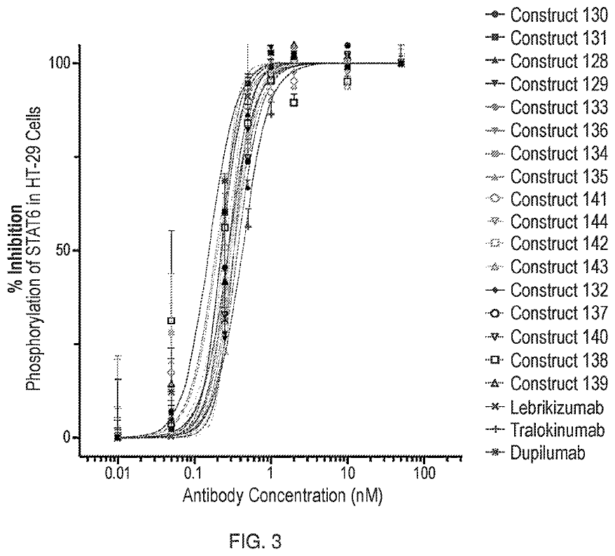

Variants of lebrikizumab with one or more amino acid substitutions in the heavy chain constant region (Constructs 128-131), variants of Constructs 15 or 98 with one or more amino acid substitutions in the heavy chain constant region (Constructs 133-136 or 137-140, respectively), and variants with one or more amino acid substitutions in the heavy chain constant region (Constructs 132 and 141-144) were also tested in the same assay (Table 15 and FIG. 3).

TABLE 15
Inhibition of IL-13-Induced Phosphorylation
of STAT6 in HT-29 Cells

| Construct ID* | pSTAT6 Inhibition IC₅₀ (nM) |
|---|---|
| Lebrikizumab | 0.23 |
| Tralokinumab | 0.41 |
| Dupilumab | 0.16 |
| Construct 128 | 0.28 |
| Construct 129 | 0.33 |
| Construct 130 | 0.28 |
| Construct 131 | 0.22 |
| Construct 132 | 0.37 |
| Construct 133 | 0.28 |
| Construct 134 | 0.20 |
| Construct 135 | 0.23 |
| Construct 136 | 0.30 |
| Construct 137 | 0.33 |
| Construct 138 | 0.19 |
| Construct 139 | 0.28 |
| Construct 140 | 0.33 |
| Constuct 141 | 0.25 |
| Construct 142 | 0.29 |
| Construct 143 | 0.36 |
| Construct 144 | 0.30 |

*See construct sequences in Tables 2-8.

### C. Inhibition of IL-13 TARC Secretion by Engineered Anti-IL13 Antibody Variants

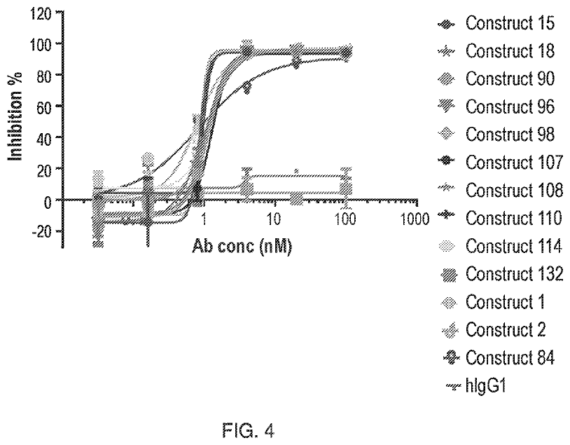

Human A549 cells express IL4Rα/IL13Rα1 receptor, which responds to binding to human IL-13 by inducing pSTAT6 phosphorylation, thereby triggering expression of downstream genes involved in TH2 type allergic response. Thymus and Activation Regulated Chemokine (TARC), also known as CCL17, is one such gene product that is secreted by multiple cell types and plays a role in attracting effector immune cells such as eosinophils that are involved in inflammation. A549 cells were contacted with engineered anti-IL-13 antibodies and TARC assays were performed (FIG. 4). Anti-IL-13 antibodies inhibited secretion of TARC as measured by ELISA. The TARC secretion IC₅₀ profiles (Table 16) were similar to lebrikizumab, thus confirming there was a preservation of potency of the anti-IL13 antibodies for IL-13 sequestrant activity in cell-based assays.

TABLE 16
TARC secretion

| | TARC |
|---|---|
| | Release |
| | IC50 |
| Construct ID* | (nM) |
| Lebrikizumab | 0.9 |
| Construct 2 | 0.9 |
| Construct 15 | 0.9 |
| Construct 19 | 1.1 |
| Construct 19 | 1.0 |
| Construct 96 | 1.0 |
| Construct 98 | 0.9 |
| Construct 107 | 1.3 |
| Construct 108 | 1.0 |
| Construct 110 | 1.0 |
| Construct 114 | 1.1 |
| Dupilumab control | 0.8 |
| Control (Irrelevant | No |
| IgG1 LAGA YTE) | Inhibition |

*See construct sequences in Tables 2-8.

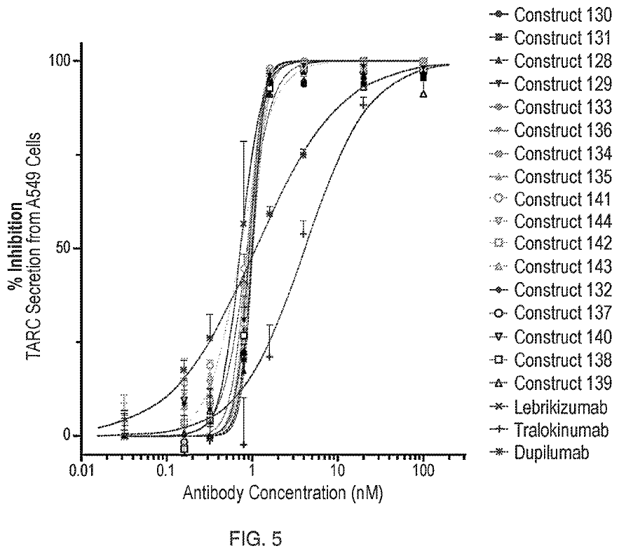

Variants of lebrikizumab with one or more amino acid substitutions in the heavy chain constant region (Constructs 128-131), variants of Constructs 15 or 98 with one or more amino acid substitutions in the heavy chain constant region (Constructs 133-136 or 137-140, respectively), and variants with one or more amino acid substitutions in the heavy chain constant region (Constructs 132 and 141-144) were tested in the same assay (Table 17 and FIG. 5). An IC₅₀ of 0.86 nM by Construct 133 for inhibiting release of TARC in A549 cells was observed, as compared to 1.11 nM for dupilumab, 0.74 nM for lebrikizumab, and 4.14 nM for tralokinumab, respectively.

TABLE 17
Inhibition of IL-13-Induced Release of TARC in A549 Cells

| Construct ID* | TARC Inhibition IC₅₀ (nM) |
|---|---|
| Lebrikizumab | 0.74 |
| Tralokinumab | 4.14 |
| Dupilumab | 1.11 |
| Construct 128 | 1.01 |
| Construct 129 | 0.92 |
| Construct 130 | 1.00 |
| Construct 131 | 0.99 |
| Construct 132 | 0.87 |
| Construct 133 | 0.86 |
| Construct 134 | 0.99 |
| Construct 135 | 0.91 |
| Construct 136 | 0.89 |
| Construct 137 | 0.87 |
| Construct 138 | 0.97 |
| Construct 139 | 1.02 |
| Construct 140 | 0.94 |
| Construct 141 | 0.73 |
| Construct 142 | 0.92 |
| Construct 143 | 0.92 |
| Construct 144 | 0.91 |

*See construct sequences in Tables 2-8.

### D. Inhibition of IL-13-Induced Proliferation of TF-1 Cells

An IC50 of 0.16 nM by Construct 133 for inhibiting proliferation of IL-13-induced TF-1 cells was observed, as compared to 0.19 nM for dupilumab, 0.20 nM lebrikizumab, and 0.59 nM for tralokinumab, respectively.

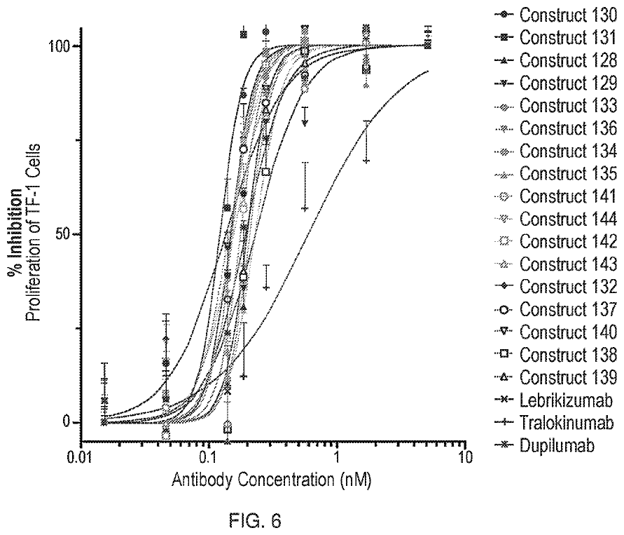

Variants of lebrikizumab with one or more acid substitutions in the heavy chain constant region (Constructs 128-131), variants of Constructs 15 or 98 with one or more amino acid substitutions in the heavy chain constant region (Constructs 133-136 or 137-140, respectively), and variants with one or more amino acid substitutions in the heavy chain constant region (Constructs 132 and 141-144) were also tested in the same assay (Table 18 and FIG. 6).

TABLE 18
Inhibition of IL-13-Induced Proliferation of TF-1 Cells

| | TF-1 Proliferation |
|---|---|
| Construct ID* | Inhibition IC₅₀ (nM) |
| Lebrikizumab | 0.20 |
| Tralokinumab | 0.59 |
| Dupilumab | 0.19 |
| Construct 128 | 0.20 |
| Construct 129 | 0.21 |
| Construct 130 | 0.15 |
| Construct 131 | 0.13 |
| Construct 132 | 0.13 |
| Construct 133 | 0.15 |
| Construct 134 | 0.18 |
| Construct 135 | 0.21 |
| Construct 136 | 0.15 |
| Construct 137 | 0.16 |
| Construct 138 | 0.23 |
| Construct 139 | 0.20 |
| Construct 140 | 0.14 |
| Construct 141 | 0.19 |
| Construct 142 | 0.20 |
| Construct 143 | 0.17 |
| Construct 144 | 0.17 |

*See construct sequences in Tables 2-8.

### E. Inhibition of IL-13-Induced Phosphorylation of STAT6 and CD23 Expression in Primary Human Lymphocytes

In primary human lymphocytes, Construct 133 potently blocked IL-13 activity in a dose-dependent manner as exhibited by an IC50 of 0.44 nM inhibiting phosphorylation of STAT6 compared to 0.38 nM for lebrikizumab and an IC50 0.85 nM in inhibiting CD23 expression compared to 0.81 nM for lebrikizumab. The results demonstrated the strong antagonistic activity that Construct 133 possessed against IL-13-mediated signalling in primary human cells.

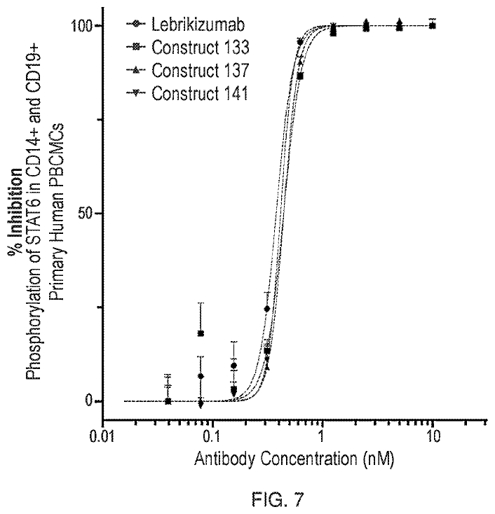

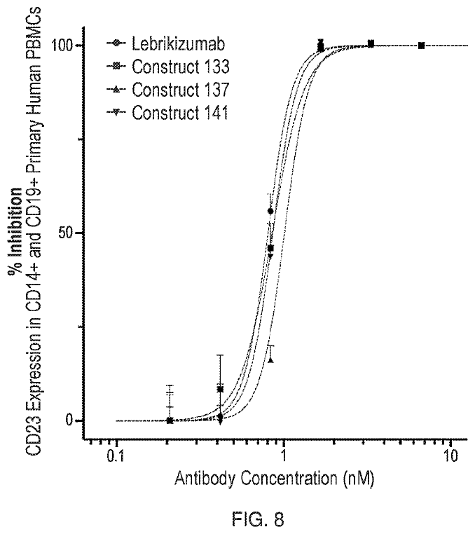

A variant of Construct 98 with one or more amino acid substitutions in the heavy chain constant region (Construct 137), and a variant with one or more amino acid substitutions in the heavy chain constant region (Construct 141) were also tested in the same assay (Table 19, FIG. 7, and FIG. 8).

TABLE 19
Inhibition of IL-13-Induced Phosphorylation
of STAT6 and CD23 in Human PBMCs

| | pSTAT6 Inhibition | CD23 Expression |
|---|---|---|
| Construct ID* | IC₅₀ (nM) | Inhibition IC₅₀ (nM) |
| Lebrikizumab | 0.38 | 0.81 |
| Construct 133 | 0.44 | 0.85 |
| Construct 137 | 0.44 | 1.01 |
| Construct 141 | 0.41 | 0.86 |

### Example 2: Expression of Engineered Anti-IL-13 Antibodies is Improved Over Lebrikizumab

Transient expression of antibodies was performed by co-transfection of paired HC and LC constructs into CHO cells using the PEI method as described above. The relative expression of the engineered antibody constructs from the CHO cell lysates compared to lebrikizumab were then determined (Table 20) in a small-scale expression screening experiment.

TABLE 20
Expression of engineered anti-IL-13 antibodies

| | Fold Improvement in |
|---|---|
| | Transient CHO Expression |
| Construct* | Over lebrikizumab |
| Lebrikizumab | 1.0 |
| Construct 2 | 1.4 |
| Construct 3 | 8.0 |
| Construct 4 | 6.4 |
| Construct 5 | 4.3 |
| Construct 6 | 5.4 |
| Construct 7 | 7.2 |
| Construct 8 | 3.7 |
| Construct 9 | 4.3 |
| Construct 10 | 2.1 |
| Construct 11 | 2.6 |
| Construct 12 | 3.5 |
| Construct 13 | 5.2 |
| Construct 14 | 3.1 |
| Construct 15 | 5.9 |
| Construct 16 | 9.1 |
| Construct 17 | 8.4 |
| Construct 18 | 6.4 |
| Construct 19 | 6.8 |
| Construct 20 | 4.7 |
| Construct 21 | 3.9 |
| Construct 22 | 4.5 |
| Construct 23 | 2.9 |
| Construct 24 | 0.2 |
| Construct 25 | 13.2 |
| Construct 26 | 9.4 |
| Construct 90 | 9.9 |
| Construct 91 | 9.7 |
| Construct 92 | 11.5 |
| Construct 93 | 8.9 |
| Construct 94 | 5.6 |
| Construct 95 | 3.3 |
| Construct 96 | 6.8 |
| Construct 97 | 5.2 |
| Construct 98 | 6.8 |
| Construct 99 | 3.5 |
| Construct 100 | 8.1 |
| Construct 101 | 12.2 |
| Construct 102 | 4.1 |
| Construct 103 | 6.2 |
| Construct 104 | 6.4 |
| Construct 105 | 3.5 |
| Construct 106 | 3.9 |
| Construct 107 | 8.5 |
| Construct 108 | 6.2 |
| Construct 109 | 6.0 |
| Construct 110 | 9.7 |
| Construct 111 | 12.4 |
| Construct 112 | 12.9 |
| Construct 113 | 0.2 |
| Construct 114 | 4.4 |
| Construct 115 | 9.1 |
| Construct 116 | 7.8 |
| Construct 117 | 5.8 |
| Construct 118 | 11.8 |
| Construct 119 | 9.1 |
| Construct 120 | 7.2 |
| Construct 121 | 5.8 |
| Construct 122 | 7.8 |
| Construct 123 | 8.9 |
| Construct 124 | 9.5 |
| Construct 125 | 5.8 |
| Construct 126 | 6.2 |
| Construct 127 | 0.9 |

*See construct sequences in Tables 2-8.

### Example 3: Thermostability of Engineered Anti-IL-13 Antibodies

Differential Scanning Fluorometry (DSF) was utilized to measure melting temperatures (Tm2) of lebrikizumab and the IL-13 antibodies described in Table 21. While the majority of variants exhibited a non-inferior Tm2 profile to lebrikizumab, there were a handful of variants that exhibited a significantly higher melting temperature, such as Construct 5 and Construct 15 (Table 21). Additionally, nearly all variants exhibited higher aggregation temperature, Tagg, compared to lebrikizumab. Tagg is a measure of the propensity of the mAb for forming higher molecules weight aggregates, and higher values are more desirable. Thus, the majority of the engineered variants had a more desirable aggregation temperature.

TABLE 21
Thermostability of engineered anti-IL-13 antibodies

| | | Tagg |
|---|---|---|
| | Tm2 | 473 |
| Construct ID* | (° C.) | (° C.) |
| Lebrikizumab | 71.8 | 68.5 |
| Construct 2 | 72.2 | 71.2 |
| Construct 3 | 73.3 | 72.1 |
| Construct 4 | 73.4 | 72.1 |
| Construct 5 | 75.9 | 76.1 |
| Construct 6 | 72.4 | 72.8 |
| Construct 7 | 72.3 | 72.8 |
| Construct 8 | 72.1 | 72.6 |
| Construct 9 | 69.1 | 72.3 |
| Construct 15 | 77.4 | 77.1 |
| Construct 16 | 72.4 | 73.0 |
| Construct 17 | 72.5 | 72.9 |
| Construct 18 | 73.1 | 73.1 |
| Construct 19 | 71.8 | 72.8 |
| Construct 20 | 71.1 | 72.0 |
| Construct 95 | 72.8 | 76.2 |
| Construct 96 | 70.8 | 71.2 |
| Construct 98 | 70.8 | 72.0 |
| Construct 104 | 68.6 | 74.1 |
| Construct 106 | 71.1 | 75.2 |
| Construct 107 | 71.2 | 74.1 |
| Construct 108 | 72.5 | 73.8 |
| Construct 110 | 71.4 | 74.0 |
| Construct 114 | 70.9 | 73.8 |

*See construct sequences in Tables 2-8.

### Example 4: Engineered Anti-IL-13 Antibodies Exhibit Reduced Hydrophobicity

Hydrophobic interaction chromatography (HIC) was performed to measure the propensity of the engineered anti-IL-13 antibodies for interaction with hydrophobic surfaces (Table 22). Shorter retention times indicate less degree of hydrophobicity. All of the novel IL-13 antibodies tested showed shorter retention times (RT) compared to lebrikizumab. Thus, all of the engineered anti-13 antibodies tested exhibited reduced hydrophobicity compared to lebrikizumab.

TABLE 22
Hydrophobicity of engineered anti-IL-13 antibodies

| | HIC |
|---|---|
| | RT |
| Construct ID* | (min) |
| Lebrikizumab | 15.2 |
| Construct 2 | 15.3 |
| Construct 15 | 13.9 |
| Construct 19 | 14.1 |
| Construct 95 | 13.3 |
| Construct 96 | 14 |
| Construct 98 | 13.7 |
| Construct 104 | 14.1 |
| Construct 106 | 13.3 |
| Construct 107 | 13.3 |
| Construct 108 | 14.1 |
| Construct 110 | 14.4 |
| Construct 114 | 13.4 |

*See construct sequences in Tables 2-8.

### Example 6: An Engineered Anti-IL13 Antibody Variant and Lebrikizumab have the Same Epitope on IL-13

Epitope binning describes a technique that characterizes whether two antibodies specific to the same target (in this case, IL-13) can each bind the target at the same time. mAb pairs are binned together if they block each other's ability to bind to the target antigen. mAb pairs that bin together typically bind to the same or overlapping epitopes on the antigen.

To characterize the binding of Construct 133, which comprises SEQ ID NOs: 3, 39, 439, and 469, vs. lebrikizumab, lebrikizumab was immobilized onto a sensor chip surface capable of measuring mAb-antigen interactions. IL-13 was first injected into the flow channel, where binding of IL-13 to lebrikizumab generated a response. Construct 133 was subsequently injected into the flow channel and the interaction response was recorded. In these studies, no response was observed after Construct 133 injection (see results in Table 23). This indicated that Construct 133 and lebrikizumab binned together and provided evidence to support that the two mAbs likely bind to a similar or the same epitope on IL-13.

TABLE 23
Antibody Binding

| Competitor Antibody or | Immobilized |
|---|---|
| Construct ID* | Lebrikizumab |
| Lebrikizumab | + |
| Tralokinumab | − |
| Cendakimab | − |
| Construct 133 | + |

+ indicates antibodies that bin with lebrikizumab.
− indicates antibodies that do not bin with lebrikizumab.
*See construct sequence 133 in Tables 2-8.

In a similar study, tralokunumab-ldrm (Adbry™) was found to have a binning response, suggesting that it has a different epitope on IL-13 than lebrikizumab.

To further characterize the epitopes of Construct 133 and lebrikizumab, hydrogen-deuterium exchange mass-spectrometry (HDX-MS) and cross-linking mass-spectrometry (XL-MS) were performed, as known in the art. Briefly, HDX-MS was performed using either IL-13 alone at a concentration of 20 uM or a mixed solution of IL-13 and purified antibody, with a final concentration of 20 uM and 40 uM, respectively. Incubation times of 15s, 60s, 180s, 600s, 1800s, and 7200s were performed before the exchange reaction was quenched and the protein mixture subject to proteolysis followed by LC-MS using a Waters Q-ToF Xevo G2-XS. Deuterium incorporation was determined for both IL-13 and the mixture, and peptide regions where deuterium incorporation was inhibited were considered likely to be involved in the binding to the purified antibody. Briefly, XL-MS was performed using a mixed solution of IL-13 and purified antibody, with a final concentration of 14.7 uM and 0.7 uM, respectively. 20 μL of this mixture was mixed with 2 μL of DSS (2 mg/mL stock in DMF) and the final solution was allowed to incubate for 180 minutes at room temperature. After this incubation, samples were subject to proteolysis using trypsin, chymotrypsin, ASP-N, elastase and thermolysin and analyzed using LC-MS using a Q-Exactive MS. Peptides were referenced against peptides identified from a previous peptide mapping experiment of IL-13 alone. IL-13 peptide regions that showed cross-linking to corresponding peptides from the antibody variable region were considered likely to be involved in the binding to the purified antibody. Both methods provided complementary data indicating specific amino acid regions where Construct 133 and lebrikizumab are likely to bind. This provided further evidence to support that the two mAbs likely bind to a highly overlapping epitope on IL-13 (FIG. 1).

These studies provide evidence that Construct 133 binds the same region on IL-13 as lebrikizumab and therefore they are more likely to have the same biological effect than if Construct 133 recognized a different region.

### Example 7: An Engineered Anti-IL-13 Antibody Variant Demonstrated Significantly Extended Half-Life in NHPs and Pharmacokinetic Analysis of Anti-IL-13 Antibodies

### Extended Half-Life of an Anti-IL-13 Antibody Variant in NHPs

To demonstrate the potential of engineered anti-IL13 antibody variants to improve dosing over current and anticipated standard of care mAbs in Alzheimer's disease (AD), among other diseases, Construct 133, which comprises SEQ ID NOs: 3, 39, 439, and 469, was studied in female non-human primates (NHPs) following a single bolus dose of 3 mg/kg, given either IV or SQ. Blood samples were collected serially starting with a sample pre-dose and subsequently at 0.167, 1, 4, 8, 24, 48, 96, 168, 336, 504, 674, 840, 1334, 1680, and 2160 hours post-dose. PK parameters of maximum observed serum concentration (Cₘₐₓ), time to maximum observed serum concentration (Tₘₐₓ), area under the serum concentration versus time curve from time 0 extrapolated to infinity (AUC_{0-inf}), clearance (CL), volume of distribution at steady-state (Vₛₛ), half-life (T_{1/2}) and absolute subcutaneous bioavailability (F) were calculated. Data was analyzed to show mean serum concentration with standard deviation over time and a regression fit was performed.

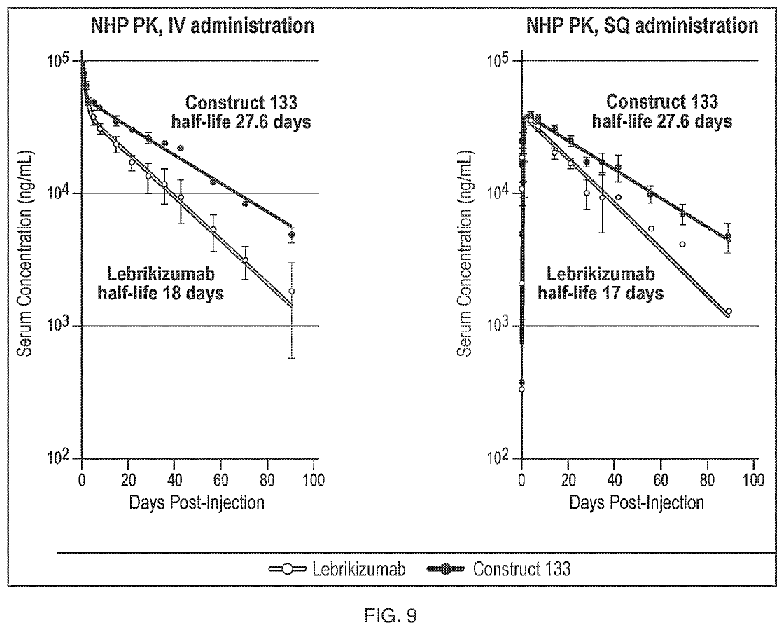

In head-to-head studies of Construct 133 versus lebrikizumab in NHPs, both IV and SQ formulations of Construct 133 showed a significantly longer half-life than lebrikizumab. In these studies, the average half-life of Construct 133 was 27.6 days, as compared to 18 days for lebrikizumab, as shown in FIG. 9. Further, Construct 133 exhibited an average clearance rate of 1.45 (mL day⁻¹ kg⁻¹) in NHPs. The steady-state volume of distribution was observed to be 55.65 (mL kg⁻¹). Construct 133 was well-absorbed, with subcutaneous bioavailability determined to be 81.22%. Lebrikizumab exhibited an average clearance rate of 2.93 (mL day⁻¹ kg⁻¹) in NHPs. The steady-state volume of distribution was observed to be 52.10 (mL kg⁻¹). Lebrikizumab was well-absorbed, with subcutaneous bioavailability determined to be 75.70%. Without being bound by theory, because Construct 133 was engineered to have a YTE amino-acid substitution in the Fc region, the half-life of the IgG may have been prolonged by increasing binding to neonatal Fc receptor (FcRn) under acidic pH conditions. FcRn-bound IgG is recycled via lysosomal salvage, resulting in the IgG returning to the circulation. Construct 133's prolonged half-life may enable less frequent dosing compared to currently available treatments, which could reduce injection burden and increase compliance for patients living with atopic dermatitis and other IL-13-driven diseases.

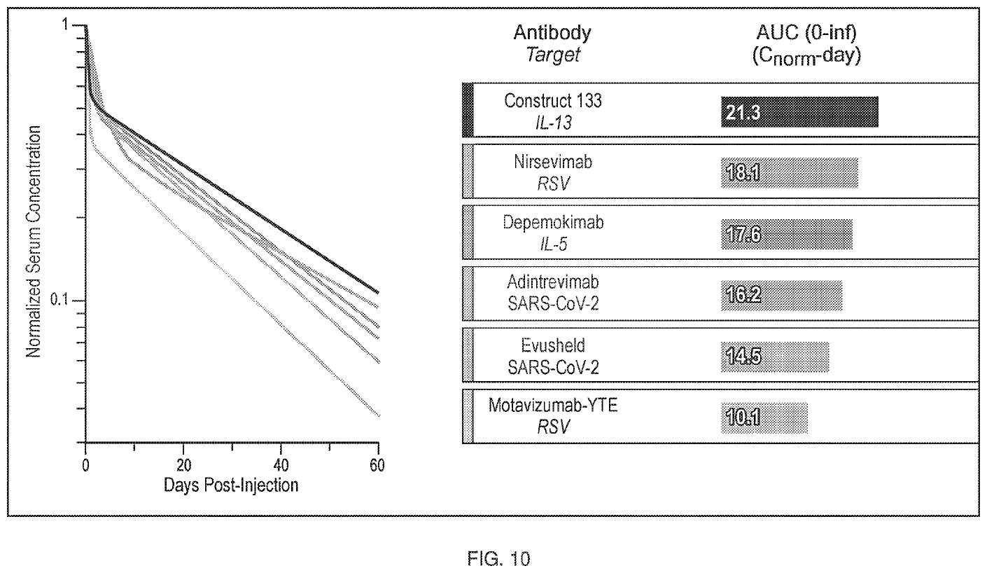

In a non-head-to-head comparison against third-party NHP data, Construct 133 demonstrated the highest normalized AUC_{0-∞} (C_{norm*day}), or area under the curve (AUC) from dosing to infinity, among antibodies with the YTE substitution, as shown in FIG. 10. Thus, the PK profile of Construct 133 appears to provide the greatest sustained concentrations, or levels of drug in the blood stream, relative to other antibodies with the YTE substitution.

The half-life extension for mAbs with YTE amino acid substitutions is dependent on the type of target (e.g., receptor vs. soluble). Therefore, the translation of NHP half-life data to human half-life data for mAbs with soluble targets was studied, and it was found that human half-life is approximately three to four times longer than NHP half-life (mean: 3.5×, median 3.1×; data not shown).

It is expected, based on this NHP half-life data, that the antibodies disclosed herein (e.g., Construct 133) will have a human half-life of approximately 80 to 110 days based on comparable mAbs with YTE amino acid substitution.

Further, based on PK modeling, with a 33-day human half-life (which, to Applicant's knowledge, would be lower than the lowest half-life for a mAb with the YTE amino acid substitutions and a soluble target reported to date), it is believed that the antibodies disclosed herein can be dosed effectively with an every two month maintenance dosing schedule. With a 50-day half-life, it is believed that the antibodies disclosed herein can be dosed effectively with an every three month maintenance dosing schedule.

To understand the maintenance dosing schedule that the antibodies disclosed herein may be able to achieve, known PK parameters for lebrikizumab were used. These PK parameters provided an understanding of how lebrikizumab was distributed throughout the body and cleared. Based on these known parameters, a two-compartment PK model with first-order absorption was built, which is standard for mAbs, to predict concentration or drug levels, over time of both lebrikizumab and the antibodies disclosed herein. Key parameters included 0.156 L/day for clearance (CL), 4.10 L for central volume (Vc), 0.239 day-1 for absorption rate (ka) and 85.6% for bioavailability.

It is believed that efficacy in inflammatory conditions, such as AD, is driven by C_{trough}, or the minimal concentration of the mAb. Therefore, based on the model described above, the target C_{trough} of the antibodies disclosed herein was set to be equal to lebrikizumab's C_{trough} in maintenance with every one month dosing, which was 31.3 mg/L. Given the overlapping epitopes of lebrikizumab and certain antibodies disclosed herein, and similarity in potency across multiple in vitro assays, the necessary exposures for potential clinical activity of the antibodies disclosed herein can be predicted. By modeling Kₑₗᵢₘᵢₙₐₜᵢₒₙ, the elimination rate constant or the fraction of drug eliminated in a given time, and half-life to maintain concentrations of the disclosed antibodies above 31.3 mg/L, at least a 33-day half-life is would be required to dose the disclosed antibodies every two months in maintenance and at least a 50-day half-life would be required to dose the disclosed antibodies every three months in maintenance assuming a dose of 300 mg.

Thus, with a 33-day human half-life, it is believed that the antibodies disclosed herein can be dosed effectively with an every two month maintenance dosing schedule. With a 50-day half-life, it is believed that the antibodies disclosed herein can be dosed effectively with an every three month maintenance dosing schedule.

### Pharmacokinetic Analysis of Anti-IL-13 Antibodies

In further experiments, multiple in vivo pharmacokinetic (PK) studies were performed where lebrikizumab, additional variants of Construct 15 or Construct 98 with one or more amino acid substitutions in the heavy chain constant region (Constructs 133-137 or 137 and 140, respectively), and/or additional variants with one or more amino acid substitutions in the heavy chain constant region (Constructs 141 and 144) were also tested.

Studies were performed using cynomolgus monkey (Macaca fascicularis), where any matching or subcutaneous (SQ)/intravenous (IV) cohorts were either all female, ranging from 1.5 kg to 2.0 kg in weight, or all males, ranging from 2.9 kg to 3.3 kg in weight. Animals were administered test agents by IV bolus and/or SQ injection on Day 0 at a dose of 3 mg/kg for each antibody and serum samples were taken regularly throughout the study.

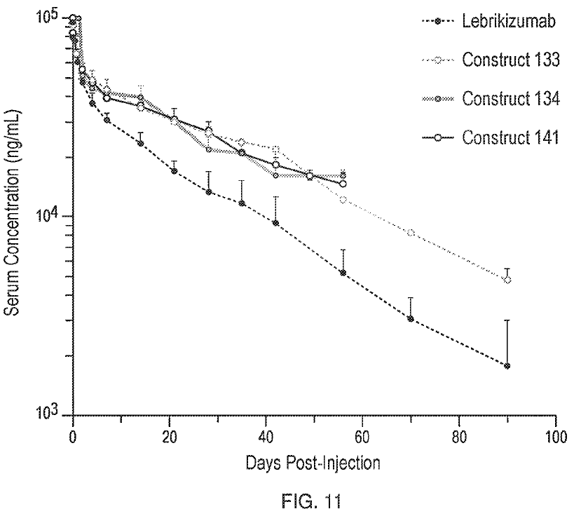

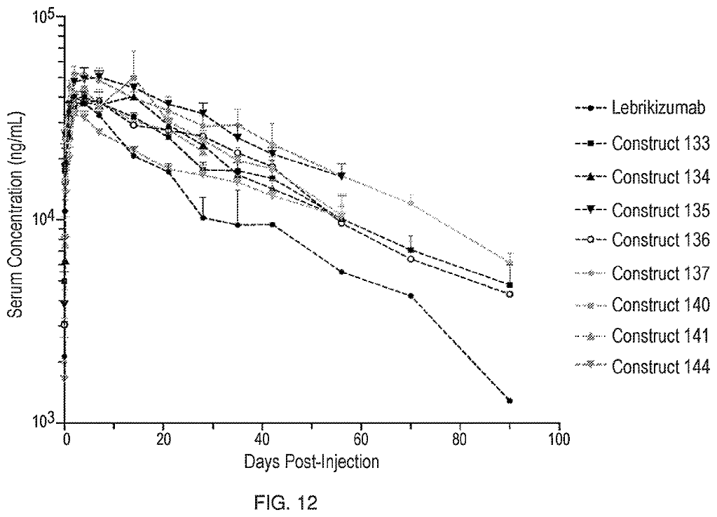

PK parameters were determined from cynomolgus serum samples up to day 56 (1334 hours), with a subset of cohorts up to day 90 (2160 hours), with average PK curves are shown in FIG. 11 for IV administration and FIG. 12 for SQ administration. The PK analysis demonstrated that Constructs 133, 134, 135, 136, 137, 140, 141, and 144 each had improved half-life and reduction in serum clearance rates compared to those of lebrikizumab as reported in Table 24. In cases where bioavailability (F) could be determined, Constructs 133, 134, and 141 were shown to possess equivalent bioavailability to that of lebrikizumab (Table 25).

TABLE 24
Half-life and Serum Clearance Rates

| | | AUC_{inf} | | CL* | |
|---|---|---|---|---|---|
| Construct ID^{#} and | | (ng · | Half- | (mL · | Vₛₛ* |
| Administration | | hours · | Life | day⁻¹ | (mL · |
| Group | Animal | mL⁻¹) | (Days) | · kg⁻¹) | kg⁻¹) |
| Lebrikizumab - IV | 1501 | 17447678.05 | 11.02 | 4.13 | 51.38 |
| | 1502 | 28812691.42 | 18.86 | 2.50 | 55.87 |
| | 1501 | 33491428.36 | 24.35 | 2.15 | 70.54 |
| Lebrikizumab - SQ | 2501 | 28290795.91 | 18.82 | 1.93 | 52.83 |
| | 2502 | 14531783.14 | 11.33 | 3.75 | 42.26 |
| | 2503 | 17545785.64 | 10.42 | 3.11 | 39.72 |
| Construct 133 - IV | 6501 | 54367740.94 | 30.25 | 1.32 | 53.97 |
| | 6502 | 49429927.46 | 28.27 | 1.46 | 56.14 |
| | 6503 | 47738132.48 | 26.22 | 1.51 | 52.07 |
| Construct 133 - SQ | 7501 | 50202816.21 | 30.85 | 1.16 | 53.73 |
| | 7502 | 31546038.62 | 22.44 | 1.85 | 60.32 |
| | 7503 | 41329262.82 | 27.65 | 1.41 | 57.68 |
| Construct 134 - IV | 3501 | 52254317.47 | 21.38 | 1.38 | 47.07 |
| | 3502 | 33366131.55 | 17.24 | 2.16 | 54.75 |
| | 3503 | 77329739.21 | 41.87 | 0.93 | 46.90 |
| Construct 134 - SQ | 4501 | 54533364.13 | 50.94 | 0.96 | 65.60 |
| | 4502 | 36701086.34 | 20.48 | 1.43 | 42.05 |
| | 4503 | 27442862.48 | 14.49 | 1.91 | 38.48 |
| Construct 135 - SQ | 5501 | 50614453.99 | 29.68 | 1.42 | 57.79 |
| | 5502 | 75200216.97 | 40.07 | 0.96 | 55.48 |
| | 5503 | 61123113.94 | 27.93 | 1.18 | 48.56 |
| Construct 136 - SQ | 8501 | 47846929.29 | 27.86 | 1.50 | 56.97 |
| | 8502 | 32127535.46 | 21.51 | 2.24 | 66.76 |
| | 8503 | 44101872.29 | 30.83 | 1.63 | 68.10 |
| Construct 137 - SQ | 9501 | 46216823.54 | 16.57 | 1.56 | 52.59 |
| | 9502 | 32421239.31 | 25.86 | 2.22 | 68.74 |
| | 9503 | 44629524.73 | 34.27 | 1.61 | 70.01 |
| Construct 140 - SQ | 10501 | 51739935.51 | 27.32 | 1.39 | 49.24 |
| | 10502 | 41305658.5 | 21.82 | 1.74 | 53.20 |
| | 10503 | 47527303.18 | 32.84 | 1.51 | 72.48 |
| Construct 141 - IV | 4089 | 45698126.79 | 27.2 | 1.58 | 60.29 |
| | 2095 | 62600360.39 | 31.99 | 1.15 | 55.01 |
| | 3027 | 53748965.77 | 33.88 | 1.34 | 63.50 |
| Construct 141 - SQ | 1131 | 56004213.41 | 45.72 | 0.97 | 66.44 |
| | 1083 | 38648918.97 | 31.47 | 1.41 | 64.56 |
| | 1127 | 27810354.27 | 21.22 | 1.96 | 63.03 |
| Construct 144 - SQ | 11501 | 28455075.58 | 21.78 | 2.53 | 85.42 |
| | 11502 | 22141956.08 | 18.99 | 3.25 | 81.00 |
| | 11503 | 45675112.33 | 43.35 | 1.58 | 103.64 |

*These values are adjusted for bioavailability in cohorts Lebrikizumab, Construct 133, Construct 134, and Construct 141.
^{#}See construct sequences in Tables 2-8.

TABLE 25
Construct Bioavailability

| Construct ID* | Bioavailability (%) |
|---|---|
| Lebrikizumab | 75.70% |
| Construct 133 | 81.22% |
| Construct 134 | 72.83% |
| Construct 141 | 75.57% |

*See construct sequences in Tables 2-8.

### Example 8. Inhibition of IL-13 Induced Secretion of CCL2 and CCL26 and Expression of NTRK1 in HaCaT Cells

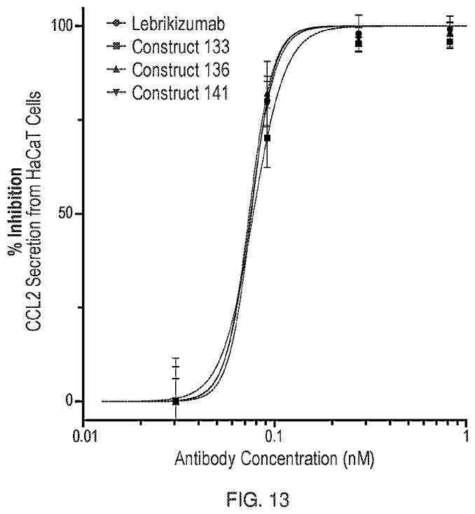

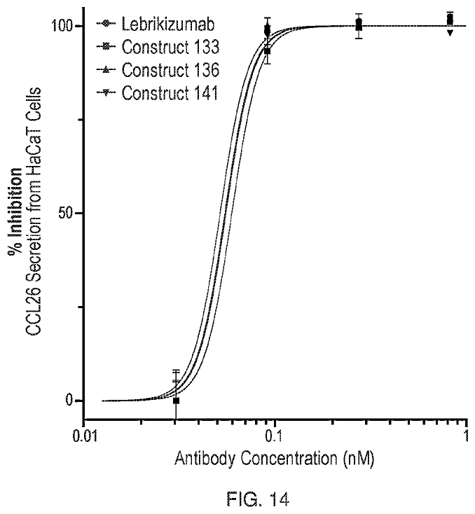

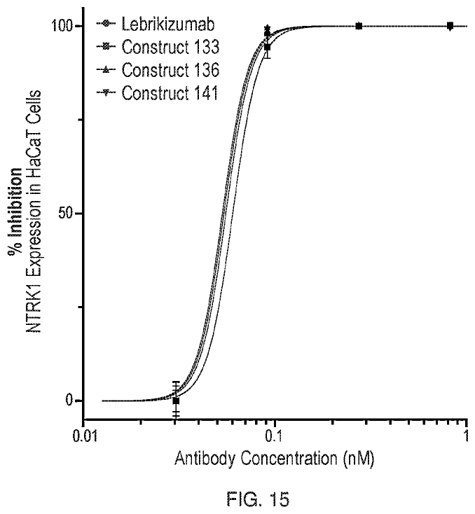

Inhibition of CCL26 (eotaxin-3) and CCL2 (MCP-1) secretion (FIG. 13 and FIG. 14, respectively) and NTRK1 expression (FIG. 15) by HaCaT cells was used to evaluate the functional activity of antibodies to block IL-13-induced biological activity. HaCaT cells were seeded at 20,000 cells in 100 uL of DMEM+10% FBS and cultured overnight at 37° C. The next day, a 150 uL mixture of hIL-13 and purified antibody were added to the wells, resulting in a final concentration of 50 ng/ml of IL-13 with 0-206.5 nM purified antibody. Cells were then further incubated at 37° C. for 48 hours. Following incubation, culture supernatant was collected and levels of secreted CCL26 and CCL2 were measured using a commercial Luminex-based immunoassay kit (R&D Systems) and analyzed according to manufacturer's instructions. Determined concentrations of CCL26 and CCL2 in each well were analyzed using GraphPad Prism. IC₅₀ values were determined as the concentration of antibody required to inhibit 50% of the maximum concentration detected with incubation of 50 ng/ml of IL-13 alone.

Cells remaining in the assay plates were lysed and mRNA extracted for analysis of NTRK1 gene expression using a commercial Quantigene kit (ThermoFisher). Levels of NTRK1 mRNA were determined according to the manufacturer's protocol and analyzed in GraphPad Prism. NTRK1 gene expression was quantified as a ratio of NTRK1 mRNA levels relative to the housekeeping gene, PPIB, and IC50 values calculated as the concentration of antibody required to inhibit 50% of the maximum gene expression detected using 50 ng/ml of hIL-13 alone. Results are summarized in Table 26.

TABLE 26
CCL26 and CCL2 secretion and NTRK1 expression

| | CCL2 | CCL26 | NTRK1 |
|---|---|---|---|
| | Secretion | Secretion | Expression |
| Construct ID* | IC₅₀ (nM) | IC₅₀ (nM) | IC₅₀ (nM) |
| Lebrikizumab | 0.076 | 0.055 | 0.056 |
| Construct 133 | 0.077 | 0.060 | 0.062 |
| Construct 136 | 0.074 | 0.052 | 0.053 |
| Construct 141 | 0.075 | 0.055 | 0.054 |

*See construct sequences in Tables 2-8.

### Example 9. Monomer Purity after Affinity Capture from Stable Pools

The monomer purity from one-step affinity capture is a determinant for the final yield and unit cost of an antibody under cGMP production. To characterize this, briefly, CHO stable pools were generated separately for each antibody in a workstream that led to master cell bank selection for cGMP production. The affinity capture step was performed using a Mabselect SuRe column. Novel IgG1 variants (e.g., Constructs 132, 133, 136, 137, 140, 141, and 144) remained a clear solution with <15% aggregate after the one-step purification. Novel IgG4 variants (Constructs 134, 135, 138, 139, 142, and 143) had an opalescent appearance with some precipitation and an aggregation sensitivity (>68% aggregate) when using an elution buffer of 50 mM sodium citrate, 150 mM sodium chloride at pH 3.0. The novel IgG4 variants had lower aggregate levels when eluted with either 50 mM acetic acid, pH 2.8 or with 100 mM sodium acetate, 800 mM arginine at pH 3.5. At the higher pH of 3.5, the arginine was needed to retain a suitable recovery.

Results are summarized in Table 27. Novel IgG1 and IgG4 variants demonstrate greater monomer purity directly out of affinity capture with optimized elution conditions when compared to lebrikizumab variants (Constructs 128-131).

TABLE 27
Anti-IL-13 Antibody Monomer Purity

| | One-Step Monomer |
|---|---|
| Construct ID* | Purity (%) |
| Construct 128 | 87.7 |
| Construct 129 | 90.5 |
| Construct 130 | 81.6 |
| Construct 131 | 82.9 |
| Construct 132 | 94.7 |
| Construct 133 | 90.9 |
| Construct 134 | 91.7 |
| Construct 135 | 91.4 |
| Construct 136 | 90.3 |
| Construct 137 | 92.7 |
| Construct 138 | 94.0 |
| Construct 139 | 92.0 |
| Construct 140 | 89.7 |
| Construct 141 | 94.1 |
| Construct 142 | 90.1 |
| Construct 143 | 91.5 |
| Construct 144 | 92.4 |

*See construct sequences in Tables 2-8.

### Example 10. Accelerated Stability

As demonstrated by this example, novel variants in an IgG1 construct had a lower propensity to aggregate and were more resistant to changes in the basic species under various stress conditions, as shown in Table 28. The proportion of basic species was determined through capillary isoelectric focusing (ciEF), performed as known in the art. The variants related to lebrikizumab (Constructs 128-131) and IgG4 variants (Constructs 134, 135, 142, and 143); as described in Examples 1-7) were more susceptible to aggregation and changes in the basic species compared to the novel IgG1 variants (e.g., Constructs 132, 133, 136, 137, 140, 141, and 144).

TABLE 28
Anti-IL-13 Antibody Stability Change

| | 40° C. | | | |
|---|---|---|---|---|
| | Stability- | 40° C. | pH 3.5 | pH 3.5 |
| | Change | Stability- | Stability - | Stability - |
| | in | Change | Change | Change |
| | Monomer | Basic | Monomer | Basic |
| Construct ID* | Purity | Species | Purity | Species |
| Construct 128 | −10.0% | +26.5% | −10.0% | +6.3% |
| Construct 129 | −3.2% | +8.2% | −12.7% | +5.7% |
| Construct 130 | −2.2% | −13.8% | −1.1% | +1.5% |
| Construct 131 | −2.1% | −11.2% | −1.6% | +0.6% |
| Construct 132 | −0.8% | +2.4% | −0.7% | +1.2% |
| Construct 133 | −2.1% | +0.9% | −1.3% | +0.9% |
| Construct 134 | −1.4% | +10.3% | −19.5% | +13.3% |
| Construct 135 | −1.8% | +2.1% | −15.7% | +8.0% |
| Construct 136 | −0.9% | +1.5% | −1.4% | +0.4% |
| Construct 137 | −1.5% | +2.4% | −1.3% | +1.6% |
| Construct 140 | −3.6% | +1.5% | −1.4% | +0.8% |
| Construct 141 | −2.5% | +0.0% | −0.5% | +0.5% |
| Construct 142 | −4.0% | +7.1% | −8.9% | +6.5% |
| Construct 143 | −4.3% | +5.1% | −11.1% | +5.1% |
| Construct 144 | −3.0% | +2.0% | −0.1% | +1.2% |

*See construct sequences in Tables 2-8.
Assessment of clones for changes in monomer purity and basic species were evaluated at day 0 and day 14 for 40° C. stability and at day 0 and day 2 for pH 3.5 stability.
N.T.—Not Tested.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Informal sequence listing

| | | SEQ ID |
|---|---|---|
| Description | Sequence | NO |
| Heavy | QVQLQESGPGLVAPSQSLSITCTVSGFSLNAYSVNWVRQP | SEQ ID |
| Chain | PGKGLEWLGMIWGDGKIVYNSALKSRLNISKDSSKSQVFL | NO: 1 |
| Variable | KMSSLQSDDTARYYCAGDGYYPYAMDNWGHGTSVTVSS | |
| domain | | |
| HC0 | | |
| Heavy | QVQLQESGPGLVAPSQSLSITCTVSGFSLNAYSVNWVRQP | SEQ ID |
| Chain | PGKGLEWLGMIWGDGKIVYNSALKSRLTISKDSSKSQVFL | NO: 2 |
| Variable | KMSSLQSDDTARYYCAGDGYYPYAMDNWGHGTSVTVSS | |
| domain | | |
| HC0_M | | |
| Heavy | EVQLQESGPGLVKPSETLSLTCTVSGFSLNAYSVNWIRQPP | SEQ ID |
| Chain | GKGLEWLGMIWGDGKIVYNSALKSRLTISKDSSKNQVSLK | NO: 3 |
| Variable | LSSVTAADTAVYYCAGDGYYPYAMDNWGQGTTVTVSS | |
| domain | | |
| HC1 | | |
| Heavy | EVQLVQSGAEVKKPGASVKVSCKASGFSLNAYSVNWVR | SEQ ID |
| Chain | QAPGQGLEWLGMIWGDGKIVYNSALKSRLTITKDSSTSTV | NO: 4 |
| Variable | YMELSSLRSEDTAVYYCAGDGYYPYAMDNWGQGTTVTV | |
| domain | SS | |
| HC2 | | |
| Heavy | EVQLVQSGAEVKKPGSSVKVSCKASGFSLNAYSVNWVRQ | SEQ ID |
| Chain | APGQGLEWLGMIWGDGKIVYNSALKSRLTITKDSSTSTVY | NO: 5 |
| Variable | MELSSLRSEDTAVYYCAGDGYYPYAMDNWGQGTTVTVS | |
| domain | S | |
| HC3 | | |
| Heavy | EVQLVESGGGLVKPGGSLRLSCAASGFSLNAYSVNWVRQ | SEQ ID |
| Chain | APGKGLEWLGMIWGDGKIVYNSALKSRLTISKDSSKNTVY | NO: 6 |
| Variable | LQMNSLKTEDTAVYYCAGDGYYPYAMDNWGQGTTVTV | |
| domain | SS | |
| HC4 | | |
| Heavy | EVQLLESGGGLVQPGGSLRLSCAASGFSLNAYSVNWVRQ | SEQ ID |
| Chain | APGKGLEWLGMIWGDGKIVYNSALKSRLTISKDSSKNTVY | NO: 7 |
| Variable | LQMNSLRAEDTAVYYCAGDGYYPYAMDNWGQGTTVTV | |
| domain | SS | |
| HC5 | | |
| Heavy | EVQLQESGPGLVKPSETLSLTCTVSGGSLNAYSVNWVRQP | SEQ ID |
| Chain | PGKGLEWLGMIWGDGKIVYNSALKSRLTISLDTSKSQVFL | NO: 8 |
| Variable | KMSSLTAADTAVYYCARDGYYPYAMDNWGQGTTVTVSS | |
| domain | | |
| HC6 | | |
| Heavy | QVQLQESGPGLVKPSETLSLTCTVSGGSLNAYSWNWVRQ | SEQ ID |
| Chain | PPGKGLEWLGYIYGDGKTNYNPALKSRLTISLDTSKSQVF | NO: 9 |
| Variable | LKMSSLTAADTAVYYCARDGYYYYAMDVWGQGTTVTV | |
| domain | SS | |
| HC7 | | |
| Heavy | EVQLLESGGGLVQPGGSLRLSCAASGYSLNAYSVNWVRQ | SEQ ID |
| Chain | APGKGLEWLGMIWGDGKIVYNSALKSRLTISKDSSKNTVY | NO: 10 |
| Variable | LQMNSLRAEDTAVYYCAGDGYYPYAMDNWGQGTTVTV | |
| domain | SS | |
| HC5m1 | | |
| Heavy | EVQLLESGGGLVQPGGSLRLSCAASGFSLRAYSVNWVRQ | SEQ ID |
| Chain | APGKGLEWLGMIWGDGKIVYNSALKSRLTISKDSSKNTVY | NO: 11 |
| Variable | LQMNSLRAEDTAVYYCAGDGYYPYAMDNWGQGTTVTV | |
| domain | SS | |
| HC5m2 | | |
| Heavy | EVQLLESGGGLVQPGGSLRLSCAASGFSLHAYSVNWVRQ | SEQ ID |
| Chain | APGKGLEWLGMIWGDGKIVYNSALKSRLTISKDSSKNTVY | NO: 12 |
| Variable | LQMNSLRAEDTAVYYCAGDGYYPYAMDNWGQGTTVTV | |
| domain | SS | |
| HC5m3 | | |
| Heavy | EVQLLESGGGLVQPGGSLRLSCAASGFSLDAYSVNWVRQ | SEQ ID |
| Chain | APGKGLEWLGMIWGDGKIVYNSALKSRLTISKDSSKNTVY | NO: 13 |
| Variable | LQMNSLRAEDTAVYYCAGDGYYPYAMDNWGQGTTVTV | |
| domain | SS | |
| HC5m4 | | |
| Heavy | EVQLLESGGGLVQPGGSLRLSCAASGFSLYAYSVNWVRQ | SEQ ID |
| Chain | APGKGLEWLGMIWGDGKIVYNSALKSRLTISKDSSKNTVY | NO: 14 |
| Variable | LQMNSLRAEDTAVYYCAGDGYYPYAMDNWGQGTTVTV | |
| domain | SS | |
| HC5m5 | | |
| Heavy | EVQLLESGGGLVQPGGSLRLSCAASGFSLSAYSVNWVRQ | SEQ ID |
| Chain | APGKGLEWLGMIWGDGKIVYNSALKSRLTISKDSSKNTVY | NO: 15 |
| Variable | LQMNSLRAEDTAVYYCAGDGYYPYAMDNWGQGTTVTV | |
| domain | SS | |
| HC5m6 | | |
| Heavy | EVQLLESGGGLVQPGGSLRLSCAASGFSLNRYSVNWVRQ | SEQ ID |
| Chain | APGKGLEWLGMIWGDGKIVYNSALKSRLTISKDSSKNTVY | NO: 16 |
| Variable | LQMNSLRAEDTAVYYCAGDGYYPYAMDNWGQGTTVTV | |
| domain | SS | |
| HC5m7 | | |
| Heavy | EVQLLESGGGLVQPGGSLRLSCAASGFSLNKYSVNWVRQ | SEQ ID |
| Chain | APGKGLEWLGMIWGDGKIVYNSALKSRLTISKDSSKNTVY | NO: 17 |
| Variable | LQMNSLRAEDTAVYYCAGDGYYPYAMDNWGQGTTVTV | |
| domain | SS | |
| HC5m8 | | |
| Heavy | EVQLLESGGGLVQPGGSLRLSCAASGFSLNHYSVNWVRQ | SEQ ID |
| Chain | APGKGLEWLGMIWGDGKIVYNSALKSRLTISKDSSKNTVY | NO: 18 |
| Variable | LQMNSLRAEDTAVYYCAGDGYYPYAMDNWGQGTTVTV | |
| domain | SS | |
| HC5m9 | | |
| Heavy | EVQLLESGGGLVQPGGSLRLSCAASGFSLNQYSVNWVRQ | SEQ ID |
| Chain | APGKGLEWLGMIWGDGKIVYNSALKSRLTISKDSSKNTVY | NO: 19 |
| Variable | LQMNSLRAEDTAVYYCAGDGYYPYAMDNWGQGTTVTV | |
| domain | SS | |
| HC5m10 | | |
| Heavy | EVQLLESGGGLVQPGGSLRLSCAASGFSLNEYSVNWVRQ | SEQ ID |
| Chain | APGKGLEWLGMIWGDGKIVYNSALKSRLTISKDSSKNTVY | NO: 20 |
| Variable | LQMNSLRAEDTAVYYCAGDGYYPYAMDNWGQGTTVTV | |
| domain | SS | |
| HC5m11 | | |
| Heavy | EVQLLESGGGLVQPGGSLRLSCAASGFSLNSYSVNWVRQ | SEQ ID |
| Chain | APGKGLEWLGMIWGDGKIVYNSALKSRLTISKDSSKNTVY | NO: 21 |
| Variable | LQMNSLRAEDTAVYYCAGDGYYPYAMDNWGQGTTVTV | |
| domain | SS | |
| HC5m12 | | |
| Heavy | EVQLLESGGGLVQPGGSLRLSCAASGFSLNYYSVNWVRQ | SEQ ID |
| Chain | APGKGLEWLGMIWGDGKIVYNSALKSRLTISKDSSKNTVY | NO: 22 |
| Variable | LQMNSLRAEDTAVYYCAGDGYYPYAMDNWGQGTTVTV | |
| domain | SS | |
| HC5m13 | | |
| Heavy | EVQLLESGGGLVQPGGSLRLSCAASGFSLNAESVNWVRQ | SEQ ID |
| Chain | APGKGLEWLGMIWGDGKIVYNSALKSRLTISKDSSKNTVY | NO: 23 |
| Variable | LQMNSLRAEDTAVYYCAGDGYYPYAMDNWGQGTTVTV | |
| domain | SS | |
| HC5m14 | | |
| Heavy | EVQLLESGGGLVQPGGSLRLSCAASGFSLNAYSVNWVRQ | SEQ ID |
| Chain | APGKGLEWLGMIWSDGKIVYNSALKSRLTISKDSSKNTVY | NO: 24 |
| Variable | LQMNSLRAEDTAVYYCAGDGYYPYAMDNWGQGTTVTV | |
| domain | SS | |
| HC5m15 | | |
| Heavy | EVQLLESGGGLVQPGGSLRLSCAASGFSLNAYSVNWVRQ | SEQ ID |
| Chain | APGKGLEWLGMIWADGKIVYNSALKSRLTISKDSSKNTVY | NO: 25 |
| Variable | LQMNSLRAEDTAVYYCAGDGYYPYAMDNWGQGTTVTV | |
| domain | SS | |
| HC5m16 | | |
| Heavy | EVQLLESGGGLVQPGGSLRLSCAASGFSLNAYSVNWVRQ | SEQ ID |
| Chain | APGKGLEWLGMIWGDGKIVYNSALKSRLTISKDSSKNTVY | NO: 26 |
| Variable | LQMNSLRAEDTAVYYCAGHGYYPYAMDNWGQGTTVTV | |
| domain | SS | |
| HC5m17 | | |
| Heavy | EVQLLESGGGLVQPGGSLRLSCAASGFSLNAYSVNWVRQ | SEQ ID |
| Chain | APGKGLEWLGMIWGDGKIVYNSALKSRLTISKDSSKNTVY | NO: 27 |
| Variable | LQMNSLRAEDTAVYYCAGDLYYPYAMDNWGQGTTVTVS | |
| domain | S | |
| HC5m18 | | |
| Heavy | EVQLLESGGGLVQPGGSLRLSCAASGFSLNAYSVNWVRQ | SEQ ID |
| Chain | APGKGLEWLGMIWGDGKIVYNSALKSRLTISKDSSKNTVY | NO: 28 |
| Variable | LQMNSLRAEDTAVYYCAGDKYYPYAMDNWGQGTTVTV | |
| domain | SS | |
| HC5m19 | | |
| Heavy | EVQLLESGGGLVQPGGSLRLSCAASGFSLNAYSVNWVRQ | SEQ ID |
| Chain | APGKGLEWLGMIWGDGKIVYNSALKSRLTISKDSSKNTVY | NO: 29 |
| Variable | LQMNSLRAEDTAVYYCAGDGYYGYAMDNWGQGTTVTV | |
| domain | SS | |
| HC5m20 | | |
| Heavy | EVQLLESGGGLVQPGGSLRLSCAASGFSLNAYSVNWVRQ | SEQ ID |
| Chain | APGKGLEWLGMIWGDGKIVYNSALKSRLTISKDSSKNTVY | NO: 30 |
| Variable | LQMNSLRAEDTAVYYCAGDGYYAYAMDNWGQGTTVTV | |
| domain | SS | |
| HC5m21 | | |
| Heavy | EVQLLESGGGLVQPGGSLRLSCAASGFSLNAYSVNWVRQ | SEQ ID |
| Chain | APGKGLEWLGMIWGDGKIVYNSALKSRLTISKDSSKNTVY | NO: 31 |
| Variable | LQMNSLRAEDTAVYYCAGDGYYSYAMDNWGQGTTVTV | |
| domain | SS | |
| HC5m22 | | |
| Heavy | EVQLLESGGGLVQPGGSLRLSCAASGFSLNAYSVNWVRQ | SEQ ID |
| Chain | APGKGLEWLGMIWGDGKIVYNSALKSRLTISKDSSKNTVY | NO: 32 |
| Variable | LQMNSLRAEDTAVYYCAGDGYYTYAMDNWGQGTTVTV | |
| domain | SS | |
| HC5m23 | | |
| Light Chain | NIVLTQSPASLAVSLGQRATISCRASKSVDSYGNSFMHWY | SEQ ID |
| Variable | QQKPGQPPKLLIYLASNLESGVPARFSGSGSRTDFTLTIDPV | NO: 33 |
| domain | EADDAASYYCQQNNEDPRTFGGGTKLEIK | |
| LC0 | | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSRTDFTLTISSL | NO: 34 |
| LC1 | QPEDFATYYCQQNNEDPRTFGGGTKVEIK | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSRTDFTLTISSL | NO: 35 |
| domain | QPEDFATYYCQQNNEDPRTFGGGTKVEIK | |
| LC2 | | |
| Light Chain | EIVLTQSPATLSVSPGERATLSCRASKSVDSYGNSFMHWY | SEQ ID |
| Variable | QQKPGQAPRLLIYLASNLESGIPARFSGSGSRTEFTLTISSL | NO: 36 |
| domain | QSEDFAVYYCQQNNEDPRTFGGGTKVEIK | |
| LC3 | | |
| Light Chain | DIVLTQSPLSLPVTPGEPASISCRASKSVDSYGNSFMHWYL | SEQ ID |
| Variable | QKPGQSPQLLIYLASNLESGVPDRFSGSGSRTDFTLKISRVE | NO: 37 |
| domain | AEDVGVYYCQQNNEDPRTFGGGTKVEIK | |
| LC4 | | |
| Light Chain | DIVLTQSPDSLAVSLGERATINCRASKSVDSYGNSFMHWY | SEQ ID |
| Variable | QQKPGQPPKLLIYLASNLESGVPDRFSGSGSGTDFTLTISSL | NO: 38 |
| domain | QAEDVAVYYCQQNNEDPRTFGGGTKVEIK | |
| LC5 | | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSGTDFTLTISSL | NO: 39 |
| domain | QPEDFATYYCQQNNEDPRTFGGGTKVEIK | |
| LC6 | | |
| Light Chain | EIVLTQSPATLSVSPGERATLSCRASKSVDSYGNSFMHWY | SEQ ID |
| Variable | QQKPGQAPRLLIYLASNLESGIPARFSGSGSGTEFTLTISSL | NO: 40 |
| domain | QSEDFAVYYCQQNNEDPRTFGGGTKVEIK | |
| LC7 | | |
| Light Chain | DIVLTQSPLSLPVTPGEPASISCRASKSVDSYGNSFMHWYL | SEQ ID |
| Variable | QKPGQSPQLLIYLASNLESGVPDRFSGSGSGTDFTLKISRVE | NO: 41 |
| domain | AEDVGVYYCQQNNEDPRTFGGGTKVEIK | |
| LC8 | | |
| Light Chain | DIVLTQSPASLAVSPGERATISCRASKSVDSYGNSFMHWY | SEQ ID |
| Variable | QQKPGQPPKLLIYLASNLESGVPDRFSGSGSGTDFTLTISRV | NO: 42 |
| domain | EADDVAVYYCQQNNEDPRTFGGGTKLEIK | |
| LC9 | | |
| Light Chain | DIVLTQSPASLAVSPGERATISCRASQSVDSNGNNFLHWY | SEQ ID |
| Variable | QQKPGQPPKLLIYLASNRESGVPDRFSGSGSGTDFTLTISR | NO: 43 |
| domain | VEADDVAVYYCQQNNHTPRTFGGGTKLEIK | |
| LC10 | | |
| Light Chain | DIQLTQSPSSLSASVGDRVTITCRASKSVDSYGNSRMHWY | SEQ ID |
| Variable | QQKPGKAPKLLIYLASNLESGVPSRFSGSGSGTDFTLTISSL | NO: 44 |
| domain | QPEDFATYYCQQNNEDPRTFGGGTKVEIK | |
| LC6_m1 | | |
| Light Chain | DIQLTQSPSSLSASVGDRVTITCRASKSVDSYGNSSMHWY | SEQ ID |
| Variable | QQKPGKAPKLLIYLASNLESGVPSRFSGSGSGTDFTLTISSL | NO: 45 |
| domain | QPEDFATYYCQQNNEDPRTFGGGTKVEIK | |
| LC6_m2 | | |
| Light Chain | DIQLTQSPSSLSASVGDRVTITCRASKSVDSYGNSFMHWY | SEQ ID |
| Variable | QQKPGKAPKLLIRLASNLESGVPSRFSGSGSGTDFTLTISSL | NO: 46 |
| domain | QPEDFATYYCQQNNEDPRTFGGGTKVEIK | |
| LC6_m3 | | |
| Light Chain | DIQLTQSPSSLSASVGDRVTITCRASKSVDSYGNSFMHWY | SEQ ID |
| Variable | QQKPGKAPKLLIFLASNLESGVPSRFSGSGSGTDFTLTISSL | NO: 47 |
| domain | QPEDFATYYCQQNNEDPRTFGGGTKVEIK | |
| LC6_m4 | | |
| Light Chain | DIQLTQSPSSLSASVGDRVTITCRASKSVDSYGNSFMHWY | SEQ ID |
| Variable | QQKPGKAPKLLIYLASHLESGVPSRFSGSGSGTDFTLTISSL | NO: 48 |
| domain | QPEDFATYYCQQNNEDPRTFGGGTKVEIK | |
| LC6_m5 | | |
| Light Chain | DIQLTQSPSSLSASVGDRVTITCRASKSVDSYGNSFMHWY | SEQ ID |
| Variable | QQKPGKAPKLLIYLASDLESGVPSRFSGSGSGTDFTLTISSL | NO: 49 |
| domain | QPEDFATYYCQQNNEDPRTFGGGTKVEIK | |
| LC6_m6 | | |
| Light Chain | DIQLTQSPSSLSASVGDRVTITCRASKSVDSYGNSFMHWY | SEQ ID |
| Variable | QQKPGKAPKLLIYLASQLESGVPSRFSGSGSGTDFTLTISSL | NO: 50 |
| domain | QPEDFATYYCQQNNEDPRTFGGGTKVEIK | |
| LC6_m7 | | |
| Light Chain | DIQLTQSPSSLSASVGDRVTITCRASKSVDSYGNSFMHWY | SEQ ID |
| Variable | QQKPGKAPKLLIYLASELESGVPSRFSGSGSGTDFTLTISSL | NO: 51 |
| domain | QPEDFATYYCQQNNEDPRTFGGGTKVEIK | |
| LC6_m8 | | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSGTDFTLTISSL | NO: 52 |
| domain | QPEDFATYYCQQNHEDPRTFGGGTKVEIK | |
| LC6_m9 | | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSGTDFTLTISSL | NO: 53 |
| domain | QPEDFATYYCQQNYEDPRTFGGGTKVEIK | |
| LC6_m10 | | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSGTDFTLTISSL | NO: 54 |
| domain | QPEDFATYYCQQNSEDPRTFGGGTKVEIK | |
| LC6_11 | | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSGTDFTLTISSL | NO: 55 |
| domain | QPEDFATYYCQQNNRDPRTFGGGTKVEIK | |
| LC6_m12 | | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSGTDFTLTISSL | NO: 56 |
| domain | QPEDFATYYCQQNNDDPRTFGGGTKVEIK | |
| LC6_m13 | | |
| Light Chain | DIQLTQSPSSLSASVGDRVTITCRASKSVDSYGNSFMHWY | SEQ ID |
| Variable | QQKPGKAPKLLIYLASNLESGVPSRFSGSGSGTDFTLTISSL | NO: 57 |
| domain | QPEDFATYYCQQNNQDPRTFGGGTKVEIK | |
| LC6_m14 | | |
| Parental | AYSVN | SEQ ID |
| Kabat | | NO: 58 |
| HCDR1 | | |
| HC5_m7 | RYSVN | SEQ ID |
| Kabat | | NO: 59 |
| HCDR1 | | |
| HC5_m8 | KYSVN | SEQ ID |
| Kabat | | NO: 60 |
| HCDR1 | | |
| HC5_m9 | HYSVN | SEQ ID |
| Kabat | | NO: 61 |
| HCDR1 | | |
| HC5_m10 | QYSVN | SEQ ID |
| Kabat | | NO: 62 |
| HCDR1 | | |
| HC5_m11 | EYSVN | SEQ ID |
| Kabat | | NO: 63 |
| HCDR1 | | |
| HC5_m12 | SYSVN | SEQ ID |
| Kabat | | NO: 64 |
| HCDR1 | | |
| HC5 m13 | YYSVN | SEQ ID |
| Kabat | | NO: 65 |
| HCDR1 | | |
| HC5_m14 | AESVN | SEQ ID |
| Kabat | | NO: 66 |
| HCDR1 | | |
| Lebrikizumab | GFSLSAY | SEQ ID |
| Chothia | | NO: 67 |
| HCDR1 | | |
| Parental | GFSLNAY | SEQ ID |
| Chothia | | NO: 68 |
| HCDR1 | | |
| HC6 | GGSLNAY | SEQ ID |
| Chothia | | NO: 69 |
| HCDR1 | | |
| HC7 | GGSLNAY | SEQ ID |
| Chothia | | NO: 70 |
| HCDR1 | | |
| HC5_m1 | GYSLNAY | SEQ ID |
| Chothia | | NO: 71 |
| HCDR1 | | |
| HC5_m2 | GFSLRAY | SEQ ID |
| Chothia | | NO: 72 |
| HCDR1 | | |
| HC5_m3 | GFSLHAY | SEQ ID |
| Chothia | | NO: 73 |
| HCDR1 | | |
| HC5_m4 | GFSLDAY | SEQ ID |
| Chothia | | NO: 74 |
| HCDR1 | | |
| HC5_m5 | GFSLYAY | SEQ ID |
| Chothia | | NO: 75 |
| HCDR1 | | |
| HC5_m7 | GFSLNRY | SEQ ID |
| Chothia | | NO: 76 |
| HCDR1 | | |
| HC5_m8 | GFSLNKY | SEQ ID |
| Chothia | | NO: 77 |
| HCDR1 | | |
| HC5_m9 | GFSLNHY | SEQ ID |
| Chothia | | NO: 78 |
| HCDR1 | | |
| HC5_m10 | GFSLNQY | SEQ ID |
| Chothia | | NO: 79 |
| HCDR1 | | |
| HC5_m11 | GFSLNEY | SEQ ID |
| Chothia | | NO: 80 |
| HCDR1 | | |
| HC5_m12 | GFSLNSY | SEQ ID |
| Chothia | | NO: 81 |
| HCDR1 | | |
| HC5_m13 | GFSLNYY | SEQ ID |
| Chothia | | NO: 82 |
| HCDR1 | | |
| HC5_m14 | GFSLNAE | SEQ ID |
| Chothia | | NO: 83 |
| HCDR1 | | |
| Lebrikizumab | GFSLSAYS | SEQ ID |
| IMGT | | NO: 84 |
| HCDR1 | | |
| Parental | GFSLNAYS | SEQ ID |
| IMGT | | NO: 85 |
| HCDR1 | | |
| HC6 | GGSLNAYS | SEQ ID |
| IMGT | | NO: 86 |
| HCDR1 | | |
| HC5_m1 | GYSLNAYS | SEQ ID |
| IMGT | | NO: 87 |
| HCDR1 | | |
| HC5_m2 | GFSLRAYS | SEQ ID |
| IMGT | | NO: 88 |
| HCDR1 | | |
| HC5_m3 | GFSLHAYS | SEQ ID |
| IMGT | | NO: 89 |
| HCDR1 | | |
| HC5_m4 | GFSLDAYS | SEQ ID |
| IMGT | | NO: 90 |
| HCDR1 | | |
| HC5_m5 | GFSLYAYS | SEQ ID |
| IMGT | | NO: 91 |
| HCDR1 | | |
| HC5_m6 | GFSLSAYS | SEQ ID |
| IMGT | | NO: 92 |
| HCDR1 | | |
| HC5_m7 | GFSLNRYS | SEQ ID |
| IMGT | | NO: 93 |
| HCDR1 | | |
| HC5_m8 | GFSLNKYS | SEQ ID |
| IMGT | | NO: 94 |
| HCDR1 | | |
| HC5_m9 | GFSLNHYS | SEQ ID |
| IMGT | | NO: 95 |
| HCDR1 | | |
| HC5_m10 | GFSLNQYS | SEQ ID |
| IMGT | | NO: 96 |
| HCDR1 | | |
| HC5_m11 | GFSLNEYS | SEQ ID |
| IMGT | | NO: 97 |
| HCDR1 | | |
| HC5_m13 | GFSLNYYS | SEQ ID |
| IMGT | | NO: 98 |
| HCDR1 | | |
| HC5_m14 | GFSLNAES | SEQ ID |
| IMGT | | NO: 99 |
| HCDR1 | | |
| Parental | MIWGDGKIVYNSALKS | SEQ ID |
| Kabat | | NO: 100 |
| HCDR2 | | |
| HC7 | YIYGDGKTNYNPALKS | SEQ ID |
| Kabat | | NO: 101 |
| HCDR2 | | |
| HC5_m15 | MIWSDGKIVYNSALKS | SEQ ID |
| Kabat | | NO: 102 |
| HCDR2 | | |
| HC5_m16 | MIWADGKIVYNSALKS | SEQ ID |
| Kabat | | NO: 103 |
| HCDR2 | | |
| Parental | WGDGK | SEQ ID |
| Chothia | | NO: 104 |
| HCDR2 | | |
| HC7 | YGDGK | SEQ ID |
| Chothia | | NO: 105 |
| HCDR2 | | |
| HC5_m15 | WSDGK | SEQ ID |
| Chothia | | NO: 106 |
| HCDR2 | | |
| HC5_m16 | WADGK | SEQ ID |
| Chothia | | NO: 107 |
| HCDR2 | | |
| Parental | IWGDGKI | SEQ ID |
| IMGT | | NO: 108 |
| HCDR2 | | |
| HC7 | IYGDGKT | SEQ ID |
| IMGT | | NO: 109 |
| HCDR2 | | |
| HC5_m15 | IWSDGKI | SEQ ID |
| IMGT | | NO: 110 |
| HCDR2 | | |
| HC5_m16 | IWADGKI | SEQ ID |
| IMGT | | NO: 111 |
| HCDR2 | | |
| Parental | DGYYPYAMDN | SEQ ID |
| Kabat and | | NO: 112 |
| Chothia | | |
| HCDR3 | | |
| HC7 | DGYYYYAMDV | SEQ ID |
| Kabat and | | NO: 113 |
| Chothia | | |
| HCDR3 | | |
| HC5_m17 | HGYYPYAMDN | SEQ ID |
| Kabat and | | NO: 114 |
| Chothia | | |
| HCDR3 | | |
| HC5_m18 | DLYYPYAMDN | SEQ ID |
| Kabat and | | NO: 115 |
| Chothia | | |
| HCDR3 | | |
| HC5_m19 | DKYYPYAMDN | SEQ ID |
| Kabat and | | NO: 116 |
| Chothia | | |
| HCDR3 | | |
| HC5_m20 | DGYYGYAMDN | SEQ ID |
| Kabat and | | NO: 117 |
| Chothia | | |
| HCDR3 | | |
| HC5_m21 | DGYYAYAMDN | SEQ ID |
| Kabat and | | NO: 118 |
| Chothia | | |
| HCDR3 | | |
| HC5_m22 | DGYYSYAMDN | SEQ ID |
| Kabat and | | NO: 119 |
| Chothia | | |
| HCDR3 | | |
| HC5_m23 | DGYYTYAMDN | SEQ ID |
| Kabat and | | NO: 120 |
| Chothia | | |
| HCDR3 | | |
| HC5_m12 | GNSLNSYS | SEQ ID |
| IMGT | | NO: 121 |
| HCDR1 | | |
| | | SEQ ID |
| | | NO: 122 |
| | | SEQ ID |
| | | NO: 123 |
| | | SEQ ID |
| | | NO: 124 |
| | | SEQ ID |
| | | NO: 125 |
| | | SEQ ID |
| | | NO: 126 |
| | | SEQ ID |
| | | NO: 127 |
| | | SEQ ID |
| | | NO: 128 |
| | | SEQ ID |
| | | NO: 129 |
| Parental | AGDGYYPYAMDN | SEQ ID |
| IMGT | | NO: 130 |
| HCDR3 | | |
| HC6 | ARDGYYPYAMDN | SEQ ID |
| IMGT | | NO: 131 |
| HCDR3 | | |
| HC7 | ARDGYYYYAMDV | SEQ ID |
| IMGT | | NO: 132 |
| HCDR3 | | |
| HC5_m17 | AGHGYYPYAMDN | SEQ ID |
| IMGT | | NO: 133 |
| HCDR3 | | |
| HC5_m18 | AGDLYYPYAMDN | SEQ ID |
| IMGT | | NO: 134 |
| HCDR3 | | |
| HC5_m19 | AGDKYYPYAMDN | SEQ ID |
| IMGT | | NO: 135 |
| HCDR3 | | |
| HC5_m20 | AGDGYYGYAMDN | SEQ ID |
| IMGT | | NO: 136 |
| HCDR3 | | |
| HC5_m21 | AGDGYYAYAMDN | SEQ ID |
| IMGT | | NO: 137 |
| HCDR3 | | |
| HC5_m22 | AGDGYYSYAMDN | SEQ ID |
| IMGT | | NO: 138 |
| HCDR3 | | |
| HC5_m23 | AGDGYYTYAMDN | SEQ ID |
| IMGT | | NO: 139 |
| HCDR3 | | |
| HC1_m6 | AGDGYYPYAMDN | SEQ ID |
| IMGT | | NO: 140 |
| HCDR3 | | |
| Parental | RASKSVDSYGNSFMH | SEQ ID |
| Kabat and | | NO: 141 |
| Chothia | | |
| LCDR1 | | |
| LC10 | RASQSVDSNGNNFLH | SEQ ID |
| Kabat and | | NO: 142 |
| Chothia | | |
| LCDR1 | | |
| LC6_m1 | RASKSVDSYGNSRMH | SEQ ID |
| Kabat and | | NO: 143 |
| Chothia | | |
| LCDR1 | | |
| LC6_m2 | RASKSVDSYGNSSMH | SEQ ID |
| Kabat and | | NO: 144 |
| Chothia | | |
| LCDR1 | | |
| | | SEQ ID |
| | | NO: 145 |
| | | SEQ ID |
| | | NO: 146 |
| | | SEQ ID |
| | | NO: 147 |
| | | SEQ ID |
| | | NO: 148 |
| Parental | KSVDSYGNSF | SEQ ID |
| IMGT | | NO: 149 |
| LCDR1 | | |
| LC10 | QSVDSNGNNF | SEQ ID |
| IMGT | | NO: 150 |
| LCDR1 | | |
| LC6_m1 | KSVDSYGNSR | SEQ ID |
| IMGT | | NO: 151 |
| LCDR1 | | |
| LC6_m2 | KSVDSYGNSS | SEQ ID |
| IMGT | | NO: 152 |
| LCDR1 | | |
| Parental | LASNLES | SEQ ID |
| Kabat and | | NO: 153 |
| Chothia | | |
| LCDR2 | | |
| LC6_m5 | LASHLES | SEQ ID |
| Kabat and | | NO: 154 |
| Chothia | | |
| LCDR2 | | |
| LC6_m6 | LASDLES | SEQ ID |
| Kabat and | | NO: 155 |
| Chothia | | |
| LCDR2 | | |
| LC6_m7 | LASQLES | SEQ ID |
| Kabat and | | NO: 156 |
| Chothia | | |
| LCDR2 | | |
| LC6_m8 | LASELES | SEQ ID |
| Kabat and | | NO: 157 |
| Chothia | | |
| LCDR2 | | |
| LC10 | LASNRES | SEQ ID |
| Kabat and | | NO: 158 |
| Chothia | | |
| LCDR2 | | |
| | | SEQ ID |
| | | NO: 159 |
| | | SEQ ID |
| | | NO: 160 |
| | | SEQ ID |
| | | NO: 161 |
| | | SEQ ID |
| | | NO: 162 |
| | | SEQ ID |
| | | NO: 163 |
| | | SEQ ID |
| | | NO: 164 |
| Parental | QQNNEDPRT | SEQ ID |
| Kabat, | | NO: 165 |
| Chothia and | | |
| IMGT | | |
| LCDR3 | | |
| LC10 | QQNNHTPRT | SEQ ID |
| Kabat, | | NO: 166 |
| Chothia and | | |
| IMGT | | |
| LCDR3 | | |
| LC6_m9 | QQNHEDPRT | SEQ ID |
| Kabat, | | NO: 167 |
| Chothia and | | |
| IMGT | | |
| LCDR3 | | |
| LC6_m10 | QQNYEDPRT | SEQ ID |
| Kabat, | | NO: 168 |
| Chothia and | | |
| IMGT | | |
| LCDR3 | | |
| LC6_m11 | QQNSEDPRT | SEQ ID |
| Kabat, | | NO: 169 |
| Chothia and | | |
| IMGT | | |
| LCDR3 | | |
| LC6_m12 | QQNNRDPRT | SEQ ID |
| Kabat, | | NO: 170 |
| Chothia and | | |
| IMGT | | |
| LCDR3 | | |
| LC6_m13 | QQNNDDPRT | SEQ ID |
| Kabat, | | NO: 171 |
| Chothia and | | |
| IMGT | | |
| LCDR3 | | |
| LC6_m14 | QQNNQDPRT | SEQ ID |
| Kabat, | | NO: 172 |
| Chothia and | | |
| IMGT | | |
| LCDR3 | | |
| | | SEQ ID |
| | | NO: 173 |
| | | SEQ ID |
| | | NO: 174 |
| | | SEQ ID |
| | | NO: 175 |
| | | SEQ ID |
| | | NO: 176 |
| | | SEQ ID |
| | | NO: 177 |
| | | SEQ ID |
| | | NO: 178 |
| | | SEQ ID |
| | | NO: 179 |
| | | SEQ ID |
| | | NO: 180 |
| | | SEQ ID |
| | | NO: 181 |
| | | SEQ ID |
| | | NO: 182 |
| | | SEQ ID |
| | | NO: 183 |
| | | SEQ ID |
| | | NO: 184 |
| | | SEQ ID |
| | | NO: 185 |
| | | SEQ ID |
| | | NO: 186 |
| | | SEQ ID |
| | | NO: 187 |
| | | SEQ ID |
| | | NO: 188 |
| HC | QVQLQESGPGLVAPSQSLSITCTVSGFSLN | SEQ ID |
| Framework | | NO: 198 |
| Region 1 | | |
| HC0 Kabat | | |
| HC | QVQLQESGPGLVAPSQSLSITCTVSGFSLN | SEQ ID |
| Framework | | NO: 198 |
| Region 1 | | |
| HC0_M | | |
| Kabat | | |
| HC | EVQLQESGPGLVKPSETLSLTCTVSGFSLN | SEQ ID |
| Framework | | NO: 200 |
| Region 1 | | |
| HC1 Kabat | | |
| HC | EVQLVQSGAEVKKPGASVKVSCKASGFSLN | SEQ ID |
| Framework | | NO: 201 |
| Region 1 | | |
| HC2 Kabat | | |
| HC | EVQLVQSGAEVKKPGSSVKVSCKASGFSLN | SEQ ID |
| Framework | | NO: 202 |
| Region 1 | | |
| HC3 Kabat | | |
| HC | EVQLVESGGGLVKPGGSLRLSCAASGFSLN | SEQ ID |
| Framework | | NO: 203 |
| Region 1 | | |
| HC4 Kabat | | |
| HC | EVQLLESGGGLVQPGGSLRLSCAASGFSLN | SEQ ID |
| Framework | | NO: 204 |
| Region 1 | | |
| HC5 Kabat | | |
| HC | EVQLQESGPGLVKPSETLSLTCTVSGGSLN | SEQ ID |
| Framework | | NO: 205 |
| Region 1 | | |
| HC6 Kabat | | |
| HC | QVQLQESGPGLVKPSETLSLTCTVSGGSLN | SEQ ID |
| Framework | | NO: 206 |
| Region 1 | | |
| HC7 Kabat | | |
| HC | EVQLLESGGGLVQPGGSLRLSCAASGYSLN | SEQ ID |
| Framework | | NO: 207 |
| Region 1 | | |
| HC5_m1 | | |
| Kabat | | |
| HC | EVQLLESGGGLVQPGGSLRLSCAASGFSLR | SEQ ID |
| Framework | | NO: 208 |
| Region 1 | | |
| HC5_m2 | | |
| Kabat | | |
| HC | EVQLLESGGGLVQPGGSLRLSCAASGFSLH | SEQ ID |
| Framework | | NO: 209 |
| Region 1 | | |
| HC5_m3 | | |
| Kabat | | |
| HC | EVQLLESGGGLVQPGGSLRLSCAASGFSLD | SEQ ID |
| Framework | | NO: 210 |
| Region 1 | | |
| HC5_m4 | | |
| Kabat | | |
| HC | EVQLLESGGGLVQPGGSLRLSCAASGFSLY | SEQ ID |
| Framework | | NO: 211 |
| Region 1 | | |
| HC5_m5 | | |
| Kabat | | |
| HC | EVQLLESGGGLVQPGGSLRLSCAASGFSLS | SEQ ID |
| Framework | | NO: 212 |
| Region 1 | | |
| HC5_m6 | | |
| Kabat | | |
| HC | EVQLLESGGGLVQPGGSLRLSCAASGFSLN | SEQ ID |
| Framework | | NO: 204 |
| Region 1 | | |
| HC5_m7 | | |
| Kabat | | |
| HC | EVQLLESGGGLVQPGGSLRLSCAASGFSLN | SEQ ID |
| Framework | | NO: 204 |
| Region 1 | | |
| HC5_m8 | | |
| Kabat | | |
| HC | EVQLLESGGGLVQPGGSLRLSCAASGFSLN | SEQ ID |
| Framework | | NO: 204 |
| Region 1 | | |
| HC5_m9 | | |
| Kabat | | |
| HC | EVQLLESGGGLVQPGGSLRLSCAASGFSLN | SEQ ID |
| Framework | | NO: 204 |
| Region 1 | | |
| HC5_m10 | | |
| Kabat | | |
| HC | EVQLLESGGGLVQPGGSLRLSCAASGFSLN | SEQ ID |
| Framework | | NO: 204 |
| Region 1 | | |
| HC5_m11 | | |
| Kabat | | |
| HC | EVQLLESGGGLVQPGGSLRLSCAASGFSLN | SEQ ID |
| Framework | | NO: 204 |
| Region 1 | | |
| HC5_m12 | | |
| Kabat | | |
| HC | EVQLLESGGGLVQPGGSLRLSCAASGFSLN | SEQ ID |
| Framework | | NO: 204 |
| Region 1 | | |
| HC5_m13 | | |
| Kabat | | |
| HC | EVQLLESGGGLVQPGGSLRLSCAASGFSLN | SEQ ID |
| Framework | | NO: 204 |
| Region 1 | | |
| HC5_m14 | | |
| Kabat | | |
| HC | EVQLLESGGGLVQPGGSLRLSCAASGFSLN | SEQ ID |
| Framework | | NO: 204 |
| Region 1 | | |
| HC5_m15 | | |
| Kabat | | |
| HC | EVQLLESGGGLVQPGGSLRLSCAASGFSLN | SEQ ID |
| Framework | | NO: 204 |
| Region 1 | | |
| HC5_m16 | | |
| Kabat | | |
| HC | EVQLLESGGGLVQPGGSLRLSCAASGFSLN | SEQ ID |
| Framework | | NO: 204 |
| Region 1 | | |
| HC5_m17 | | |
| Kabat | | |
| HC | EVQLLESGGGLVQPGGSLRLSCAASGFSLN | SEQ ID |
| Framework | | NO: 204 |
| Region 1 | | |
| HC5_m18 | | |
| Kabat | | |
| HC | EVQLLESGGGLVQPGGSLRLSCAASGFSLN | SEQ ID |
| Framework | | NO: 204 |
| Region 1 | | |
| HC5_m19 | | |
| Kabat | | |
| HC | EVQLLESGGGLVQPGGSLRLSCAASGFSLN | SEQ ID |
| Framework | | NO: 204 |
| Region 1 | | |
| HC5_m20 | | |
| Kabat | | |
| HC | EVQLLESGGGLVQPGGSLRLSCAASGFSLN | SEQ ID |
| Framework | | NO: 204 |
| Region 1 | | |
| HC5_m21 | | |
| Kabat | | |
| HC | EVQLLESGGGLVQPGGSLRLSCAASGFSLN | SEQ ID |
| Framework | | NO: 204 |
| Region 1 | | |
| HC5_m22 | | |
| Kabat | | |
| HC | EVQLLESGGGLVQPGGSLRLSCAASGFSLN | SEQ ID |
| Framework | | NO: 204 |
| Region 1 | | |
| HC5_m24 | | |
| Kabat | | |
| LC | NIVLTQSPASLAVSLGQRATISC | SEQ ID |
| Framework | | NO: 230 |
| Region | | |
| 1LC0 Kabat | | |
| LC | DIQLTQSPSSLSASVGDRVTITC | SEQ ID |
| Framework | | NO: 231 |
| Region 1 | | |
| LC1 Kabat | | |
| LC | DIQLTQSPSSLSASVGDRVTITC | SEQ ID |
| Framework | | NO: 231 |
| Region 1 | | |
| LC2 Kabat | | |
| LC | EIVLTQSPATLSVSPGERATLSC | SEQ ID |
| Framework | | NO: 233 |
| Region 1 | | |
| LC3 Kabat | | |
| LC | DIVLTQSPLSLPVTPGEPASISC | SEQ ID |
| Framework | | NO: 234 |
| Region 1 | | |
| LC4 Kabat | | |
| LC | DIVLTQSPDSLAVSLGERATINC | SEQ ID |
| Framework | | NO: 235 |
| Region 1 | | |
| LC5 Kabat | | |
| LC | DIQLTQSPSSLSASVGDRVTITC | SEQ ID |
| Framework | | NO: 231 |
| Region 1 | | |
| LC6 Kabat | | |
| LC | EIVLTQSPATLSVSPGERATLSC | SEQ ID |
| Framework | | NO: 233 |
| Region 1 | | |
| LC7 Kabat | | |
| LC | DIVLTQSPLSLPVTPGEPASISC | SEQ ID |
| Framework | | NO: 234 |
| Region 1 | | |
| LC8 Kabat | | |
| LC | DIVLTQSPASLAVSPGERATISC | SEQ ID |
| Framework | | NO: 239 |
| Region 1 | | |
| LC9 Kabat | | |
| LC | DIVLTQSPASLAVSPGERATISC | SEQ ID |
| Framework | | NO: 239 |
| Region 1 | | |
| LC10 Kabat | | |
| LC | DIQLTQSPSSLSASVGDRVTITC | SEQ ID |
| Framework | | NO: 231 |
| Region 1 | | |
| LC6_m1 | | |
| Kabat | | |
| LC | DIQLTQSPSSLSASVGDRVTITC | SEQ ID |
| Framework | | NO: 231 |
| Region 1 | | |
| LC6_m2 | | |
| Kabat | | |
| LC | DIQLTQSPSSLSASVGDRVTITC | SEQ ID |
| Framework | | NO: 231 |
| Region 1 | | |
| LC6_m3 | | |
| Kabat | | |
| LC | DIQLTQSPSSLSASVGDRVTITC | SEQ ID |
| Framework | | NO: 231 |
| Region 1 | | |
| LC6_m4 | | |
| Kabat | | |
| LC | DIQLTQSPSSLSASVGDRVTITC | SEQ ID |
| Framework | | NO: 231 |
| Region 1 | | |
| LC6_m5 | | |
| Kabat | | |
| LC | DIQLTQSPSSLSASVGDRVTITC | SEQ ID |
| Framework | | NO: 231 |
| Region 1 | | |
| LC6_m6 | | |
| Kabat | | |
| LC | DIQLTQSPSSLSASVGDRVTITC | SEQ ID |
| Framework | | NO: 231 |
| Region 1 | | |
| LC6_m7 | | |
| Kabat | | |
| LC | DIQLTQSPSSLSASVGDRVTITC | SEQ ID |
| Framework | | NO: 231 |
| Region 1 | | |
| LC6_m8 | | |
| Kabat | | |
| LC | DIQLTQSPSSLSASVGDRVTITC | SEQ ID |
| Framework | | NO: 231 |
| Region 1 | | |
| LC6_m9 | | |
| Kabat | | |
| LC | DIQLTQSPSSLSASVGDRVTITC | SEQ ID |
| Framework | | NO: 231 |
| Region 1 | | |
| LC6_m10 | | |
| Kabat | | |
| LC | DIQLTQSPSSLSASVGDRVTITC | SEQ ID |
| Framework | | NO: 231 |
| Region 1 | | |
| LC6_m11 | | |
| Kabat | | |
| LC | DIQLTQSPSSLSASVGDRVTITC | SEQ ID |
| Framework | | NO: 231 |
| Region 1 | | |
| LC6_m12 | | |
| Kabat | | |
| LC | DIQLTQSPSSLSASVGDRVTITC | SEQ ID |
| Framework | | NO: 231 |
| Region 1 | | |
| LC6_m13 | | |
| Kabat | | |
| LC | DIQLTQSPSSLSASVGDRVTITC | SEQ ID |
| Framework | | NO: 231 |
| Region 1 | | |
| LC6_m14 | | |
| Kabat | | |
| Lebrikizumab- | WIRQPPGKALEWLA | SEQ ID |
| HC | | NO: 255 |
| HC | WVRQPPGKGLEWLG | SEQ ID |
| Framework | | NO: 256 |
| Region 2 | | |
| HC0 | | |
| HC | WVRQPPGKGLEWLG | SEQ ID |
| Framework | | NO: 256 |
| Region 2 | | |
| HC0_M | | |
| Kabat | | |
| HC | WIRQPPGKGLEWLG | SEQ ID |
| Framework | | NO: 258 |
| Region 2 | | |
| HC1 Kabat | | |
| HC | WVRQAPGQGLEWLG | SEQ ID |
| Framework | | NO: 259 |
| Region 2 | | |
| HC2 Kabat | | |
| HC | WVRQAPGQGLEWLG | SEQ ID |
| Framework | | NO: 259 |
| Region 2 | | |
| HC3 Kabat | | |
| HC | WVRQAPGKGLEWLG | SEQ ID |
| Framework | | NO: 262 |
| Region 2 | | |
| HC4 Kabat | | |
| HC | WVRQAPGKGLEWLG | SEQ ID |
| Framework | | NO: 262 |
| Region 2 | | |
| HC5 Kabat | | |
| HC | WVRQPPGKGLEWLG | SEQ ID |
| Framework | | NO: 263 |
| Region 2 | | |
| HC6 Kabat | | |
| HC | WVRQPPGKGLEWLG | SEQ ID |
| Framework | | NO: 263 |
| Region 2 | | |
| HC7 Kabat | | |
| HC | WVRQAPGKGLEWLG | SEQ ID |
| Framework | | NO: 262 |
| Region 2 | | |
| HC5_m1 | | |
| Kabat | | |
| HC | WVRQAPGKGLEWLG | SEQ ID |
| Framework | | NO: 262 |
| Region 2 | | |
| HC5_m2 | | |
| Kabat | | |
| HC | WVRQAPGKGLEWLG | SEQ ID |
| Framework | | NO: 262 |
| Region 2 | | |
| HC5_m3 | | |
| Kabat | | |
| HC | WVRQAPGKGLEWLG | SEQ ID |
| Framework | | NO: 262 |
| Region 2 | | |
| HC5_m4 | | |
| Kabat | | |
| HC | WVRQAPGKGLEWLG | SEQ ID |
| Framework | | NO: 262 |
| Region 2 | | |
| HC5_m5 | | |
| Kabat | | |
| HC | WVRQAPGKGLEWLG | SEQ ID |
| Framework | | NO: 262 |
| Region 2 | | |
| HC5_m6 | | |
| Kabat | | |
| HC | WVRQAPGKGLEWLG | SEQ ID |
| Framework | | NO: 262 |
| Region 2 | | |
| HC5_m7 | | |
| Kabat | | |
| HC | WVRQAPGKGLEWLG | SEQ ID |
| Framework | | NO: 262 |
| Region 2 | | |
| HC5_m8 | | |
| Kabat | | |
| HC | WVRQAPGKGLEWLG | SEQ ID |
| Framework | | NO: 262 |
| Region 2 | | |
| HC5_m9 | | |
| Kabat | | |
| HC | WVRQAPGKGLEWLG | SEQ ID |
| Framework | | NO: 262 |
| Region 2 | | |
| HC5_m10 | | |
| Kabat | | |
| HC | WVRQAPGKGLEWLG | SEQ ID |
| Framework | | NO: 262 |
| Region 2 | | |
| HC5_m11 | | |
| Kabat | | |
| HC | WVRQAPGKGLEWLG | SEQ ID |
| Framework | | NO: 262 |
| Region 2 | | |
| HC5_m12 | | |
| Kabat | | |
| HC | WVRQAPGKGLEWLG | SEQ ID |
| Framework | | NO: 262 |
| Region 2 | | |
| HC5_m13 | | |
| Kabat | | |
| HC | WVRQAPGKGLEWLG | SEQ ID |
| Framework | | NO: 262 |
| Region 2 | | |
| HC5_m14 | | |
| Kabat | | |
| HC | WVRQAPGKGLEWLG | SEQ ID |
| Framework | | NO: 262 |
| Region 2 | | |
| HC5_m15 | | |
| Kabat | | |
| HC | WVRQAPGKGLEWLG | SEQ ID |
| Framework | | NO: 262 |
| Region 2 | | |
| HC5_m16 | | |
| Kabat | | |
| HC | WVRQAPGKGLEWLG | SEQ ID |
| Framework | | NO: 262 |
| Region 2 | | |
| HC5_m17 | | |
| Kabat | | |
| HC | WVRQAPGKGLEWLG | SEQ ID |
| Framework | | NO: 262 |
| Region 2 | | |
| HC5_m18 | | |
| Kabat H | | |
| HC | WVRQAPGKGLEWLG | SEQ ID |
| Framework | | NO: 262 |
| Region 2 | | |
| HC5_m19 | | |
| Kabat | | |
| HC | WVRQAPGKGLEWLG | SEQ ID |
| Framework | | NO: 262 |
| Region 2 | | |
| HC5_m20 | | |
| Kabat | | |
| HC | WVRQAPGKGLEWLG | SEQ ID |
| Framework | | NO: 262 |
| Region 2 | | |
| HC5_m21 | | |
| Kabat | | |
| HC | WVRQAPGKGLEWLG | SEQ ID |
| Framework | | NO: 262 |
| Region 2 | | |
| HC5_m22 | | |
| Kabat | | |
| HC | WVRQAPGKGLEWLG | SEQ ID |
| Framework | | NO: 262 |
| Region 2 | | |
| HC5_m24 | | |
| Kabat | | |
| Lebrikizumab- | WYQQKPGQPPKLLIY | SEQ ID |
| LC | | NO: 286 |
| LC | WYQQKPGQPPKLLIY | SEQ ID |
| Framework | | NO: 286 |
| Region 2 | | |
| LC0 Kabat | | |
| LC | WYQQKPGKAPKLLIY | SEQ ID |
| Framework | | NO: 288 |
| Region 2 | | |
| LC1 Kabat | | |
| LC | WYQQKPGKAPKLLIY | SEQ ID |
| Framework | | NO: 288 |
| Region 2 | | |
| LC2 Kabat | | |
| LC | WYQQKPGQAPRLLIY | SEQ ID |
| Framework | | NO: 290 |
| Region 2 | | |
| LC3 Kabat | | |
| LC | WYLQKPGQSPQLLIY | SEQ ID |
| Framework | | NO: 291 |
| Region 2 | | |
| LC4 Kabat | | |
| LC | WYQQKPGQPPKLLIY | SEQ ID |
| Framework | | NO: 286 |
| Region 2 | | |
| LC5 Kabat | | |
| LC | WYQQKPGKAPKLLIY | SEQ ID |
| Framework | | NO: 288 |
| Region 2 | | |
| LC6 Kabat | | |
| LC | WYQQKPGQAPRLLIY | SEQ ID |
| Framework | | NO: 290 |
| Region 2 | | |
| LC7 Kabat | | |
| LC | WYLQKPGQSPQLLIY | SEQ ID |
| Framework | | NO: 291 |
| Region 2 | | |
| LC8 Kabat | | |
| LC | WYQQKPGQPPKLLIY | SEQ ID |
| Framework | | NO: 292 |
| Region 2 | | |
| LC9 Kabat | | |
| LC | WYQQKPGQPPKLLIY | SEQ ID |
| Framework | | NO: 286 |
| Region 2 | | |
| LC10 Kabat | | |
| LC | WYQQKPGKAPKLLIY | SEQ ID |
| Framework | | NO: 288 |
| Region 2 | | |
| LC6_m1 | | |
| Kabat | | |
| LC | WYQQKPGKAPKLLIY | SEQ ID |
| Framework | | NO: 288 |
| Region 2 | | |
| LC6_m2 | | |
| Kabat | | |
| LC | WYQQKPGKAPKLLIR | SEQ ID |
| Framework | | NO: 300 |
| Region 2 | | |
| LC6_m3 | | |
| Kabat | | |
| LC | WYQQKPGKAPKLLIF | SEQ ID |
| Framework | | NO: 301 |
| Region 2 | | |
| LC6_m4 | | |
| Kabat | | |
| LC | WYQQKPGKAPKLLIY | SEQ ID |
| Framework | | NO: 288 |
| Region 2 | | |
| LC6_m5 | | |
| Kabat | | |
| LC | WYQQKPGKAPKLLIY | SEQ ID |
| Framework | | NO: 288 |
| Region 2 | | |
| LC6_m6 | | |
| Kabat | | |
| LC | WYQQKPGKAPKLLIY | SEQ ID |
| Framework | | NO: 288 |
| Region 2 | | |
| LC6_m7 | | |
| Kabat | | |
| LC | WYQQKPGKAPKLLIY | SEQ ID |
| Framework | | NO: 288 |
| Region 2 | | |
| LC6_m8 | | |
| Kabat | | |
| LC | WYQQKPGKAPKLLIY | SEQ ID |
| Framework | | NO: 288 |
| Region 2 | | |
| LC6_m9 | | |
| Kabat | | |
| LC | WYQQKPGKAPKLLIY | SEQ ID |
| Framework | | NO: 288 |
| Region 2 | | |
| LC6_m10 | | |
| Kabat | | |
| LC | WYQQKPGKAPKLLIY | SEQ ID |
| Framework | | NO: 288 |
| Region 2 | | |
| LC6_m11 | | |
| Kabat | | |
| LC | WYQQKPGKAPKLLIY | SEQ ID |
| Framework | | NO: 288 |
| Region 2 | | |
| LC6_m12 | | |
| Kabat | | |
| LC | WYQQKPGKAPKLLIY | SEQ ID |
| Framework | | NO: 288 |
| Region 2 | | |
| LC6_m13 | | |
| Kabat | | |
| LC | WYQQKPGKAPKLLIY | SEQ ID |
| Framework | | NO: 288 |
| Region 2 | | |
| LC6_m14 | | |
| Kabat | | |
| Lebrikizumab- | RLTISKDTSKNQVVLTMTNMDPVDTATYYCAG | SEQ ID |
| HC | | NO: 311 |
| HC | RLNISKDSSKSQVFLKMSSLQSDDTARYYCAG | SEQ ID |
| Framework | | NO: 312 |
| Region 3 | | |
| HC0 Kabat | | |
| HC | RLTISKDSSKSQVFLKMSSLQSDDTARYYCAG | SEQ ID |
| Framework | | NO: 313 |
| Region 3 | | |
| HC0_M | | |
| Kabat | | |
| HC | RLTISKDSSKNQVSLKLSSVTAADTAVYYCAG | SEQ ID |
| Framework | | NO: 314 |
| Region 3 | | |
| HC1 Kabat | | |
| HC | RLTITKDSSTSTVYMELSSLRSEDTAVYYCAG | SEQ ID |
| Framework | | NO: 315 |
| Region 3 | | |
| HC2 Kabat | | |
| HC | RLTITKDSSTSTVYMELSSLRSEDTAVYYCAG | SEQ ID |
| Framework | | NO: 315 |
| Region 3 | | |
| HC3 Kabat | | |
| HC | RLTISKDSSKNTVYLQMNSLKTEDTAVYYCAG | SEQ ID |
| Framework | | NO: 317 |
| Region 3 | | |
| HC4 Kabat | | |
| HC | RLTISKDSSKNTVYLQMNSLRAEDTAVYYCAG | SEQ ID |
| Framework | | NO: 318 |
| Region 3 | | |
| HC5 Kabat | | |
| HC | RLTISLDTSKSQVFLKMSSLTAADTAVYYCAR | SEQ ID |
| Framework | | NO: 319 |
| Region 3 | | |
| HC6 Kabat | | |
| HC | RLTISLDTSKSQVFLKMSSLTAADTAVYYCAR | SEQ ID |
| Framework | | NO: 317 |
| Region 3 | | |
| HC7 Kabat | | |
| HC | RLTISKDSSKNTVYLQMNSLRAEDTAVYYCAG | SEQ ID |
| Framework | | NO: 318 |
| Region 3 | | |
| HC5_m1 | | |
| Kabat | | |
| HC | RLTISKDSSKNTVYLQMNSLRAEDTAVYYCAG | SEQ ID |
| Framework | | NO: 318 |
| Region 3 | | |
| HC5_m2 | | |
| Kabat | | |
| HC | RLTISKDSSKNTVYLQMNSLRAEDTAVYYCAG | SEQ ID |
| Framework | | NO: 318 |
| Region 3 | | |
| HC5_m3 | | |
| Kabat | | |
| HC | RLTISKDSSKNTVYLQMNSLRAEDTAVYYCAG | SEQ ID |
| Framework | | NO: 318 |
| Region 3 | | |
| HC5_m4 | | |
| Kabat | | |
| HC | RLTISKDSSKNTVYLQMNSLRAEDTAVYYCAG | SEQ ID |
| Framework | | NO: 318 |
| Region 3 | | |
| HC5_m5 | | |
| Kabat | | |
| HC | RLTISKDSSKNTVYLQMNSLRAEDTAVYYCAG | SEQ ID |
| Framework | | NO: 318 |
| Region 3 | | |
| HC5_m6 | | |
| Kabat | | |
| HC | RLTISKDSSKNTVYLQMNSLRAEDTAVYYCAG | SEQ ID |
| Framework | | NO: 318 |
| Region 3 | | |
| HC5_m7 | | |
| Kabat | | |
| HC | RLTISKDSSKNTVYLQMNSLRAEDTAVYYCAG | SEQ ID |
| Framework | | NO: 318 |
| Region 3 | | |
| HC5_m8 | | |
| Kabat | | |
| HC | RLTISKDSSKNTVYLQMNSLRAEDTAVYYCAG | SEQ ID |
| Framework | | NO: 318 |
| Region 3 | | |
| HC5_m9 | | |
| Kabat | | |
| HC | RLTISKDSSKNTVYLQMNSLRAEDTAVYYCAG | SEQ ID |
| Framework | | NO: 318 |
| Region 3 | | |
| HC5_m10 | | |
| Kabat | | |
| HC | RLTISKDSSKNTVYLQMNSLRAEDTAVYYCAG | SEQ ID |
| Framework | | NO: 318 |
| Region 3 | | |
| HC5_m11 | | |
| Kabat | | |
| HC | RLTISKDSSKNTVYLQMNSLRAEDTAVYYCAG | SEQ ID |
| Framework | | NO: 318 |
| Region 3 | | |
| HC5_m12 | | |
| Kabat | | |
| HC | RLTISKDSSKNTVYLQMNSLRAEDTAVYYCAG | SEQ ID |
| Framework | | NO: 318 |
| Region 3 | | |
| HC5_m13 | | |
| Kabat | | |
| HC | RLTISKDSSKNTVYLQMNSLRAEDTAVYYCAG | SEQ ID |
| Framework | | NO: 318 |
| Region 3 | | |
| HC5_m14 | | |
| Kabat | | |
| HC | RLTISKDSSKNTVYLQMNSLRAEDTAVYYCAG | SEQ ID |
| Framework | | NO: 318 |
| Region 3 | | |
| HC5_m15 | | |
| Kabat | | |
| HC | RLTISKDSSKNTVYLQMNSLRAEDTAVYYCAG | SEQ ID |
| Framework | | NO: 318 |
| Region 3 | | |
| HC5_m16 | | |
| Kabat | | |
| HC | RLTISKDSSKNTVYLQMNSLRAEDTAVYYCAG | SEQ ID |
| Framework | | NO: 318 |
| Region 3 | | |
| HC5_m17 | | |
| Kabat | | |
| HC | RLTISKDSSKNTVYLQMNSLRAEDTAVYYCAG | SEQ ID |
| Framework | | NO: 318 |
| Region 3 | | |
| HC5_m18 | | |
| Kabat | | |
| HC | RLTISKDSSKNTVYLQMNSLRAEDTAVYYCAG | SEQ ID |
| Framework | | NO: 318 |
| Region 3 | | |
| HC5_m19 | | |
| Kabat | | |
| HC | RLTISKDSSKNTVYLQMNSLRAEDTAVYYCAG | SEQ ID |
| Framework | | NO: 318 |
| Region 3 | | |
| HC5_m20 | | |
| Kabat | | |
| HC | RLTISKDSSKNTVYLQMNSLRAEDTAVYYCAG | SEQ ID |
| Framework | | NO: 318 |
| Region 3 | | |
| HC5_m21 | | |
| Kabat | | |
| HC | RLTISKDSSKNTVYLQMNSLRAEDTAVYYCAG | SEQ ID |
| Framework | | NO: 318 |
| Region 3 | | |
| HC5_m22 | | |
| Kabat | | |
| HC | RLTISKDSSKNTVYLQMNSLRAEDTAVYYCAG | SEQ ID |
| Framework | | NO: 318 |
| Region 3 | | |
| HC5_m24 | | |
| Kabat | | |
| Lebrikizumab- | GVPDRFSGSGSGTDFTLTISSLQAEDVAVYYC | SEQ ID |
| LC | | NO: 343 |
| LC | GVPARFSGSGSRTDFTLTIDPVEADDAASYYC | SEQ ID |
| Framework | | NO: 344 |
| Region 3 | | |
| LC0 Kabat | | |
| LC | GVPSRFSGSGSRTDFTLTISSLQPEDFATYYC | SEQ ID |
| Framework | | NO: 345 |
| Region 3 | | |
| LC1 Kabat | | |
| LC | GVPSRFSGSGSRTDFTLTISSLQPEDFATYYC | SEQ ID |
| Framework | | NO: 345 |
| Region 3 | | |
| LC2 Kabat | | |
| LC | GIPARFSGSGSRTEFTLTISSLQSEDFAVYYC | SEQ ID |
| Framework | | NO: 347 |
| Region 3 | | |
| LC3 Kabat | | |
| LC | GVPDRFSGSGSRTDFTLKISRVEAEDVGVYYC | SEQ ID |
| Framework | | NO: 348 |
| Region 3 | | |
| LC4 Kabat | | |
| LC | GVPDRFSGSGSGTDFTLTISSLQAEDVAVYYC | SEQ ID |
| Framework | | NO: 349 |
| Region 3 | | |
| LC6 Kabat | | |
| LC | GIPARFSGSGSGTEFTLTISSLQSEDFAVYYC | SEQ ID |
| Framework | | NO: 351 |
| Region 3 | | |
| LC7 Kabat | | |
| LC | GVPDRFSGSGSGTDFTLKISRVEAEDVGVYYC | SEQ ID |
| Framework | | NO: 352 |
| Region 3 | | |
| LC8 Kabat | | |
| LC | GVPDRFSGSGSGTDFTLTISRVEADDVAVYYC | SEQ ID |
| Framework | | NO: 353 |
| Region 3 | | |
| LC9 Kabat | | |
| LC | GVPDRFSGSGSGTDFTLTISRVEADDVAVYYC | SEQ ID |
| Framework | | NO: 353 |
| Region 3 | | |
| LC10 Kabat | | |
| LC | GVPSRFSGSGSGTDFTLTISSLQPEDFATYYC | SEQ ID |
| Framework | | NO: 349 |
| Region 3 | | |
| LC6_m1 | | |
| Kabat | | |
| LC | GVPSRFSGSGSGTDFTLTISSLQPEDFATYYC | SEQ ID |
| Framework | | NO: 349 |
| Region 3 | | |
| LC6_m2 | | |
| Kabat | | |
| LC | GVPSRFSGSGSGTDFTLTISSLQPEDFATYYC | SEQ ID |
| Framework | | NO: 349 |
| Region 3 | | |
| LC6_m3 | | |
| Kabat | | |
| LC | GVPSRFSGSGSGTDFTLTISSLQPEDFATYYC | SEQ ID |
| Framework | | NO: 349 |
| Region 3 | | |
| LC6_m4 | | |
| Kabat | | |
| LC | GVPSRFSGSGSGTDFTLTISSLQPEDFATYYC | SEQ ID |
| Framework | | NO: 349 |
| Region 3 | | |
| LC6_m5 | | |
| Kabat | | |
| LC | GVPSRFSGSGSGTDFTLTISSLQPEDFATYYC | SEQ ID |
| Framework | | NO: 349 |
| Region 3 | | |
| LC6_m6 | | |
| Kabat | | |
| LC | GVPSRFSGSGSGTDFTLTISSLQPEDFATYYC | SEQ ID |
| Framework | | NO: 349 |
| Region 3 | | |
| LC6_m7 | | |
| Kabat | | |
| LC | GVPSRFSGSGSGTDFTLTISSLQPEDFATYYC | SEQ ID |
| Framework | | NO: 349 |
| Region 3 | | |
| LC6_m8 | | |
| Kabat | | |
| LC | GVPSRFSGSGSGTDFTLTISSLQPEDFATYYC | SEQ ID |
| Framework | | NO: 349 |
| Region 3 | | |
| LC6_m9 | | |
| Kabat | | |
| LC | GVPSRFSGSGSGTDFTLTISSLQPEDFATYYC | SEQ ID |
| Framework | | NO: 349 |
| Region 3 | | |
| LC6_m10 | | |
| Kabat | | |
| LC | GVPSRFSGSGSGTDFTLTISSLQPEDFATYYC | SEQ ID |
| Framework | | NO: 349 |
| Region 3 | | |
| LC6_m11 | | |
| Kabat | | |
| LC | GVPSRFSGSGSGTDFTLTISSLQPEDFATYYC | SEQ ID |
| Framework | | NO: 349 |
| Region 3 | | |
| LC6_m12 | | |
| Kabat | | |
| LC | GVPSRFSGSGSGTDFTLTISSLQPEDFATYYC | SEQ ID |
| Framework | | NO: 349 |
| Region 3 | | |
| LC6_m13 | | |
| Kabat | | |
| LC | GVPSRFSGSGSGTDFTLTISSLQPEDFATYYC | SEQ ID |
| Framework | | NO: 349 |
| Region 3 | | |
| LC6_m14 | | |
| Kabat | | |
| Lebrikizumab- | WGQGSLVTVSS | SEQ ID |
| HC | | NO: 368 |
| HC | WGHGTSVTVSS | SEQ ID |
| Framework | | NO: 369 |
| Region 4 | | |
| HC0 | | |
| HC | WGHGTSVTVSS | SEQ ID |
| Framework | | NO: 369 |
| Region 4 | | |
| HC0_M | | |
| Kabat | | |
| HC | WGQGTTVTVSS | SEQ ID |
| Framework | | NO: 371 |
| Region 4 | | |
| HC1 Kabat | | |
| HC | WGQGTTVTVSS | SEQ ID |
| Framework | | NO: 371 |
| Region 4 | | |
| HC2 Kabat | | |
| HC | WGQGTTVTVSS | SEQ ID |
| Framework | | NO: 371 |
| Region 4 | | |
| HC3 Kabat | | |
| HC | WGQGTTVTVSS | SEQ ID |
| Framework | | NO: 371 |
| Region 4 | | |
| HC4 Kabat | | |
| HC | WGQGTTVTVSS | SEQ ID |
| Framework | | NO: 371 |
| Region 4 | | |
| HC5 Kabat | | |
| HC | WGQGTTVTVSS | SEQ ID |
| Framework | | NO: 371 |
| Region 4 | | |
| HC6 Kabat | | |
| HC | WGQGTTVTVSS | SEQ ID |
| Framework | | NO: 371 |
| Region 4 | | |
| HC7 Kabat | | |
| HC | WGQGTTVTVSS | SEQ ID |
| Framework | | NO: 371 |
| Region 4 | | |
| HC5_m1 | | |
| Kabat | | |
| HC | WGQGTTVTVSS | SEQ ID |
| Framework | | NO: 371 |
| Region 4 | | |
| HC5_m2 | | |
| Kabat | | |
| HC | WGQGTTVTVSS | SEQ ID |
| Framework | | NO: 371 |
| Region 4 | | |
| HC5_m3 | | |
| Kabat | | |
| HC | WGQGTTVTVSS | SEQ ID |
| Framework | | NO: 371 |
| Region 4 | | |
| HC5_m4 | | |
| Kabat | | |
| HC | WGQGTTVTVSS | SEQ ID |
| Framework | | NO: 371 |
| Region 4 | | |
| HC5_m5 | | |
| Kabat | | |
| HC | WGQGTTVTVSS | SEQ ID |
| Framework | | NO: 371 |
| Region 4 | | |
| HC5_m6 | | |
| Kabat | | |
| HC | WGQGTTVTVSS | SEQ ID |
| Framework | | NO: 371 |
| Region 4 | | |
| HC5_m7 | | |
| Kabat | | |
| HC | WGQGTTVTVSS | SEQ ID |
| Framework | | NO: 371 |
| Region 4 | | |
| HC5_m8 | | |
| Kabat | | |
| HC | WGQGTTVTVSS | SEQ ID |
| Framework | | NO: 371 |
| Region 4 | | |
| HC5_m9 | | |
| Kabat | | |
| HC | WGQGTTVTVSS | SEQ ID |
| Framework | | NO: 371 |
| Region 4 | | |
| HC5_m10 | | |
| Kabat | | |
| HC | WGQGTTVTVSS | SEQ ID |
| Framework | | NO: 371 |
| Region 4 | | |
| HC5_m11 | | |
| Kabat | | |
| HC | WGQGTTVTVSS | SEQ ID |
| Framework | | NO: 371 |
| Region 4 | | |
| HC5_m12 | | |
| Kabat | | |
| HC | WGQGTTVTVSS | SEQ ID |
| Framework | | NO: 371 |
| Region 4 | | |
| HC5_m13 | | |
| Kabat | | |
| HC | WGQGTTVTVSS | SEQ ID |
| Framework | | NO: 371 |
| Region 4 | | |
| HC5_m14 | | |
| Kabat | | |
| HC | WGQGTTVTVSS | SEQ ID |
| Framework | | NO: 371 |
| Region 4 | | |
| HC5_m15 | | |
| Kabat | | |
| HC | WGQGTTVTVSS | SEQ ID |
| Framework | | NO: 371 |
| Region 4 | | |
| HC5_m16 | | |
| Kabat | | |
| HC | WGQGTTVTVSS | SEQ ID |
| Framework | | NO: 371 |
| Region 4 | | |
| HC5_m17 | | |
| Kabat | | |
| HC | WGQGTTVTVSS | SEQ ID |
| Framework | | NO: 371 |
| Region 4 | | |
| HC5_m18 | | |
| Kabat | | |
| HC | WGQGTTVTVSS | SEQ ID |
| Framework | | NO: 371 |
| Region 4 | | |
| HC5_m19 | | |
| Kabat | | |
| HC | WGQGTTVTVSS | SEQ ID |
| Framework | | NO: 371 |
| Region 4 | | |
| HC5_m20 | | |
| Kabat | | |
| HC | WGQGTTVTVSS | SEQ ID |
| Framework | | NO: 371 |
| Region 4 | | |
| HC5_m21 | | |
| Kabat | | |
| HC | WGQGTTVTVSS | SEQ ID |
| Framework | | NO: 371 |
| Region 4 | | |
| HC5_m22 | | |
| Kabat | | |
| HC | WGQGTTVTVSS | SEQ ID |
| Framework | | NO: 371 |
| Region 4 | | |
| HC5_m24 | | |
| Kabat | | |
| Lebrikizumab- | FGGGTKVEIK | SEQ ID |
| LC | | NO: 400 |
| LC | FGGGTKLEIK | SEQ ID |
| Framework | | NO: 401 |
| Region 4 | | |
| LC0 Kabat | | |
| LC | FGGGTKVEIK | SEQ ID |
| Framework | | NO: 400 |
| Region 4 | | |
| LC1 Kabat | | |
| LC | FGGGTKVEIK | SEQ ID |
| Framework | | NO: 400 |
| Region 4 | | |
| LC2 Kabat | | |
| LC | FGGGTKVEIK | SEQ ID |
| Framework | | NO: 400 |
| Region 4 | | |
| LC3 Kabat | | |
| LC | FGGGTKVEIK | SEQ ID |
| Framework | | NO: 400 |
| Region 4 | | |
| LC4 Kabat | | |
| LC | FGGGTKVEIK | SEQ ID |
| Framework | | NO: 400 |
| Region 4 | | |
| LC5 Kabat | | |
| LC | FGGGTKVEIK | SEQ ID |
| Framework | | NO: 400 |
| Region 4 | | |
| LC6 Kabat | | |
| LC | FGGGTKVEIK | SEQ ID |
| Framework | | NO: 400 |
| Region 4 | | |
| LC7 Kabat | | |
| LC | FGGGTKVEIK | SEQ ID |
| Framework | | NO: 400 |
| Region 4 | | |
| LC8 Kabat | | |
| LC | FGGGTKLEIK | SEQ ID |
| Framework | | NO: 401 |
| Region 4 | | |
| LC9 Kabat | | |
| LC | FGGGTKLEIK | SEQ ID |
| Framework | | NO: 401 |
| Region 4 | | |
| LC10 Kabat | | |
| LC | FGGGTKVEIK | SEQ ID |
| Framework | | NO: 400 |
| Region 4 | | |
| LC6_m1 | | |
| Kabat | | |
| LC | FGGGTKVEIK | SEQ ID |
| Framework | | NO: 400 |
| Region 4 | | |
| LC6_m2 | | |
| Kabat | | |
| LC | FGGGTKVEIK | SEQ ID |
| Framework | | NO: 400 |
| Region 4 | | |
| LC6_m3 | | |
| Kabat | | |
| LC | FGGGTKVEIK | SEQ ID |
| Framework | | NO: 400 |
| Region 4 | | |
| LC6_m4 | | |
| Kabat | | |
| LC | FGGGTKVEIK | SEQ ID |
| Framework | | NO: 400 |
| Region 4 | | |
| LC6_m5 | | |
| Kabat | | |
| LC | FGGGTKVEIK | SEQ ID |
| Framework | | NO: 400 |
| Region 4 | | |
| LC6_m6 | | |
| Kabat | | |
| LC | FGGGTKVEIK | SEQ ID |
| Framework | | NO: 400 |
| Region 4 | | |
| LC6_m7 | | |
| Kabat | | |
| LC | FGGGTKVEIK | SEQ ID |
| Framework | | NO: 400 |
| Region 4 | | |
| LC6_m8 | | |
| Kabat | | |
| LC | FGGGTKVEIK | SEQ ID |
| Framework | | NO: 400 |
| Region 4 | | |
| LC6_m9 | | |
| Kabat | | |
| LC | FGGGTKVEIK | SEQ ID |
| Framework | | NO: 400 |
| Region 4 | | |
| LC6_m10 | | |
| Kabat | | |
| LC | FGGGTKVEIK | SEQ ID |
| Framework | | NO: 400 |
| Region 4 | | |
| LC6_m11 | | |
| Kabat | | |
| LC | FGGGTKVEIK | SEQ ID |
| Framework | | NO: 400 |
| Region 4 | | |
| LC6_m12 | | |
| Kabat | | |
| LC | FGGGTKVEIK | SEQ ID |
| Framework | | NO: 400 |
| Region 4 | | |
| LC6_m13 | | |
| Kabat | | |
| LC | FGGGTKVEIK | SEQ ID |
| Framework | | NO: 400 |
| Region 4 | | |
| LC6 m14 | | |
| Kabat | | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 425 |
| hIgG1 | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 426 |
| hIgG4 | GTKTYTCNVDHKPSNTKVDKRVESKYGPPCPSCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHN | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 427 |
| IgG4-SP | GTKTYTCNVDHKPSNTKVDKRVESKYGPPCPPCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHN | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 428 |
| IgG4-SPLE | GTKTYTCNVDHKPSNTKVDKRVESKYGPPCPPCPAPEELG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHN | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSNF | NO: 429 |
| IgG2 | GTQTYTCNVDHKPSNTKVDKTVERKCCVECPPCPAPPVA | |
| | GPSVFLFPPKPKDTLMISRTPEVTCVVVDVSHEDPEVQFN | |
| | WYVDGVEVHNAKTKPREEQFNSTFRVVSVLTVVHQDWL | |
| | NGKEYKCKVSNKGLPAPIEKTISKTKGQPREPQVYTLPPSR | |
| | EEMTKNQVSLTCLVKGFYPSDIA | |
| | VEWESNGQPENNYKTTPPMLDSDGSFFLYSKLTVDKSRW | |
| | QQGNVFSCSVMHEALHNHYTQKSLSLSP | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 430 |
| hIgG1- | GTQTYICNVNHKPSNTKVDKKVEPKSCDKTHTCPPCPAPE | |
| N297A | LLGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 431 |
| hIgG1- | GTQTYICNVNHKPSNTKVDKKVEPKSCDKTHTCPPCPAPE | |
| D265A | LLGGPSVFLFPPKPKDTLMISRTPEVTCVVVAVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 432 |
| hIgG1- | GTQTYICNVNHKPSNTKVDKKVEPKSCDKTHTCPPCPAPE | |
| LALA | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 433 |
| hIgG1- | GTQTYICNVNHKPSNTKVDKKVEPKSCDKTHTCPPCPAPE | |
| LAGA | LAGAPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 434 |
| hIgG1- | GTQTYICNVNHKPSNTKVDKKVEPKSCDKTHTCPPCPAPE | |
| LALAGA | AAGAPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 435 |
| hIgG1- | GTQTYICNVNHKPSNTKVDKKVEPKSCDKTHTCPPCPAPE | |
| LALAPG | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALGAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 436 |
| hIgG1-YTE | GTQTYICNVNHKPSNTKVDKKVEPKSCDKTHTCPPCPAPE | |
| | LLGGPSVFLFPPKPKDTLYITREPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 437 |
| hIgG1- | GTQTYICNVNHKPSNTKVDKKVEPKSCDKTHTCPPCPAPE | |
| N297A/YTE | LLGGPSVFLFPPKPKDTLYITREPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 438 |
| hIgG1- | GTQTYICNVNHKPSNTKVDKKVEPKSCDKTHTCPPCPAPE | |
| D265A/YTE | LLGGPSVFLFPPKPKDTLYITREPEVTCVVVAVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 439 |
| hIgG1- | GTQTYICNVNHKPSNTKVDKKVEPKSCDKTHTCPPCPAPE | |
| LALA/YTE | AAGGPSVFLFPPKPKDTLYITREPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 440 |
| hIgG1- | GTQTYICNVNHKPSNTKVDKKVEPKSCDKTHTCPPCPAPE | |
| LAGA/YTE | LAGAPSVFLFPPKPKDTLYITREPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 441 |
| hIgG1- | GTQTYICNVNHKPSNTKVDKKVEPKSCDKTHTCPPCPAPE | |
| LALAGA/YTE | AAGAPSVFLFPPKPKDTLYITREPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 442 |
| hIgG1- | GTQTYICNVNHKPSNTKVDKKVEPKSCDKTHTCPPCPAPE | |
| LALAPG/YTE | AAGGPSVFLFPPKPKDTLYITREPEVTCVVVDVSHEDPEVK | |
| | FNWYVDGVEVHNAKTKPREEQYNSTYRVVSVLTVLHQD | |
| | WLNGKEYKCKVSNKALGAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 443 |
| hIgG1-LS | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVLHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 444 |
| hIgG1- | GTQTYICNVNHKPSNTKVDKKVEPKSCDKTHTCPPCPAPE | |
| N297A/LS | LLGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVLHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 445 |
| hIgG1- | GTQTYICNVNHKPSNTKVDKKVEPKSCDKTHTCPPCPAPE | |
| D265A/LS | LLGGPSVFLFPPKPKDTLMISRTPEVTCVVVAVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVLHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 446 |
| hIgG1- | GTQTYICNVNHKPSNTKVDKKVEPKSCDKTHTCPPCPAPE | |
| LALA/LS | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVLHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 447 |
| hIgG1- | GTQTYICNVNHKPSNTKVDKKVEPKSCDKTHTCPPCPAPE | |
| LAGA/LS | LAGAPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVLHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 448 |
| hIgG1- | GTQTYICNVNHKPSNTKVDKKVEPKSCDKTHTCPPCPAPE | |
| LALAGA/LS | AAGAPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVLHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 449 |
| hIgG1- | GTQTYICNVNHKPSNTKVDKKVEPKSCDKTHTCPPCPAPE | |
| LALAPG/LS | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALGAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVLHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 450 |
| hIgG1-DHS | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYNSTYRVVSVLTVDHHD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 451 |
| hIgG1- | GTQTYICNVNHKPSNTKVDKKVEPKSCDKTHTCPPCPAPE | |
| N297A/DHS | LLGGPSVFLFPPKPKDTLMISRTPEVTCVVVDVSHEDPEVK | |
| | FNWYVDGVEVHNAKTKPREEQYASTYRVVSVLTVDHHD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 452 |
| hIgG1- | GTQTYICNVNHKPSNTKVDKKVEPKSCDKTHTCPPCPAPE | |
| D265A/DHS | LLGGPSVFLFPPKPKDTLMISRTPEVTCVVVAVSHEDPEVK | |
| | FNWYVDGVEVHNAKTKPREEQYNSTYRVVSVLTVDHHD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 453 |
| hIgG1- | GTQTYICNVNHKPSNTKVDKKVEPKSCDKTHTCPPCPAPE | |
| LALA/DHS | AAGGPSVFLFPPKPKDTLMISRTPEVTCVVVDVSHEDPEV | |
| | KFNWYVDGVEVHNAKTKPREEQYNSTYRVVSVLTVDHH | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVMHEALHSHYTQKSLSLSP | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 454 |
| hIgG1- | GTQTYICNVNHKPSNTKVDKKVEPKSCDKTHTCPPCPAPE | |
| LAGA/DHS | LAGAPSVFLFPPKPKDTLMISRTPEVTCVVVDVSHEDPEVK | |
| | FNWYVDGVEVHNAKTKPREEQYNSTYRVVSVLTVDHHD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSC | |
| | SVMHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 455 |
| hIgG1- | GTQTYICNVNHKPSNTKVDKKVEPKSCDKTHTCPPCPAPE | |
| LALAGA/DHS | AAGAPSVFLFPPKPKDTLMISRTPEVTCVVVDVSHEDPEV | |
| | KFNWYVDGVEVHNAKTKPREEQYNSTYRVVSVLTVDHH | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVMHEALHSHYTQKSLSLSPG | |
| Heavy | ASTKGPSVFPLAPSSKSTSGGTAALGCLVKDYFPEP | SEQ ID |
| Chain | VTVSWNSGALTSGVHTFPAVLQSSGLYSLSSVVTVPSSSL | NO: 456 |
| hIgG1- | GTQTYICNVNHKPSNTKVDKKVEPKSCDKTHTCPPCPAPE | |
| LALAPG/DHS | AAGGPSVFLFPPKPKDTLMISRTPEVTCVVVDVSHEDPEV | |
| | KFNWYVDGVEVHNAKTKPREEQYNSTYRVVSVLTVDHH | |
| | DWLNGKEYKCKVSNKALGAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSC | |
| | SVMHEALHSHYTQKSLSLSPG | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 457 |
| IgG4-YTE | GTKTYTCNVDHKPSNTKVDKRVESKYGPPCPSCPAPEFLG | |
| | GPSVFLFPPKPKDTLYITREPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHN | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 458 |
| IgG4- | GTKTYTCNVDHKPSNTKVDKRVESKYGPPCPPCPAPEFLG | |
| SP/YTE | GPSVFLFPPKPKDTLYITREPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHN | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 459 |
| IgG4- | GTKTYTCNVDHKPSNTKVDKRVESKYGPPCPPCPAPEELG | |
| SPLE/YTE | GPSVFLFPPKPKDTLYITREPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHN | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 460 |
| IgG4-LS | GTKTYTCNVDHKPSNTKVDKRVESKYGPPCPSCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVLHEALHSY | |
| | TQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 461 |
| IgG4-SP/LS | GTKTYTCNVDHKPSNTKVDKRVESKYGPPCPPCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVLHEALHSH | |
| | YTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 462 |
| IgG4- | GTKTYTCNVDHKPSNTKVDKRVESKYGPPCPPCPAPEELG | |
| SPLE/LS | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVLHEALHSH | |
| | YTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 463 |
| IgG4-DHS | GTKTYTCNVDHKPSNTKVDKRVESKYGPPCPSCPAPEFLG | |
| | GPSVFLFPPKPKDTLMISRTPEVTCVVVDVSQEDPEVQFN | |
| | WYVDGVEVHNAKTKPREEQFNSTYRVVSVLTVDHHDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHS | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 464 |
| IgG4- | GTKTYTCNVDHKPSNTKVDKRVESKYGPPCPPCPAPEFLG | |
| SP/DHS | GPSVFLFPPKPKDTLMISRTPEVTCVVVDVSQEDPEVQFN | |
| | WYVDGVEVHNAKTKPREEQFNSTYRVVSVLTVDHHDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHS | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSSL | NO: 465 |
| IgG4- | GTKTYTCNVDHKPSNTKVDKRVESKYGPPCPPCPAPEELG | |
| SPLE/DHS | GPSVFLFPPKPKDTLMISRTPEVTCVVVDVSQEDPEVQFN | |
| | WYVDGVEVHNAKTKPREEQFNSTYRVVSVLTVDHHDWL | |
| | NGKEYKCKVSNKGLPSSIEKTISKAKGQPREPQVYTLPPSQ | |
| | EEMTKNQVSLTCLVKGFYPSDIAVEWESNGQPENNYKTTP | |
| | PVLDSDGSFFLYSRLTVDKSRWQEGNVFSCSVMHEALHS | |
| | HYTQKSLSLSLGK | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSNF | NO: 466 |
| IgG2-YTE | GTQTYTCNVDHKPSNTKVDKTVERKCCVECPPCPAPPVA | |
| | GPSVFLFPPKPKDTLYITREPEVTCVVVDVSHEDPEVQFN | |
| | WYVDGVEVHNAKTKPREEQFNSTFRVVSVLTVVHQDWL | |
| | NGKEYKCKVSNKGLPAPIEKTISKTKGQPREPQVYTLPPSR | |
| | EEMTKNQVSLTCLVKGFYPSDIA | |
| | VEWESNGQPENNYKTTPPMLDSDGSFFLYSKLTVDKSRW | |
| | QQGNVFSCSVLHEALHSHYTQKSLSLSP | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSNF | NO: 467 |
| IgG2-LS | GTQTYTCNVDHKPSNTKVDKTVERKCCVECPPCPAPPVA | |
| | GPSVFLFPPKPKDTLMISRTPEVTCVVVDVSHEDPEVQFN | |
| | WYVDGVEVHNAKTKPREEQFNSTFRVVSVLTVVHQDWL | |
| | NGKEYKCKVSNKGLPAPIEKTISKTKGQPREPQVYTLPPSR | |
| | EEMTKNQVSLTCLVKGFYPSDIA | |
| | VEWESNGQPENNYKTTPPMLDSDGSFFLYSKLTVDKSRW | |
| | QQGNVFSCSVMHEALHNHYTQKSLSLSP | |
| Heavy | ASTKGPSVFPLAPCSRSTSESTAALGCLVKDYFPEP | SEQ ID |
| Chain | VTVSWNSGALTSGVHTFPAVLQSSGLYSLSSVVTVPSSNF | NO: 468 |
| IgG2-DHS | GTQTYTCNVDHKPSNTKVDKTVERKCCVECPPCPAPPVA | |
| | GPSVFLFPPKPKDTLMISRTPEVTCVVVDVSHEDPEVQFN | |
| | WYVDGVEVHNAKTKPREEQFNSTFRVVSVLTVDHHDWL | |
| | NGKEYKCKVSNKGLPAPIEKTISKTKGQPREPQVYTLPPSR | |
| | EEMTKNQVSLTCLVKGFYPSDIAVEWESNGQPENNYKTTP | |
| | PMLDSDGSFFLYSKLTVDKSRWQQGNVFSCSVMHEALHS | |
| | HYTQKSLSLSP | |
| Light Chain | RTVAAPSVFIFPPSDEQLKSGTASVVCLLNNFYPRE | SEQ ID |
| Human | AKVQWKVDNALQSGNSQESVTEQDSKDSTYSLSSTLTLS | NO: 469 |
| kappa LC | KADYEKHKVYACEVTHQGLSSPVTKSFNRGEC | |
| Lebrikizumab | QVTLRESGPALVKPTQTLTLTCTVSGFSLSAYSVNW | SEQ ID |
| Heavy | IRQPPGKALEWLAMIWGDGKIVYNSALKSRLTISKDTSKN | NO: 470 |
| Chain | QVVLTMTNMDPVDTATYYCAGDGYYPYAMDNWGQGSL | |
| Variable | VTVSS | |
| Region | | |
| Lebrikizumab | DIVMTQSPDSLSVSLGERATINCRASKSVDSYGNSF | SEQ ID |
| Light Chain | MHWYQQKPGQPPKLLIYLASNLESGVPDRFSGSGSGTDFT | NO: 471 |
| Variable | LTISSLQAEDVAVYYCQQNNEDPRTFGGGTKVEIK | |
| Region | | |
| Human IL- | MHPLLNPLLLALGLMALLLTTVIALTCLGGFASPGP | SEQ ID |
| 13 | VPPSTALRELIEELVNITQNQKAPLCNGSMVWSINLTAGM | NO: 472 |
| Full | YCAALESLINVSGCSAIEKTQRMLSGFCPHKVSAGQFSSLH | |
| sequence | VRDTKIEVAQFVKDLLLHLKKLFREGRFN | |
| Cynomolgus | MALLLTMVIALTCLGGFASPSPVPPSTALKELIEELV | SEQ ID |
| Monkey IL- | NITQNQKAPLCNGSMVWSINLTAGVYCAALESLINVSGCS | NO: 473 |
| 13 | AIEKTQRMLNGFCPHKVSAGQFSSLRVRDTKIEVAQFVKD | |
| Full | LLVHLKKLFREGQFN | |
| sequence | | |
| Mouse IL- | MALWVTAVLALACLGGLAAPGPVPRSVSLPLTLKE | SEQ ID |
| 13 | LIEELSNITQDQTPLCNGSMVWSVDLAAGGFCVALDSLTNI | NO: 474 |
| Full | SNCNAIYRTQRILHGLCNRKAPTTVSSLPDTKIEVAHFITKL | |
| sequence | LSYTKQLFRHGPF | |
| Rat IL-13 | MALWVTAVLALACLGGLATPGPVRRSTSPPVALRE | SEQ ID |
| Full | LIEELSNITQDQKTSLCNSSMVWSVDLTAGGFCAALESLTN | NO: 475 |
| sequence | ISSCNAIHRTQRILNGLCNQKASDVASSPPDTKIEVAQFISK | |
| | LLNYSKQLFRYGH | |
| Human IL- | MHPLLNPLLLALGLMALLLTTVIA | SEQ ID |
| 13 | | NO: 476 |
| Leader | | |
| sequence | | |
| Cynomolgus | MALLLTMVIALTCLGGFA | SEQ ID |
| Monkey IL- | | NO: 477 |
| 13 | | |
| Leader | | |
| sequence | | |
| Mouse IL- | MALWVTAVLALACLGGLA | SEQ ID |
| 13 | | NO: 478 |
| Leader | | |
| sequence | | |
| Rat IL-13 | MALWVTAVLALACLGGLA | SEQ ID |
| Leader | | NO: 479 |
| sequence | | |
| Human IL- | LTCLGGFASPGPVPPSTALRELIEELVNITQNQKAPL | SEQ ID |
| 13 | CNGSMVWSINLTAGMYCAALESLINVSGCSAIEKTQRMLS | NO: 480 |
| Main chain | GFCPHKVSAGQFSSLHVRDTKIEVAQFVKDLLLHLKKLFR | |
| sequence | EGRFN | |
| Cynomolgus | SPSPVPPSTALKELIEELVNITQNQKAPLCNGSMVW | SEQ ID |
| Monkey IL- | SINLTAGVYCAALESLINVSGCSAIEKTQRMLNGFCPHKVS | NO: 481 |
| 13 | AGQFSSLRVRDTKIEVAQFVKDLLVHLKKLFREGQFN | |
| Main chain | | |
| sequence | | |
| Mouse IL- | APGPVPRSVSLPLTLKELIEELSNITQDQTPLCNGSM | SEQ ID |
| 13 | VWSVDLAAGGFCVALDSLTNISNCNAIYRTQRILHGLCNR | NO: 482 |
| Main chain | KAPTTVSSLPDTKIEVAHFITKLLSYTKQLFRHGPF | |
| sequence | | |
| Rat IL-13 | TPGPVRRSTSPPVALRELIEELSNITQDQKTSLCNSS | SEQ ID |
| Main chain | MVWSVDLTAGGFCAALESLTNISSCNAIHRTQRILNGLCN | NO: 483 |
| sequence | QKASDVASSPPDTKIEVAQFISKLLNYSKQLFRYGH | |
| hIgG1-LA | ASTKGPSVFPLAPSSKSTSGGTAALGCLVKDYFPEP | SEQ ID |
| | VTVSWNSGALTSGVHTFPAVLQSSGLYSLSSVVTVPSSSL | NO: 484 |
| | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVLHEAL | |
| | HAHYTQKSLSLSPG | |
| hIgG1- | ASTKGPSVFPLAPSSKSTSGGTAALGCLVKDYFPEP | SEQ ID |
| N297A/LA | VTVSWNSGALTSGVHTFPAVLQSSGLYSLSSVVTVPSSSL | NO: 485 |
| | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVLHEAL | |
| | HAHYTQKSLSLSPG | |
| hIgG1- | ASTKGPSVFPLAPSSKSTSGGTAALGCLVKDYFPEP | SEQ ID |
| D265A/LA | VTVSWNSGALTSGVHTFPAVLQSSGLYSLSSVVTVPSSSL | NO: 486 |
| | GTQTYICNVNHKPSNTKVDKKVEPKSCDKTHTCPPCPAPE | |
| | LLGGPSVFLFPPKPKDTLMISRTPEVTCVVVAVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVLHEAL | |
| | HAHYTQKSLSLSPG | |
| hIgG1- | ASTKGPSVFPLAPSSKSTSGGTAALGCLVKDYFPEP | SEQ ID |
| LALA/LA | VTVSWNSGALTSGVHTFPAVLQSSGLYSLSSVVTVPSSSL | NO: 487 |
| | GTQTYICNVNHKPSNTKVDKKVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVLHEA | |
| | LHAHYTQKSLSLSPG | |
| hIgG1- | ASTKGPSVFPLAPSSKSTSGGTAALGCLVKDYFPEP | SEQ ID |
| LAGA/LA | VTVSWNSGALTSGVHTFPAVLQSSGLYSLSSVVTVPSSSL | NO: 488 |
| | GTQTYICNVNHKPSNTKVDKKVEPKSCDKTHTCPPCPAPE | |
| | LAGAPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVLHEAL | |
| | HAHYTQKSLSLSPG | |
| hIgG1- | ASTKGPSVFPLAPSSKSTSGGTAALGCLVKDYFPEP | SEQ ID |
| LALAGA/LA | VTVSWNSGALTSGVHTFPAVLQSSGLYSLSSVVTVPSSSL | NO: 489 |
| | GTQTYICNVNHKPSNTKVDKKVEPKSCDKTHTCPPCPAPE | |
| | AAGAPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVLHEA | |
| | LHAHYTQKSLSLSPG | |
| hIgG1- | ASTKGPSVFPLAPSSKSTSGGTAALGCLVKDYFPEP | SEQ ID |
| LALAPG/LA | VTVSWNSGALTSGVHTFPAVLQSSGLYSLSSVVTVPSSSL | NO: 490 |
| | GTQTYICNVNHKPSNTKVDKKVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALGAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVLHEA | |
| | LHAHYTQKSLSLSPG | |
| hIgG1- | ASTKGPSVFPLAPSSKSTSGGTAALGCLVKDYFPEP | SEQ ID |
| N434A | VTVSWNSGALTSGVHTFPAVLQSSGLYSLSSVVTVPSSSL | NO: 491 |
| | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEAL | |
| | HAHYTQKSLSLSPG | |
| hIgG1- | ASTKGPSVFPLAPSSKSTSGGTAALGCLVKDYFPEP | SEQ ID |
| N297A/ | VTVSWNSGALTSGVHTFPAVLQSSGLYSLSSVVTVPSSSL | NO: 492 |
| N434A | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEAL | |
| | HAHYTQKSLSLSPG | |
| hIgG1- | ASTKGPSVFPLAPSSKSTSGGTAALGCLVKDYFPEP | SEQ ID |
| D265A/ | VTVSWNSGALTSGVHTFPAVLQSSGLYSLSSVVTVPSSSL | NO: 493 |
| N434A | GTQTYICNVNHKPSNTKVDKKVEPKSCDKTHTCPPCPAPE | |
| | LLGGPSVFLFPPKPKDTLMISRTPEVTCVVVAVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEAL | |
| | HAHYTQKSLSLSPG | |
| hIgG1- | ASTKGPSVFPLAPSSKSTSGGTAALGCLVKDYFPEP | SEQ ID |
| LALA/ | VTVSWNSGALTSGVHTFPAVLQSSGLYSLSSVVTVPSSSL | NO: 494 |
| N434A | GTQTYICNVNHKPSNTKVDKKVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVMHE | |
| | ALHAHYTQKSLSLSPG | |
| hIgG1- | ASTKGPSVFPLAPSSKSTSGGTAALGCLVKDYFPEP | SEQ ID |
| LAGA/ | VTVSWNSGALTSGVHTFPAVLQSSGLYSLSSVVTVPSSSL | NO: 495 |
| N434A | GTQTYICNVNHKPSNTKVDKKVEPKSCDKTHTCPPCPAPE | |
| | LAGAPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEAL | |
| | HAHYTQKSLSLSPG | |
| hIgG1- | ASTKGPSVFPLAPSSKSTSGGTAALGCLVKDYFPEP | SEQ ID |
| LALAGA/ | VTVSWNSGALTSGVHTFPAVLQSSGLYSLSSVVTVPSSSL | NO: 496 |
| N434A | GTQTYICNVNHKPSNTKVDKKVEPKSCDKTHTCPPCPAPE | |
| | AAGAPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVMHE | |
| | ALHAHYTQKSLSLSPG | |
| hIgG1- | ASTKGPSVFPLAPSSKSTSGGTAALGCLVKDYFPEP | SEQ ID |
| LALAPG/ | VTVSWNSGALTSGVHTFPAVLQSSGLYSLSSVVTVPSSSL | NO: 497 |
| N434A | GTQTYICNVNHKPSNTKVDKKVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALGAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVMHE | |
| | ALHAHYTQKSLSLSPG | |
| hIgG1- | ASTKGPSVFPLAPSSKSTSGGTAALGCLVKDYFPEP | SEQ ID |
| N434W | VTVSWNSGALTSGVHTFPAVLQSSGLYSLSSVVTVPSSSL | NO: 498 |
| | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEAL | |
| | HWHYTQKSLSLSPG | |
| hIgG1- | ASTKGPSVFPLAPSSKSTSGGTAALGCLVKDYFPEP | SEQ ID |
| N297A/ | VTVSWNSGALTSGVHTFPAVLQSSGLYSLSSVVTVPSSSL | NO: 499 |
| N434W | GTQTYICNVNHKPSNTKVDKKVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYASTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEAL | |
| | HWHYTQKSLSLSPG | |
| hIgG1- | ASTKGPSVFPLAPSSKSTSGGTAALGCLVKDYFPEP | SEQ ID |
| D265A/ | VTVSWNSGALTSGVHTFPAVLQSSGLYSLSSVVTVPSSSL | NO: 500 |
| N434W | GTQTYICNVNHKPSNTKVDKKVEPKSCDKTHTCPPCPAPE | |
| | LLGGPSVFLFPPKPKDTLMISRTPEVTCVVVAVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEAL | |
| | HWHYTQKSLSLSPG | |
| hIgG1- | ASTKGPSVFPLAPSSKSTSGGTAALGCLVKDYFPEP | SEQ ID |
| LALA/ | VTVSWNSGALTSGVHTFPAVLQSSGLYSLSSVVTVPSSSL | NO: 501 |
| N434W | GTQTYICNVNHKPSNTKVDKKVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVMHE | |
| | ALHWHYTQKSLSLSPG | |
| hIgG1- | ASTKGPSVFPLAPSSKSTSGGTAALGCLVKDYFPEP | SEQ ID |
| LAGA/ | VTVSWNSGALTSGVHTFPAVLQSSGLYSLSSVVTVPSSSL | NO: 502 |
| N434W | GTQTYICNVNHKPSNTKVDKKVEPKSCDKTHTCPPCPAPE | |
| | LAGAPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLTCLVKGFYPSDIAVEWESNGQPENNYK | |
| | TTPPVLDSDGSFFLYSKLTVDKSRWQQGNVFSCSVMHEAL | |
| | HWHYTQKSLSLSPG | |
| hIgG1- | ASTKGPSVFPLAPSSKSTSGGTAALGCLVKDYFPEP | SEQ ID |
| LALAGA/ | VTVSWNSGALTSGVHTFPAVLQSSGLYSLSSVVTVPSSSL | NO: 503 |
| N434W | GTQTYICNVNHKPSNTKVDKKVEPKSCDKTHTCPPCPAPE | |
| | AAGAPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVMHE | |
| | ALHWHYTQKSLSLSPG | |
| hIgG1- | ASTKGPSVFPLAPSSKSTSGGTAALGCLVKDYFPEP | SEQ ID |
| LALAPG/ | VTVSWNSGALTSGVHTFPAVLQSSGLYSLSSVVTVPSSSL | NO: 504 |
| N434W | GTQTYICNVNHKPSNTKVDKKVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALGAPIEKTISKAKGQPREPQVYTL | |
| | PPSRDELTKNQVSLTCLVKGFYPSDIAVEWESNGQPENNY | |
| | KTTPPVLDSDGSFFLYSKLTVDKSRWQQGNVFSCSVMHE | |
| | ALHWHYTQKSLSLSPG | |
| hIgG1/DQ | ASTKGPSVFPLAPSSKSTSGGTAALGCLVKDYFPEPVTVS | SEQ ID |
| | WNSGALTSGVHTFPAVLQSSGLYSLSSVVTVPSSSLGTQT | NO: 505 |
| | YICNVNHKPSNTKVDKKVEPKSCDKTHTCPPCPAPELLGG | |
| | PSVFLFPPKPKDTLMISRDPEVTCVVVDVSHEDPEVKFNW | |
| | YVDGVEVHNAKTKPREEQYNSTYRVVSVLQVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| N297A/DQ | WNSGALTSGVHTFPAVLQSSGLYSLSSVVTVPSSSLGTQT | NO: 506 |
| | YICNVNHKPSNTKVDKKVEPKSCDKTHTCPPCPAPELLGG | |
| | PSVFLFPPKPKDTLMISRDPEVTCVVVDVSHEDPEVKFNW | |
| | YVDGVEVHNAKTKPREEQYASTYRVVSVLQVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| D265A/DQ | WNSGALTSGVHTFPAVLQSSGLYSLSSVVTVPSSSLGTQT | NO: 507 |
| | YICNVNHKPSNTKVDKKVEPKSCDKTHTCPPCPAPELLGG | |
| | PSVFLFPPKPKDTLMISRDPEVTCVVVAVSHEDPEVKFNW | |
| | YVDGVEVHNAKTKPREEQYNSTYRVVSVLQVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALA/DQ | WNSGALTSGVHTFPAVLQSSGLYSLSSVVTVPSSSLGTQT | NO: 508 |
| | YICNVNHKPSNTKVDKKVEPKSCDKTHTCPPCPAPEAAGG | |
| | PSVFLFPPKPKDTLMISRDPEVTCVVVDVSHEDPEVKFNW | |
| | YVDGVEVHNAKTKPREEQYNSTYRVVSVLQVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LAGA/DQ | WNSGALTSGVHTFPAVLQSSGLYSLSSVVTVPSSSLGTQT | NO: 509 |
| | YICNVNHKPSNTKVDKKVEPKSCDKTHTCPPCPAPELAGA | |
| | PSVFLFPPKPKDTLMISRDPEVTCVVVDVSHEDPEVKFNW | |
| | YVDGVEVHNAKTKPREEQYNSTYRVVSVLQVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAGA/DQ | WNSGALTSGVHTFPAVLQSSGLYSLSSVVTVPSSSLGTQT | NO: 510 |
| | YICNVNHKPSNTKVDKKVEPKSCDKTHTCPPCPAPEAAGA | |
| | PSVFLFPPKPKDTLMISRDPEVTCVVVDVSHEDPEVKFNW | |
| | YVDGVEVHNAKTKPREEQYNSTYRVVSVLQVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAPG/DQ | WNSGALTSGVHTFPAVLQSSGLYSLSSVVTVPSSSLGTQT | NO: 511 |
| | YICNVNHKPSNTKVDKKVEPKSCDKTHTCPPCPAPEAAGG | |
| | PSVFLFPPKPKDTLMISRDPEVTCVVVDVSHEDPEVKFNW | |
| | YVDGVEVHNAKTKPREEQYNSTYRVVSVLQVLHQDWLN | |
| | GKEYKCKVSNKALGAPIEKTISKAKGQPREPQVYTLPPSR | |
| | DELTKNQVSLTCLVKGFYPSDIAVEWESNGQPENNYKTTP | |
| | PVLDSDGSFFLYSKLTVDKSRWQQGNVFSCSVMHEALHN | |
| | HYTQKSLSLSPG | |
| hIgG1/DW | ASTKGPSVFPLAPSSKSTSGGTAALGCLVKDYFPEPVTVS | SEQ ID |
| | WNSGALTSGVHTFPAVLQSSGLYSLSSVVTVPSSSLGTQT | NO: 512 |
| | YICNVNHKPSNTKVDKKVEPKSCDKTHTCPPCPAPELLGG | |
| | PSVFLFPPKPKDTLMISRDPEVTCVVVDVSHEDPEVKFNW | |
| | YVDGVEVHNAKTKPREEQYNSTYRVVSVLWVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| N297A/DW | WNSGALTSGVHTFPAVLQSSGLYSLSSVVTVPSSSLGTQT | NO: 513 |
| | YICNVNHKPSNTKVDKKVEPKSCDKTHTCPPCPAPELLGG | |
| | PSVFLFPPKPKDTLMISRDPEVTCVVVDVSHEDPEVKFNW | |
| | YVDGVEVHNAKTKPREEQYASTYRVVSVLWVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| D265A/DW | WNSGALTSGVHTFPAVLQSSGLYSLSSVVTVPSSSLGTQT | NO: 514 |
| | YICNVNHKPSNTKVDKKVEPKSCDKTHTCPPCPAPELLGG | |
| | PSVFLFPPKPKDTLMISRDPEVTCVVVAVSHEDPEVKFNW | |
| | YVDGVEVHNAKTKPREEQYNSTYRVVSVLWVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALA/DW | WNSGALTSGVHTFPAVLQSSGLYSLSSVVTVPSSSLGTQT | NO: 515 |
| | YICNVNHKPSNTKVDKKVEPKSCDKTHTCPPCPAPEAAGG | |
| | PSVFLFPPKPKDTLMISRDPEVTCVVVDVSHEDPEVKFNW | |
| | YVDGVEVHNAKTKPREEQYNSTYRVVSVLWVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LAGA/DW | WNSGALTSGVHTFPAVLQSSGLYSLSSVVTVPSSSLGTQT | NO: 516 |
| | YICNVNHKPSNTKVDKKVEPKSCDKTHTCPPCPAPELAGA | |
| | PSVFLFPPKPKDTLMISRDPEVTCVVVDVSHEDPEVKFNW | |
| | YVDGVEVHNAKTKPREEQYNSTYRVVSVLWVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAGA/DW | WNSGALTSGVHTFPAVLQSSGLYSLSSVVTVPSSSLGTQT | NO: 517 |
| | YICNVNHKPSNTKVDKKVEPKSCDKTHTCPPCPAPEAAGA | |
| | PSVFLFPPKPKDTLMISRDPEVTCVVVDVSHEDPEVKFNW | |
| | YVDGVEVHNAKTKPREEQYNSTYRVVSVLWVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAPG/DW | WNSGALTSGVHTFPAVLQSSGLYSLSSVVTVPSSSLGTQT | NO: 518 |
| | YICNVNHKPSNTKVDKKVEPKSCDKTHTCPPCPAPEAAGG | |
| | PSVFLFPPKPKDTLMISRDPEVTCVVVDVSHEDPEVKFNW | |
| | YVDGVEVHNAKTKPREEQYNSTYRVVSVLWVLHQDWLN | |
| | GKEYKCKVSNKALGAPIEKTISKAKGQPREPQVYTLPPSR | |
| | DELTKNQVSLTCLVKGFYPSDIAVEWESNGQPENNYKTTP | |
| | PVLDSDGSFFLYSKLTVDKSRWQQGNVFSCSVMHEALHN | |
| | HYTQKSLSLSPG | |
| hIgG1/YD | ASTKGPSVFPLAPSSKSTSGGTAALGCLVKDYFPEPVTVS | SEQ ID |
| | WNSGALTSGVHTFPAVLQSSGLYSLSSVVTVPSSSLGTQT | NO: 519 |
| | YICNVNHKPSNTKVDKKVEPKSCDKTHTCPPCPAPELLGG | |
| | PSVFLFPPKPKDTLYISRDPEVTCVVVDVSHEDPEVKFNW | |
| | YVDGVEVHNAKTKPREEQYNSTYRVVSVLTVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| N297A/YD | WNSGALTSGVHTFPAVLQSSGLYSLSSVVTVPSSSLGTQT | NO: 520 |
| | YICNVNHKPSNTKVDKKVEPKSCDKTHTCPPCPAPELLGG | |
| | PSVFLFPPKPKDTLYISRDPEVTCVVVDVSHEDPEVKFNW | |
| | YVDGVEVHNAKTKPREEQYASTYRVVSVLTVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| D265A/YD | WNSGALTSGVHTFPAVLQSSGLYSLSSVVTVPSSSLGTQT | NO: 521 |
| | YICNVNHKPSNTKVDKKVEPKSCDKTHTCPPCPAPELLGG | |
| | PSVFLFPPKPKDTLYISRDPEVTCVVVAVSHEDPEVKFNW | |
| | YVDGVEVHNAKTKPREEQYNSTYRVVSVLTVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALA/YD | WNSGALTSGVHTFPAVLQSSGLYSLSSVVTVPSSSLGTQT | NO: 522 |
| | YICNVNHKPSNTKVDKKVEPKSCDKTHTCPPCPAPEAAGG | |
| | PSVFLFPPKPKDTLYISRDPEVTCVVVDVSHEDPEVKFNW | |
| | YVDGVEVHNAKTKPREEQYNSTYRVVSVLTVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LAGA/YD | WNSGALTSGVHTFPAVLQSSGLYSLSSVVTVPSSSLGTQT | NO: 523 |
| | YICNVNHKPSNTKVDKKVEPKSCDKTHTCPPCPAPELAGA | |
| | PSVFLFPPKPKDTLYISRDPEVTCVVVDVSHEDPEVKFNW | |
| | YVDGVEVHNAKTKPREEQYNSTYRVVSVLTVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAGA/YD | WNSGALTSGVHTFPAVLQSSGLYSLSSVVTVPSSSLGTQT | NO: 524 |
| | YICNVNHKPSNTKVDKKVEPKSCDKTHTCPPCPAPEAAGA | |
| | PSVFLFPPKPKDTLYISRDPEVTCVVVDVSHEDPEVKFNW | |
| | YVDGVEVHNAKTKPREEQYNSTYRVVSVLTVLHQDWLN | |
| | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAPG/YD | WNSGALTSGVHTFPAVLQSSGLYSLSSVVTVPSSSLGTQT | NO: 525 |
| | YICNVNHKPSNTKVDKKVEPKSCDKTHTCPPCPAPEAAGG | |
| | PSVFLFPPKPKDTLYISRDPEVTCVVVDVSHEDPEVKFNW | |
| | YVDGVEVHNAKTKPREEQYNSTYRVVSVLTVLHQDWLN | |
| | GKEYKCKVSNKALGAPIEKTISKAKGQPREPQVYTLPPSR | |
| | DELTKNQVSLTCLVKGFYPSDIAVEWESNGQPENNYKTTP | |
| | PVLDSDGSFFLYSKLTVDKSRWQQGNVFSCSVMHEALHN | |
| | HYTQKSLSLSPG | |
| hIgG1/QVV | ASTKGPSVFPLAPSSKSTSGGTAALGCLVKDYFPEPVTVS | SEQ ID |
| | WNSGALTSGVHTFPAVLQSSGLYSLSSVVTVPSSSLGTQT | NO: 526 |
| | YICNVNHKPSNTKVDKKVEPKSCDKTHTCPPCPAPELLGG | |
| | PSVFLFPPKPKDTLMISRTPEVTCVVVDVSHEDPEVKFNW | |
| | YVDGVEVHNAKTKPREEQYNSTYRVVSVLQVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| N297A/QVV | WNSGALTSGVHTFPAVLQSSGLYSLSSVVTVPSSSLGTQT | NO: 527 |
| | YICNVNHKPSNTKVDKKVEPKSCDKTHTCPPCPAPELLGG | |
| | PSVFLFPPKPKDTLMISRTPEVTCVVVDVSHEDPEVKFNW | |
| | YVDGVEVHNAKTKPREEQYASTYRVVSVLQVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| D265A/QVV | WNSGALTSGVHTFPAVLQSSGLYSLSSVVTVPSSSLGTQT | NO: 528 |
| | YICNVNHKPSNTKVDKKVEPKSCDKTHTCPPCPAPELLGG | |
| | PSVFLFPPKPKDTLMISRTPEVTCVVVAVSHEDPEVKFNW | |
| | YVDGVEVHNAKTKPREEQYNSTYRVVSVLQVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALA/QVV | WNSGALTSGVHTFPAVLQSSGLYSLSSVVTVPSSSLGTQT | NO: 529 |
| | YICNVNHKPSNTKVDKKVEPKSCDKTHTCPPCPAPEAAGG | |
| | PSVFLFPPKPKDTLMISRTPEVTCVVVDVSHEDPEVKFNW | |
| | YVDGVEVHNAKTKPREEQYNSTYRVVSVLQVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LAGA/QVV | WNSGALTSGVHTFPAVLQSSGLYSLSSVVTVPSSSLGTQT | NO: 530 |
| | YICNVNHKPSNTKVDKKVEPKSCDKTHTCPPCPAPELAGA | |
| | PSVFLFPPKPKDTLMISRTPEVTCVVVDVSHEDPEVKFNW | |
| | YVDGVEVHNAKTKPREEQYNSTYRVVSVLQVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAGA/QVV | WNSGALTSGVHTFPAVLQSSGLYSLSSVVTVPSSSLGTQT | NO: 531 |
| | YICNVNHKPSNTKVDKKVEPKSCDKTHTCPPCPAPEAAGA | |
| | PSVFLFPPKPKDTLMISRTPEVTCVVVDVSHEDPEVKFNW | |
| | YVDGVEVHNAKTKPREEQYNSTYRVVSVLQVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAPG/QVV | WNSGALTSGVHTFPAVLQSSGLYSLSSVVTVPSSSLGTQT | NO: 532 |
| | YICNVNHKPSNTKVDKKVEPKSCDKTHTCPPCPAPEAAGG | |
| | PSVFLFPPKPKDTLMISRTPEVTCVVVDVSHEDPEVKFNW | |
| | YVDGVEVHNAKTKPREEQYNSTYRVVSVLQVLHVDWLN | |
| | GKEYKCKVSNKALGAPIEKTISKAKGQPREPQVYTLPPSR | |
| | DELTKNQVSLTCLVKGFYPSDIVVEWESNGQPENNYKTTP | |
| | PVLDSDGSFFLYSKLTVDKSRWQQGNVFSCSVMHEALHN | |
| | HYTQKSLSLSPG | |
| hIgG1/DDR | ASTKGPSVFPLAPSSKSTSGGTAALGCLVKDYFPEPVTVS | SEQ ID |
| VV | WNSGALTSGVHTFPAVLQSSGLYSLSSVVTVPSSSLGTQT | NO: 533 |
| | YICNVNHKPSNTKVDKKVEPKSCDKTHTCPPCPAPELLGG | |
| | PSVFLFPPKPKDTLMISRDPEVTCVVVDVSHEDPEVKFNW | |
| | YVDGVEVDNAKTKPREEQYNSTYRVVSVLRVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| N297A/DD | WNSGALTSGVHTFPAVLQSSGLYSLSSVVTVPSSSLGTQT | NO: 534 |
| RVV | YICNVNHKPSNTKVDKKVEPKSCDKTHTCPPCPAPELLGG | |
| | PSVFLFPPKPKDTLMISRDPEVTCVVVDVSHEDPEVKFNW | |
| | YVDGVEVDNAKTKPREEQYASTYRVVSVLRVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| D265A/DD | WNSGALTSGVHTFPAVLQSSGLYSLSSVVTVPSSSLGTQT | NO: 535 |
| RVV | YICNVNHKPSNTKVDKKVEPKSCDKTHTCPPCPAPELLGG | |
| | PSVFLFPPKPKDTLMISRDPEVTCVVVAVSHEDPEVKFNW | |
| | YVDGVEVDNAKTKPREEQYNSTYRVVSVLRVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALA/DDR | WNSGALTSGVHTFPAVLQSSGLYSLSSVVTVPSSSLGTQT | NO: 536 |
| VV | YICNVNHKPSNTKVDKKVEPKSCDKTHTCPPCPAPEAAGG | |
| | PSVFLFPPKPKDTLMISRDPEVTCVVVDVSHEDPEVKFNW | |
| | YVDGVEVDNAKTKPREEQYNSTYRVVSVLRVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LAGA/DD | WNSGALTSGVHTFPAVLQSSGLYSLSSVVTVPSSSLGTQT | NO: 537 |
| RVV | YICNVNHKPSNTKVDKKVEPKSCDKTHTCPPCPAPELAGA | |
| | PSVFLFPPKPKDTLMISRDPEVTCVVVDVSHEDPEVKFNW | |
| | YVDGVEVDNAKTKPREEQYNSTYRVVSVLRVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | |
| LALAGA/D | WNSGALTSGVHTFPAVLQSSGLYSLSSVVTVPSSSLGTQT | SEQ ID |
| DRVV | YICNVNHKPSNTKVDKKVEPKSCDKTHTCPPCPAPEAAGA | NO: 538 |
| | PSVFLFPPKPKDTLMISRDPEVTCVVVDVSHEDPEVKFNW | |
| | YVDGVEVDNAKTKPREEQYNSTYRVVSVLRVLHVDWLN | |
| | GKEYKCKVSNKALPAPIEKTISKAKGQPREPQVYTLPPSRD | |
| | ELTKNQVSLTCLVKGFYPSDIVVEWESNGQPENNYKTTPP | |
| | VLDSDGSFFLYSKLTVDKSRWQQGNVFSCSVMHEALHNH | |
| | YTQKSLSLSPG | |
| hIgG1- | ASTKGPSVFPLAPSSKSTSGGTAALGCLVKDYFPEPVTVS | SEQ ID |
| LALAPG/D | WNSGALTSGVHTFPAVLQSSGLYSLSSVVTVPSSSLGTQT | NO: 539 |
| DRVV | YICNVNHKPSNTKVDKKVEPKSCDKTHTCPPCPAPEAAGG | |
| | PSVFLFPPKPKDTLMISRDPEVTCVVVDVSHEDPEVKFNW | |
| | YVDGVEVDNAKTKPREEQYNSTYRVVSVLRVLHVDWLN | |
| | GKEYKCKVSNKALGAPIEKTISKAKGQPREPQVYTLPPSR | |
| | DELTKNQVSLTCLVKGFYPSDIVVEWESNGQPENNYKTTP | |
| | PVLDSDGSFFLYSKLTVDKSRWQQGNVFSCSVMHEALHN | |
| | HYTQKSLSLSPG | |

1-271. (canceled)

272. An isolated antibody that binds Interleukin 13 (IL-13), wherein the antibody comprises:
a CDR-H1 comprising the sequence set forth in SEQ ID NO: 58;
a CDR-H2 comprising the sequence set forth in SEQ ID NO: 100;
a CDR-H3 comprising the sequence set forth in SEQ ID NO: 112;
a CDR-L1 comprising the sequence set forth in SEQ ID NO: 141;
a CDR-L2 comprising the sequence set forth in SEQ ID NO: 153; and
a CDR-L3 comprising the sequence set forth in SEQ ID NO: 165;
and wherein the human Fc region of the antibody comprises a human IgG1 Fc with LALA and/or YTE mutations.

273. An isolated antibody that binds IL-13, wherein the antibody comprises:
a CDR-H1 comprising the sequence set forth in SEQ ID NO: 58;
a CDR-H2 comprising the sequence set forth in SEQ ID NO: 100;
a CDR-H3 comprising the sequence set forth in SEQ ID NO: 112;
a CDR-L1 comprising the sequence set forth in SEQ ID NO: 141;
a CDR-L2 comprising the sequence set forth in SEQ ID NO: 153; and
a CDR-L3 comprising the sequence set forth in SEQ ID NO: 165;
wherein the Fc region comprises one or more amino acid substitutions that result in increased antibody half-life, and wherein the one or more amino acid substitutions is selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, N434W, M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LA), M428L/N434A L234A/G237A (LALA), L234A/L235A/G237A, L234A/L235A/P329G, N297A, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

274. The isolated antibody of claim 272, wherein the LALA mutations are at positions 235 and 236 (L235A/L236A) and the YTE mutations are at positions 253, 255, and 257 (M253Y/S255T/T257E).

275. An isolated antibody that binds IL-13, wherein the antibody comprises:
a CDR-H1 comprising the sequence set forth in SEQ ID NO: 58;
a CDR-H2 comprising the sequence set forth in SEQ ID NO: 100;
a CDR-H3 comprising the sequence set forth in SEQ ID NO: 112;
a CDR-L1 comprising the sequence set forth in SEQ ID NO: 141;
a CDR-L2 comprising the sequence set forth in SEQ ID NO: 153; and
a CDR-L3 comprising the sequence set forth in SEQ ID NO: 165,
wherein the antibody binds to an IL-13 sequence selected from a sequence set forth in SEQ ID NOs: 472-475 with a K_{D} of less than or equal to about 1×10⁻¹⁰ M, as measured by SPR and wherein the antibody has a half-life in humans of about 80 to about 110 days.

276. The isolated antibody of claim 272, wherein the antibody comprises a heavy chain variable region sequence set forth in SEQ ID NO: 3 and a light chain variable region sequence set forth in SEQ ID NO: 39.

277. The isolated antibody of claim 272, wherein the antibody comprises a constant heavy chain sequence set forth in SEQ ID NO: 439 and a constant light chain sequence set forth in SEQ ID NO: 469.

278. An isolated antibody that binds IL-13, wherein the antibody comprises a heavy chain variable region sequence set forth in SEQ ID NO: 470 and a light chain variable region sequence set forth in SEQ ID NO: 471, wherein the human Fc region of the antibody comprises a human IgG1 Fc with LALA and/or YTE mutations.

279. The isolated antibody of claim 272, wherein the antibody has a half-life in humans of about 80 to about 110 days.

280. The isolated antibody of claim 272, wherein the antibody is a humanized, human, or chimeric antibody.

281. The isolated antibody of claim 280, wherein the antibody is a humanized antibody.

282. The isolated antibody of claim 281, wherein the human Fc region of the antibody comprises a human IgG1 Fc.

283. The isolated antibody of claim 272, wherein the antibody is a monoclonal antibody.

284. The isolated antibody of claim 272, wherein the antibody binds an IL-13 sequence selected from a sequence set forth in SEQ ID NOs: 472-475.

285. The isolated antibody of claim 272, wherein the antibody binds to an IL-13 sequence selected from a sequence set forth in SEQ ID NOs: 472-475 with a K_{D} of less than or equal to about 1, 2, 3, 4, 5, 6, 7, 8, 9×10⁻⁹ M, as measured by surface plasmon resonance (SPR).

286. The isolated antibody of claim 272, wherein the antibody binds to an IL-13 sequence selected from a sequence set forth in SEQ ID NOs: 472-475 with a K_{D} of less than or equal to about 1×10⁻¹⁰ M, as measured by SPR.

287. The isolated antibody of claim 272, wherein the antibody binds to human IL-13 with a K_{D} of less than or equal to about 1×10⁻⁹M, as measured by SPR.

288. The isolated antibody of claim 272, wherein the antibody is formulated for subcutaneous injection.

289. The isolated antibody of claim 272, wherein the antibody is formulated for intravenous injection.

290. A composition comprising the isolated antibody of claim 272 and a pharmaceutically acceptable excipient.

291. A method for treating an inflammatory disorder or disease in a mammalian subject in need thereof, the method comprising administering to the mammalian subject the isolated antibody of claim 272.

292. The method of claim 290, wherein the inflammatory disorder or disease is atopic dermatitis.

293. The method of claim 292, wherein the treatment reduces disease severity in the patient and wherein disease severity is assessed by an Atopic Dermatitis Disease Severity Outcome Measure.

294. The method of claim 291, wherein the inflammatory disorder or disease is selected from the group consisting of asthma, idiopathic pulmonary fibrosis, alopecia areata, chronic sinusitis with nasal polyps, Chronic Rhinosinusitis without Nasal Polyps (CRSsNP), Eosinophilic gastrointestinal disorder or disease (ENID), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), and Chronic Obstructive Pulmonary Disease (COPD).

295. The method of claim 294, wherein the ENID is selected from the group consisting of eosinophilic esophagitis (EoE), Eosinophilic Gastritis (EoG), Eosinophilic Enteritis (EoN), Eosinophilic Colitis (EoC), and Eosinophilic Gastroenteritis (EGE).

296. A kit for treating an inflammatory disorder or disease in a human patient, the kit comprising:
(a) a dose of the isolated antibody that binds IL-13 of claim 272; and
(b) instructions.

297. The kit of claim 296, for use in treating an inflammatory disorder or disease selected from the group consisting of atopic dermatitis, asthma, idiopathic pulmonary fibrosis, alopecia areata, chronic sinusitis with nasal polyps, Chronic Rhinosinusitis without Nasal Polyps (CRSsNP), an Eosinophilic gastrointestinal disorder or disease (ENID), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), and Chronic Obstructive Pulmonary Disease (COPD).


SEQUENCE LISTING (609 sequences)

SEQ ID NO: 1 - protein, 118 aa, synthetic construct
QVQLQESGPGLVAPSQSLSITCTVSGFSLNAYSVNWVRQPPGKGLEWLGMIWGDGKIVYNSALKSRLNISKDSSKSQVFLKMSSLQSDDTARYYCAGDGYYPYAMDNWGHGTSVTVSS

SEQ ID NO: 2 - protein, 118 aa, synthetic construct
QVQLQESGPGLVAPSQSLSITCTVSGFSLNAYSVNWVRQPPGKGLEWLGMIWGDGKIVYNSALKSRLTISKDSSKSQVFLKMSSLQSDDTARYYCAGDGYYPYAMDNWGHGTSVTVSS

SEQ ID NO: 3 - protein, 118 aa, synthetic construct
EVQLQESGPGLVKPSETLSLTCTVSGFSLNAYSVNWIRQPPGKGLEWLGMIWGDGKIVYNSALKSRLTISKDSSKNQVSLKLSSVTAADTAVYYCAGDGYYPYAMDNWGQGTTVTVSS

SEQ ID NO: 4 - protein, 118 aa, synthetic construct
EVQLVQSGAEVKKPGASVKVSCKASGFSLNAYSVNWVRQAPGQGLEWLGMIWGDGKIVYNSALKSRLTITKDSSTSTVYMELSSLRSEDTAVYYCAGDGYYPYAMDNWGQGTTVTVSS

SEQ ID NO: 5 - protein, 118 aa, synthetic construct
EVQLVQSGAEVKKPGSSVKVSCKASGFSLNAYSVNWVRQAPGQGLEWLGMIWGDGKIVYNSALKSRLTITKDSSTSTVYMELSSLRSEDTAVYYCAGDGYYPYAMDNWGQGTTVTVSS

SEQ ID NO: 6 - protein, 118 aa, synthetic construct
EVQLVESGGGLVKPGGSLRLSCAASGFSLNAYSVNWVRQAPGKGLEWLGMIWGDGKIVYNSALKSRLTISKDSSKNTVYLQMNSLKTEDTAVYYCAGDGYYPYAMDNWGQGTTVTVSS

SEQ ID NO: 7 - protein, 118 aa, synthetic construct
EVQLLESGGGLVQPGGSLRLSCAASGFSLNAYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 8 - protein, 118 aa, synthetic construct
EVQLQESGPGLVKPSETLSLTCTVSGGSLNAYSVNWVRQPPGKGLEWLGMIWGDGKIVYNSALKSRLTISLDTSKSQVFLKMSSLTAADTAVYYCARDGYYPYAMDNWGQGTTVTVSS

SEQ ID NO: 9 - protein, 118 aa, synthetic construct
QVQLQESGPGLVKPSETLSLTCTVSGGSLNAYSWNWVRQPPGKGLEWLGYIYGDGKTNYNPALKSRLTISLDTSKSQVFLKMSSLTAADTAVYYCARDGYYYYAMDVWGQGTTVTVSS

SEQ ID NO: 10 - protein, 118 aa, synthetic construct
EVQLLESGGGLVQPGGSLRLSCAASGYSLNAYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 11 - protein, 118 aa, synthetic construct
EVQLLESGGGLVQPGGSLRLSCAASGFSLRAYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 12 - protein, 118 aa, synthetic construct
EVQLLESGGGLVQPGGSLRLSCAASGFSLHAYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 13 - protein, 118 aa, synthetic construct
EVQLLESGGGLVQPGGSLRLSCAASGFSLDAYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 14 - protein, 118 aa, synthetic construct
EVQLLESGGGLVQPGGSLRLSCAASGFSLYAYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 15 - protein, 118 aa, synthetic construct
EVQLLESGGGLVQPGGSLRLSCAASGFSLSAYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 16 - protein, 118 aa, synthetic construct
EVQLLESGGGLVQPGGSLRLSCAASGFSLNRYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 17 - protein, 118 aa, synthetic construct
EVQLLESGGGLVQPGGSLRLSCAASGFSLNKYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 18 - protein, 118 aa, synthetic construct
EVQLLESGGGLVQPGGSLRLSCAASGFSLNHYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 19 - protein, 118 aa, synthetic construct
EVQLLESGGGLVQPGGSLRLSCAASGFSLNQYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 20 - protein, 118 aa, synthetic construct
EVQLLESGGGLVQPGGSLRLSCAASGFSLNEYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 21 - protein, 118 aa, synthetic construct
EVQLLESGGGLVQPGGSLRLSCAASGFSLNSYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 22 - protein, 118 aa, synthetic construct
EVQLLESGGGLVQPGGSLRLSCAASGFSLNYYSVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 23 - protein, 118 aa, synthetic construct
EVQLLESGGGLVQPGGSLRLSCAASGFSLNAESVNWVRQAPGKGLEWLGMIWGDGKIVYNSALKSRLTISKDSSKNTVYLQMNSLRAEDTAVYYCAGDGYYPYAMDNWGQGTTVTVSS

SEQ ID NO: 24 - protein, 118 aa, synthetic construct
EVQLLESGGGLVQPGGSLRLSCAASGFSLNAYSVNWVRQAPGKGLEWLGMIWSDGKIVYNSALKSRLTISKDSSKNTVYLQMNSLRAEDTAVYYCAGDGYYPYAMDNWGQGTTVTVSS

SEQ ID NO: 25 - protein, 118 aa, synthetic construct
EVQLLESGGGLVQPGGSLRLSCAASGFSLNAYSVNWVRQAPGKGLEWLGMIWADGKIVYNSALKSRLTISKDSSKNTVYLQMNSLRAEDTAVYYCAGDGYYPYAMDNWGQGTTVTVSS

SEQ ID NO: 26 - protein, 118 aa, synthetic construct
EVQLLESGGGLVQPGGSLRLSCAASGFSLNAYSVNWVRQAPGKGLEWLGMIWGDGKIVYNSALKSRLTISKDSSKNTVYLQMNSLRAEDTAVYYCAGHGYYPYAMDNWGQGTTVTVSS

SEQ ID NO: 27 - protein, 118 aa, synthetic construct
EVQLLESGGGLVQPGGSLRLSCAASGFSLNAYSVNWVRQAPGKGLEWLGMIWGDGKIVYNSALKSRLTISKDSSKNTVYLQMNSLRAEDTAVYYCAGDLYYPYAMDNWGQGTTVTVSS

SEQ ID NO: 28 - protein, 118 aa, synthetic construct
EVQLLESGGGLVQPGGSLRLSCAASGFSLNAYSVNWVRQAPGKGLEWLGMIWGDGKIVYNSALKSRLTISKDSSKNTVYLQMNSLRAEDTAVYYCAGDKYYPYAMDNWGQGTTVTVSS

SEQ ID NO: 29 - protein, 118 aa, synthetic construct
EVQLLESGGGLVQPGGSLRLSCAASGFSLNAYSVNWVRQAPGKGLEWLGMIWGDGKIVYNSALKSRLTISKDSSKNTVYLQMNSLRAEDTAVYYCAGDGYYGYAMDNWGQGTTVTVSS

SEQ ID NO: 30 - protein, 118 aa, synthetic construct
EVQLLESGGGLVQPGGSLRLSCAASGFSLNAYSVNWVRQAPGKGLEWLGMIWGDGKIVYNSALKSRLTISKDSSKNTVYLQMNSLRAEDTAVYYCAGDGYYAYAMDNWGQGTTVTVSS

SEQ ID NO: 31 - protein, 118 aa, synthetic construct
EVQLLESGGGLVQPGGSLRLSCAASGFSLNAYSVNWVRQAPGKGLEWLGMIWGDGKIVYNSALKSRLTISKDSSKNTVYLQMNSLRAEDTAVYYCAGDGYYSYAMDNWGQGTTVTVSS

SEQ ID NO: 32 - protein, 118 aa, synthetic construct
EVQLLESGGGLVQPGGSLRLSCAASGFSLNAYSVNWVRQAPGKGLEWLGMIWGDGKIVYNSALKSRLTISKDSSKNTVYLQMNSLRAEDTAVYYCAGDGYYTYAMDNWGQGTTVTVSS

SEQ ID NO: 33 - protein, 111 aa, synthetic construct
NIVLTQSPASLAVSLGQRATISCRASKSVDSYGNSFMHWYQQKPGQPPKLLIYLASNLESGVPARFSGSGSRTDFTLTIDPVEADDAASYYCQQNNEDPRTFGGGTKLEIK

SEQ ID NO: 34 - protein, 111 aa, synthetic construct
DIQLTQSPSSLSASVGDRVTITCRASKSVDSYGNSFMHWYQQKPGKAPKLLIYLASNLESGVPSRFSGSGSRTDFTLTISSLQPEDFATYYCQQNNEDPRTFGGGTKVEIK

SEQ ID NO: 35 - protein, 111 aa, synthetic construct; identical to SEQ ID NO: 34

SEQ ID NO: 36 - protein, 111 aa, synthetic construct
EIVLTQSPATLSVSPGERATLSCRASKSVDSYGNSFMHWYQQKPGQAPRLLIYLASNLESGIPARFSGSGSRTEFTLTISSLQSEDFAVYYCQQNNEDPRTFGGGTKVEIK

SEQ ID NO: 37 - protein, 111 aa, synthetic construct
DIVLTQSPLSLPVTPGEPASISCRASKSVDSYGNSFMHWYLQKPGQSPQLLIYLASNLESGVPDRFSGSGSRTDFTLKISRVEAEDVGVYYCQQNNEDPRTFGGGTKVEIK

SEQ ID NO: 38 - protein, 111 aa, synthetic construct
DIVLTQSPDSLAVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIK

SEQ ID NO: 39 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNNEDPRTFGGGTKVEIK

SEQ ID NO: 40 - protein, 111 aa, synthetic construct
EIVLTQSPATLSVSPGERATLSCRASKSVDSYGNSFMHWYQQKPGQAPRLLIYLASNLESGIPARFSGSGSGTEFTLTISSLQSEDFAVYYCQQNNEDPRTFGGGTKVEIK

SEQ ID NO: 41 - protein, 111 aa, synthetic construct
DIVLTQSPLSLPVTPGEPASISCRASKSVDSYGNSFMHWYLQKPGQSPQLLIYLASNLESGVPDRFSGSGSGTDFTLKISRVEAEDVGVYYCQQNNEDPRTFGGGTKVEIK

SEQ ID NO: 42 - protein, 111 aa, synthetic construct
DIVLTQSPASLAVSPGERATISCRASKSVDSYGNSFMHWYQQKPGQPPKLLIYLASNLESGVPDRFSGSGSGTDFTLTISRVEADDVAVYYCQQNNEDPRTFGGGTKLEIK

SEQ ID NO: 43 - protein, 111 aa, synthetic construct
DIVLTQSPASLAVSPGERATISCRASQSVDSNGNNFLHWYQQKPGQPPKLLIYLASNRESGVPDRFSGSGSGTDFTLTISRVEADDVAVYYCQQNNHTPRTFGGGTKLEIK

SEQ ID NO: 44 - protein, 111 aa, synthetic construct
DIQLTQSPSSLSASVGDRVTITCRASKSVDSYGNSRMHWYQQKPGKAPKLLIYLASNLESGVPSRFSGSGSGTDFTLTISSLQPEDFATYYCQQNNEDPRTFGGGTKVEIK

SEQ ID NO: 45 - protein, 111 aa, synthetic construct
DIQLTQSPSSLSASVGDRVTITCRASKSVDSYGNSSMHWYQQKPGKAPKLLIYLASNLESGVPSRFSGSGSGTDFTLTISSLQPEDFATYYCQQNNEDPRTFGGGTKVEIK

SEQ ID NO: 46 - protein, 111 aa, synthetic construct
DIQLTQSPSSLSASVGDRVTITCRASKSVDSYGNSFMHWYQQKPGKAPKLLIRLASNLESGVPSRFSGSGSGTDFTLTISSLQPEDFATYYCQQNNEDPRTFGGGTKVEIK

SEQ ID NO: 47 - protein, 111 aa, synthetic construct
DIQLTQSPSSLSASVGDRVTITCRASKSVDSYGNSFMHWYQQKPGKAPKLLIFLASNLESGVPSRFSGSGSGTDFTLTISSLQPEDFATYYCQQNNEDPRTFGGGTKVEIK

SEQ ID NO: 48 - protein, 111 aa, synthetic construct
DIQLTQSPSSLSASVGDRVTITCRASKSVDSYGNSFMHWYQQKPGKAPKLLIYLASHLESGVPSRFSGSGSGTDFTLTISSLQPEDFATYYCQQNNEDPRTFGGGTKVEIK

SEQ ID NO: 49 - protein, 111 aa, synthetic construct
DIQLTQSPSSLSASVGDRVTITCRASKSVDSYGNSFMHWYQQKPGKAPKLLIYLASDLESGVPSRFSGSGSGTDFTLTISSLQPEDFATYYCQQNNEDPRTFGGGTKVEIK

SEQ ID NO: 50 - protein, 111 aa, synthetic construct
DIQLTQSPSSLSASVGDRVTITCRASKSVDSYGNSFMHWYQQKPGKAPKLLIYLASQLESGVPSRFSGSGSGTDFTLTISSLQPEDFATYYCQQNNEDPRTFGGGTKVEIK

SEQ ID NO: 51 - protein, 111 aa, synthetic construct
DIQLTQSPSSLSASVGDRVTITCRASKSVDSYGNSFMHWYQQKPGKAPKLLIYLASELESGVPSRFSGSGSGTDFTLTISSLQPEDFATYYCQQNNEDPRTFGGGTKVEIK

SEQ ID NO: 52 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNHEDPRTFGGGTKVEIK

SEQ ID NO: 53 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNYEDPRTFGGGTKVEIK

SEQ ID NO: 54 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNSEDPRTFGGGTKVEIK

SEQ ID NO: 55 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNNRDPRTFGGGTKVEIK

SEQ ID NO: 56 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNNDDPRTFGGGTKVEIK

SEQ ID NO: 57 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNNQDPRTFGGGTKVEIK

SEQ ID NO: 58 - protein, 5 aa, synthetic construct
AYSVN

SEQ ID NO: 59 - protein, 5 aa, synthetic construct
RYSVN

SEQ ID NO: 60 - protein, 5 aa, synthetic construct
KYSVN

SEQ ID NO: 61 - protein, 5 aa, synthetic construct
HYSVN

SEQ ID NO: 62 - protein, 5 aa, synthetic construct
QYSVN

SEQ ID NO: 63 - protein, 5 aa, synthetic construct
EYSVN

SEQ ID NO: 64 - protein, 5 aa, synthetic construct
SYSVN

SEQ ID NO: 65 - protein, 5 aa, synthetic construct
YYSVN

SEQ ID NO: 66 - protein, 5 aa, synthetic construct
AESVN

SEQ ID NO: 67 - protein, 7 aa, synthetic construct
GFSLSAY

SEQ ID NO: 68 - protein, 7 aa, synthetic construct
GFSLNAY

SEQ ID NO: 69 - protein, 7 aa, synthetic construct
GGSLNAY

SEQ ID NO: 70 - protein, 7 aa, synthetic construct
GGSLNAY

SEQ ID NO: 71 - protein, 7 aa, synthetic construct
GYSLNAY

SEQ ID NO: 72 - protein, 7 aa, synthetic construct
GFSLRAY

SEQ ID NO: 73 - protein, 7 aa, synthetic construct
GFSLHAY

SEQ ID NO: 74 - protein, 7 aa, synthetic construct
GFSLDAY

SEQ ID NO: 75 - protein, 7 aa, synthetic construct
GFSLYAY

SEQ ID NO: 76 - protein, 7 aa, synthetic construct
GFSLNRY

SEQ ID NO: 77 - protein, 7 aa, synthetic construct
GFSLNKY

SEQ ID NO: 78 - protein, 7 aa, synthetic construct
GFSLNHY

SEQ ID NO: 79 - protein, 7 aa, synthetic construct
GFSLNQY

SEQ ID NO: 80 - protein, 7 aa, synthetic construct
GFSLNEY

SEQ ID NO: 81 - protein, 7 aa, synthetic construct
GFSLNSY

SEQ ID NO: 82 - protein, 7 aa, synthetic construct
GFSLNYY

SEQ ID NO: 83 - protein, 7 aa, synthetic construct
GFSLNAE

SEQ ID NO: 84 - protein, 8 aa, synthetic construct
GFSLSAYS

SEQ ID NO: 85 - protein, 8 aa, synthetic construct
GFSLNAYS

SEQ ID NO: 86 - protein, 8 aa, synthetic construct
GGSLNAYS

SEQ ID NO: 87 - protein, 8 aa, synthetic construct
GYSLNAYS

SEQ ID NO: 88 - protein, 8 aa, synthetic construct
GFSLRAYS

SEQ ID NO: 89 - protein, 8 aa, synthetic construct
GFSLHAYS

SEQ ID NO: 90 - protein, 8 aa, synthetic construct
GFSLDAYS

SEQ ID NO: 91 - protein, 8 aa, synthetic construct
GFSLYAYS

SEQ ID NO: 92 - protein, 8 aa, synthetic construct
GFSLSAYS

SEQ ID NO: 93 - protein, 8 aa, synthetic construct
GFSLNRYS

SEQ ID NO: 94 - protein, 8 aa, synthetic construct
GFSLNKYS

SEQ ID NO: 95 - protein, 8 aa, synthetic construct
GFSLNHYS

SEQ ID NO: 96 - protein, 8 aa, synthetic construct
GFSLNQYS

SEQ ID NO: 97 - protein, 8 aa, synthetic construct
GFSLNEYS

SEQ ID NO: 98 - protein, 8 aa, synthetic construct
GFSLNYYS

SEQ ID NO: 99 - protein, 8 aa, synthetic construct
GFSLNAES

SEQ ID NO: 100 - protein, 16 aa, synthetic construct
MIWGDGKIVYNSALKS

SEQ ID NO: 101 - protein, 16 aa, synthetic construct
YIYGDGKTNYNPALKS

SEQ ID NO: 102 - protein, 16 aa, synthetic construct
MIWSDGKIVYNSALKS

SEQ ID NO: 103 - protein, 16 aa, synthetic construct
MIWADGKIVYNSALKS

SEQ ID NO: 104 - protein, 5 aa, synthetic construct
WGDGK

SEQ ID NO: 105 - protein, 5 aa, synthetic construct
YGDGK

SEQ ID NO: 106 - protein, 5 aa, synthetic construct
WSDGK

SEQ ID NO: 107 - protein, 5 aa, synthetic construct
WADGK

SEQ ID NO: 108 - protein, 7 aa, synthetic construct
IWGDGKI

SEQ ID NO: 109 - protein, 7 aa, synthetic construct
IYGDGKT

SEQ ID NO: 110 - protein, 7 aa, synthetic construct
IWSDGKI

SEQ ID NO: 111 - protein, 7 aa, synthetic construct
IWADGKI

SEQ ID NO: 112 - protein, 10 aa, synthetic construct
DGYYPYAMDN

SEQ ID NO: 113 - protein, 10 aa, synthetic construct
DGYYYYAMDV

SEQ ID NO: 114 - protein, 10 aa, synthetic construct
HGYYPYAMDN

SEQ ID NO: 115 - protein, 10 aa, synthetic construct
DLYYPYAMDN

SEQ ID NO: 116 - protein, 10 aa, synthetic construct
DKYYPYAMDN

SEQ ID NO: 117 - protein, 10 aa, synthetic construct
DGYYGYAMDN

SEQ ID NO: 118 - protein, 10 aa, synthetic construct
DGYYAYAMDN

SEQ ID NO: 119 - protein, 10 aa, synthetic construct
DGYYSYAMDN

SEQ ID NO: 120 - protein, 10 aa, synthetic construct
DGYYTYAMDN

SEQ ID NO: 121 - protein, 8 aa, synthetic construct
GNSLNSYS

SEQ ID NO: 122-129 - skipped (8 entries)

SEQ ID NO: 130 - protein, 12 aa, synthetic construct
AGDGYYPYAMDN

SEQ ID NO: 131 - protein, 12 aa, synthetic construct
ARDGYYPYAMDN

SEQ ID NO: 132 - protein, 12 aa, synthetic construct
ARDGYYYYAMDV

SEQ ID NO: 133 - protein, 12 aa, synthetic construct
AGHGYYPYAMDN

SEQ ID NO: 134 - protein, 12 aa, synthetic construct
AGDLYYPYAMDN

SEQ ID NO: 135 - protein, 12 aa, synthetic construct
AGDKYYPYAMDN

SEQ ID NO: 136 - protein, 12 aa, synthetic construct
AGDGYYGYAMDN

SEQ ID NO: 137 - protein, 12 aa, synthetic construct
AGDGYYAYAMDN

SEQ ID NO: 138 - protein, 12 aa, synthetic construct
AGDGYYSYAMDN

SEQ ID NO: 139 - protein, 12 aa, synthetic construct
AGDGYYTYAMDN

SEQ ID NO: 140 - protein, 12 aa, synthetic construct
AGDGYYPYAMDN

SEQ ID NO: 141 - protein, 15 aa, synthetic construct
RASKSVDSYGNSFMH

SEQ ID NO: 142 - protein, 15 aa, synthetic construct
RASQSVDSNGNNFLH

SEQ ID NO: 143 - protein, 15 aa, synthetic construct
RASKSVDSYGNSRMH

SEQ ID NO: 144 - protein, 15 aa, synthetic construct
RASKSVDSYGNSSMH

SEQ ID NO: 145-148 - skipped (4 entries)

SEQ ID NO: 149 - protein, 10 aa, synthetic construct
KSVDSYGNSF

SEQ ID NO: 150 - protein, 10 aa, synthetic construct
QSVDSNGNNF

SEQ ID NO: 151 - protein, 10 aa, synthetic construct
KSVDSYGNSR

SEQ ID NO: 152 - protein, 10 aa, synthetic construct
KSVDSYGNSS

SEQ ID NO: 153 - protein, 7 aa, synthetic construct
LASNLES

SEQ ID NO: 154 - protein, 7 aa, synthetic construct
LASHLES

SEQ ID NO: 155 - protein, 7 aa, synthetic construct
LASDLES

SEQ ID NO: 156 - protein, 7 aa, synthetic construct
LASQLES

SEQ ID NO: 157 - protein, 7 aa, synthetic construct
LASELES

SEQ ID NO: 158 - protein, 7 aa, synthetic construct
LASNRES

SEQ ID NO: 159-164 - skipped (6 entries)

SEQ ID NO: 165 - protein, 9 aa, synthetic construct
QQNNEDPRT

SEQ ID NO: 166 - protein, 9 aa, synthetic construct
QQNNHTPRT

SEQ ID NO: 167 - protein, 9 aa, synthetic construct
QQNHEDPRT

SEQ ID NO: 168 - protein, 9 aa, synthetic construct
QQNYEDPRT

SEQ ID NO: 169 - protein, 9 aa, synthetic construct
QQNSEDPRT

SEQ ID NO: 170 - protein, 9 aa, synthetic construct
QQNNRDPRT

SEQ ID NO: 171 - protein, 9 aa, synthetic construct
QQNNDDPRT

SEQ ID NO: 172 - protein, 9 aa, synthetic construct
QQNNQDPRT

SEQ ID NO: 173-197 - skipped (25 entries)

SEQ ID NO: 198 - protein, 30 aa, synthetic construct
QVQLQESGPGLVAPSQSLSITCTVSGFSLN

SEQ ID NO: 199 - skipped

SEQ ID NO: 200 - protein, 30 aa, synthetic construct
EVQLQESGPGLVKPSETLSLTCTVSGFSLN

SEQ ID NO: 201 - protein, 30 aa, synthetic construct
EVQLVQSGAEVKKPGASVKVSCKASGFSLN

SEQ ID NO: 202 - protein, 30 aa, synthetic construct
EVQLVQSGAEVKKPGSSVKVSCKASGFSLN

SEQ ID NO: 203 - protein, 30 aa, synthetic construct
EVQLVESGGGLVKPGGSLRLSCAASGFSLN

SEQ ID NO: 204 - protein, 30 aa, synthetic construct
EVQLLESGGGLVQPGGSLRLSCAASGFSLN

SEQ ID NO: 205 - protein, 30 aa, synthetic construct
EVQLQESGPGLVKPSETLSLTCTVSGGSLN

SEQ ID NO: 206 - protein, 30 aa, synthetic construct
QVQLQESGPGLVKPSETLSLTCTVSGGSLN

SEQ ID NO: 207 - protein, 30 aa, synthetic construct
EVQLLESGGGLVQPGGSLRLSCAASGYSLN

SEQ ID NO: 208 - protein, 30 aa, synthetic construct
EVQLLESGGGLVQPGGSLRLSCAASGFSLR

SEQ ID NO: 209 - protein, 30 aa, synthetic construct
EVQLLESGGGLVQPGGSLRLSCAASGFSLH

SEQ ID NO: 210 - protein, 30 aa, synthetic construct
EVQLLESGGGLVQPGGSLRLSCAASGFSLD

SEQ ID NO: 211 - protein, 30 aa, synthetic construct
EVQLLESGGGLVQPGGSLRLSCAASGFSLY

SEQ ID NO: 212 - protein, 30 aa, synthetic construct
EVQLLESGGGLVQPGGSLRLSCAASGFSLS

SEQ ID NO: 213-229 - skipped (17 entries)

SEQ ID NO: 230 - protein, 23 aa, synthetic construct
NIVLTQSPASLAVSLGQRATISC

SEQ ID NO: 231 - protein, 23 aa, synthetic construct
DIQLTQSPSSLSASVGDRVTITC

SEQ ID NO: 232 - skipped

SEQ ID NO: 233 - protein, 23 aa, synthetic construct
EIVLTQSPATLSVSPGERATLSC

SEQ ID NO: 234 - protein, 23 aa, synthetic construct
DIVLTQSPLSLPVTPGEPASISC

SEQ ID NO: 235 - protein, 23 aa, synthetic construct
DIVLTQSPDSLAVSLGERATINC

SEQ ID NO: 236-238 - skipped (3 entries)

SEQ ID NO: 239 - protein, 23 aa, synthetic construct
DIVLTQSPASLAVSPGERATISC

SEQ ID NO: 240 - protein, 23 aa, synthetic construct
DIVLTQSPASLAVSPGERATISC

SEQ ID NO: 241-254 - skipped (14 entries)

SEQ ID NO: 255 - protein, 14 aa, synthetic construct
WIRQPPGKALEWLA

SEQ ID NO: 256 - protein, 14 aa, synthetic construct
WVRQPPGKGLEWLG

SEQ ID NO: 257 - skipped

SEQ ID NO: 258 - protein, 14 aa, synthetic construct
WIRQPPGKGLEWLG

SEQ ID NO: 259 - protein, 14 aa, synthetic construct
WVRQAPGQGLEWLG

SEQ ID NO: 260-261 - skipped (2 entries)

SEQ ID NO: 262 - protein, 14 aa, synthetic construct
WVRQAPGKGLEWLG

SEQ ID NO: 263 - protein, 14 aa, synthetic construct
WVRQPPGKGLEWLG

SEQ ID NO: 264-285 - skipped (22 entries)

SEQ ID NO: 286 - protein, 15 aa, synthetic construct
WYQQKPGQPPKLLIY

SEQ ID NO: 287 - skipped

SEQ ID NO: 288 - protein, 15 aa, synthetic construct
WYQQKPGKAPKLLIY

SEQ ID NO: 289 - skipped

SEQ ID NO: 290 - protein, 15 aa, synthetic construct
WYQQKPGQAPRLLIY

SEQ ID NO: 291 - protein, 15 aa, synthetic construct
WYLQKPGQSPQLLIY

SEQ ID NO: 292-299 - skipped (8 entries)

SEQ ID NO: 300 - protein, 15 aa, synthetic construct
WYQQKPGKAPKLLIR

SEQ ID NO: 301 - protein, 15 aa, synthetic construct
WYQQKPGKAPKLLIF

SEQ ID NO: 302-310 - skipped (9 entries)

SEQ ID NO: 311 - protein, 32 aa, synthetic construct
RLTISKDTSKNQVVLTMTNMDPVDTATYYCAG

SEQ ID NO: 312 - protein, 32 aa, synthetic construct
RLNISKDSSKSQVFLKMSSLQSDDTARYYCAG

SEQ ID NO: 313 - protein, 32 aa, synthetic construct
RLTISKDSSKSQVFLKMSSLQSDDTARYYCAG

SEQ ID NO: 314 - protein, 32 aa, synthetic construct
RLTISKDSSKNQVSLKLSSVTAADTAVYYCAG

SEQ ID NO: 315 - protein, 32 aa, synthetic construct
RLTITKDSSTSTVYMELSSLRSEDTAVYYCAG

SEQ ID NO: 316 - skipped

SEQ ID NO: 317 - protein, 32 aa, synthetic construct
RLTISKDSSKNTVYLQMNSLKTEDTAVYYCAG

SEQ ID NO: 318 - protein, 32 aa, synthetic construct
RLTISKDSSKNTVYLQMNSLRAEDTAVYYCAG

SEQ ID NO: 319 - protein, 32 aa, synthetic construct
RLTISLDTSKSQVFLKMSSLTAADTAVYYCAR

SEQ ID NO: 320-342 - skipped (23 entries)

SEQ ID NO: 343 - protein, 32 aa, synthetic construct
GVPDRFSGSGSGTDFTLTISSLQAEDVAVYYC

SEQ ID NO: 344 - protein, 32 aa, synthetic construct
GVPARFSGSGSRTDFTLTIDPVEADDAASYYC

SEQ ID NO: 345 - protein, 32 aa, synthetic construct
GVPSRFSGSGSRTDFTLTISSLQPEDFATYYC

SEQ ID NO: 346 - skipped

SEQ ID NO: 347 - protein, 32 aa, synthetic construct
GIPARFSGSGSRTEFTLTISSLQSEDFAVYYC

SEQ ID NO: 348 - protein, 32 aa, synthetic construct
GVPDRFSGSGSRTDFTLKISRVEAEDVGVYYC

SEQ ID NO: 349 - protein, 32 aa, synthetic construct
GVPSRFSGSGSGTDFTLTISSLQPEDFATYYC

SEQ ID NO: 350 - skipped

SEQ ID NO: 351 - protein, 32 aa, synthetic construct
GIPARFSGSGSGTEFTLTISSLQSEDFAVYYC

SEQ ID NO: 352 - protein, 32 aa, synthetic construct
GVPDRFSGSGSGTDFTLKISRVEAEDVGVYYC

SEQ ID NO: 353 - protein, 32 aa, synthetic construct
GVPDRFSGSGSGTDFTLTISRVEADDVAVYYC

SEQ ID NO: 354-367 - skipped (14 entries)

SEQ ID NO: 368 - protein, 11 aa, synthetic construct
WGQGSLVTVSS

SEQ ID NO: 369 - protein, 11 aa, synthetic construct
WGHGTSVTVSS

SEQ ID NO: 370 - skipped

SEQ ID NO: 371 - protein, 11 aa, synthetic construct
WGQGTTVTVSS

SEQ ID NO: 372-399 - skipped (28 entries)

SEQ ID NO: 400 - protein, 10 aa, synthetic construct
FGGGTKVEIK

SEQ ID NO: 401 - protein, 10 aa, synthetic construct
FGGGTKLEIK

SEQ ID NO: 402-424 - skipped (23 entries)

SEQ ID NO: 425 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 426 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 427 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 428 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 429 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 430 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 431 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 432 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 433 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 434 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 435 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 436 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 437 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 438 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 439 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 440 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 441 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 442 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 443 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 444 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 445 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 446 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 447 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 448 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 449 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 450 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 451 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 452 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 453 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSP

SEQ ID NO: 454 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 455 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 456 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 457 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 458 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 459 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 460 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSYTQKSLSLSLGK

SEQ ID NO: 461 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 462 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 463 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 464 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 465 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 466 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSP

SEQ ID NO: 467 - protein, 324 aa, synthetic construct; identical to SEQ ID NO: 429

SEQ ID NO: 468 - protein, 324 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSP

SEQ ID NO: 469 - protein, 107 aa, synthetic construct [sequence omitted]

SEQ ID NO: 470 - protein, 118 aa, synthetic construct [sequence omitted]

SEQ ID NO: 471 - protein, 111 aa, synthetic construct [sequence omitted]

SEQ ID NO: 472 - protein, 146 aa, Homo sapiens [sequence omitted]

SEQ ID NO: 473 - protein, 132 aa, Macaca fascicularis
MALLLTMVIALTCLGGFASPSPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 474 - protein, 131 aa, Mus sp.
MALWVTAVLALACLGGLAAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 475 - protein, 131 aa, Rattus norvegicus
MALWVTAVLALACLGGLATPGPVRRSTSPPVALRELIEELSNITQDQKTSLCNSSMVWSVDLTAGGFCAALESLTNISSCNAIHRTQRILNGLCNQKASDVASSPPDTKIEVAQFISKLLNYSKQLFRYGH

SEQ ID NO: 476 - protein, 24 aa, Homo sapiens
MHPLLNPLLLALGLMALLLTTVIA

SEQ ID NO: 477 - protein, 18 aa, Macaca fascicularis
MALLLTMVIALTCLGGFA

SEQ ID NO: 478 - protein, 18 aa, Mus sp.
MALWVTAVLALACLGGLA

SEQ ID NO: 479 - protein, 18 aa, Rattus norvegicus
MALWVTAVLALACLGGLA

SEQ ID NO: 480 - protein, 122 aa, Homo sapiens
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 481 - protein, 114 aa, Macaca fascicularis
SPSPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 482 - protein, 113 aa, Mus sp.
APGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 483 - protein, 113 aa, Rattus norvegicus
TPGPVRRSTSPPVALRELIEELSNITQDQKTSLCNSSMVWSVDLTAGGFCAALESLTNISSCNAIHRTQRILNGLCNQKASDVASSPPDTKIEVAQFISKLLNYSKQLFRYGH

SEQ ID NO: 484 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 485 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 486 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 487 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 488 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 489 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 490 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 491 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 492 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 493 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 494 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 495 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 496 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 497 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 498 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 499 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 500 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 501 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 502 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 503 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 504 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 505 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 506 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 507 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 508 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 509 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 510 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 511 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 512 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 513 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 514 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 515 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 516 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 517 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 518 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 519 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 520 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 521 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 522 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 523 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 524 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 525 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 526 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 527 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 528 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 529 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 530 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 531 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 532 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 533 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 534 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 535 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 536 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 537 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 538 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 539 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 540 - protein, 30 aa, synthetic construct
QVTLRESGPALVKPTQTLTLTCTVSGFSLS

SEQ ID NO: 541 - protein, 5 aa, synthetic construct
AYSWN

SEQ ID NO: 542 - protein, 25 aa, synthetic construct
QVTLRESGPALVKPTQTLTLTCTVS

SEQ ID NO: 543 - protein, 25 aa, synthetic construct
QVQLQESGPGLVAPSQSLSITCTVS

SEQ ID NO: 544 - protein, 25 aa, synthetic construct
EVQLQESGPGLVKPSETLSLTCTVS

SEQ ID NO: 545 - protein, 25 aa, synthetic construct
EVQLVQSGAEVKKPGASVKVSCKAS

SEQ ID NO: 546 - protein, 25 aa, synthetic construct
EVQLVQSGAEVKKPGSSVKVSCKAS

SEQ ID NO: 547 - protein, 25 aa, synthetic construct
EVQLVESGGGLVKPGGSLRLSCAAS

SEQ ID NO: 548 - protein, 25 aa, synthetic construct
EVQLLESGGGLVQPGGSLRLSCAAS

SEQ ID NO: 549 - protein, 25 aa, synthetic construct
QVQLQESGPGLVKPSETLSLTCTVS

SEQ ID NO: 550 - protein, 19 aa, synthetic construct
SVNWIRQPPGKALEWLAMI

SEQ ID NO: 551 - protein, 19 aa, synthetic construct
SVNWVRQPPGKGLEWLGMI

SEQ ID NO: 552 - protein, 19 aa, synthetic construct
SVNWIRQPPGKGLEWLGMI

SEQ ID NO: 553 - protein, 19 aa, synthetic construct
SVNWVRQAPGQGLEWLGMI

SEQ ID NO: 554 - protein, 19 aa, synthetic construct
SVNWVRQAPGKGLEWLGMI

SEQ ID NO: 555 - protein, 19 aa, synthetic construct
SWNWVRQPPGKGLEWLGYI

SEQ ID NO: 556 - protein, 41 aa, synthetic construct
IVYNSALKSRLTISKDTSKNQVVLTMTNMDPVDTATYYCAG

SEQ ID NO: 557 - protein, 41 aa, synthetic construct
IVYNSALKSRLNISKDSSKSQVFLKMSSLQSDDTARYYCAG

SEQ ID NO: 558 - protein, 41 aa, synthetic construct
IVYNSALKSRLTISKDSSKSQVFLKMSSLQSDDTARYYCAG

SEQ ID NO: 559 - protein, 41 aa, synthetic construct
IVYNSALKSRLTISKDSSKNQVSLKLSSVTAADTAVYYCAG

SEQ ID NO: 560 - protein, 41 aa, synthetic construct
IVYNSALKSRLTITKDSSTSTVYMELSSLRSEDTAVYYCAG

SEQ ID NO: 561 - protein, 41 aa, synthetic construct
IVYNSALKSRLTISKDSSKNTVYLQMNSLKTEDTAVYYCAG

SEQ ID NO: 562 - protein, 41 aa, synthetic construct
IVYNSALKSRLTISKDSSKNTVYLQMNSLRAEDTAVYYCAG

SEQ ID NO: 563 - protein, 41 aa, synthetic construct
IVYNSALKSRLTISLDTSKSQVFLKMSSLTAADTAVYYCAR

SEQ ID NO: 564 - protein, 41 aa, synthetic construct
TNYNPALKSRLTISLDTSKSQVFLKMSSLTAADTAVYYCAR

SEQ ID NO: 565 - protein, 8 aa, synthetic construct
GFSLNSYS

SEQ ID NO: 566 - protein, 17 aa, synthetic construct
VNWIRQPPGKALEWLAM

SEQ ID NO: 567 - protein, 17 aa, synthetic construct
VNWVRQPPGKGLEWLGM

SEQ ID NO: 568 - protein, 17 aa, synthetic construct
VNWIRQPPGKGLEWLGM

SEQ ID NO: 569 - protein, 17 aa, synthetic construct
VNWVRQAPGQGLEWLGM

SEQ ID NO: 570 - protein, 17 aa, synthetic construct
VNWVRQAPGKGLEWLGM

SEQ ID NO: 571 - protein, 17 aa, synthetic construct
WNWVRQPPGKGLEWLGY

SEQ ID NO: 572 - protein, 38 aa, synthetic construct
VYNSALKSRLTISKDTSKNQVVLTMTNMDPVDTATYYC

SEQ ID NO: 573 - protein, 38 aa, synthetic construct
VYNSALKSRLNISKDSSKSQVFLKMSSLQSDDTARYYC

SEQ ID NO: 574 - protein, 38 aa, synthetic construct
VYNSALKSRLTISKDSSKSQVFLKMSSLQSDDTARYYC

SEQ ID NO: 575 - protein, 38 aa, synthetic construct
VYNSALKSRLTISKDSSKNQVSLKLSSVTAADTAVYYC

SEQ ID NO: 576 - protein, 38 aa, synthetic construct
VYNSALKSRLTITKDSSTSTVYMELSSLRSEDTAVYYC

SEQ ID NO: 577 - protein, 38 aa, synthetic construct
VYNSALKSRLTISKDSSKNTVYLQMNSLKTEDTAVYYC

SEQ ID NO: 578 - protein, 38 aa, synthetic construct
VYNSALKSRLTISKDSSKNTVYLQMNSLRAEDTAVYYC

SEQ ID NO: 579 - protein, 38 aa, synthetic construct
VYNSALKSRLTISLDTSKSQVFLKMSSLTAADTAVYYC

SEQ ID NO: 580 - protein, 38 aa, synthetic construct
NYNPALKSRLTISLDTSKSQVFLKMSSLTAADTAVYYC

SEQ ID NO: 581 - protein, 23 aa, synthetic construct
DIVMTQSPDSLSVSLGERATINC

SEQ ID NO: 582 - protein, 26 aa, synthetic construct
DIVMTQSPDSLSVSLGERATINCRAS

SEQ ID NO: 583 - protein, 26 aa, synthetic construct
NIVLTQSPASLAVSLGQRATISCRAS

SEQ ID NO: 584 - protein, 26 aa, synthetic construct [sequence omitted]

SEQ ID NO: 585 - protein, 26 aa, synthetic construct
EIVLTQSPATLSVSPGERATLSCRAS

SEQ ID NO: 586 - protein, 26 aa, synthetic construct
DIVLTQSPLSLPVTPGEPASISCRAS

SEQ ID NO: 587 - protein, 26 aa, synthetic construct
DIVLTQSPDSLAVSLGERATINCRAS

SEQ ID NO: 588 - protein, 26 aa, synthetic construct
DIVLTQSPASLAVSPGERATISCRAS

SEQ ID NO: 589 - protein, 17 aa, synthetic construct
MHWYQQKPGQPPKLLIY

SEQ ID NO: 590 - protein, 17 aa, synthetic construct
MHWYQQKPGKAPKLLIY

SEQ ID NO: 591 - protein, 17 aa, synthetic construct
MHWYQQKPGQAPRLLIY

SEQ ID NO: 592 - protein, 17 aa, synthetic construct
MHWYLQKPGQSPQLLIY

SEQ ID NO: 593 - protein, 17 aa, synthetic construct
LHWYQQKPGQPPKLLIY

SEQ ID NO: 594 - protein, 17 aa, synthetic construct
MHWYQQKPGKAPKLLIR

SEQ ID NO: 595 - protein, 17 aa, synthetic construct
MHWYQQKPGKAPKLLIF

SEQ ID NO: 596 - protein, 36 aa, synthetic construct
NLESGVPDRFSGSGSGTDFTLTISSLQAEDVAVYYC

SEQ ID NO: 597 - protein, 36 aa, synthetic construct
NLESGVPARFSGSGSRTDFTLTIDPVEADDAASYYC

SEQ ID NO: 598 - protein, 36 aa, synthetic construct
NLESGVPSRFSGSGSRTDFTLTISSLQPEDFATYYC

SEQ ID NO: 599 - protein, 36 aa, synthetic construct
NLESGIPARFSGSGSRTEFTLTISSLQSEDFAVYYC

SEQ ID NO: 600 - protein, 36 aa, synthetic construct
NLESGVPDRFSGSGSRTDFTLKISRVEAEDVGVYYC

SEQ ID NO: 601 - protein, 36 aa, synthetic construct
NLESGVPSRFSGSGSGTDFTLTISSLQPEDFATYYC

SEQ ID NO: 602 - protein, 36 aa, synthetic construct
NLESGIPARFSGSGSGTEFTLTISSLQSEDFAVYYC

SEQ ID NO: 603 - protein, 36 aa, synthetic construct
NLESGVPDRFSGSGSGTDFTLKISRVEAEDVGVYYC

SEQ ID NO: 604 - protein, 36 aa, synthetic construct
NLESGVPDRFSGSGSGTDFTLTISRVEADDVAVYYC

SEQ ID NO: 605 - protein, 36 aa, synthetic construct
NRESGVPDRFSGSGSGTDFTLTISRVEADDVAVYYC

SEQ ID NO: 606 - protein, 36 aa, synthetic construct
HLESGVPSRFSGSGSGTDFTLTISSLQPEDFATYYC

SEQ ID NO: 607 - protein, 36 aa, synthetic construct
DLESGVPSRFSGSGSGTDFTLTISSLQPEDFATYYC

SEQ ID NO: 608 - protein, 36 aa, synthetic construct
QLESGVPSRFSGSGSGTDFTLTISSLQPEDFATYYC

SEQ ID NO: 609 - protein, 36 aa, synthetic construct
ELESGVPSRFSGSGSGTDFTLTISSLQPEDFATYYC